(19) United States
(12) Patent Application Publication
(10) Pub. No.: US 2025/0388739 A1
(43) Pub. Date: Dec. 25, 2025

(54) COMPOSITION, CURED SUBSTANCE, AND OPTICAL MEMBER

(71) Applicant: FUJIFILM Corporation, Tokyo (JP)

(72) Inventors: Atsushi AZUMA, Shizuoka (JP); Daisuke SASAKI, Shizuoka (JP); Yusuke SAKAI, Shizuoka (JP)

(73) Assignee: FUJIFILM Corporation, Tokyo (JP)

(21) Appl. No.: 19/307,005

(22) Filed: Aug. 21, 2025

Related U.S. Application Data
(63) Continuation of application No. PCT/JP2024/010892, filed on Mar. 21, 2024.

(30) Foreign Application Priority Data
Mar. 27, 2023 (JP) 2023-049720
Oct. 11, 2023 (JP) 2023-175968
Feb. 9, 2024 (JP) 2024-018416

(51) Int. Cl. C08K 5/45; C08F 265/06

(52) U.S. Cl. CPC C08K 5/45; C08F 265/06; C08K 2201/014

(57) ABSTRACT

A composition includes: an ultraviolet absorber; a curable compound; and an anti-fading agent, in which the ultraviolet absorber includes at least one selected from a compound represented by Formula (1) or a polymer including a structure derived from the compound represented by Formula (1), and the anti-fading agent includes at least one selected from an amine compound, a phenol compound, a hydroquinone compound, a catechol compound, an ascorbic acid compound, a carotenoid compound, a metal complex compound, or a benzolactone compound. A cured substance and an optical member, which are formed of the above-described composition.

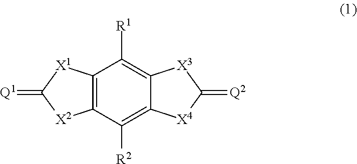

### CROSS-REFERENCE TO RELATED APPLICATIONS

This application is a Continuation of PCT International Application No. PCT/JP2024/010892 filed on Mar. 21, 2024, which claims priority under 35 U.S.C § 119(a) to Japanese Patent Application No. 2023-049720 filed on Mar. 27, 2023, Japanese Patent Application No. 2023-175968 filed on Oct. 11, 2023, and Japanese Patent Application No. 2024-018416 filed on Feb. 9, 2024. Each of the above application(s) is hereby expressly incorporated by reference, in its entirety, into the present application.

### BACKGROUND OF THE INVENTION

### 1. Field of the Invention

The present invention relates to a composition containing an ultraviolet absorber. In addition, the present invention also relates to a cured substance and an optical member, which are formed of a composition containing the ultraviolet absorber.

### 2. Description of the Related Art

A benzobisdithiol compound has excellent absorption of ultraviolet rays, and has been used as an ultraviolet absorber or the like. For example, JP2009-067984A discloses use of a specific benzobisdithiol as an ultraviolet absorber.

### SUMMARY OF THE INVENTION

In the ultraviolet absorber, as one of required characteristics, it is required that the ultraviolet absorber has little coloration. In addition, in recent years, there has been a demand for having a high absorption ability for ultraviolet rays having a longer wavelength in the vicinity of a wavelength of 400 nm.

In addition, the ultraviolet absorption performance of the ultraviolet absorber may be degraded with time due to irradiation with light. In particular, an ultraviolet absorber having a maximal absorption wavelength on a longer wavelength side in an ultraviolet region has a tendency that light resistance is poor and the ultraviolet absorption ability thereof is likely to be degraded with time. Therefore, in recent years, there has been a demand for further improvement of performance in light resistance of the ultraviolet absorber.

Therefore, an object of the present invention is to provide a composition with which a cured substance having excellent absorption ability of ultraviolet rays in a vicinity of a wavelength of 400 nm, little coloration, and excellent light resistance can be produced. Further, another object of the present invention is to provide a cured substance and an optical member.

As a result of intensive studies on a compound having a skeleton represented by Formula (1), the present inventor has found that a compound having a structure in which Q¹ and Q² in Formula (1) are in a specific combination is a compound having excellent absorption ability of ultraviolet rays in a vicinity of a wavelength of 400 nm, little coloration, and excellent light resistance. As a result of further studies, it was found that a film with little coloration can be obtained even after a long period of light irradiation by using this compound and a predetermined anti-fading agent in combination, and thus the present invention was completed. Therefore, the present invention provides the following.

<1> A composition comprising: an ultraviolet absorber; a curable compound; and an anti-fading agent,
in which the ultraviolet absorber includes at least one selected from a compound represented by Formula (1) or a polymer including a structure derived from the compound represented by Formula (1),
the anti-fading agent includes at least one selected from an amine compound, a phenol compound, a hydroquinone compound, a catechol compound, an ascorbic acid compound, a carotenoid compound, a metal complex compound, or a benzolactone compound.

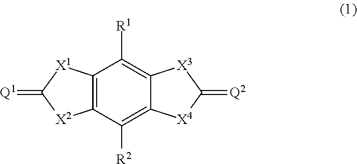

in Formula (1), Q¹ represents a group represented by Formula (Q-1),
Q² represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring, provided that, in a case where R^{q2} and R^{q3} are bonded to each other to form a ring, ═CR^{q2}R^{q3} does not have the same structure as Q¹,
R¹ and R² each independently represent a hydrogen atom or a substituent, and
X¹ to X⁴ each independently represent —S—, —NR^{X1}—, or —SO₂—, and R^{X1} represents a hydrogen atom or an alkyl group,

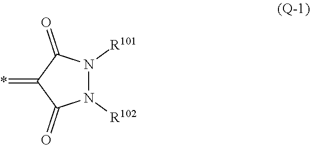

in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other represents an alkyl group, an aralkyl group, an aryl group, a heterocyclic group, or a group including a polymerizable group having an ethylenically unsaturated bond, and
in a case where any one of R¹⁰¹ or R¹⁰² is a methyl group, the other represents a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other represents a hydrogen atom, an alkyl group, an aralkyl group, an aryl group having a substituent, a heterocyclic group, or a group including a polymerizable group having an ethylenically unsaturated bond.

<2> The composition according to <1>, in which the compound represented by Formula (1) is a compound represented by Formula (2),

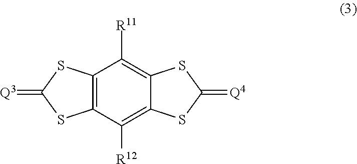

in Formula (3), Q³ represents the group represented by Formula (Q-1),
Q⁴ represents ═O, ═S, ═NR^{q11}, or ═CR^{q12}R¹³, R^{q11} to R^{q13} each independently represent a hydrogen atom or a substituent, and R^{q12} and R^{q13} may be bonded to each other to form a ring, provided that, in a case where R^{q12} and R^{q13} are bonded to each other to form a ring, ═CR^{q12}R^{q13} does not have the same structure as Q³, and
R¹¹ and R¹² each independently represent —OH, —O—Y¹¹, —OC(═O)—Y¹¹, —OC(═O)O—Y¹¹, —OC(═O)NR^{y11}—Y¹¹, —OSO₂—Y¹¹, or a group including a polymerizable group having an ethylenically unsaturated bond, where R^{y11} represents a hydrogen atom, an alkyl group, an aralkyl group, or an aryl group, and Y¹¹ represents an alkyl group, an aralkyl group, or an aryl group.

<3> The composition according to <1> or <2>, in which the anti-fading agent includes at least one selected from a compound represented by Formula (Ao1-1) or a compound represented by Formula (Ao2-1),

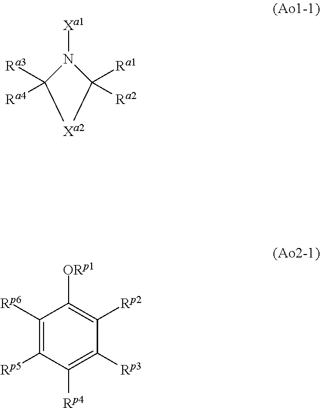

in Formula (Ao1-1), R^{a1} to R^{a4} each independently represent a hydrogen atom, an alkyl group, or an alkenyl group,
X^{a1} represents a hydrogen atom, an alkyl group, an alkenyl group, an alkoxy group, an alkenyloxy group, an alkoxycarbonyl group, an alkenyloxycarbonyl group, an aryloxycarbonyl group, an acyl group, an acyloxy group, an alkoxycarbonyloxy group, an alkenyloxycarbonyloxy group, an aryloxycarbonyloxy group, an alkylsulfonyl group, an alkenylsulfonyl group, an arylsulfonyl group, an alkylsulfinyl group, an alkenylsulfinyl group, an arylsulfinyl group, a sulfamoyl group, a carbamoyl group, a hydroxy group, or an oxyradical group,
X^{a2} represents an atomic group necessary to form a 5- to 7-membered ring,
in Formula (Ao2-1), R^{p1} represents a hydrogen atom, an alkyl group, an alkenyl group, an aryl group, a heterocyclic group, an acyl group, an alkoxycarbonyl group, an alkenyloxycarbonyl group, an aryloxycarbonyl group, an alkylsulfonyl group, an arylsulfonyl group, or —Si(R^{p101})(R^{p102})(R^{p103}), where R^{p101} to R^{p103} each independently represent an alkyl group, an alkenyl group, an aryl group, an alkoxy group, an alkenyloxy group, or an aryloxy group,
R^{p2} to R^{p6} each independently represent a hydrogen atom or a substituent, and
two adjacent groups among R^{p1} to R^{p6} may be bonded to each other to form a ring,
provided that, all of R^{p1} to R^{p6} are not hydrogen atoms.

<4> The composition according to <1> or <2>, in which the anti-fading agent includes at least one selected from a compound represented by Formula (Ao1-2) or a compound represented by Formula (Ao2-2),

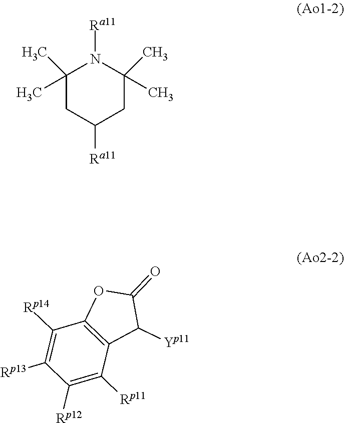

in Formula (Ao1-2), X^{a11} represents a hydrogen atom, an alkyl group, an alkenyl group, an alkoxy group, an alkenyloxy group, an alkoxycarbonyl group, an alkenyloxycarbonyl group, an aryloxycarbonyl group, an acyl group, an acyloxy group, an alkoxycarbonyloxy group, an alkenyloxycarbonyloxy group, an aryloxycarbonyloxy group, an alkylsulfonyl group, an alkenylsulfonyl group, an arylsulfonyl group, an alkylsulfinyl group, an alkenylsulfinyl group, an arylsulfinyl group, a sulfamoyl group, a carbamoyl group, a hydroxy group, or an oxyradical group, and
R^{a11} represents a substituent,
in Formula (Ao2-2), R^{p11} to R^{p14} each independently represent a hydrogen atom, an alkyl group, or an alkenyl group, and
Y^{p11} represents an aryl group, a heteroaryl group, or an ethylenically unsaturated bond-containing group.

<5> The composition according to any one of <1> to <4>, in which the curable compound includes at least one selected from a resin or a polymerizable compound.

<6> The composition according to any one of <1> to <4>, in which the curable compound includes a resin, and the resin is at least one selected from a (meth)acrylic resin, a polystyrene resin, a polyester resin, a polyurethane resin, a thiourethane resin, a polyimide resin, an epoxy resin, a polycarbonate resin, a phthalate resin, a cellulose acylate resin, or a cyclic olefin resin.

<7> A cured substance formed of the resin composition according to any one of <1> to <6>.

<8> An optical member comprising:
the cured substance according to <7>.

According to the present invention, it is possible to provide a composition with which a cured substance having excellent absorption ability of ultraviolet rays in a vicinity of a wavelength of 400 nm, little coloration, and excellent light resistance can be produced. Further, according to the present invention, it is possible to provide a cured substance and an optical member.

### DESCRIPTION OF THE PREFERRED EMBODIMENTS

Hereinafter, the contents of the present invention will be described in detail.

In a case where substitution or unsubstitution is not specified in the notation of a group (atomic group) in the present specification, the group includes both a group which has no substituent and a group which has a substituent. For example, “alkyl group” includes not only an alkyl group having no substituent (unsubstituted alkyl group), but also an alkyl group having a substituent (substituted alkyl group).

In the present specification, a numerical range represented by “to” means a range including numerical values before and after “to” as a lower limit value and an upper limit value.

In the present specification, a total solid content denotes the total amount of all components of the resin composition, excluding a solvent.

In the present specification, “(meth)acrylate” represents either or both of acrylate and methacrylate, “(meth)acryl” represents either or both of acryl and methacryl, “(meth)allyl” represents either or both of allyl and methallyl, and “(meth)acryloyl” represents either or both of acryloyl and methacryloyl.

In the present specification, the meaning of the term “step” includes not only an independent step but also a step whose intended purpose is achieved even in a case where the step is not clearly distinguished from other steps.

In the present specification, the weight-average molecular weight (Mw) and the number average molecular weight (Mn) are defined as values in terms of polystyrene, measured by gel permeation chromatography (GPC).

### <Composition>

The composition according to the embodiment of the present invention contains an ultraviolet absorber, a curable compound, and an anti-fading agent,
in which the ultraviolet absorber includes at least one selected from a compound represented by Formula (1) or a polymer including a structure derived from the compound represented by Formula (1), and
the anti-fading agent includes at least one selected from an amine compound, a phenol compound, a hydroquinone compound, a catechol compound, an ascorbic acid compound, or a benzolactone compound.

The compound represented by Formula (1) and a polymer including a structure derived from the compound represented by Formula (1) are compounds which have excellent absorption ability of ultraviolet rays in the vicinity of a wavelength of 400 nm, has little coloration, and has excellent light resistance in which decomposition or the like due to irradiation with light is less likely to occur. The composition according to the embodiment of the present invention, which contains at least one selected from a compound represented by Formula (1) or a polymer including a structure derived from the compound represented by Formula (1) as an ultraviolet absorber, can be produced a cured substance having an excellent absorption ability of ultraviolet rays in the vicinity of a wavelength of 400 nm, little coloration, and excellent light resistance. In addition, the composition according to the embodiment of the present invention further includes at least one anti-fading agent selected from an amine compound, a phenol compound, a hydroquinone compound, a catechol compound, an ascorbic acid compound, or a benzolactone compound, and thus, even in a case where the film obtained by using the composition is irradiated with light for a long period of time, it is possible to suppress the decomposition of the compound represented by Formula (1) or the polymer including the structure derived from the compound represented by Formula (1). Therefore, according to the composition of the embodiment of the present invention, it is possible to obtain a film having little coloration even after a long period of light irradiation.

Therefore, the composition according to the embodiment of the present invention can be used to produce a cured substance having excellent absorption ability of ultraviolet rays in the vicinity of a wavelength of 400 nm, little coloration, and excellent light resistance.

The composition according to the embodiment of the present invention may be a solution-state composition containing a solvent. In addition, the composition according to the embodiment of the present invention may be a kneaded material. In the present specification, the kneaded material is a material obtained by kneading a resin and an ultraviolet absorber including at least one selected from a compound represented by Formula (1) or a polymer including a structure derived from the compound represented by Formula (1). That is, the kneaded material in the present specification is a material in which an ultraviolet absorber is mixed and dispersed in a resin, and is different from a solution in which an ultraviolet absorber and a resin are dissolved or dispersed in a solvent. In addition, in a case where the composition according to the embodiment of the present invention is a kneaded material, a curable compound including a resin is used as the curable compound.

The kneaded material is also preferably in a form of pellet. In the present specification, the pellet is a material obtained by granulating (pelletizing) the kneaded material into a certain shape such as a spherical shape, an ellipsoidal shape, a cylindrical shape, and a prismatic shape. In addition, it is also preferable that the pellet is a master pellet (masterbatch). In addition, the master pellet (the masterbatch) is a material obtained by dispersing an additive such as an ultraviolet absorber having a high concentration in a resin, and is used by being mixed with the resin or the like at a specified magnification in a case of forming a molded body.

Hereinafter, each component of the composition according to the embodiment of the present invention will be described.

### <<Ultraviolet Absorber>>

The composition according to the embodiment of the present invention contains an ultraviolet absorber. As the ultraviolet absorber, an ultraviolet absorber including at least one selected from a compound represented by Formula (1) or a polymer including a structure derived from a compound represented by Formula (1) is used. The ultraviolet absorber preferably includes a compound represented by Formula (1). Hereinafter, the compound represented by Formula (1) is also referred to as a specific compound. In addition, the polymer including the structure derived from the compound represented by Formula (1) is also referred to as a specific polymer. In addition, the specific compound and the specific polymer are also collectively referred to as a specific ultraviolet absorber.

### <<Specific Ultraviolet Absorber>>>

### (Compound (Specific Compound) Represented by Formula (1))

First, the compound (specific compound) represented by Formula (1) will be described.

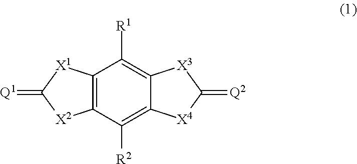

in Formula (1), Q¹ represents a group represented by Formula (Q-1),
Q² represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring, provided that, in a case where R^{q2} and R^{q3} are bonded to each other to form a ring, ═CR^{q2}R^{q3} does not have the same structure as Q¹,
R¹ and R² each independently represent a hydrogen atom or a substituent, and
X¹ to X⁴ each independently represent —S—, —NR^{X1}—, or —SO₂—, and R^{X1} represents a hydrogen atom or an alkyl group,

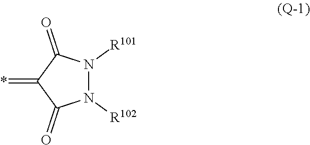

in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other represents an alkyl group, an aralkyl group, an aryl group, a heterocyclic group, or a group including a polymerizable group having an ethylenically unsaturated bond, and
in a case where any one of R¹⁰¹ or R¹⁰² is a methyl group, the other represents a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other represents a hydrogen atom, an alkyl group, an aralkyl group, an aryl group having a substituent, a heterocyclic group, or a group including a polymerizable group having an ethylenically unsaturated bond.
   —Regarding R¹ and R²—

Examples of the substituent represented by R¹ and R² in Formula (1) include an alkyl group, an aryl group, an aralkyl group, a heterocyclic group, a group including a polymerizable group having an ethylenically unsaturated bond, —OH, —O—Y¹¹, —OC(═O)—Y¹¹, —OC(═O)O—Y¹¹, —OC(═O)NR^{y11}—Y¹¹, —OSO₂—Y¹¹, a cyano group, a halogen atom, and a nitro group. R^{y11} represents a hydrogen atom, an alkyl group, an aralkyl group, or an aryl group, and Y¹¹ represents an alkyl group, an aralkyl group, or an aryl group.

The number of carbon atoms in the above-described alkyl group is preferably 1 to 30, more preferably 1 to 20, still more preferably 1 to 15, particularly preferably 1 to 10, and most preferably 1 to 8. The alkyl group may be linear, branched, or cyclic, and preferably linear or branched. The alkyl group may have a substituent. Examples of the substituent include groups described in the section of the substituent T described below.

The number of carbon atoms in the above-described aryl group is preferably 6 to 30, more preferably 6 to 20, still more preferably 6 to 15, particularly preferably 6 to 10, and most preferably 6 to 8. The aryl group may have a substituent. Examples of the substituent include groups described in the section of the substituent T described below.

The number of carbon atoms in an alkyl moiety of the above-described aralkyl group is preferably 1 to 10, more preferably 1 to 5, and still more preferably 1 to 3. The number of carbon atoms in an aryl moiety of the above-described aralkyl group is preferably 6 to 30, more preferably 6 to 20, still more preferably 6 to 15, particularly preferably 6 to 10, and most preferably 6 to 8. The aralkyl group may have a substituent. Examples of the substituent include groups described in the section of the substituent T described below. Specific examples of the aralkyl group include a benzyl group.

A heterocyclic ring in the above-described heterocyclic group preferably includes a 5-membered or 6-membered saturated or unsaturated heterocyclic ring. The heterocyclic ring may be fused with an aliphatic ring, an aromatic ring, or another heterocyclic ring. Examples of the heteroatom constituting the ring of the heterocyclic ring include B, N, O, S, Se, and Te. Among these, N, O and S are preferable. It is preferable that the carbon atom of the heterocyclic ring has a free valence (monovalent) (the heterocyclic group is bonded at the carbon atom). The number of carbon atoms of the heterocyclic group is preferably 1 to 40, more preferably 1 to 30, and still more preferably 1 to 20. Examples of the saturated heterocyclic ring in the heterocyclic group include a pyrrolidine ring, a morpholine ring, a 2-bora-1,3-dioxolane ring, and a 1,3-thiazolidine ring. Examples of the unsaturated heterocyclic ring in the heterocyclic group include an imidazole ring, a thiazole ring, a benzothiazole ring, a benzoxazole ring, a benzotriazole ring, a benzoselenazole ring, a pyridine ring, a pyrimidine ring, and a quinoline ring.

Examples of the above-described halogen atom include a chlorine atom, a bromine atom, and an iodine atom.

Examples of the polymerizable group having an ethylenically unsaturated bond in the above-described group including a polymerizable group having an ethylenically unsaturated bond include a vinyl group, a (meth)allyl group, a (meth)acryloyl group, a (meth)acryloyloxy group, a (meth)acryloylamino group, and a vinylphenyl group; and a (meth)acryloyloxy group or a vinylphenyl group is preferable.

Examples of the group including a polymerizable group having an ethylenically unsaturated bond include a group represented by Formula (T1).

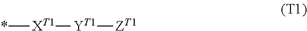

In Formula (T1), X^{T1} represents a single bond, —O—, —OC(═O)—, —OC(═O)O—, or —OC(═O)NR^{X1}—, and R^{X1} represents a hydrogen atom, an alkyl group, or an aryl group,
Y^{T1} represents a single bond or a divalent linking group, and
Z^{T1} represents a polymerizable group having an ethylenically unsaturated bond.

As the alkyl group represented by R^{X1}, an alkyl group having 1 to 30 carbon atoms is preferable. Specific examples thereof include a methyl group, an ethyl group, an n-propyl group, an isopropyl group, and an n-butyl. The aryl group represented by R^{X1} is preferably a substituted or unsubstituted aryl group having 6 to 30 carbon atoms. Specific examples thereof include a phenyl group, a p-tolyl group, and a naphthyl group. It is preferable that R^{X1} represents a hydrogen atom.

X^{T1} is preferably —O—, —OC(═O)—, or —OC(═O)NH—; and from the viewpoint of synthesis, more preferably —OC(═O)—.

Examples of the divalent linking group represented by Y^{T1} include a hydrocarbon group and a group in which two or more hydrocarbon groups are bonded to each other by a single bond or a linking group. Examples of the hydrocarbon group include an aliphatic hydrocarbon group and an aromatic hydrocarbon group, and an aliphatic hydrocarbon group is preferable. The number of carbon atoms of the aliphatic hydrocarbon group is preferably 1 to 30, more preferably 1 to 20, and still more preferably 1 to 15. The aliphatic hydrocarbon group may be linear, branched, or cyclic. Further, the cyclic aliphatic hydrocarbon group may be a monocycle or a fused ring. Further, the cyclic aliphatic hydrocarbon group may have a crosslinking structure. The number of carbon atoms of the aromatic hydrocarbon group is preferably 6 to 30, more preferably 6 to 20, and still more preferably 6 to 10. The hydrocarbon group may have a substituent. Examples of the substituent include the substituent T described below. Examples of the substituent include a hydroxy group.

Examples of the linking group which links two or more of the above-described hydrocarbon groups include —NH—, —S(═O)₂—, —O—, —C(═O)—, —OC(═O)—, —C(═O)O—, —NHC(═O)—, and —C(═O)NH—. Among these, —O—, —C(═O)—, —OC(═O)—, —C(═O)O—, —NHC(═O)—, or —C(═O)NH— is preferable.

Examples of the polymerizable group having an ethylenically unsaturated bond, represented by Z^{T1}, include a vinyl group, an allyl group, a (meth)acryloyl group, a (meth)acryloyloxy group, a (meth)acryloylamino group, and a vinylphenyl group; and a (meth)acryloyloxy group or a vinylphenyl group is preferable.

Specific examples of the group represented by Formula (T1) include groups represented by T-1 to T-32. In the following structural formulae, Me represents a methyl group and * represents a bonding site.

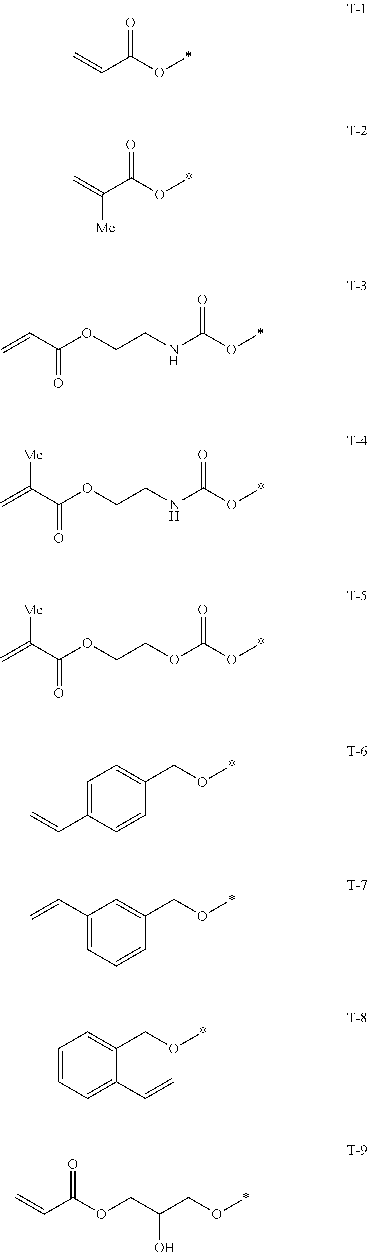

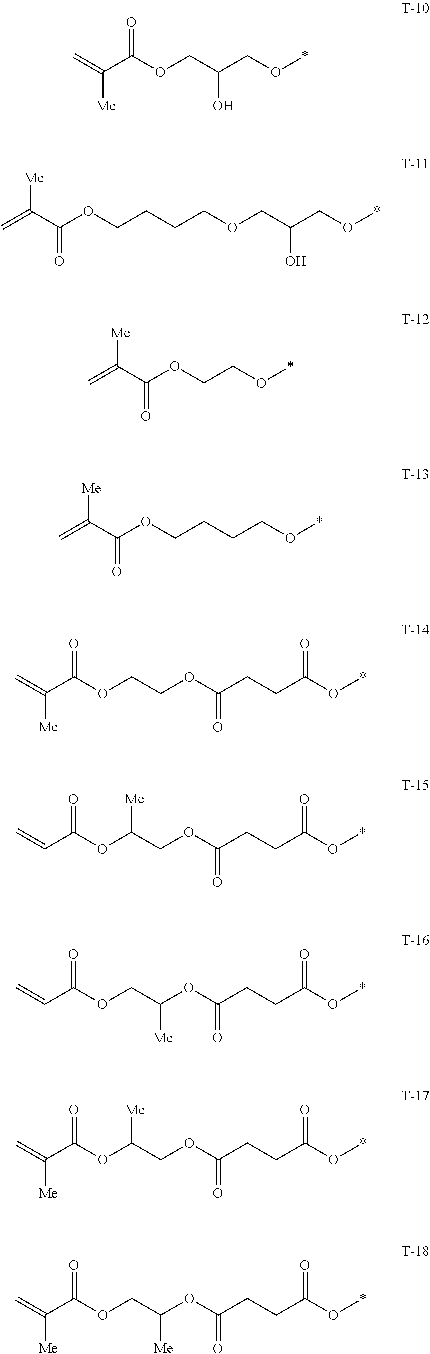

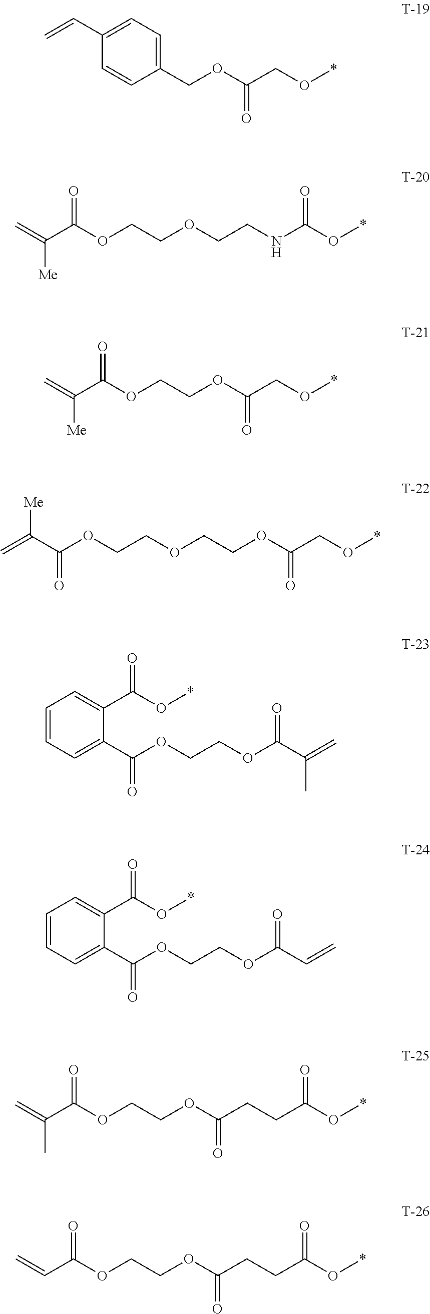

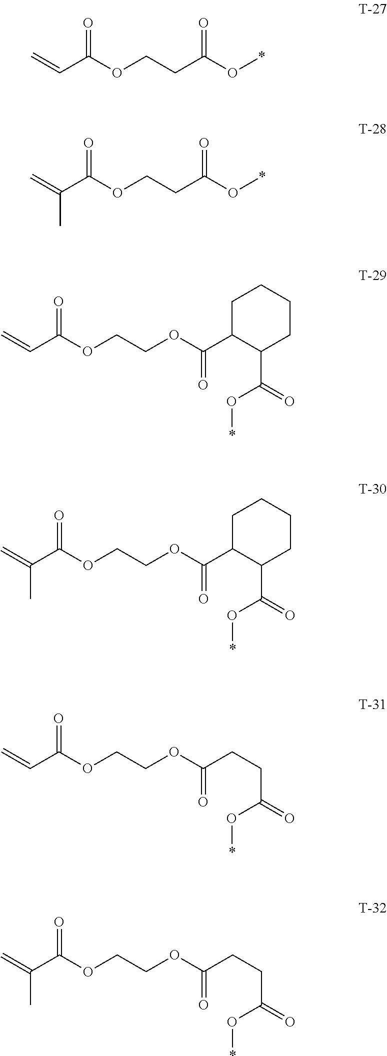 [SMILES: *OC(=O)CCOC(=O)C=C ; *OC(=O)CCOC(=O)C(=C)C ; *OC(=O)C1CCCCC1C(=O)OCCOC(=O)C=C ; *OC(=O)C1CCCCC1C(=O)OCCOC(=O)C(=C)C ; *OC(=O)CCC(=O)OCCOC(=O)C=C ; *OC(=O)CCC(=O)OCCOC(=O)C(=C)C]

It is preferable that R¹ and R² in Formula (1) each independently represent —OH, —O—Y¹¹, —OC(═O)—Y¹¹, —OC(═O)O—Y¹¹, —OC(═O)NR^{y11}—Y¹¹, —OSO₂—Y¹¹, or the group including a polymerizable group having an ethylenically unsaturated bond. R¹ and R² in Formula (1) may be the same group or different groups.

Examples of one aspect thereof include an aspect in which R¹ and R² in Formula (1) are each independently —OH, —O—Y¹¹, —OC(═O)—Y¹¹, —OC(═O)O—Y¹¹, —OC(═O)NR^{y11}—Y¹¹, or —OSO₂—Y¹¹. In this aspect, from the reason that stability is excellent, it is more preferable that R¹ and R² are each independently —OC(═O)—Y¹¹, —O—Y¹¹, or —OC(═O)NR^{y11}—Y¹¹. R^{y11} is preferably a hydrogen atom or an alkyl group and more preferably a hydrogen atom. From the reason of excellent solubility, Y¹¹ is preferably an alkyl group, more preferably a linear or branched alkyl group, and still more preferably a branched alkyl group.

Examples of another aspect thereof include an aspect in which at least one of R¹ or R² is a group including the group including a polymerizable group having an ethylenically unsaturated bond. According to this aspect, the effect of being capable of suppressing bleed-out in a film is obtained.

Examples of another aspect thereof include an aspect in which one of R¹ or R² is —O—Y¹¹ and the other is —OC(═O)—Y¹¹.

Examples of another aspect include an aspect in which one of R¹ or R² is —O—Y¹¹ or —OC(═O)—Y¹¹ and the other is a group including a polymerizable group having an ethylenically unsaturated bond.

—Regarding X¹ to X⁴—

X¹ to X⁴ in Formula (1) each independently represent —S—, —NR^{X1}—, or —SO₂—, and R^{X1} represents a hydrogen atom or an alkyl group. A preferred range of the alkyl group represented by R^{X1} is the same as that for the above-described alkyl group. R^{X1} is preferably a hydrogen atom.

From the reason that the effect of the present invention is more remarkable, X¹ to X⁴ in Formula (1) are preferably —S—.

### —Regarding Q¹—

Q¹ in Formula (1) represents a group represented by Formula (Q-1).

R¹⁰¹ and R¹⁰² in Formula (Q-1) each independently represent a hydrogen atom, an alkyl group, an aralkyl group, an aryl group, a heterocyclic group, or a group including a polymerizable group having an ethylenically unsaturated bond.

However, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond; in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond; and in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.

The number of carbon atoms in the alkyl group represented by R¹⁰¹ and R¹⁰² is preferably 1 to 30. The upper limit thereof is preferably 20 or less, more preferably 15 or less, still more preferably 10 or less, and even more preferably 8 or less. The lower limit thereof is preferably 2 or more and more preferably 3 or more. The alkyl group may be linear, branched, or cyclic, and preferably linear or branched. The alkyl group may have a substituent. Examples of the substituent include groups described in the section of the substituent T described below.

The number of carbon atoms in the aryl group represented by R¹⁰¹ and R¹⁰² is preferably 6 to 30, more preferably 6 to 20, still more preferably 6 to 15, particularly preferably 6 to 10, and most preferably 6 to 8. The aryl group may have a substituent. Examples of the substituent include groups described in the section of the substituent T described below.

The number of carbon atoms in an alkyl moiety of the aralkyl group represented by R¹⁰¹ and R¹⁰² is preferably 1 to 10, more preferably 1 to 5, and still more preferably 1 to 3.

The number of carbon atoms in an aryl moiety of the above-described aralkyl group is preferably 6 to 30, more preferably 6 to 20, still more preferably 6 to 15, particularly preferably 6 to 10, and most preferably 6 to 8. The aralkyl group may have a substituent.

Examples of the substituent include groups described in the section of the substituent T described below.

Examples of the heterocyclic group represented by R¹⁰¹ and R¹⁰² include the above-described heterocyclic groups.

Examples of the group including a polymerizable group having an ethylenically unsaturated bond, represented by as R¹⁰¹ and R¹⁰², include a group represented by Formula (V1).

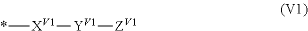

In Formula (V1), X^{V1} represents a single bond, —O—, —C(═O)—, —C(═O)O—, or —C(═O)NRx²—, and Rx² represents a hydrogen atom, an alkyl group, or an aryl group,
Y^{V1} represents a single bond or a divalent linking group, and
Z^{V1} represents a polymerizable group having an ethylenically unsaturated bond.

The alkyl group and aryl group represented by Rx² have the same meaning as the alkyl group and aryl group represented by Rx¹ of the group represented by Formula (T1), and preferred ranges thereof are also the same. Rx² is preferably a hydrogen atom.
X^{V1} is preferably a single bond or —C(═O)—, and more preferably a single bond.

Examples of the divalent linking group represented by Y^{V1} include the groups described as the divalent linking group represented by Y^{T1} of the group represented by Formula (T1), and a preferred range thereof is also the same.

Examples of the polymerizable group having an ethylenically unsaturated bond, represented by Z^{V1}, include a vinyl group, an allyl group, a (meth)acryloyl group, a (meth)acryloyloxy group, a (meth)acryloylamino group, and a vinylphenyl group; and a (meth)acryloyloxy group or a vinylphenyl group is preferable.

Specific examples of the group represented by Formula (V1) include groups represented by V-1 to V-12. In the following structural formulae, * is a bonding site.

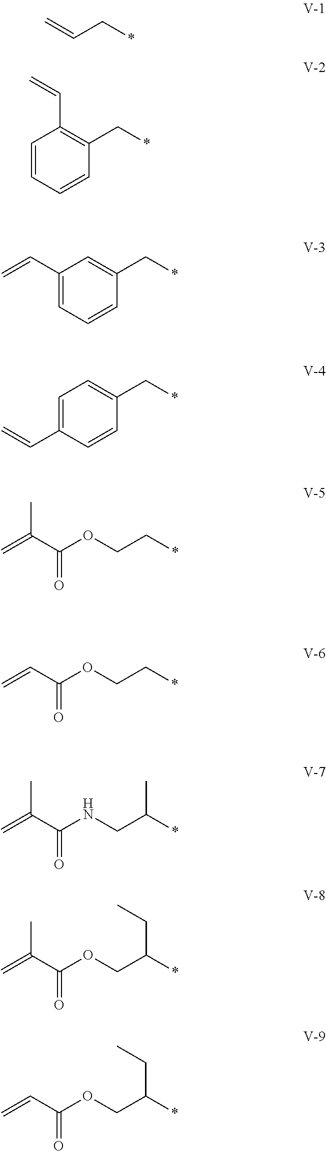

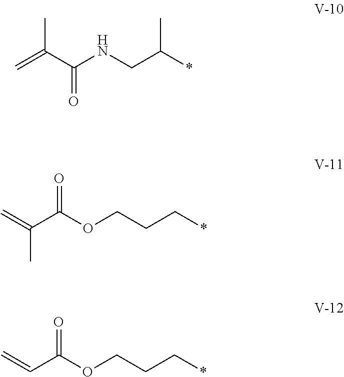

Examples of one aspect of R¹⁰¹ and R¹⁰² in Formula (Q-1) includes an aspect in which R¹⁰¹ and R¹⁰² each independently represent a hydrogen atom, an alkyl group, an aralkyl group, an aryl group, or a heterocyclic group,
in a case where any one of R¹⁰¹ or R¹⁰² is a hydrogen atom, the other represents an alkyl group, an aralkyl group, an aryl group, or a heterocyclic group,
in a case where any one of R¹⁰¹ or R¹⁰² is a methyl group, the other represents a hydrogen atom, an alkyl group having 2 or more carbon atoms, an aralkyl group, an aryl group, or a heterocyclic group, and
in a case where any one of R¹⁰¹ or R¹⁰² is a phenyl group, the other represents a hydrogen atom, an alkyl group, an aralkyl group, an aryl group having a substituent, or a heterocyclic group.
R¹⁰¹ and R¹⁰² in Formula (Q-1) are each independently preferably an alkyl group or an aralkyl group, and more preferably an aralkyl group.

In a case where R¹⁰¹ and R¹⁰² are alkyl groups, it is preferable that the alkyl groups represented by R¹⁰¹ and R¹⁰² are each independently an alkyl group having 2 or more carbon atoms.

Examples of another aspect of R¹⁰¹ and R¹⁰² in Formula (Q-1) include an aspect in which at least one of R¹⁰¹ or R¹⁰² in Formula (Q-1) is the group including a polymerizable group having an ethylenically unsaturated bond. According to this aspect, the effect of being capable of suppressing bleed-out in a film is obtained.

### —Regarding Q²—

Q² in Formula (1) represents ═O, ═S, ═NR^{g1}, or ═CR^{q2}R^{q3}, R^{q1} to R³ each independently represent a hydrogen atom or a substituent, and R^{q2} and R^{q3} may be bonded to each other to form a ring. However, in a case where R^{q2} and R^{q3} are bonded to each other to form a ring, ═CR^{q2}R^{q3} does not have the same structure as Q¹.

Examples of the substituent represented by R^{q1} to R³ include a cyano group, a carbamoyl group, a sulfamoyl group, a nitro group, an acyl group, an alkylsulfonyl group, an arylsulfonyl group, an alkylsulfinyl group, an arylsulfinyl group, an alkoxycarbonyl group, an aryloxycarbonyl group, an alkyl group, an aryl group, a heterocyclic group, and a group including a polymerizable group having an ethylenically unsaturated bond. These groups may further have a substituent. Examples of the substituent include groups exemplified in the substituent T described later.

Examples of the carbamoyl group include a carbamoyl group having 1 to 10 carbon atoms; and a carbamoyl group having 2 to 8 carbon atoms is preferable, and a carbamoyl group having 2 to 5 carbon atoms is more preferable.

Examples of the sulfamoyl group include a sulfamoyl group having 0 to 10 carbon atoms; and a sulfamoyl group having 2 to 8 carbon atoms is preferable, and a sulfamoyl group having 2 to 5 carbon atoms is more preferable.

Examples of the acyl group include an acyl group having 1 to 20 carbon atoms; and an acyl group having 1 to 12 carbon atoms is preferable, and an acyl group having 1 to 8 carbon atoms is more preferable.

Examples of the alkylsulfonyl group include an alkylsulfonyl group having 1 to 20 carbon atoms; and an alkylsulfonyl group having 1 to 10 carbon atoms is preferable, and an alkylsulfonyl group having 1 to 8 carbon atoms is more preferable.

Examples of the arylsulfonyl group include an arylsulfonyl group having 6 to 20 carbon atoms, and an arylsulfonyl group having 6 to 10 carbon atoms is preferable.

Examples of the alkylsulfinyl group include an alkylsulfinyl group having 1 to 20 carbon atoms; and an alkylsulfinyl group having 1 to 10 carbon atoms is preferable, and an alkylsulfinyl group having 1 to 8 carbon atoms is more preferable.

Examples of the arylsulfinyl group include an arylsulfinyl group having 6 to 20 carbon atoms, and an arylsulfinyl group having 6 to 10 carbon atoms is preferable.

Examples of the alkoxycarbonyl group include an alkoxycarbonyl group having 2 to 20 carbon atoms; and an alkoxycarbonyl group having 2 to 12 carbon atoms is preferable, and an alkoxycarbonyl group having 2 to 8 carbon atoms is more preferable.

Examples of the aryloxycarbonyl group include an aryloxycarbonyl group having 6 to 20 carbon atoms; and an aryloxycarbonyl group having 6 to 12 carbon atoms is preferable, and an aryloxycarbonyl group having 6 to 8 carbon atoms is more preferable.

Examples of the alkyl group include an alkyl group having 1 to 18 carbon atoms; and an alkyl group having 1 to 10 carbon atoms is preferable and an alkyl group having 1 to 5 carbon atoms is more preferable.

Examples of the aryl group include an aryl group having 6 to 20 carbon atoms; and an aryl group having 6 to 15 carbon atoms is preferable, and an aryl group having 6 to 10 carbon atoms is more preferable.

It is preferable that the heterocyclic ring in the heterocyclic group includes a 5- or 6-membered saturated or unsaturated heterocyclic ring. The heterocyclic ring may be fused with an aliphatic ring, an aromatic ring, or another heterocyclic ring. Examples of the heteroatom constituting the ring of the heterocyclic ring include B, N, O, S, Se, and Te.

Among these, N, O and S are preferable. It is preferable that the carbon atom of the heterocyclic ring has a free valence (monovalent) (the heterocyclic group is bonded at the carbon atom). The number of carbon atoms of the heterocyclic group is preferably 1 to 40, more preferably 1 to 30, and still more preferably 1 to 20.

Examples of the group including a polymerizable group having an ethylenically unsaturated bond include a group represented by Formula (U1).

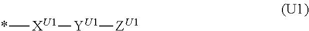

In Formula (U1), X^{U1} represents a single bond, —C(═O)—, —C(═O)O—, or —C(═O)NRx³—, and Rx³ represents a hydrogen atom, an alkyl group, or an aryl group,
Y^{U1} represents a single bond or a divalent linking group, and
Z^{U1} represents a polymerizable group having an ethylenically unsaturated bond.

The alkyl group and aryl group represented by Rx³ have the same meaning as the alkyl group and aryl group represented by R^{X1} of the group represented by Formula (T1), and preferred ranges thereof are also the same. Rx³ is preferably a hydrogen atom.

X^{U1} is preferably —C(═O)O— or —C(═O)NRx³—; and from the viewpoint of synthesis, more preferably —C(═O)O—.

Examples of the divalent linking group represented by Y^{U1} include the groups described as the divalent linking group represented by Y^{T1} of the group represented by Formula (T1), and a preferred range thereof is also the same.

Examples of the polymerizable group having an ethylenically unsaturated bond, represented by Z^{U1}, include a vinyl group, an allyl group, a (meth)acryloyl group, a (meth)acryloyloxy group, a (meth)acryloylamino group, and a vinylphenyl group; and a (meth)acryloyloxy group or a vinylphenyl group is preferable.

Specific examples of the group represented by Formula (U1) include groups represented by U-1 to U-11. In the following structural formulae, * is a bonding site.

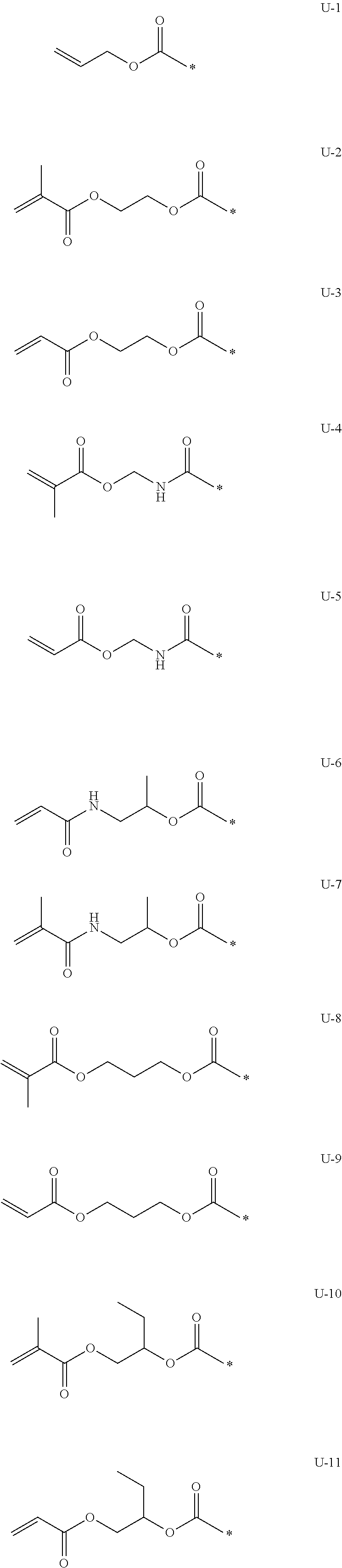

From the reason that the effect of the present invention is more remarkable, Q² in Formula (1) is preferably ═CR^{q2}R^{q3}. In addition, it is preferable that at least one of R^{q2} or R^{q3} is an electron withdrawing group, and it is more preferable that R^{q2} and R^{q3} are electron withdrawing groups.

It is also preferable that at least one of R^{q2} or R^{q3} is the group including a polymerizable group having an ethylenically unsaturated bond. In this aspect, it is also preferable that one of R^{q2} or R^{q3} is the group including a polymerizable group having an ethylenically unsaturated bond, and the other is an electron withdrawing group.

Examples of the electron withdrawing group include a substituent having the Hammett's substituent constant σp value of 0.2 or more. The Hammett's substituent constant σp value will be described. The Hammett's rule is an empirical rule advocated by L. P. Hammett in 1935 so as to quantitatively discuss the effect of substituent on the reaction or equilibrium of benzene derivatives and its propriety is widely admitted at present. Substituent constants obtained by the Hammett's rule are an σp value and an am value, and these values can be found in many general books. For example, these values are specifically described in “Lange's Handbook of Chemistry”, edited by J. A. Dean, 12th edition, 1979 (McGraw-Hill), “Chemistry Region”, extra edition, No. 122, pp. 96 to 103, 1979 (Nankodo, Co., Ltd.), and Chem. Rev., 1991, Vol. 91, pp. 165 to 195. In the present specification, a substituent having a Hammett's substituent constant σp value of 0.2 or more is denoted as the electron withdrawing group. The electron withdrawing group is preferably a group having a Hammett's substituent constant σp value of 0.25 or more, more preferably a group having a Hammett's substituent constant σp value of 0.3 or more, and still more preferably a group having a Hammett's substituent constant σp value of 0.35 or more.

Specific examples of the group having a Hammett's substituent constant σp value of 0.2 or more include a cyano group (σp value=0.66), a carboxy group (—COOH; σp value=0.45), an alkoxycarbonyl group (—COOMe; σp value=0.45), an aryloxycarbonyl group (—COOPh; σp value=0.44), a carbamoyl group (—CONH₂; σp value=0.36), an alkylcarbonyl group (—COMe; σp value=0.50), an arylcarbonyl group (—COPh; σp value=0.43), an alkylsulfonyl group (—SO₂Me; σp value=0.72), and an arylsulfonyl group (—SO₂Ph; σp value=0.68). Me represents a methyl group and Ph represents a phenyl group. The values in the parentheses are representative σp values of the substituents extracted from “Chem. Rev.” vol. 91, pp. 165 to 195, 1991.

It is preferable that R^{q2} and R^{q3} are each independently a hydrogen atom, a cyano group, a carbamoyl group, a sulfamoyl group, an acyl group, an alkylsulfonyl group, an arylsulfonyl group, an alkylsulfinyl group, an arylsulfinyl group, a nitro group, an alkoxycarbonyl group, an aryloxycarbonyl group, or a group including a polymerizable group having an ethylenically unsaturated bond.

Examples of one aspect thereof include an aspect in which R^{q2} and R^{q3} are each independently a hydrogen atom, a cyano group, a carbamoyl group, a sulfamoyl group, an acyl group, an alkylsulfonyl group, an arylsulfonyl group, an alkylsulfinyl group, an arylsulfinyl group, a nitro group, an alkoxycarbonyl group, or an aryloxycarbonyl group. Among these, it is preferable that at least one of R^{q2} or R^{q3} is a cyano group, an alkoxycarbonyl group, a nitro group, or an alkylsulfonyl group, and it is more preferable that R^{q2} and R^{q3} are each independently a cyano group or an alkoxycarbonyl group. Examples of a preferred aspect thereof include an aspect in which R^{q2} and R^{q3} are cyano groups. Examples of another preferred aspect include an aspect in which one of R^{q2} or R^{q3} is a cyano group and the other is an alkoxycarbonyl group.

As another aspect, it is also preferable that at least one of R^{q2} or R^{q3} is the group including a polymerizable group having an ethylenically unsaturated bond. R^{q2} and R^{q3} may be each independently the group including a polymerizable group having an ethylenically unsaturated bond, and one of R^{q2} or R^{q3} may be the group including a polymerizable group having an ethylenically unsaturated bond and the other may be the electron withdrawing group.

In the present specification, the expression “in a case where R^{q2} and R^{q3} are bonded to each other to form a ring, ═CR^{q2}R^{q3} does not have the same structure as Q” includes not only a case of forming a ring other than the structure represented by Formula (Q-1), but also a case of forming a ring having a structure which is the structure represented by Formula (Q-1), in which the types of R^{q1} and R^{q2} in Formula (Q-1) are different from the type of Q¹. That is, Q² is a group having a structure different from that of Q¹.

In a case where R^{q2} and R³ of ═CR^{q2}R^{q3} are bonded to each other to form a ring, from the reason that the effect of the present invention is more remarkable, the formed ring is preferably a ring other than the structure represented by Formula (Q-1). Examples of the ring other than the structure represented by Formula (Q-1) include a cyclopropane ring, a cyclobutane ring, a cyclopentane ring, a cyclohexane ring, a cycloheptane ring, a pyrrolidine ring, a tetrahydrofuran ring, a tetrahydrothiophene ring, an oxazoline ring, a thiazoline ring, a pyrroline ring, a pyrazoline ring, an imidazoline ring, an imidazolidine ring, a piperidine ring, a piperazine ring, a pyran ring, and indandione ring. These rings may have a substituent at any position.

It is preferable that at least one of R¹, R², Q¹, or Q² in Formula (1) includes the group including a polymerizable group having an ethylenically unsaturated bond, and it is more preferable that one or two of R¹, R², Q¹, and Q² include the group including a polymerizable group having an ethylenically unsaturated bond.

In addition, it is preferable that, in Formula (1), Q² is ═CR^{q2}R^{q3} and at least one of R¹, R², R^{q2}, R^{q3}, R¹⁰¹, or R¹⁰² is the group including a polymerizable group having an ethylenically unsaturated bond, and it is more preferable that one or two of R¹, R², R^{q2}, R^{q3}, R¹⁰¹, or R¹⁰² are the group including a polymerizable group having an ethylenically unsaturated bond.

In addition, the number of the polymerizable groups having an ethylenically unsaturated bond included in Formula (1) is preferably 1 or 2.

The specific compound is preferably a compound represented by Formula (3).

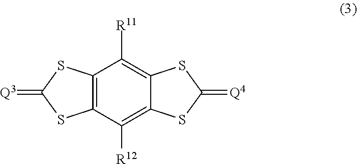

in Formula (3), Q³ represents the group represented by Formula (Q-1),
Q⁴ represents ═O, ═S, ═NR^{q11}, or ═CR^{q12}R¹³, R¹¹ to R^{q13} each independently represent a hydrogen atom or a substituent, and R^{q12} and R^{q13} may be bonded to each other to form a ring, provided that, in a case where R^{q12} and R^{q13} are bonded to each other to form a ring, ═CR^{q12}R^{q13} does not have the same structure as Q³; and
R¹¹ and R¹² each independently represent —OH, —O—Y¹¹, —OC(═O)—Y¹¹, —OC(═O)O—Y¹¹, —OC(═O)NR^{y11}—Y¹¹, —OSO₂—Y¹¹, or a group including a polymerizable group having an ethylenically unsaturated bond, where R^{y11} represents a hydrogen atom, an alkyl group, an aralkyl group, or an aryl group, and Y¹¹ represents an alkyl group, an aralkyl group, or an aryl group.
Q³ and Q⁴ in Formula (3) have the same meaning as Q¹ and Q² in Formula (1), and preferred ranges thereof are also the same. In addition, preferred ranges of R^{y11} and Y¹¹ in Formula (3) are the same as those for R^{y11} and Y¹¹ described in Formula (1).

Examples of one aspect thereof include an aspect in which R¹¹ and R¹² in Formula (3) are each independently —OH, —O—Y¹¹, —OC(═O)—Y¹¹, —OC(═O)O—Y¹¹, —OC(═O)NR^{y11}—Y¹¹, or —OSO₂—Y¹¹. In this aspect, it is preferable that R¹¹ and R¹² are each independently —OC(═O)—Y¹¹, —O—Y¹¹, or —OC(═O)NR^{y11}—Y¹¹.

Examples of another aspect thereof include an aspect in which at least one of R¹¹ or R¹² is a group including the group including a polymerizable group having an ethylenically unsaturated bond.

Examples of another aspect thereof include an aspect in which one of R¹¹ and R¹² is —O—Y¹¹ and the other is —OC(═O)—Y¹¹.

Examples of another aspect include an aspect in which one of R¹¹ or R¹² is —O—Y¹¹ or —OC(═O)—Y¹¹ and the other is a group including a polymerizable group having an ethylenically unsaturated bond.

It is preferable that at least one of R¹¹, R¹², Q³, or Q⁴ in Formula (3) includes the group including a polymerizable group having an ethylenically unsaturated bond, and it is more preferable that one or two of R¹¹, R¹², Q³, and Q⁴ include the group including a polymerizable group having an ethylenically unsaturated bond.

In addition, it is preferable that, in Formula (3), Q⁴ is ═CR^{q12}R^{q13} and at least one of R¹¹, R¹², R^{q12}, R^{q13}, R¹⁰¹, or R¹⁰² is the group including a polymerizable group having an ethylenically unsaturated bond, and it is more preferable that one or two of R¹¹, R¹², R^{q12}, R^{q13}, R¹⁰¹ or R¹⁰² are the group including a polymerizable group having an ethylenically unsaturated bond.

The number of the polymerizable groups having an ethylenically unsaturated bond included in Formula (3) is preferably 1 or 2.

The specific compound is preferably a compound represented by Formula (6).

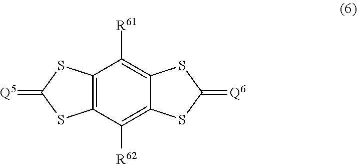

In Formula (6), Q⁵ represents the group represented by Formula (Q-1),
Q⁶ represents ═O, ═S, ═NR^{q21}, or ═CR^{q22}R^{q23}
R^{q21} to R^{q23} each independently represent a hydrogen atom or a substituent, and R^{q22} and R^{q23} may be bonded to each other to form a ring; provided that, in a case where R^{q22} and R^{q23} are bonded to each other to form a ring, ═CR^{q22}R^{q23} is not the same structure as Q⁵, and
R⁶¹ and R⁶² each independently represent —O—Y⁶¹, —OC(═O)—Y⁶¹, —OC(═O)O—Y⁶¹, —OC(═O)NR⁶¹—Y⁶¹, or —OSO₂—Y⁶¹, where R^{y61} represents a hydrogen atom, an alkyl group, an aralkyl group, or an aryl group, and Y⁶¹ represents an alkyl group, an aralkyl group, or an aryl group.
Q⁵ and Q⁶ in Formula (6) have the same meaning as Q¹ and Q² in Formula (1), and preferred ranges thereof are also the same. In addition, preferred ranges of R^{y61} and Y⁶¹ in Formula (6) are the same as those for R^{y11} and Y¹¹ described in Formula (1).

It is preferable that R⁶¹ and R⁶² in Formula (6) are each independently —OC(═O)—Y¹¹, —O—Y¹¹, or —OC(═O)NR^{y11}—Y¹¹.

R⁶¹ and R⁶² in Formula (6) may be the same group or different groups.

### (Substituent T)

Examples of the substituent T include the following groups.

Examples thereof include a halogen atom (such as a chlorine atom, a bromine atom, or an iodine atom),
an alkyl group [a linear, branched, or cyclic alkyl group, specific examples thereof include a linear or branched alkyl group (preferably a linear or branched alkyl group having 1 to 30 carbon atoms, and examples thereof include a methyl group, an ethyl group, an n-propyl group, an isopropyl group, a t-butyl group, an n-octyl group, an eicosyl group, a 2-chloroethyl group, a 2-cyanoethyl group, and a 2-ethylhexyl group), a cycloalkyl group (preferably a cycloalkyl group having 3 to 30 carbon atoms, and examples thereof include a cyclohexyl group, a cyclopentyl group, and a 4-n-dodecylcyclohexyl group), a bicycloalkyl group (preferably a bicycloalkyl group having 5 to 30 carbon atoms, that is, a monovalent group obtained by removing one hydrogen atom from a bicycloalkane having 5 to 30 carbon atoms, and examples thereof include a bicyclo[1,2,2]heptane-2-yl group and a bicyclo[2,2,2]octane-3-yl group), and those having a tricyclo structure with a plurality of ring structures, and alkyl groups in the substituents described below (for example, an alkyl group in an alkylthio group) are alkyl groups of such a concept],
an alkenyl group [linear, branched, or cyclic alkenyl group, specific examples thereof include a linear or branched alkenyl group (preferably a linear or branched alkenyl group having 2 to 30 carbon atoms, and examples thereof include a vinyl group, an allyl group, a prenyl group, a geranyl group, and an oleyl group), a cycloalkenyl group (preferably a cycloalkenyl group having 3 to 30 carbon atoms, that is, a monovalent group obtained by removing one hydrogen atom from a cycloalkene having 3 to 30 carbon atoms, and examples thereof include a 2-cyclopentene-1-yl group and a 2-cyclohexene-1-yl group), and a bicycloalkenyl group (preferably a bicycloalkenyl group having 5 to 30 carbon atoms, that is, a monovalent group obtained by removing one hydrogen atom from a bicycloalkene having one double bond, and examples thereof include a bicyclo[2,2,1]hepto-2-en-1-yl group and a bicyclo[2,2,2]octo-2-en-4-yl group)],
an alkynyl group (preferably a linear or branched alkynyl group having 2 to 30 carbon atoms, examples thereof include an ethynyl group and a propargyl group),
an aryl group (preferably an aryl group having 6 to 30 carbon atoms, and examples thereof include a phenyl group, a p-tolyl group, a naphthyl group, an m-chlorophenyl group, an o-hexadecanoylaminophenyl group),
a heterocyclic group (preferably a monovalent group obtained by removing one hydrogen atom from a 5- or 6-membered aromatic or non-aromatic heterocyclic compound and more preferably a 5- or 6-membered aromatic heterocyclic group having 3 to 30 carbon atoms, and examples thereof include a 2-furyl group, a 2-thienyl group, a 2-pyrimidinyl group, and a 2-benzothiazolyl group),
a cyano group,
a hydroxy group,
a nitro group;
a carboxy group;
an alkoxy group (preferably a linear or branched alkoxy group having 1 to 30 carbon atoms, and examples thereof include a methoxy group, an ethoxy group, an isopropoxy group, a t-butoxy group, an n-octyloxy group, and a 2-methoxyethoxy group),
an aryloxy group (preferably an aryloxy group having 6 to 30 carbon atoms, and examples thereof include a phenoxy group, a 2-methylphenoxy group, a 4-t-butylphenoxy group, a 3-nitrophenoxy group, and a 2-tetradecanoylaminophenoxy group),
a heterocyclic oxy group (preferably a heterocyclic oxy group having 2 to 30 carbon atoms, and examples thereof include a 1-phenyltetrazole-5-oxy group and a 2-tetrahydropyranyloxy group),
an acyloxy group (preferably a formyloxy group, an alkylcarbonyloxy group having 2 to 30 carbon atoms, or an arylcarbonyloxy group having 6 to 30 carbon atoms, and examples thereof include a formyloxy group, an acetyloxy group, a pivaloyloxy group, a stearoyloxy group, a benzoyloxy group, and a p-methoxyphenylcarbonyloxy group),
a carbamoyloxy group (preferably a carbamoyloxy group having 1 to 30 carbon atoms, and examples thereof include a N,N-dimethylcarbamoyloxy group, a N,N-diethylcarbamoyloxy group, a morpholinocarbonyloxy group, a N,N-di-n-octylaminocarbonyloxy group, and a N-n-octylcarbamoyloxy group),
an alkoxycarbonyloxy group (preferably an alkoxycarbonyloxy group having 2 to 30 carbon atoms, and examples thereof include a methoxycarbonyloxy group, an ethoxycarbonyloxy group, a t-butoxycarbonyloxy group, and an n-octylcarbonyloxy group),
an aryloxycarbonyloxy group (preferably an aryloxycarbonyloxy group having 7 to 30 carbon atoms, and examples thereof include a phenoxycarbonyloxy group, a p-methoxyphenoxycarbonyloxy group, and a p-n-hexadecyloxyphenoxycarbonyloxy group),
an amino group (preferably an amino group, an alkylamino group having 1 to 30 carbon atoms, or an anilino group having 6 to 30 carbon atoms, and examples thereof include an amino group, a methylamino group, a dimethylamino group, an anilino group, a N-methyl-anilino group, and a diphenylamino group),
an acylamino group (preferably a formylamino group, an alkylcarbonylamino group having 2 to 30 carbon atoms, or an arylcarbonylamino group having 6 to 30 carbon atoms, and examples thereof include a formylamino group, an acetylamino group, a pivaloylamino group, a lauroylamino group, a benzoylamino group, and a 3,4,5-tri-n-octyloxyphenylcarbonylamino group),
an aminocarbonylamino group (preferably an aminocarbonylamino group having 1 to carbon atoms, and examples thereof include a carbamoylamino group, a N,N-dimethylaminocarbonylamino group, a N,N-diethylaminocarbonylamino group, and a morpholinocarbonylamino group),
an alkoxycarbonylamino group (preferably an alkoxycarbonylamino group having 2 to 30 carbon atoms, and examples thereof include a methoxycarbonylamino group, an ethoxycarbonylamino group, a t-butoxycarbonylamino group, an n-octadecyloxycarbonylamino group, and a N-methyl-methoxycarbonylamino group),
an aryloxycarbonylamino group (preferably an aryloxycarbonylamino group having 7 to 30 carbon atoms, and examples thereof include a phenoxycarbonylamino group, a p-chlorophenoxycarbonylamino group, and an m-n-octyloxyphenoxycarbonylamino group),
a sulfamoylamino group (preferably a sulfamoylamino group having 0 to 30 carbon atoms, and examples thereof include a sulfamoylamino group, a N,N-dimethylaminosulfonylamino group, and a N-n-octylaminosulfonylamino group),
an alkyl or arylsulfonylamino group (preferably an alkyl sulfonylamino group having 1 to 30 carbon atoms or an arylsulfonylamino group having 6 to 30 carbon atoms, and examples thereof include a methylsulfonylamino group, a butylsulfonylamino group, a phenylsulfonylamino group, a 2,3,5-trichlorophenylsulfonylamino group, and a p-methylphenylsulfonylamino group),
a mercapto group,
an alkylthio group (preferably an alkylthio group having 1 to 30 carbon atoms, and examples thereof include a methylthio group, an ethylthio group, and an n-hexadecylthio group),
an arylthio group (preferably an arylthio group having 6 to 30 carbon atoms, and examples thereof include a phenylthio group, a p-chlorophenylthio group, and an m-methoxyphenylthio group),
a heterocyclic thio group (preferably a heterocyclic thio group having 2 to 30 carbon atoms, and examples thereof include a 2-benzothiazolylthio group and a 1-phenyltetrazole-5-ylthio group),
a sulfamoyl group (preferably a sulfamoyl group having 0 to 30 carbon atoms, and examples thereof include a N-ethylsulfamoyl group, a N-(3-dodecyloxypropyl)sulfamoyl group, a N,N-dimethylsulfamoyl group, a N-acetylsulfamoyl group, a N-benzoylsulfamoyl group, a N—(N′-phenylcarbamoyl)sulfamoyl group),
a sulfo group,
an alkyl or arylsulfinyl group (preferably an alkyl sulfinyl group having 1 to 30 carbon atoms or an arylsulfinyl group having 6 to 30 carbon atoms, and examples thereof include a methylsulfinyl group, an ethylsulfinyl group, a phenylsulfinyl group, and a p-methylphenylsulfinyl group),
an alkyl or arylsulfonyl group (preferably an alkylsulfonyl group having 1 to 30 carbon atoms or an arylsulfonyl group having 6 to 30 carbon atoms, and examples thereof include a methylsulfonyl group, an ethylsulfonyl group, a phenylsulfonyl group, and a p-methylphenylsulfonyl group),
an acyl group (preferably a formyl group, an alkylcarbonyl group having 2 to 30 carbon atoms, an arylcarbonyl group having 7 to 30 carbon atoms, or a heterocyclic carbonyl group having 4 to 30 carbon atoms and bonded to a carbonyl group, and examples include an acetyl group, a pivaloyl group, a 2-chloroacetyl group, a stearoyl group, a benzoyl group, a p-n-octyloxyphenylcarbonyl group, a 2-pyridylcarbonyl group, and a 2-furylcarbonyl group),
an aryloxycarbonyl group (preferably an aryloxycarbonyl group having 7 to 30 carbon atoms, and examples thereof include a phenoxycarbonyl group, an o-chlorophenoxycarbonyl group, an m-nitrophenoxycarbonyl group, and a p-t-butylphenoxycarbonyl group),
an alkoxycarbonyl group (preferably an alkoxycarbonyl group having 2 to 30 carbon atoms, and examples thereof include a methoxycarbonyl group, an ethoxycarbonyl group, a t-butoxycarbonyl group, and an n-octadecyloxycarbonyl group);
a carbamoyl group (preferably a carbamoyl group having 1 to 30 carbon atoms, and examples thereof include a carbamoyl group, a N-methylcarbamoyl group, a N,N-dimethylcarbamoyl group, a N,N-di-n-octylcarbamoyl group, and a N-(methylsulfonyl)carbamoyl group),
an aryl or heterocyclic azo group (preferably an arylazo group having 6 to 30 carbon atoms or a heterocyclic azo group having 3 to 30 carbon atoms, and examples thereof include a phenylazo group, a p-chlorophenylazo group, and a 5-ethylthio-1,3,4-thiadiazole-2-ylazo group),
an imide group (preferably a N-succinimide group or a N-phthalimide group),
a phosphino group (preferably a phosphino group having 2 to 30 carbon atoms, and examples thereof include a dimethylphosphino group, a diphenylphosphino group, and a methylphenoxyphosphino group),
a phosphinyl group (preferably a phosphinyl group having 2 to 30 carbon atoms, and examples thereof include a phosphinyl group, a dioctyloxyphosphinyl group, and a diethoxyphosphinyl group),
a phosphinyloxy group (preferably a phosphinyloxy group having 2 to 30 carbon atoms, and examples thereof include a diphenoxyphosphinyloxy group and a dioctyloxyphosphinyloxy group), and
a phosphinylamino group (preferably a phosphinylamino group having 2 to 30 carbon atoms, and examples thereof include a dimethoxyphosphinylamino group and a dimethylaminophosphinylamino group), and

Among the groups described above, one or more hydrogen atoms of groups having hydrogen atoms may be substituted with the above-described substituents T. Examples of such substituents include an alkylcarbonylaminosulfonyl group, an arylcarbonylaminosulfonyl group, an alkylsulfonylaminocarbonyl group, and an arylsulfonylaminocarbonyl group.

Specific examples include a methylsulfonylaminocarbonyl group, a p-methylphenylsulfonylaminocarbonyl group, an acetylaminosulfonyl group, and a benzoylaminosulfonyl group.

Specific examples of the specific compound include compounds having the following structures. In the structural formulae shown below, Et is an ethyl group, Me is a methyl group, ⁿBu is an n-butyl group, ^{t}Bu is a tert-butyl group, and Ph is a phenyl group.

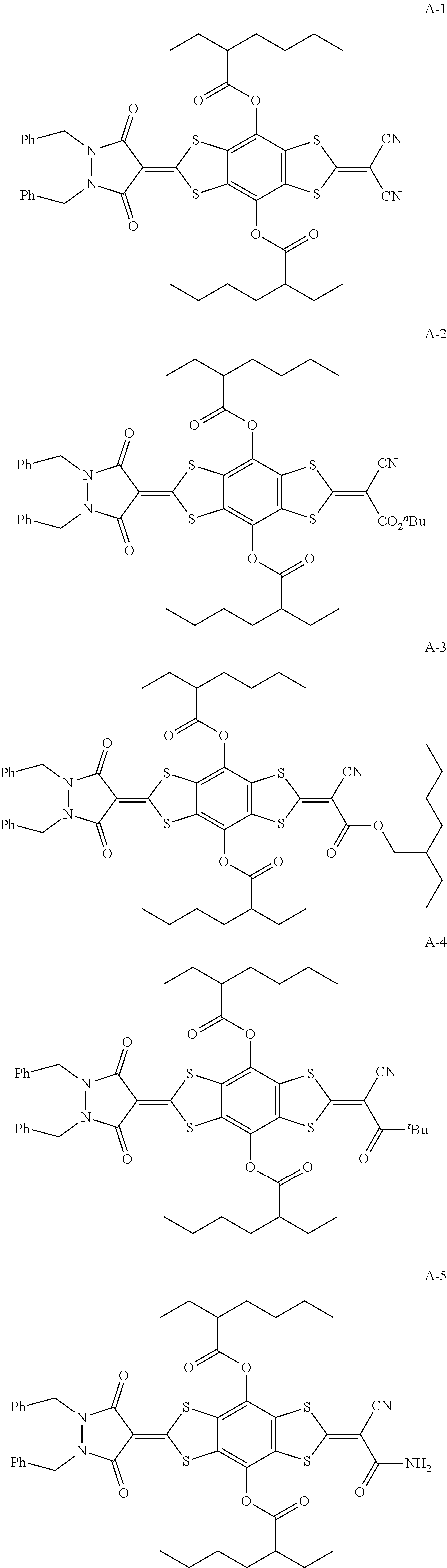

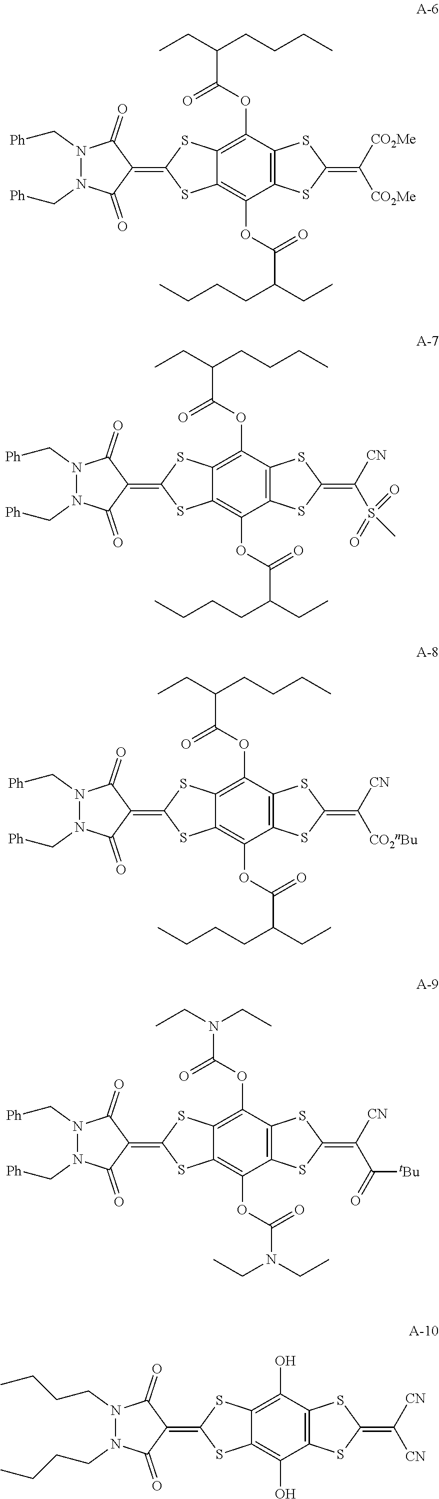

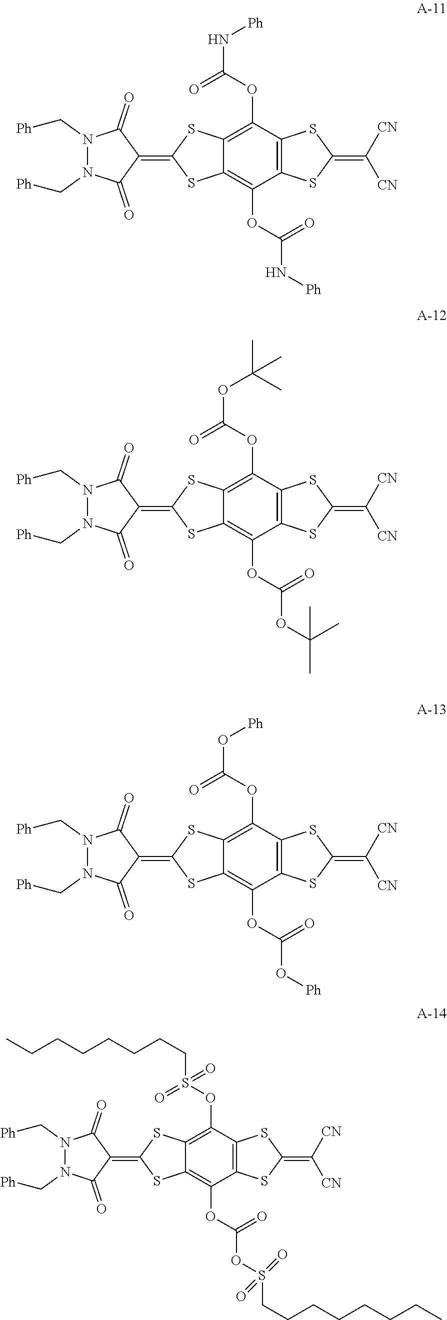 [SMILES: N#CC(C#N)=C1Sc2c(OC(=O)Nc3ccccc3)c3c(c(OC(=O)Nc4ccccc4)c2S1)SC(=C1C(=O)N(Cc2ccccc2)N(Cc2ccccc2)C1=O)S3 ; CC(C)(C)OC(=O)Oc1c2c(c(OC(=O)OC(C)(C)C)c3c1SC(=C1C(=O)N(Cc4ccccc4)N(Cc4ccccc4)C1=O)S3)SC(=C(C#N)C#N)S2 ; N#CC(C#N)=C1Sc2c(OC(=O)Oc3ccccc3)c3c(c(OC(=O)Oc4ccccc4)c2S1)SC(=C1C(=O)N(Cc2ccccc2)N(Cc2ccccc2)C1=O)S3 ; CCCCCCCCS(=O)(=O)OC(=O)Oc1c2c(c(OS(=O)(=O)CCCCCCCC)c3c1SC(=C1C(=O)N(Cc4ccccc4)N(Cc4ccccc4)C1=O)S3)SC(=C(C#N)C#N)S2]

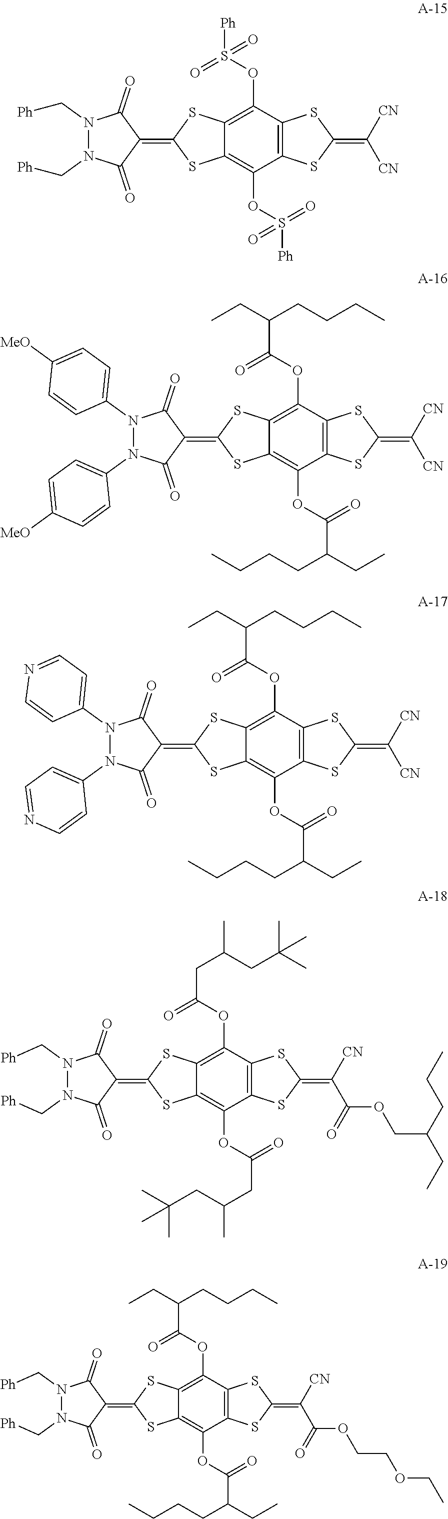

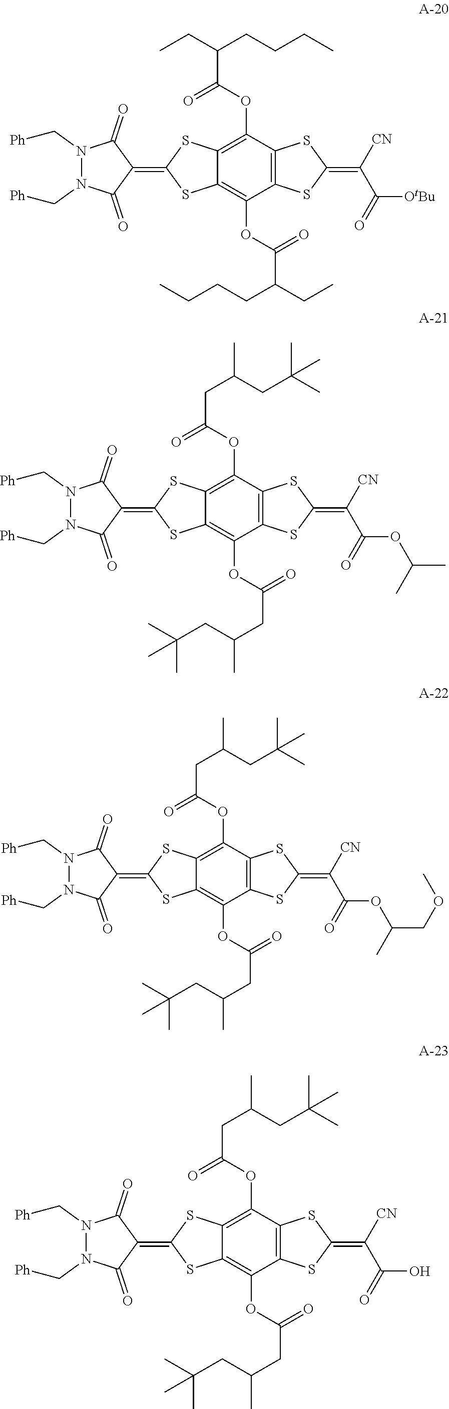

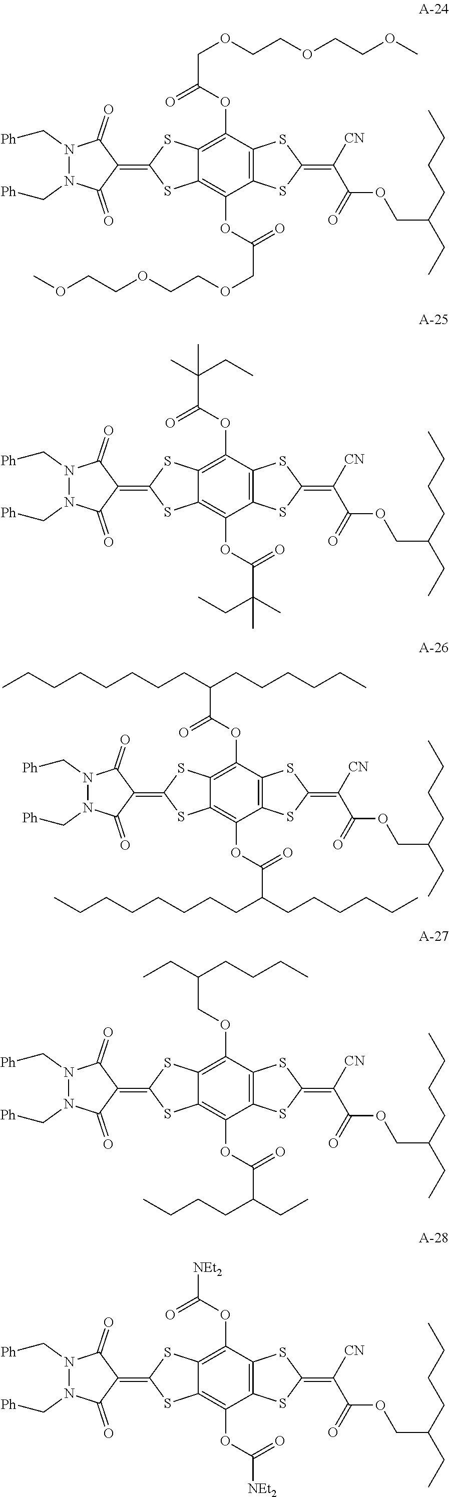

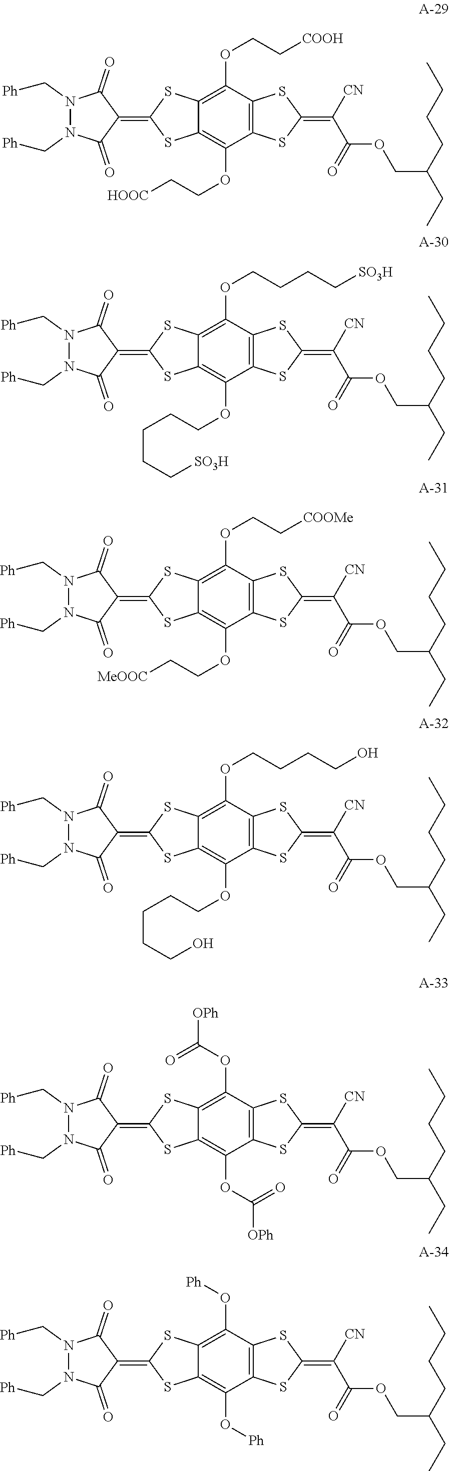

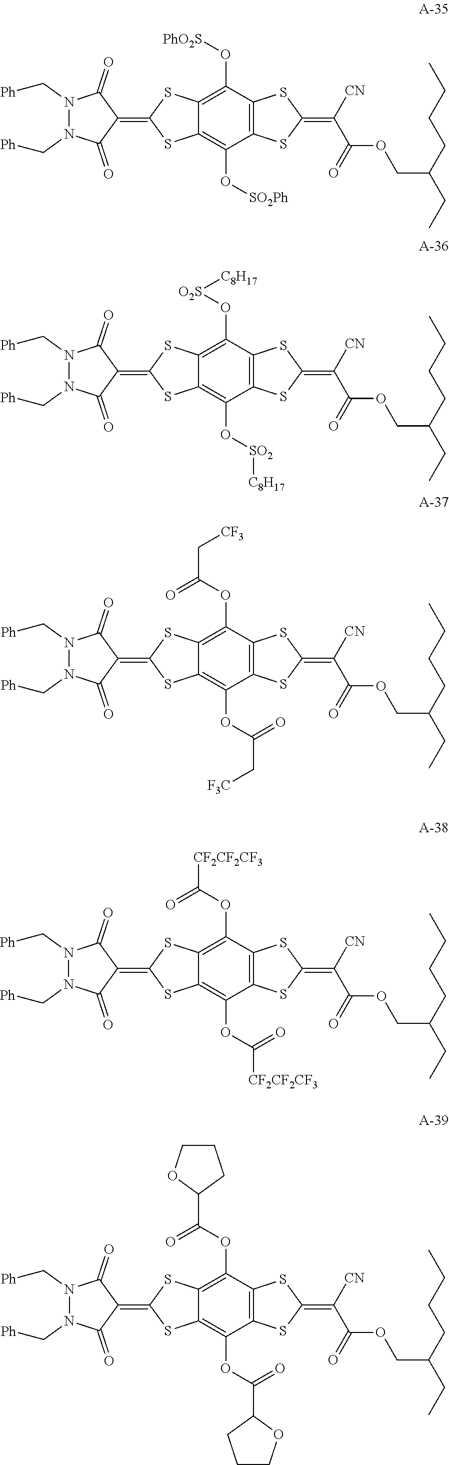

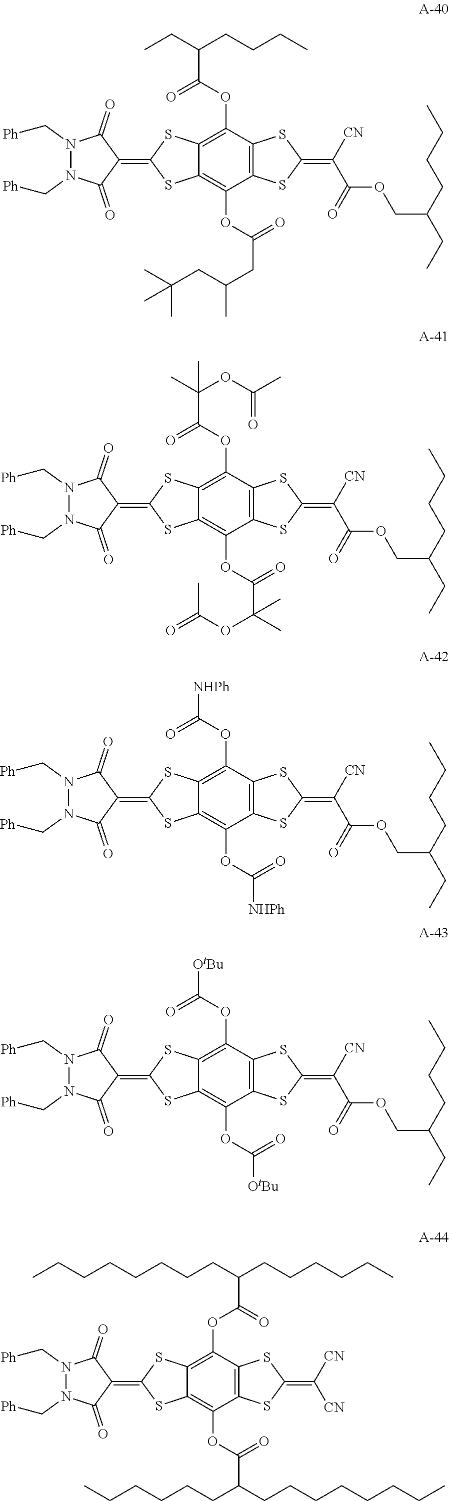

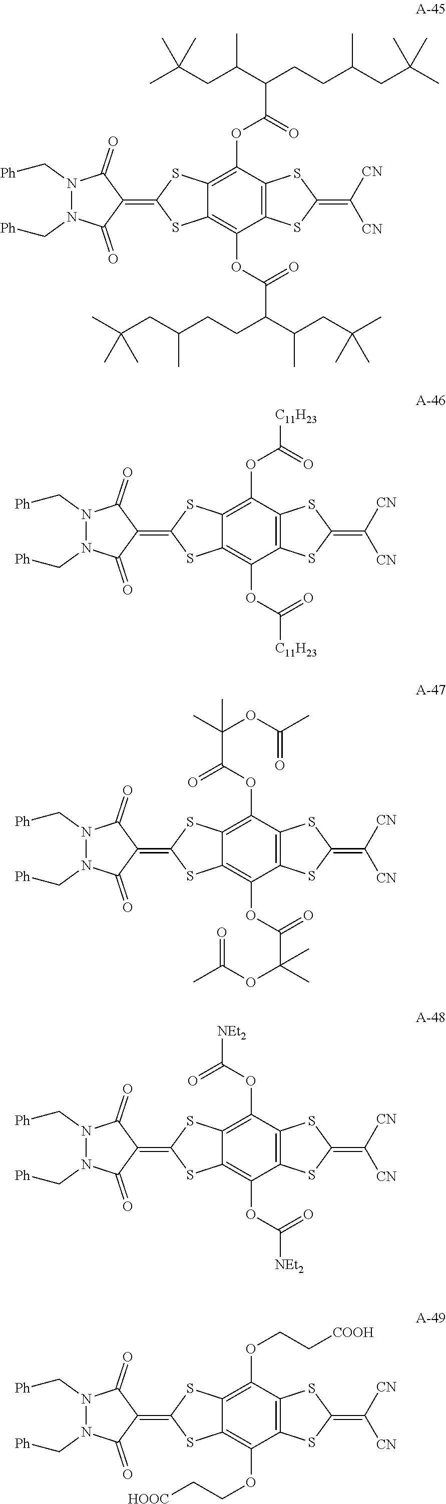

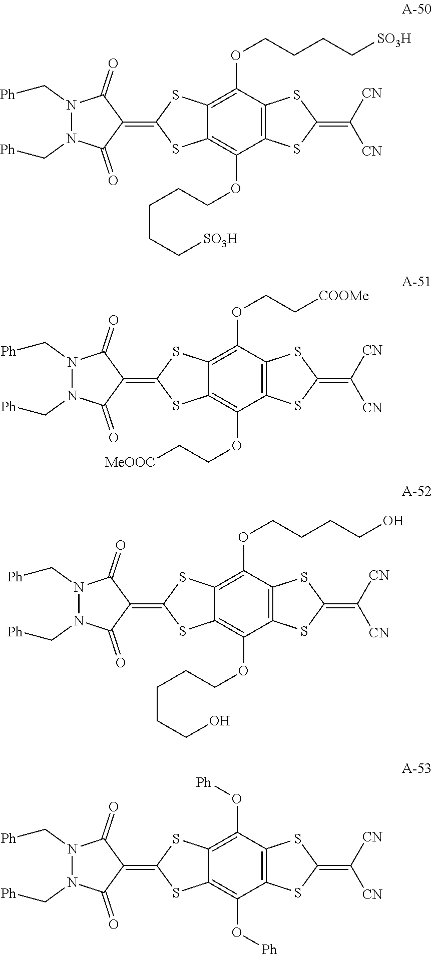 [SMILES: N#CC(C#N)=C1Sc2c(OCCCCCS(=O)(=O)O)c3c(c(OCCCCS(=O)(=O)O)c2S1)SC(=C1C(=O)N(Cc2ccccc2)N(Cc2ccccc2)C1=O)S3 ; COC(=O)CCOc1c2c(c(OCCC(=O)OC)c3c1SC(=C1C(=O)N(Cc4ccccc4)N(Cc4ccccc4)C1=O)S3)SC(=C(C#N)C#N)S2 ; N#CC(C#N)=C1Sc2c(OCCCCO)c3c(c(OCCCCCO)c2S1)SC(=C1C(=O)N(Cc2ccccc2)N(Cc2ccccc2)C1=O)S3 ; N#CC(C#N)=C1Sc2c(Oc3ccccc3)c3c(c(Oc4ccccc4)c2S1)SC(=C1C(=O)N(Cc2ccccc2)N(Cc2ccccc2)C1=O)S3]

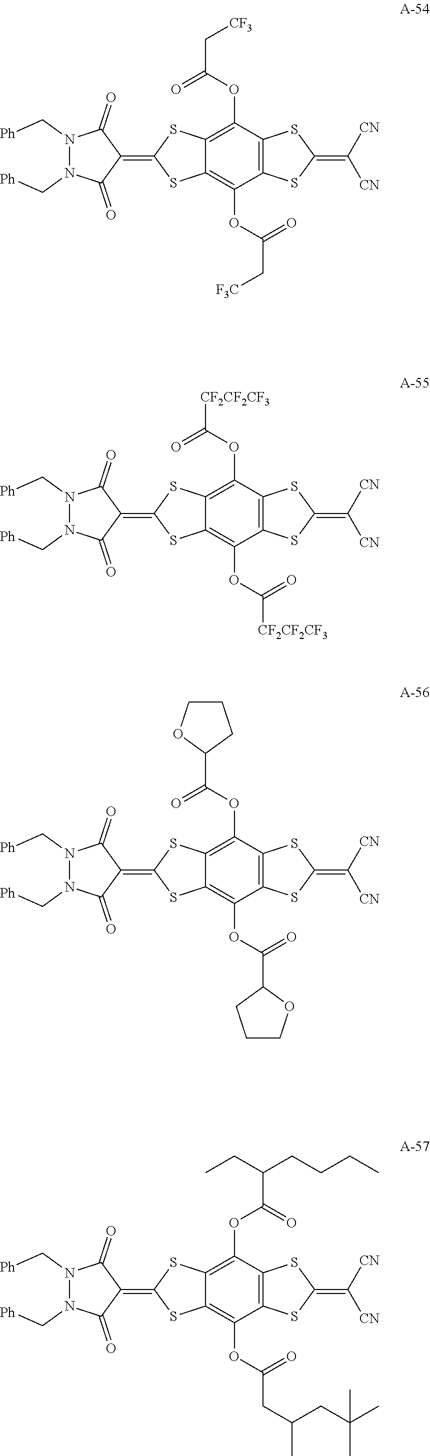

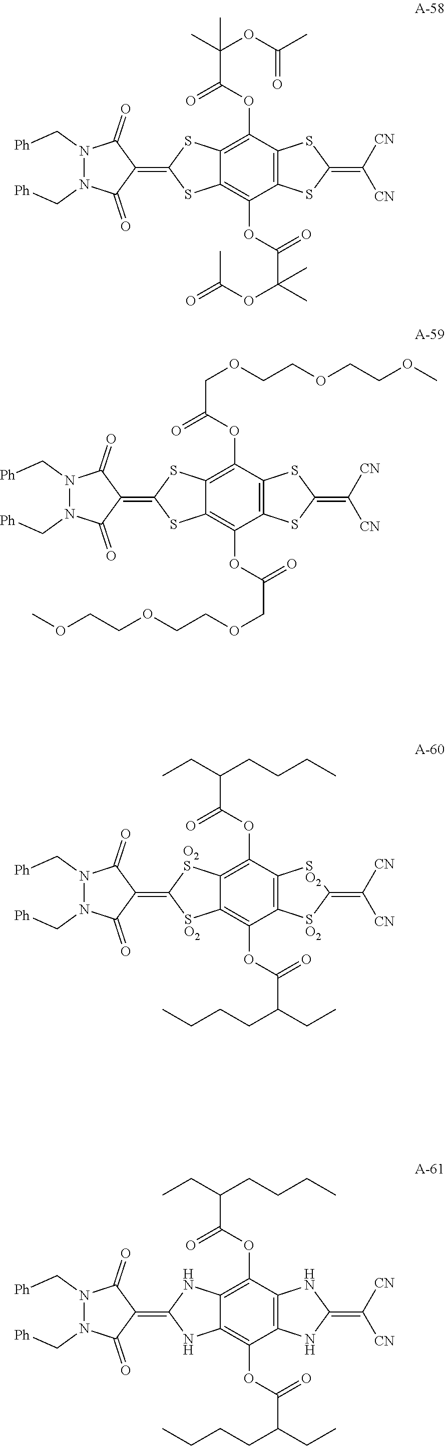 [SMILES: CC(=O)OC(C)(C)C(=O)Oc1c2c(c(OC(=O)C(C)(C)OC(C)=O)c3c1SC(=C1C(=O)N(Cc4ccccc4)N(Cc4ccccc4)C1=O)S3)SC(=C(C#N)C#N)S2 ; COCCOCCOCC(=O)Oc1c2c(c(OC(=O)COCCOCCOC)c3c1SC(=C1C(=O)N(Cc4ccccc4)N(Cc4ccccc4)C1=O)S3)SC(=C(C#N)C#N)S2 ; CCCCC(CC)C(=O)Oc1c2c(c(OC(=O)C(CC)CCCC)c3c1S(=O)(=O)C(=C1C(=O)N(Cc4ccccc4)N(Cc4ccccc4)C1=O)S3(=O)=O)S(=O)(=O)C(=C(C#N)C#N)S2(=O)=O ; CCCCC(CC)C(=O)Oc1c2c(c(OC(=O)C(CC)CCCC)c3c1NC(=C1C(=O)N(Cc4ccccc4)N(Cc4ccccc4)C1=O)N3)NC(=C(C#N)C#N)N2]

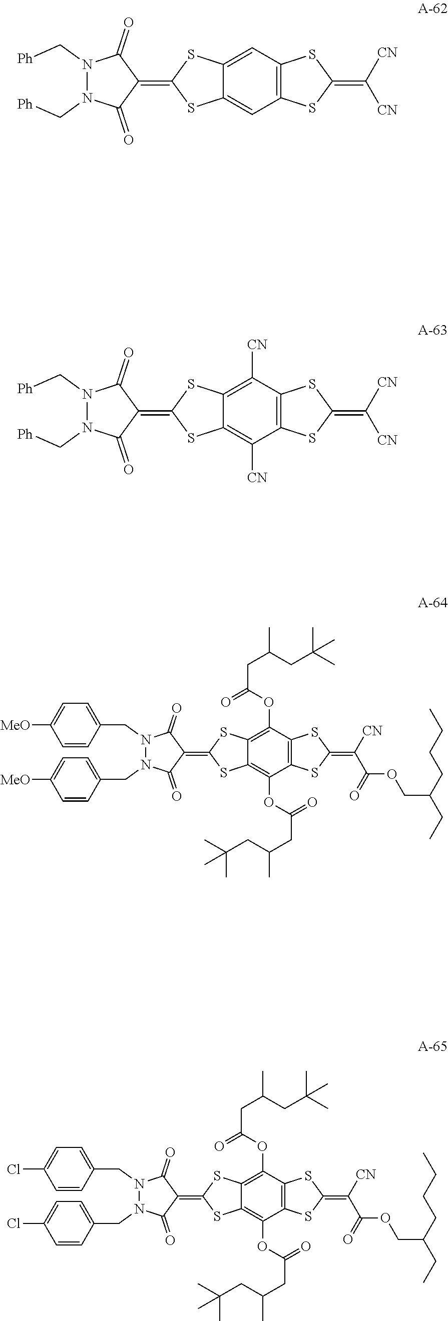 [SMILES: N#CC(C#N)=C1Sc2cc3c(cc2S1)SC(=C1C(=O)N(Cc2ccccc2)N(Cc2ccccc2)C1=O)S3 ; N#CC(C#N)=C1Sc2c(C#N)c3c(c(C#N)c2S1)SC(=C1C(=O)N(Cc2ccccc2)N(Cc2ccccc2)C1=O)S3 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccc(OC)cc2)N(Cc2ccc(OC)cc2)C1=O)S3 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccc(Cl)cc2)N(Cc2ccc(Cl)cc2)C1=O)S3]

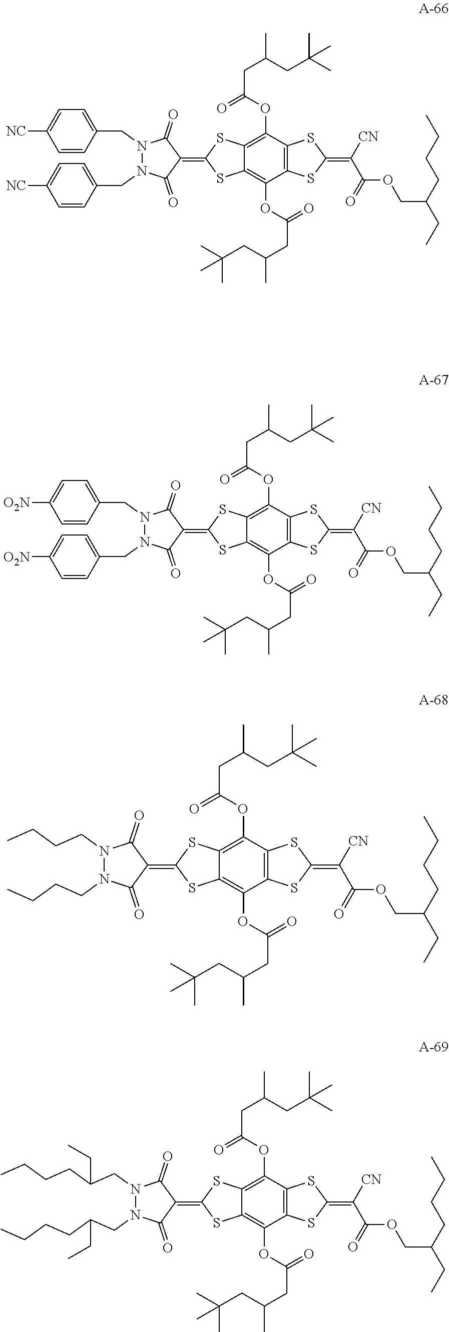

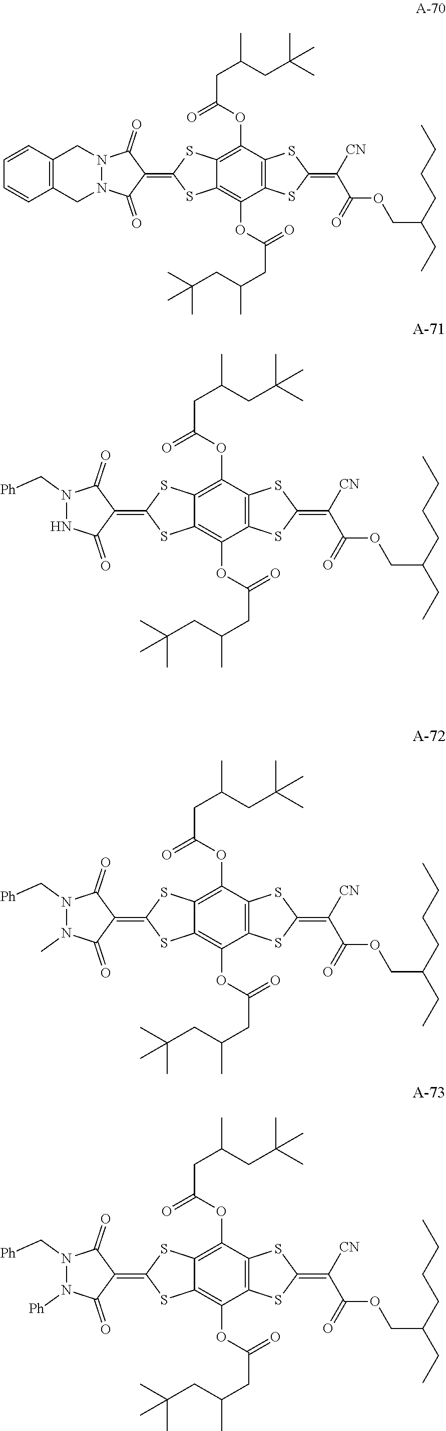

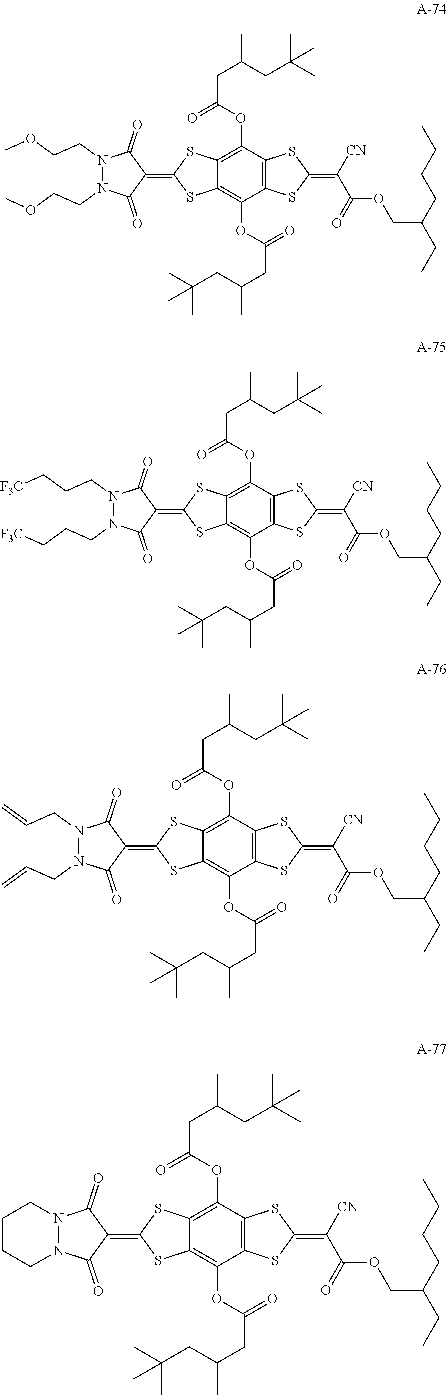 [SMILES: CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(CCOC)N(CCOC)C1=O)S3 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(CCCC(F)(F)F)N(CCCC(F)(F)F)C1=O)S3 ; C=CCN1C(=O)C(=C2Sc3c(OC(=O)CC(C)CC(C)(C)C)c4c(c(OC(=O)CC(C)CC(C)(C)C)c3S2)SC(=C(C#N)C(=O)OCC(CC)CCCC)S4)C(=O)N1CC=C ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N2CCCCN2C1=O)S3]

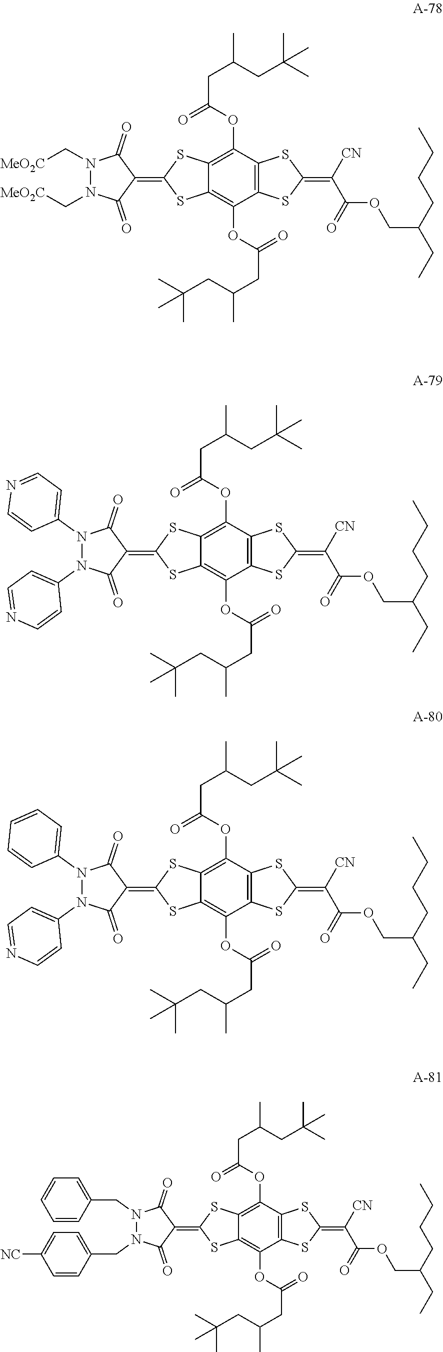

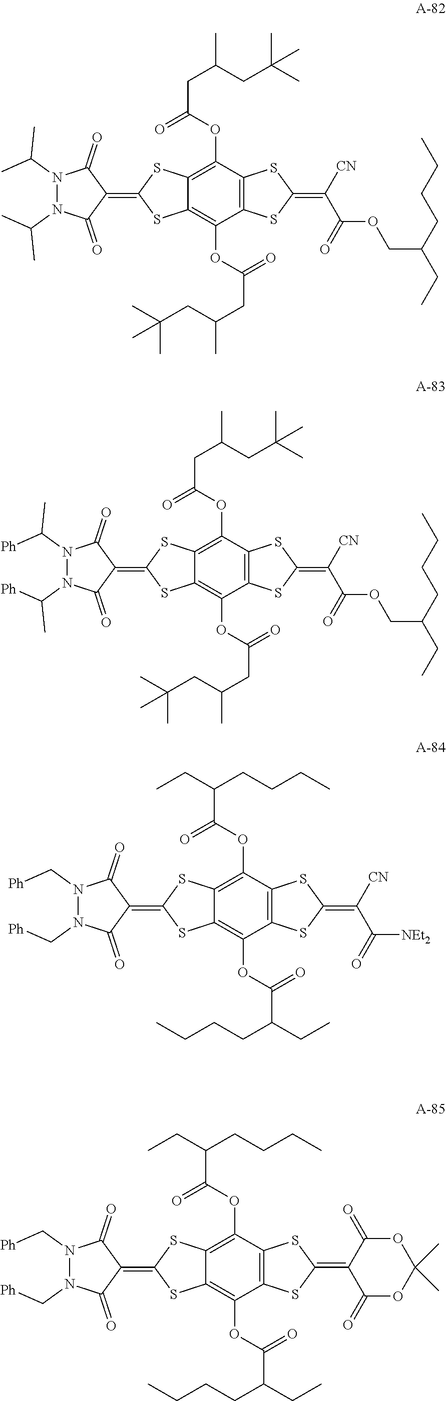

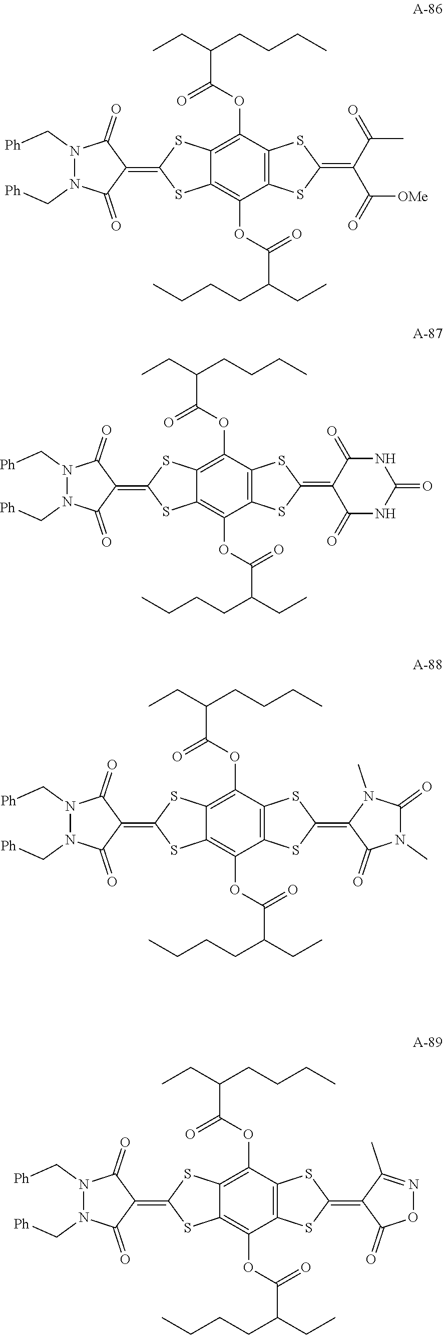 [SMILES: CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C(C(C)=O)C(=O)OC)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C1C(=O)NC(=O)NC1=O)S2 ; CCCCC(CC)C(=O)Oc1c2c(c(OC(=O)C(CC)CCCC)c3c1SC(=C1C(=O)N(C)C(=O)N1C)S3)SC(=C1C(=O)N(Cc3ccccc3)N(Cc3ccccc3)C1=O)S2 ; CCCCC(CC)C(=O)Oc1c2c(c(OC(=O)C(CC)CCCC)c3c1SC(=C1C(=O)ON=C1C)S3)SC(=C1C(=O)N(Cc3ccccc3)N(Cc3ccccc3)C1=O)S2]

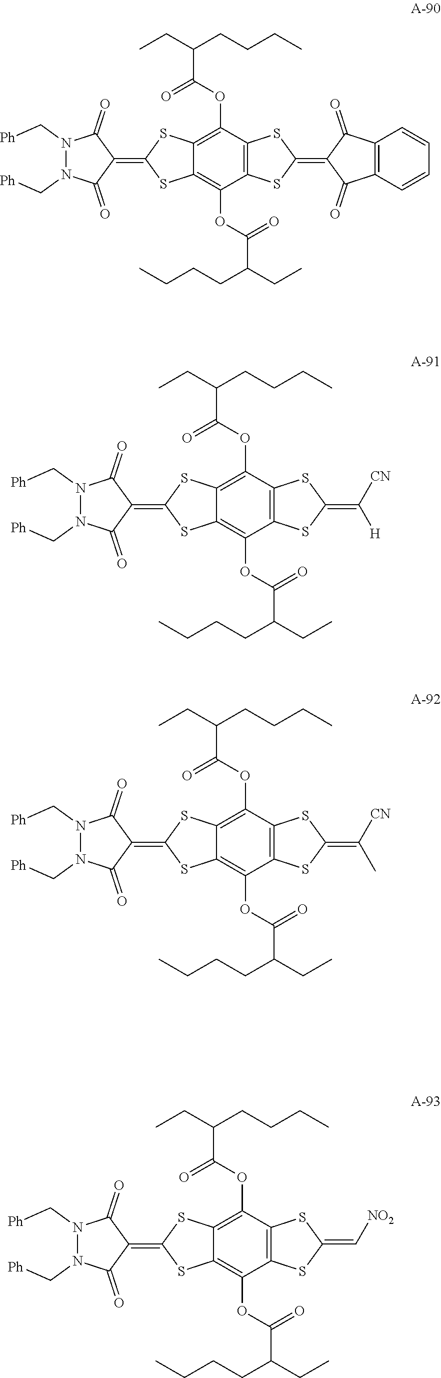 [SMILES: CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C1C(=O)c3ccccc3C1=O)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=CC#N)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C(C)C#N)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C[N+](=O)[O-])S2]

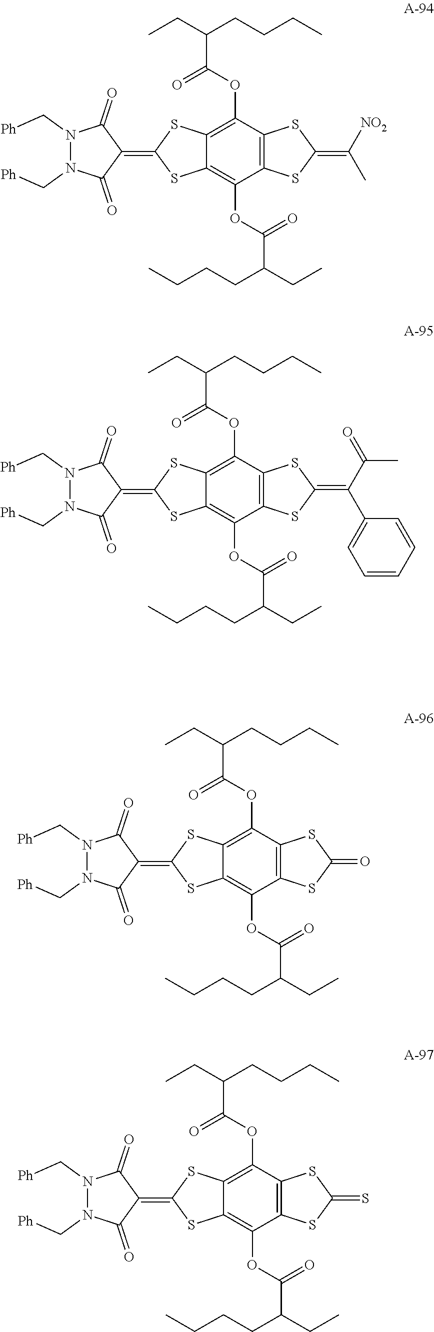 [SMILES: CCCCC(CC)C(=O)Oc1c2c(c(OC(=O)C(CC)CCCC)c3c1SC(=C(C)[N+](=O)[O-])S3)SC(=C1C(=O)N(Cc3ccccc3)N(Cc3ccccc3)C1=O)S2 ; CCCCC(CC)C(=O)Oc1c2c(c(OC(=O)C(CC)CCCC)c3c1SC(=C(C(C)=O)c1ccccc1)S3)SC(=C1C(=O)N(Cc3ccccc3)N(Cc3ccccc3)C1=O)S2 ; CCCCC(CC)C(=O)Oc1c2c(c(OC(=O)C(CC)CCCC)c3sc(=O)sc13)SC(=C1C(=O)N(Cc3ccccc3)N(Cc3ccccc3)C1=O)S2 ; CCCCC(CC)C(=O)Oc1c2c(c(OC(=O)C(CC)CCCC)c3sc(=S)sc13)SC(=C1C(=O)N(Cc3ccccc3)N(Cc3ccccc3)C1=O)S2]

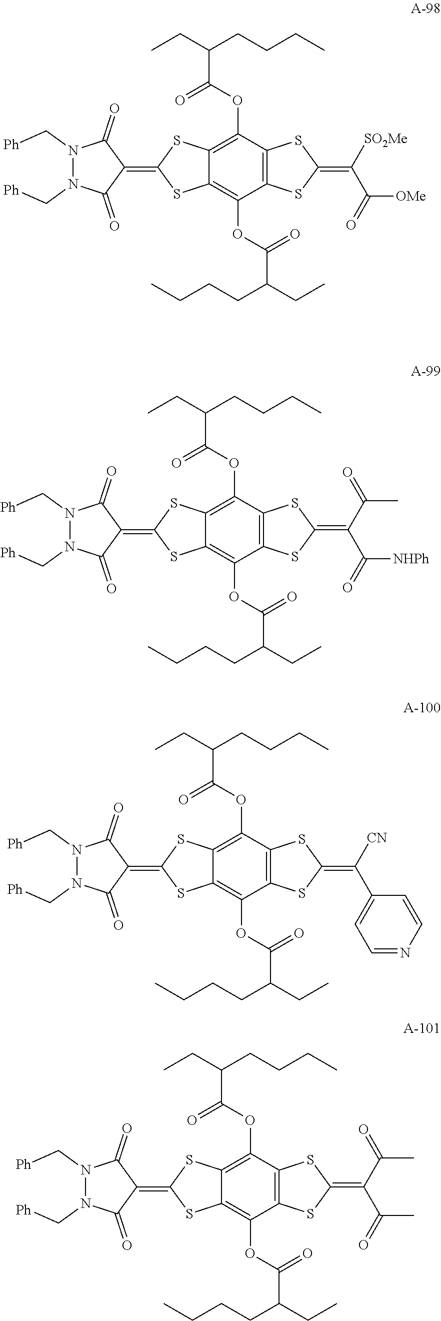

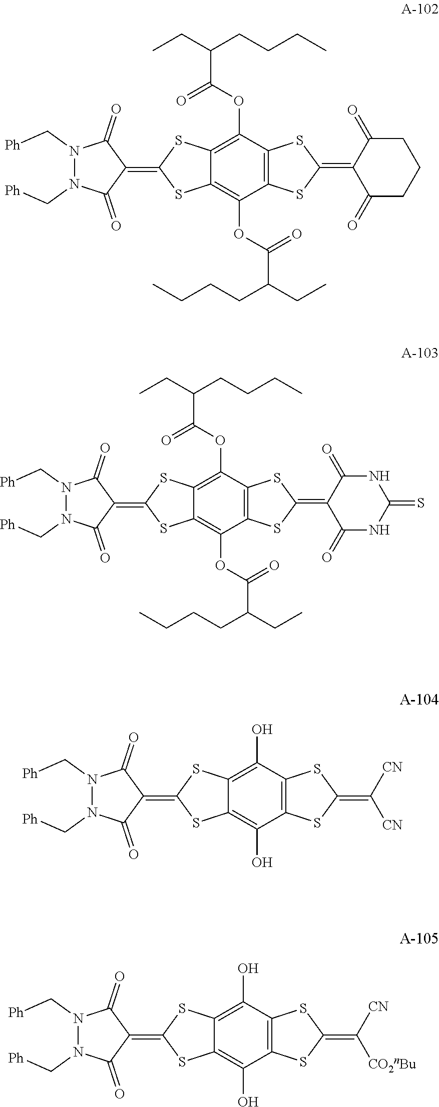

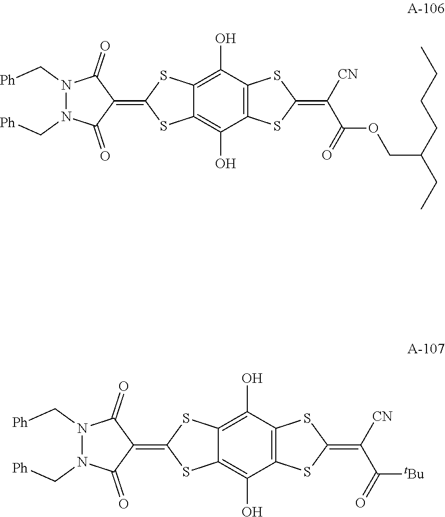 [SMILES: CCCCC(CC)COC(=O)C(C#N)=C1Sc2c(O)c3c(c(O)c2S1)SC(=C1C(=O)N(Cc2ccccc2)N(Cc2ccccc2)C1=O)S3 ; CC(C)(C)C(=O)C(C#N)=C1Sc2c(O)c3c(c(O)c2S1)SC(=C1C(=O)N(Cc2ccccc2)N(Cc2ccccc2)C1=O)S3]

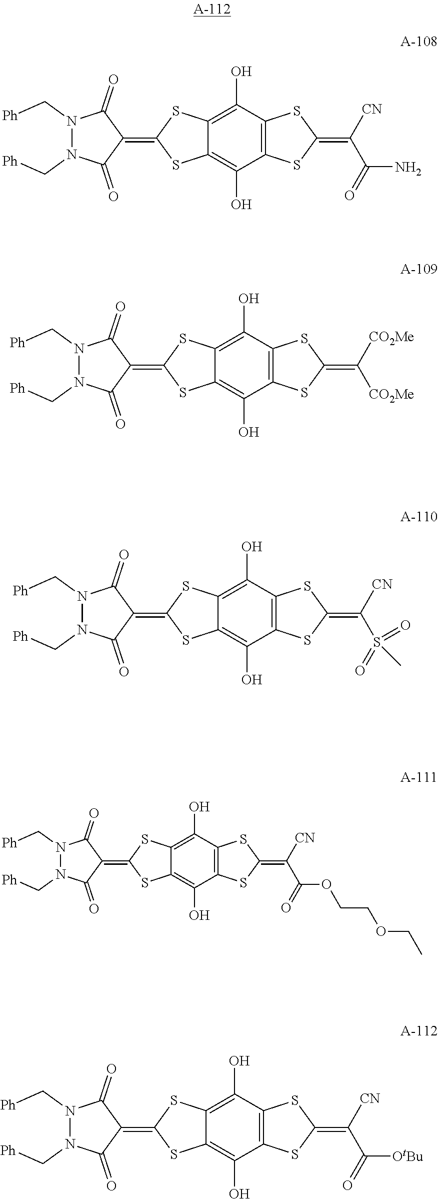 [SMILES: N#CC(C(N)=O)=C1Sc2c(O)c3c(c(O)c2S1)SC(=C1C(=O)N(Cc2ccccc2)N(Cc2ccccc2)C1=O)S3 ; COC(=O)C(C(=O)OC)=C1Sc2c(O)c3c(c(O)c2S1)SC(=C1C(=O)N(Cc2ccccc2)N(Cc2ccccc2)C1=O)S3 ; CS(=O)(=O)C(C#N)=C1Sc2c(O)c3c(c(O)c2S1)SC(=C1C(=O)N(Cc2ccccc2)N(Cc2ccccc2)C1=O)S3 ; CCOCCOC(=O)C(C#N)=C1Sc2c(O)c3c(c(O)c2S1)SC(=C1C(=O)N(Cc2ccccc2)N(Cc2ccccc2)C1=O)S3 ; CC(C)(C)OC(=O)C(C#N)=C1Sc2c(O)c3c(c(O)c2S1)SC(=C1C(=O)N(Cc2ccccc2)N(Cc2ccccc2)C1=O)S3]

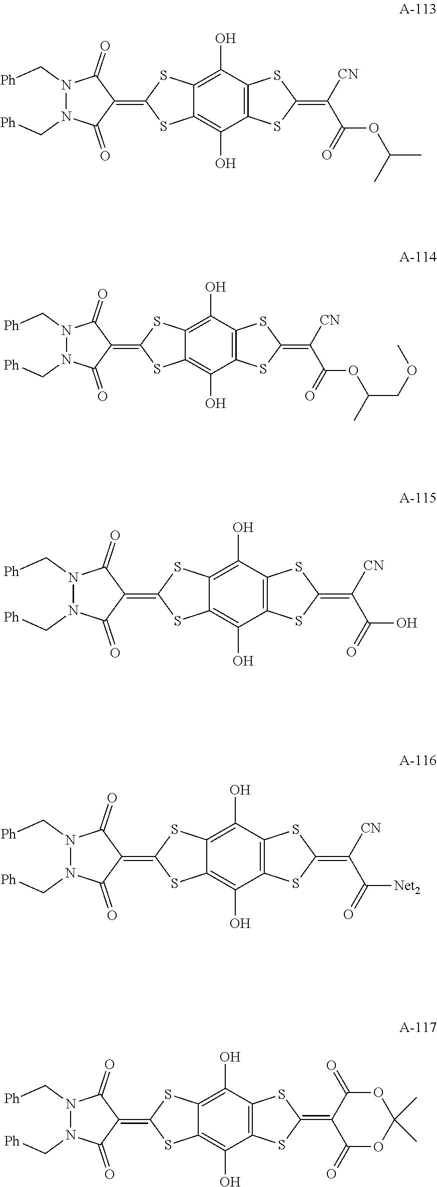

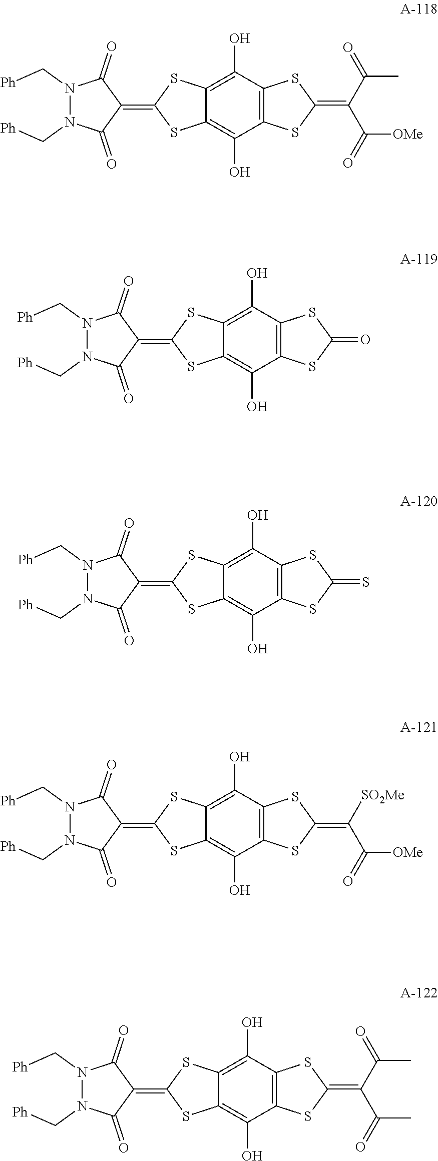 [SMILES: COC(=O)C(C(C)=O)=C1Sc2c(O)c3c(c(O)c2S1)SC(=C1C(=O)N(Cc2ccccc2)N(Cc2ccccc2)C1=O)S3 ; O=C1C(=C2Sc3c(c(O)c4sc(=O)sc4c3O)S2)C(=O)N(Cc2ccccc2)N1Cc1ccccc1 ; O=C1C(=C2Sc3c(c(O)c4sc(=S)sc4c3O)S2)C(=O)N(Cc2ccccc2)N1Cc1ccccc1 ; COC(=O)C(=C1Sc2c(O)c3c(c(O)c2S1)SC(=C1C(=O)N(Cc2ccccc2)N(Cc2ccccc2)C1=O)S3)S(C)(=O)=O ; CC(=O)C(C(C)=O)=C1Sc2c(O)c3c(c(O)c2S1)SC(=C1C(=O)N(Cc2ccccc2)N(Cc2ccccc2)C1=O)S3]

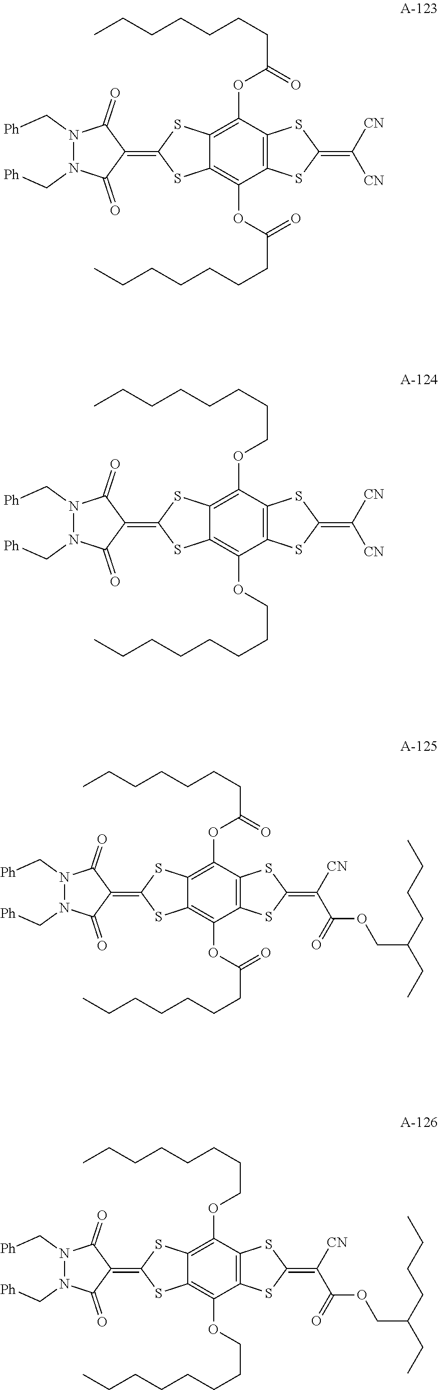 [SMILES: CCCCCCCC(=O)Oc1c2c(c(OC(=O)CCCCCCC)c3c1SC(=C1C(=O)N(Cc4ccccc4)N(Cc4ccccc4)C1=O)S3)SC(=C(C#N)C#N)S2 ; CCCCCCCCOc1c2c(c(OCCCCCCCC)c3c1SC(=C1C(=O)N(Cc4ccccc4)N(Cc4ccccc4)C1=O)S3)SC(=C(C#N)C#N)S2 ; CCCCCCCC(=O)Oc1c2c(c(OC(=O)CCCCCCC)c3c1SC(=C1C(=O)N(Cc4ccccc4)N(Cc4ccccc4)C1=O)S3)SC(=C(C#N)C(=O)OCC(CC)CCCC)S2 ; CCCCCCCCOc1c2c(c(OCCCCCCCC)c3c1SC(=C1C(=O)N(Cc4ccccc4)N(Cc4ccccc4)C1=O)S3)SC(=C(C#N)C(=O)OCC(CC)CCCC)S2]

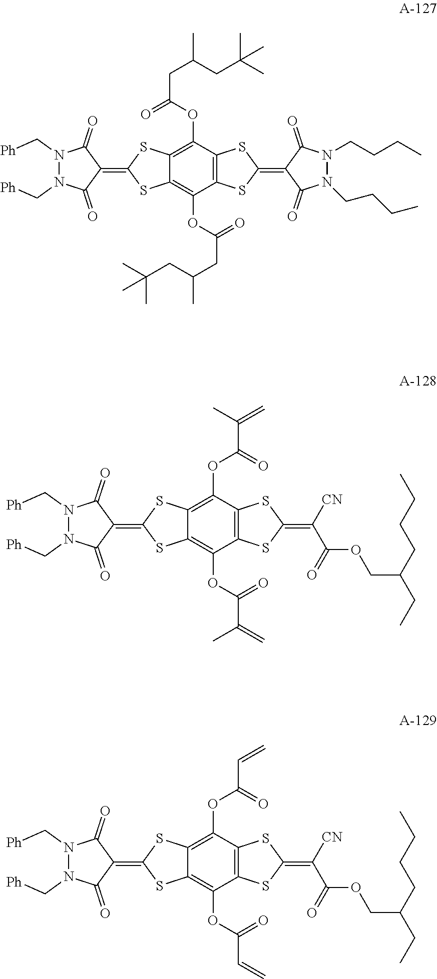 [SMILES: CCCCN1C(=O)C(=C2Sc3c(OC(=O)CC(C)CC(C)(C)C)c4c(c(OC(=O)CC(C)CC(C)(C)C)c3S2)SC(=C2C(=O)N(Cc3ccccc3)N(Cc3ccccc3)C2=O)S4)C(=O)N1CCCC ; C=C(C)C(=O)Oc1c2c(c(OC(=O)C(=C)C)c3c1SC(=C1C(=O)N(Cc4ccccc4)N(Cc4ccccc4)C1=O)S3)SC(=C(C#N)C(=O)OCC(CC)CCCC)S2 ; C=CC(=O)Oc1c2c(c(OC(=O)C=C)c3c1SC(=C1C(=O)N(Cc4ccccc4)N(Cc4ccccc4)C1=O)S3)SC(=C(C#N)C(=O)OCC(CC)CCCC)S2]

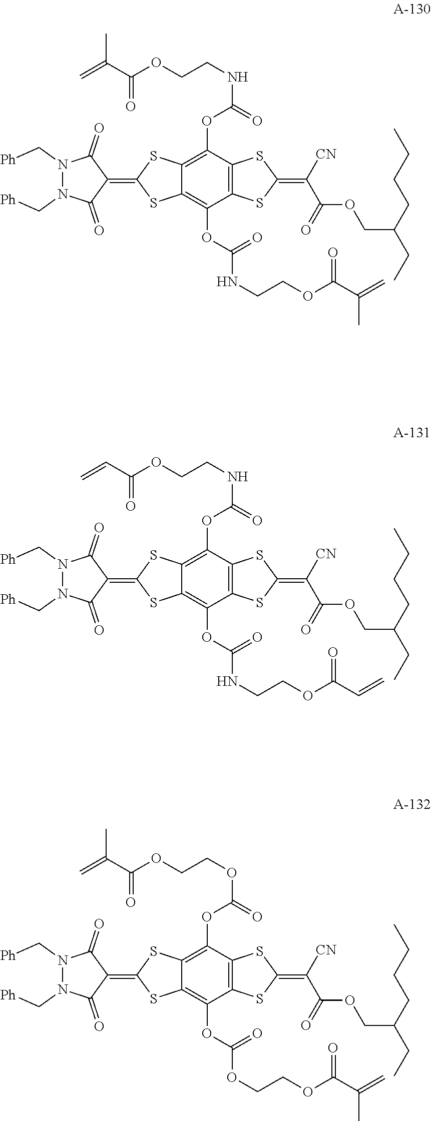 [SMILES: C=C(C)C(=O)OCCNC(=O)Oc1c2c(c(OC(=O)NCCOC(=O)C(=C)C)c3c1SC(=C1C(=O)N(Cc4ccccc4)N(Cc4ccccc4)C1=O)S3)SC(=C(C#N)C(=O)OCC(CC)CCCC)S2 ; C=CC(=O)OCCNC(=O)Oc1c2c(c(OC(=O)NCCOC(=O)C=C)c3c1SC(=C1C(=O)N(Cc4ccccc4)N(Cc4ccccc4)C1=O)S3)SC(=C(C#N)C(=O)OCC(CC)CCCC)S2 ; C=C(C)C(=O)OCCOC(=O)Oc1c2c(c(OC(=O)OCCOC(=O)C(=C)C)c3c1SC(=C1C(=O)N(Cc4ccccc4)N(Cc4ccccc4)C1=O)S3)SC(=C(C#N)C(=O)OCC(CC)CCCC)S2]

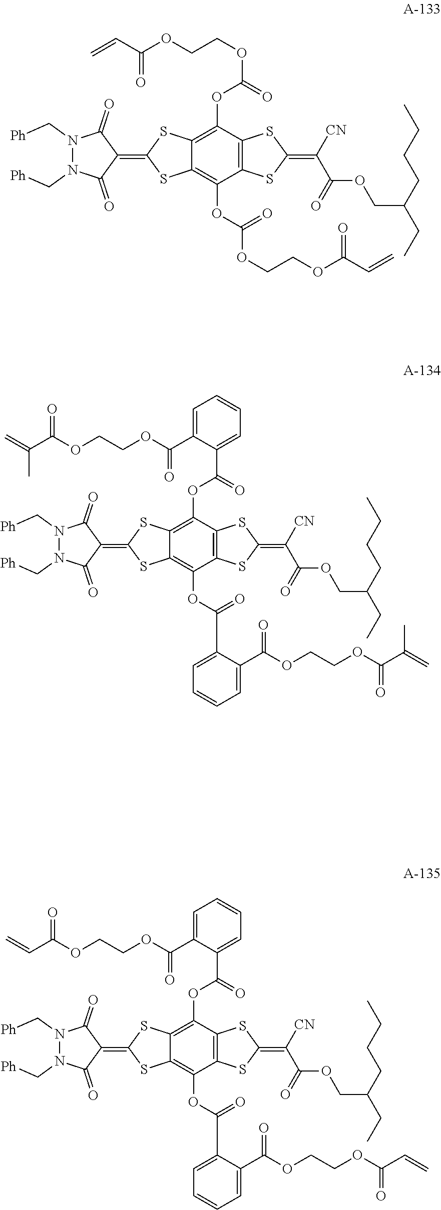

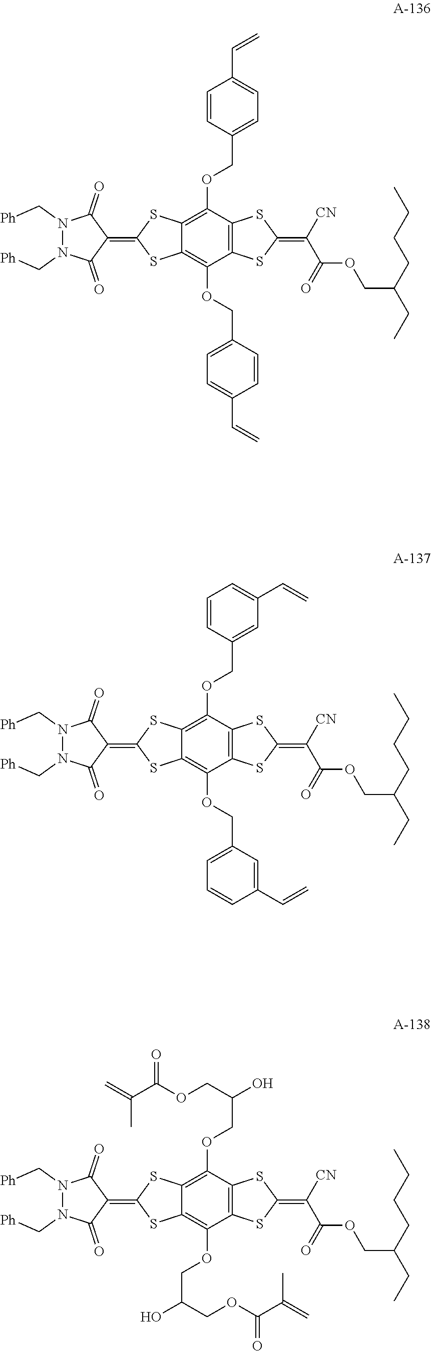 [SMILES: C=Cc1ccc(COc2c3c(c(OCc4ccc(C=C)cc4)c4c2SC(=C2C(=O)N(Cc5ccccc5)N(Cc5ccccc5)C2=O)S4)SC(=C(C#N)C(=O)OCC(CC)CCCC)S3)cc1 ; C=Cc1cccc(COc2c3c(c(OCc4cccc(C=C)c4)c4c2SC(=C2C(=O)N(Cc5ccccc5)N(Cc5ccccc5)C2=O)S4)SC(=C(C#N)C(=O)OCC(CC)CCCC)S3)c1 ; C=C(C)C(=O)OCC(O)COc1c2c(c(OCC(O)COC(=O)C(=C)C)c3c1SC(=C1C(=O)N(Cc4ccccc4)N(Cc4ccccc4)C1=O)S3)SC(=C(C#N)C(=O)OCC(CC)CCCC)S2]

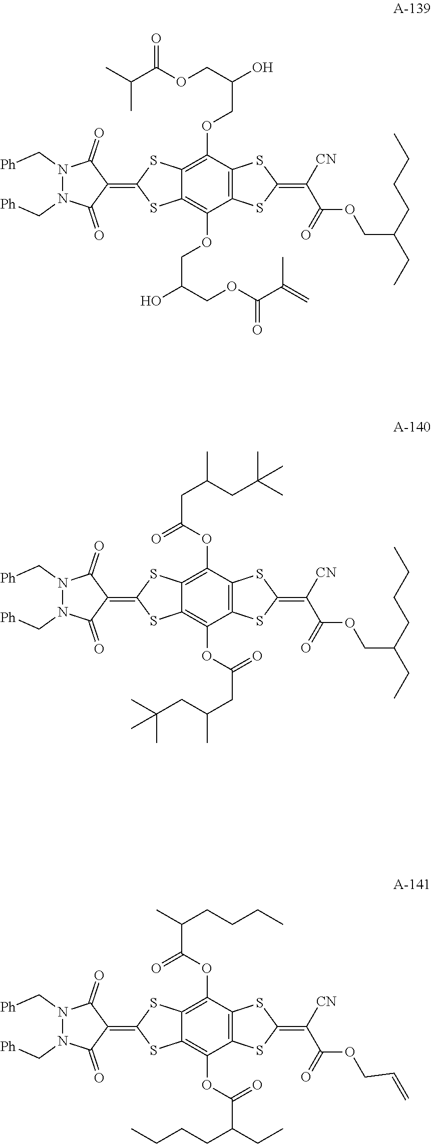 [SMILES: C=C(C)C(=O)OCC(O)COc1c2c(c(OCC(O)COC(=O)C(C)C)c3c1S/C(=C(/C#N)C(=O)OCC(CC)CCCC)S3)SC(=C1C(=O)N(Cc3ccccc3)N(Cc3ccccc3)C1=O)S2 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3 ; C=CCOC(=O)/C(C#N)=C1/Sc2c(OC(=O)C(C)CCCC)c3c(c(OC(=O)C(CC)CCCC)c2S1)SC(=C1C(=O)N(Cc2ccccc2)N(Cc2ccccc2)C1=O)S3]

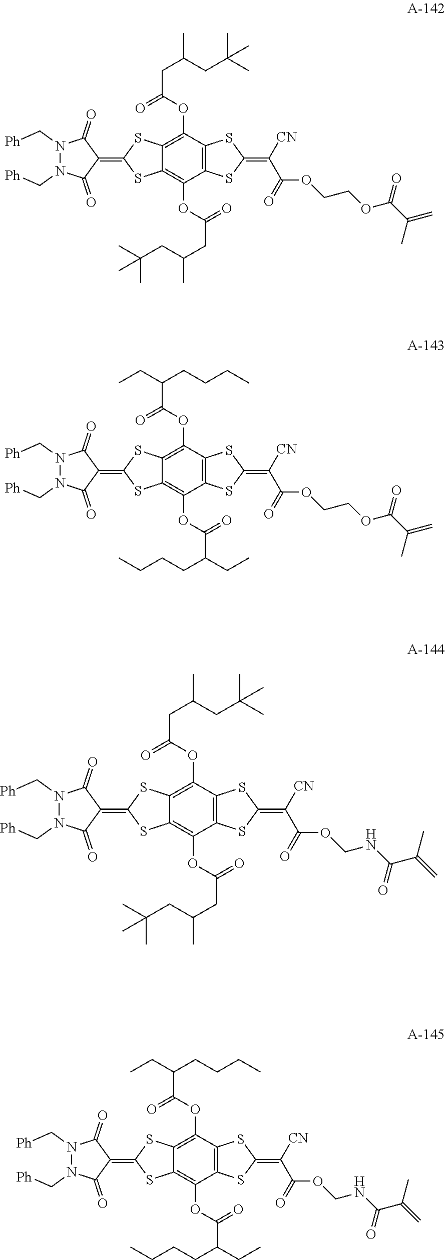

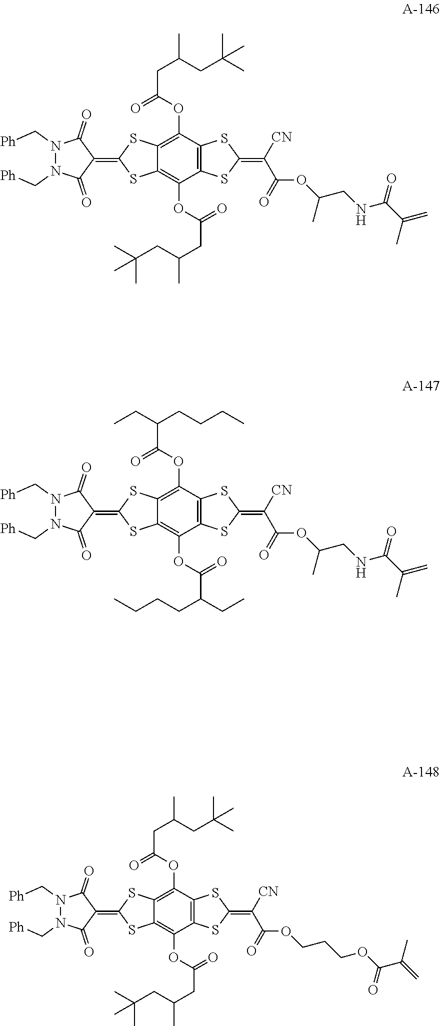 [SMILES: C=C(C)C(=O)NCC(C)OC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3 ; C=C(C)C(=O)NCC(C)OC(=O)C(C#N)=C1Sc2c(OC(=O)C(CC)CCCC)c3c(c(OC(=O)C(CC)CCCC)c2S1)SC(=C1C(=O)N(Cc2ccccc2)N(Cc2ccccc2)C1=O)S3 ; C=C(C)C(=O)OCCCOC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3]

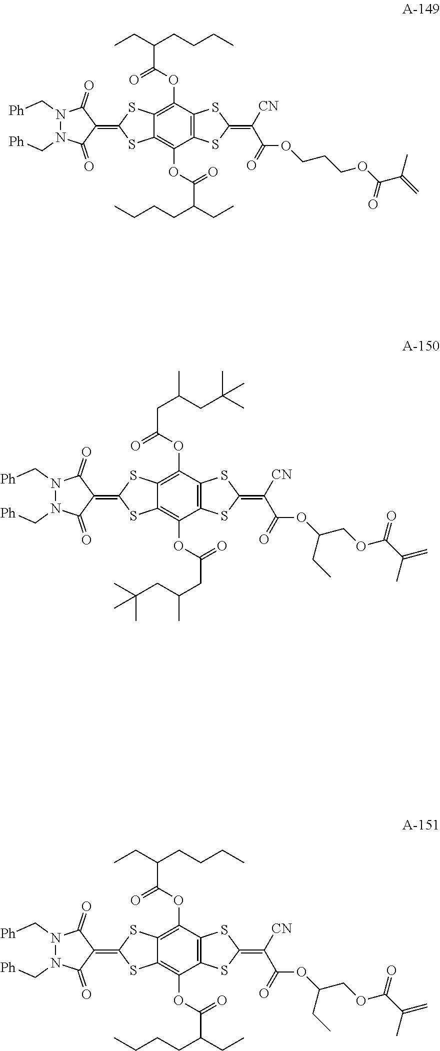 [SMILES: C=C(C)C(=O)OCCCOC(=O)C(C#N)=C1Sc2c(OC(=O)C(CC)CCCC)c3c(c(OC(=O)C(CC)CCCC)c2S1)SC(=C1C(=O)N(Cc2ccccc2)N(Cc2ccccc2)C1=O)S3 ; C=C(C)C(=O)OCC(CC)OC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3 ; C=C(C)C(=O)OCC(CC)OC(=O)C(C#N)=C1Sc2c(OC(=O)C(CC)CCCC)c3c(c(OC(=O)C(CC)CCCC)c2S1)SC(=C1C(=O)N(Cc2ccccc2)N(Cc2ccccc2)C1=O)S3]

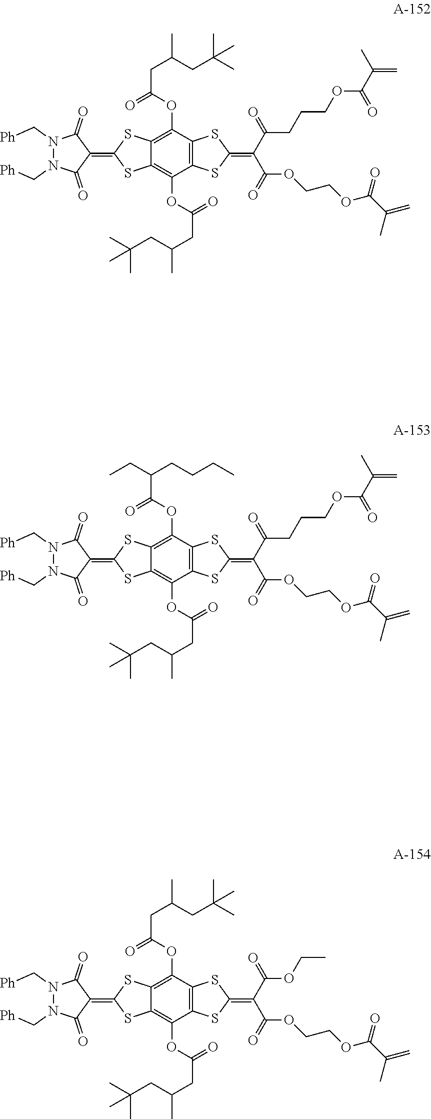 [SMILES: C=C(C)C(=O)OCCCC(=O)C(C(=O)OCCOC(=O)C(=C)C)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3 ; C=C(C)C(=O)OCCCC(=O)/C(C(=O)OCCOC(=O)C(=C)C)=C1/Sc2c(OC(=O)CC(C)CC(C)(C)C)c3c(c(OC(=O)C(CC)CCCC)c2S1)SC(=C1C(=O)N(Cc2ccccc2)N(Cc2ccccc2)C1=O)S3 ; C=C(C)C(=O)OCCOC(=O)C(C(=O)OCC)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3]

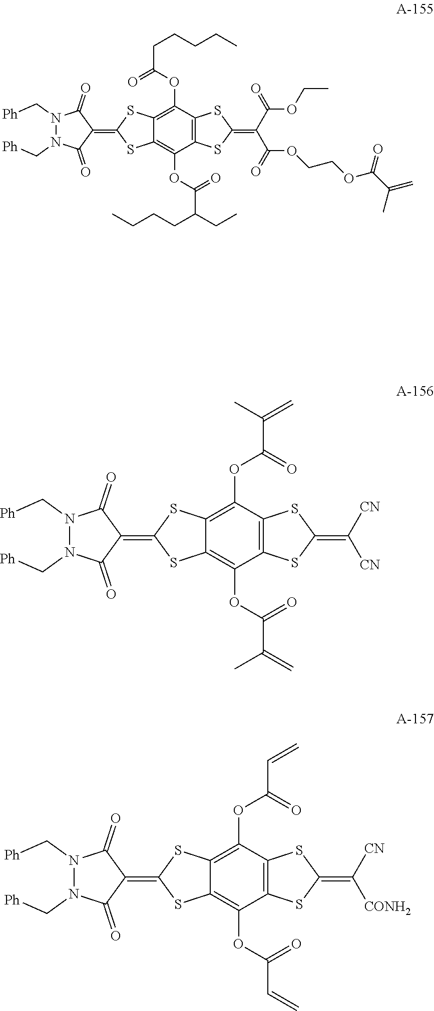 [SMILES: C=C(C)C(=O)OCCOC(=O)/C(C(=O)OCC)=C1/Sc2c(OC(=O)CCCCC)c3c(c(OC(=O)C(CC)CCCC)c2S1)SC(=C1C(=O)N(Cc2ccccc2)N(Cc2ccccc2)C1=O)S3 ; C=C(C)C(=O)Oc1c2c(c(OC(=O)C(=C)C)c3c1SC(=C1C(=O)N(Cc4ccccc4)N(Cc4ccccc4)C1=O)S3)SC(=C(C#N)C#N)S2 ; C=CC(=O)Oc1c2c(c(OC(=O)C=C)c3c1SC(=C1C(=O)N(Cc4ccccc4)N(Cc4ccccc4)C1=O)S3)SC(=C(C#N)C(N)=O)S2]

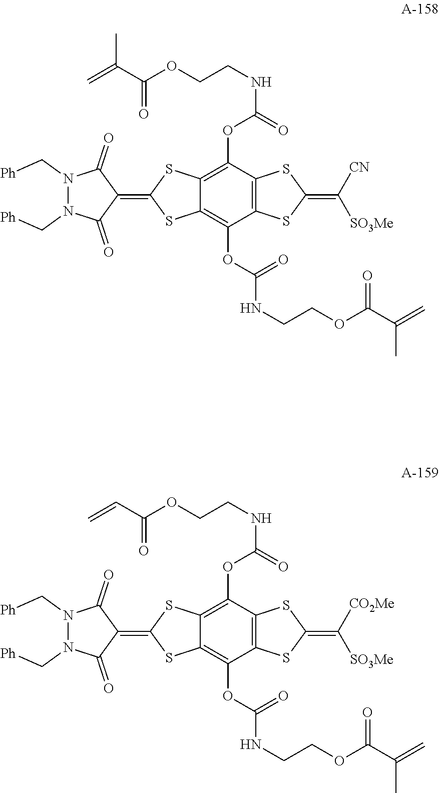

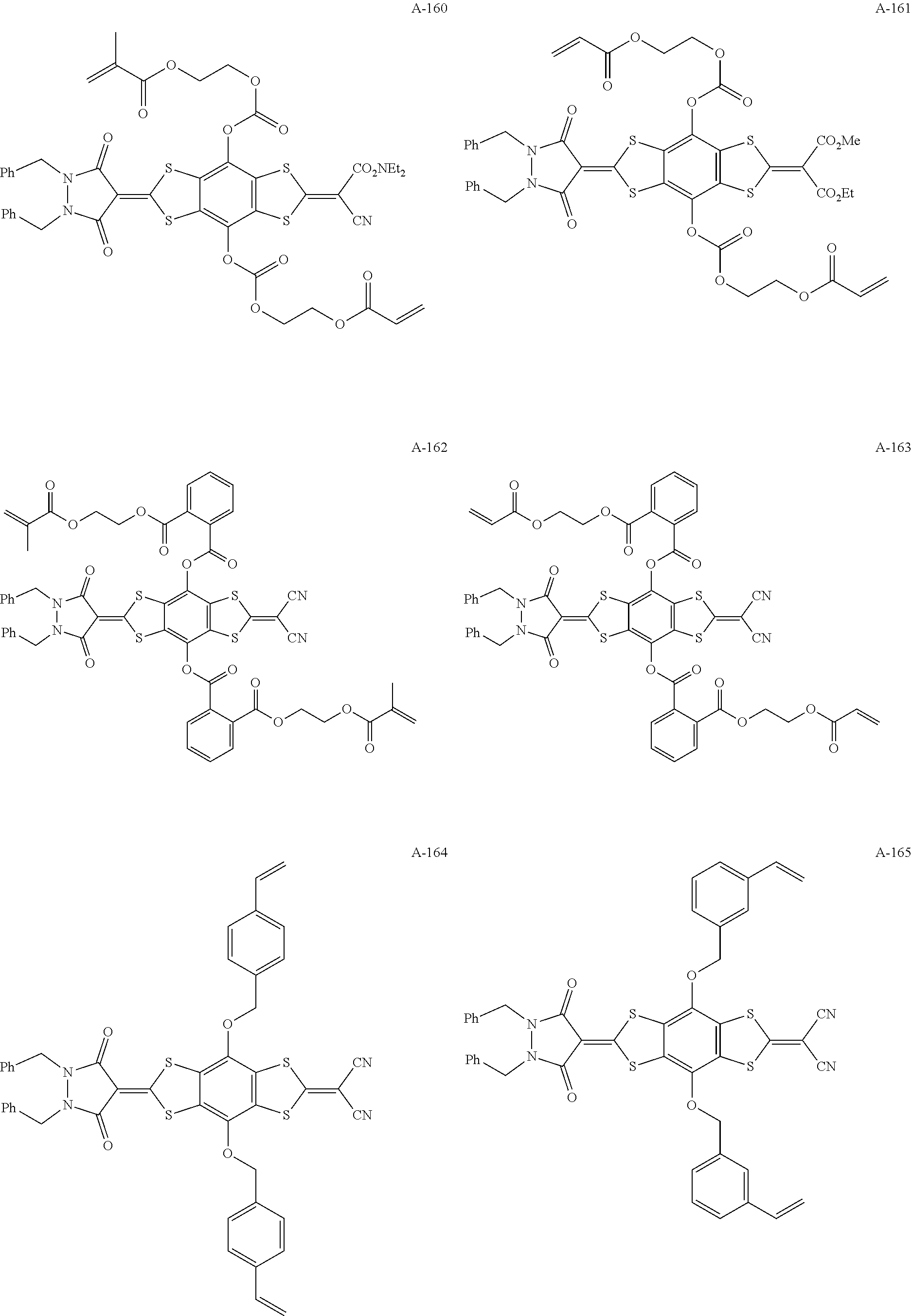

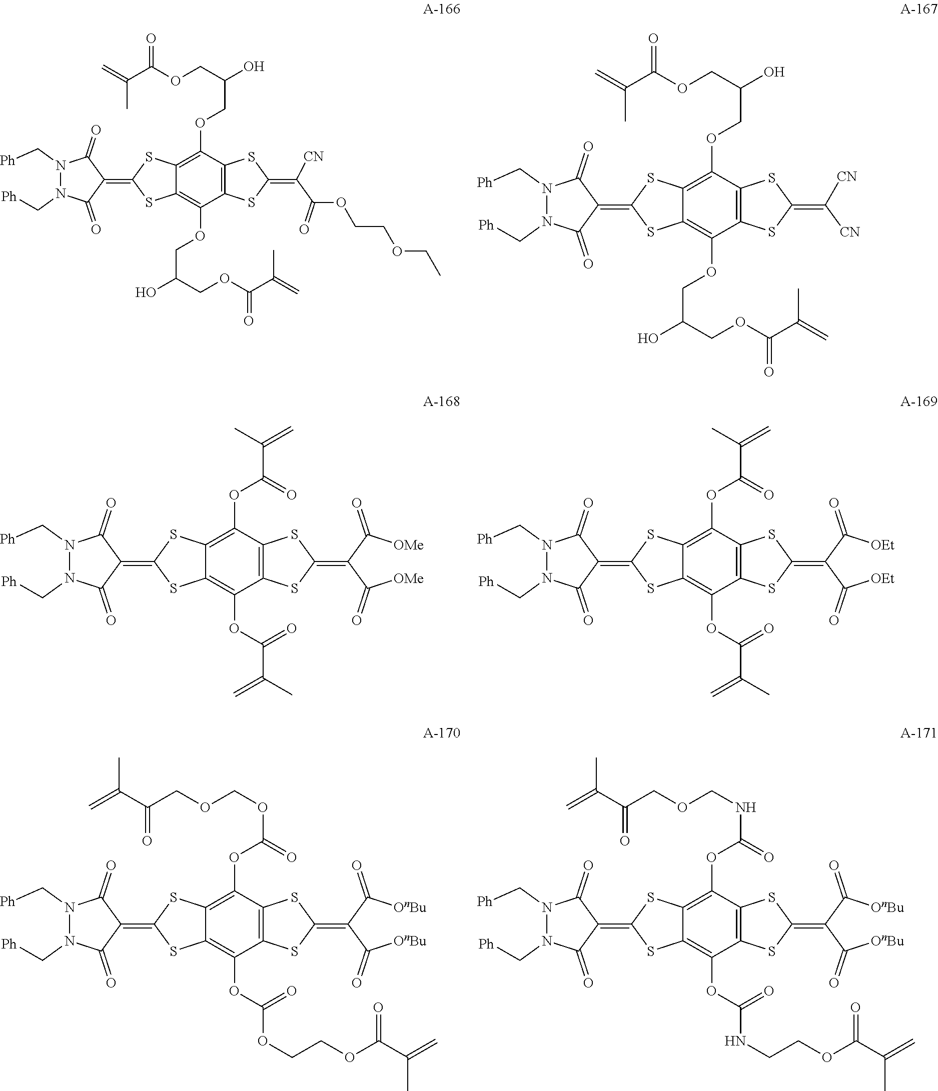

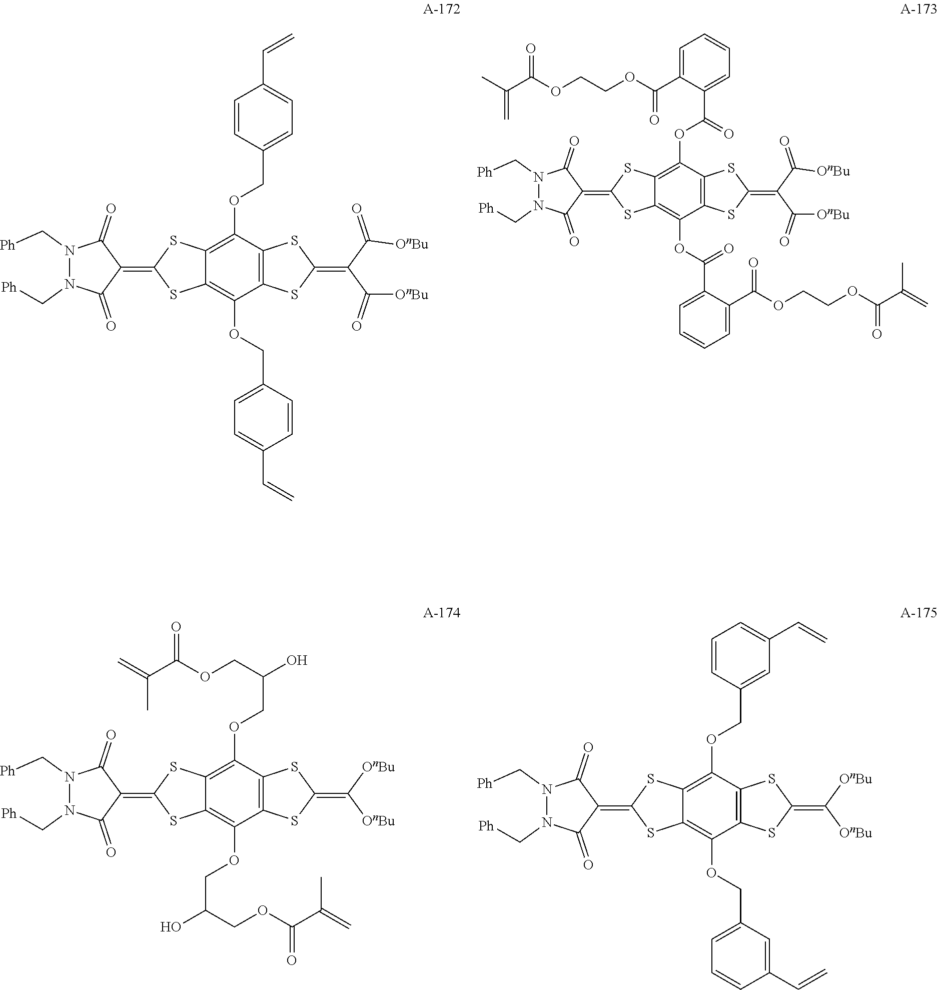

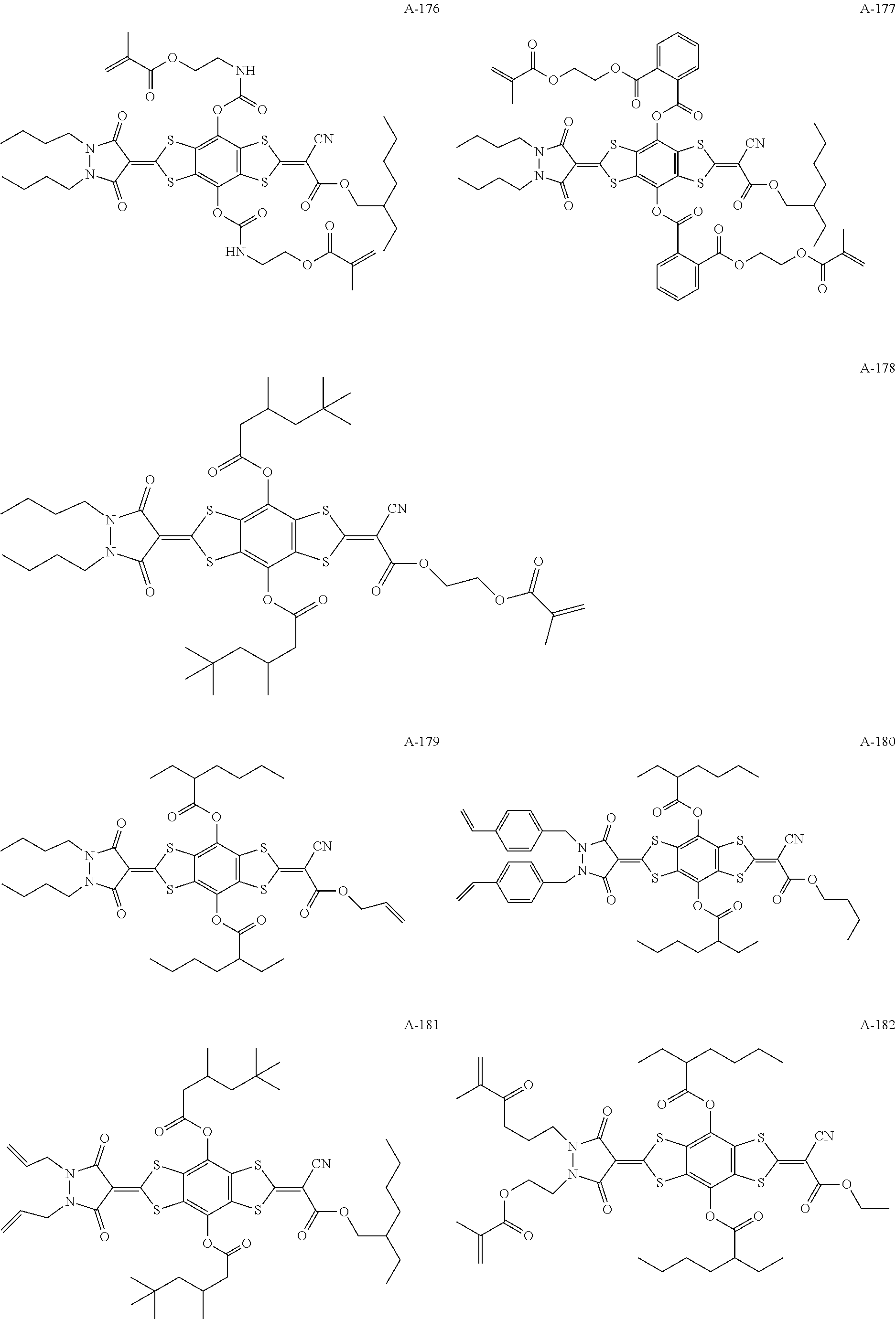

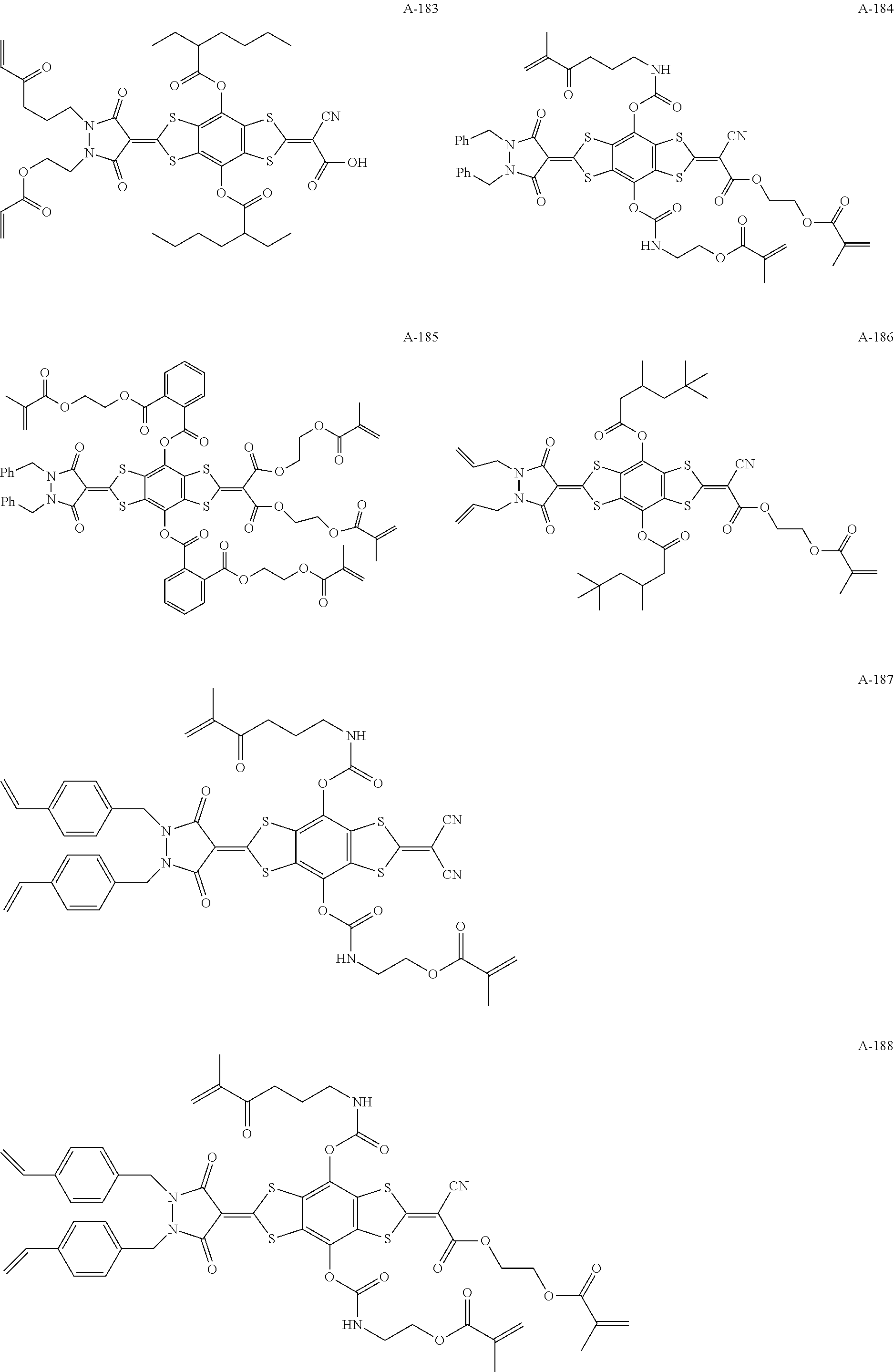

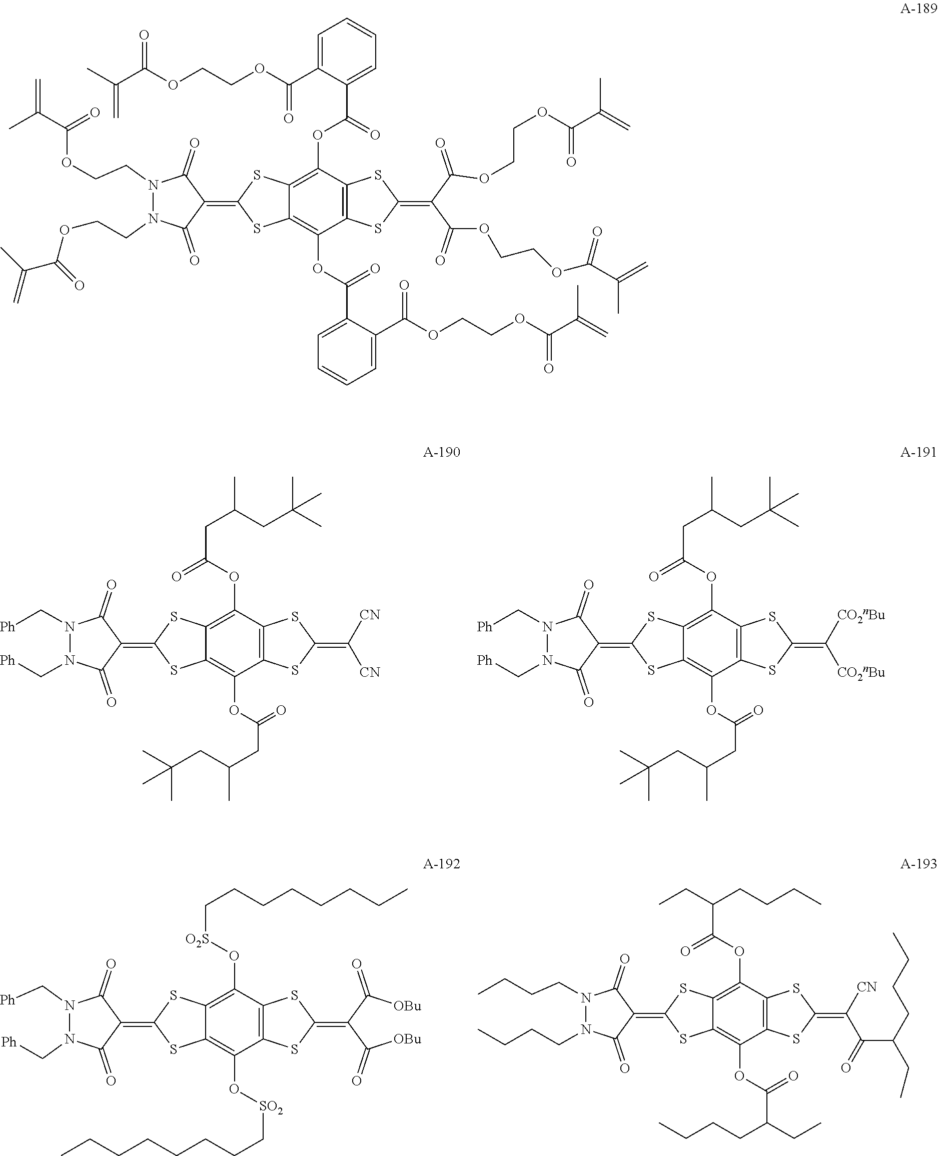

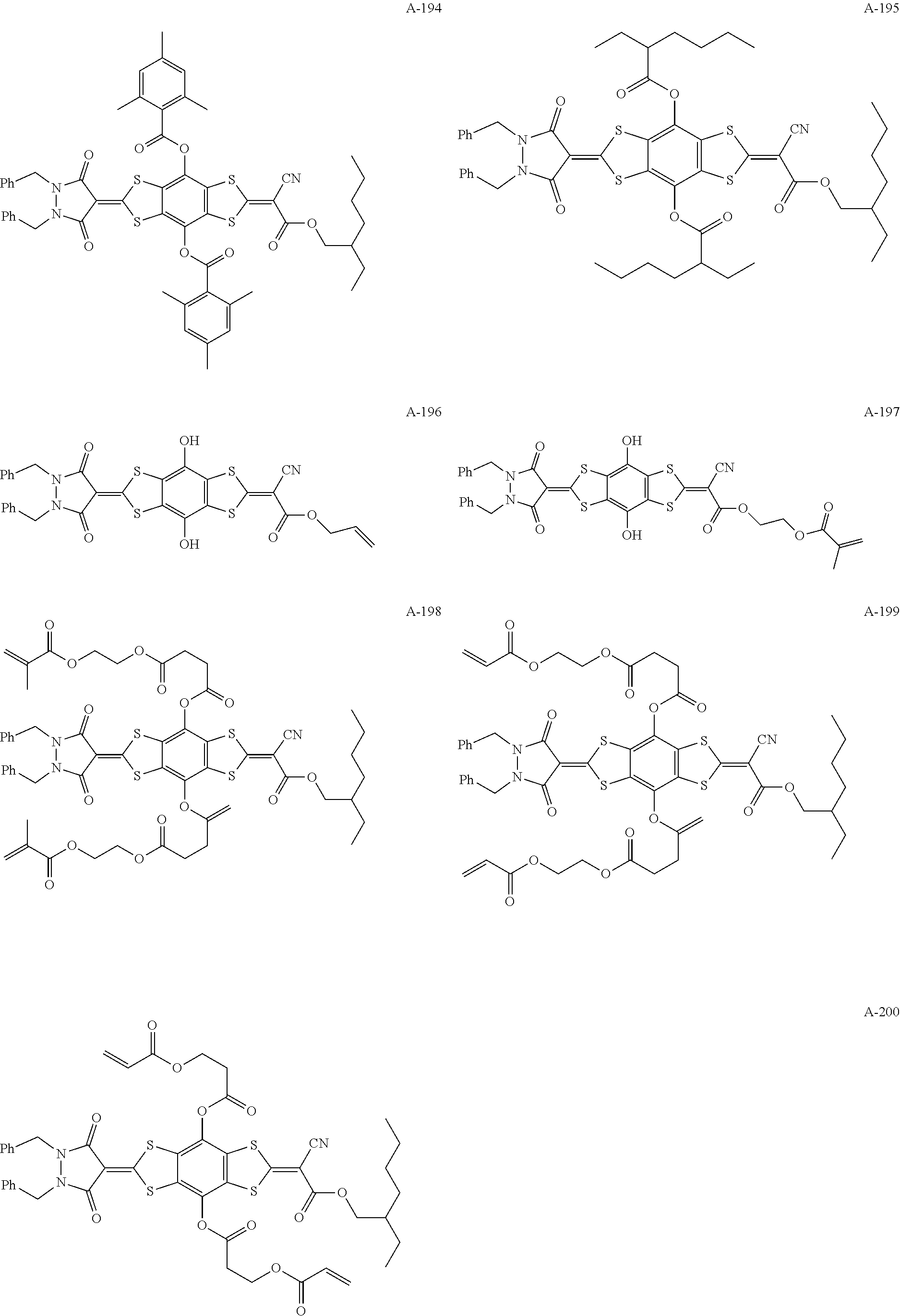

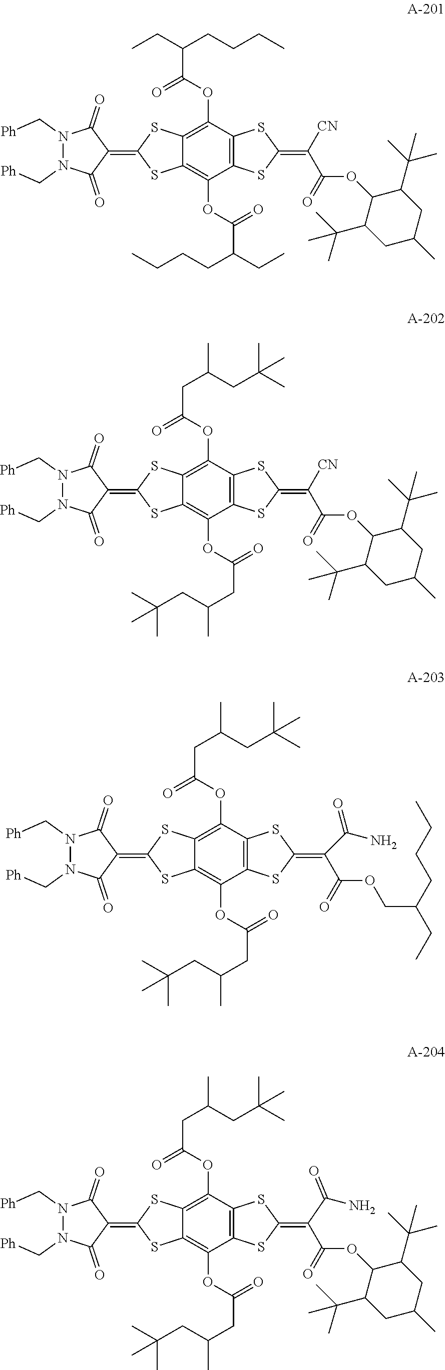

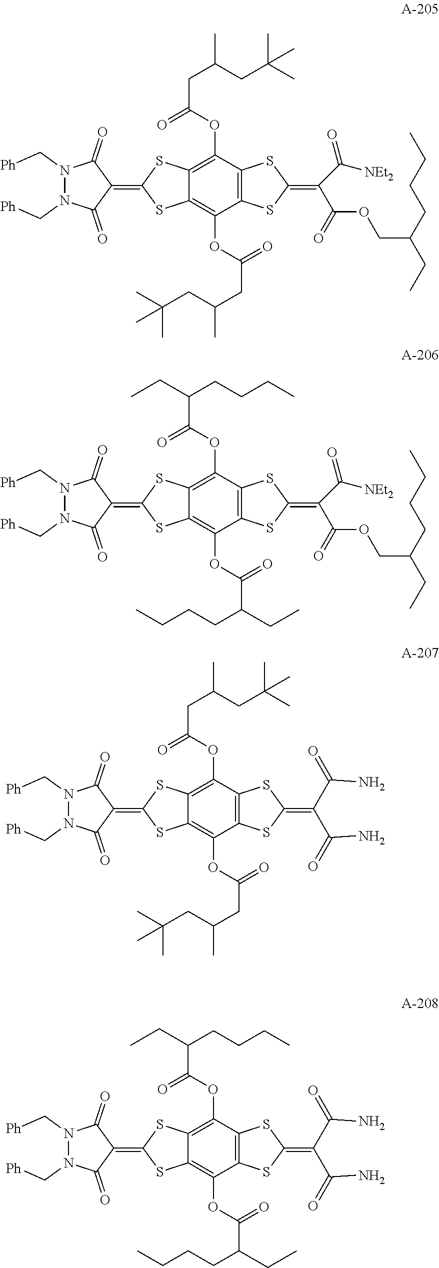

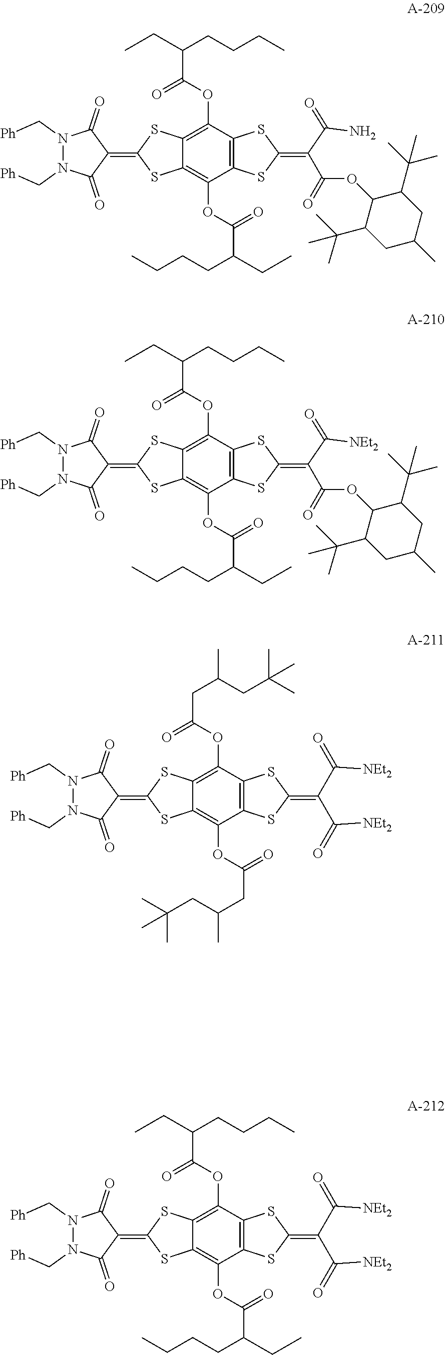

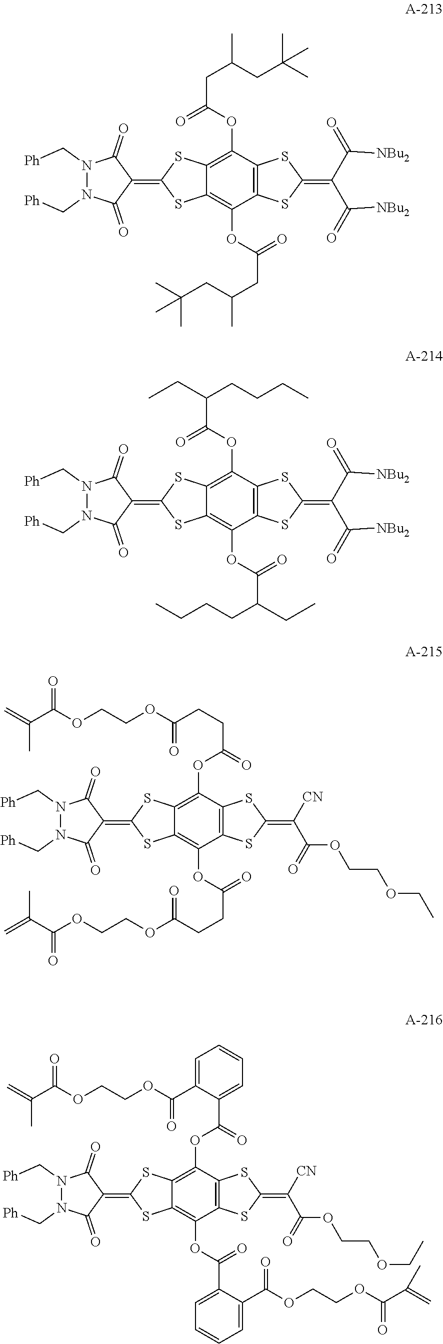

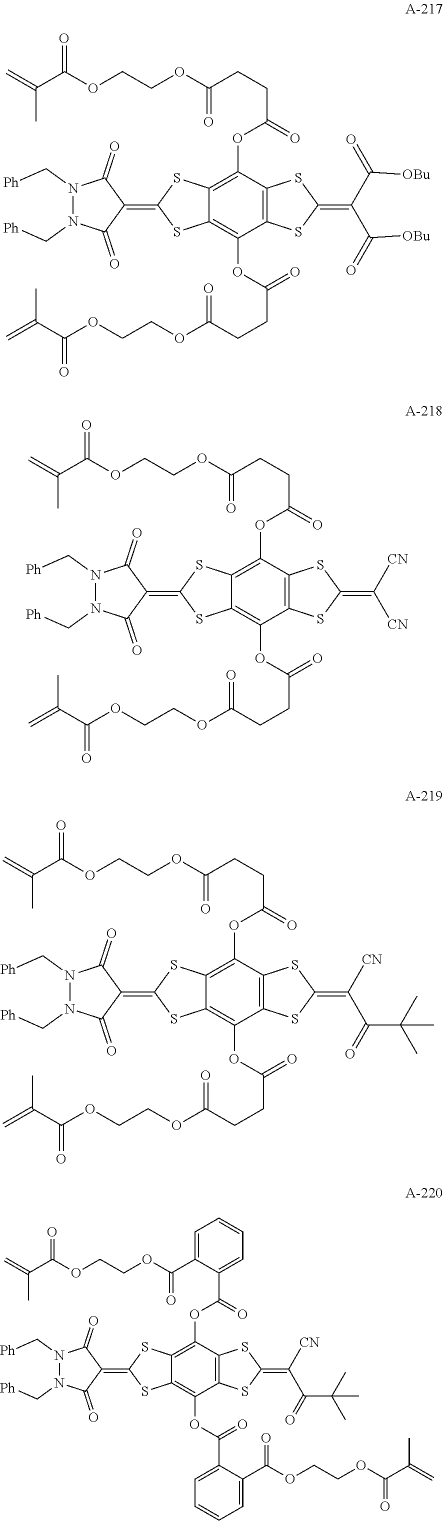

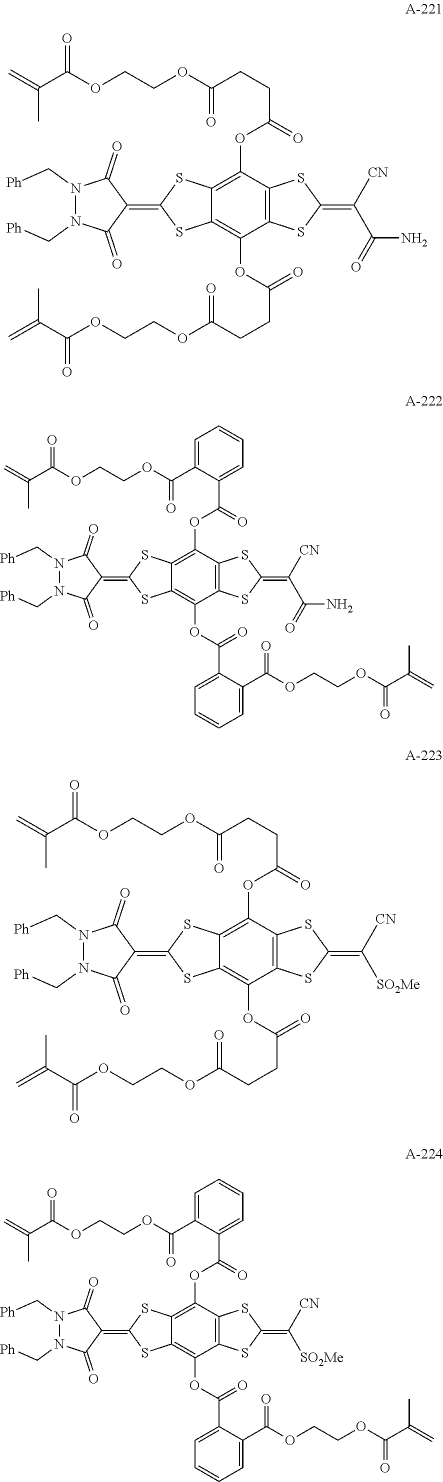

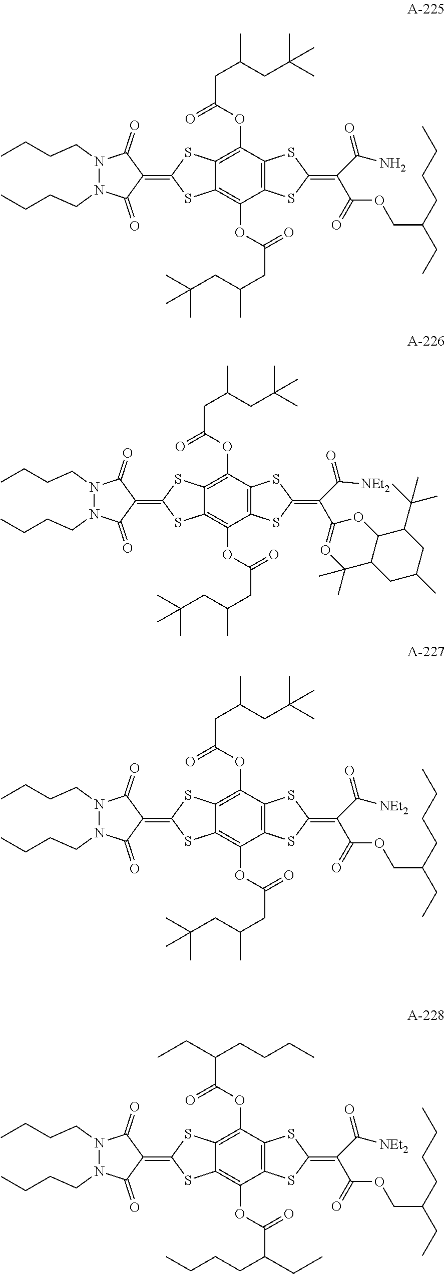

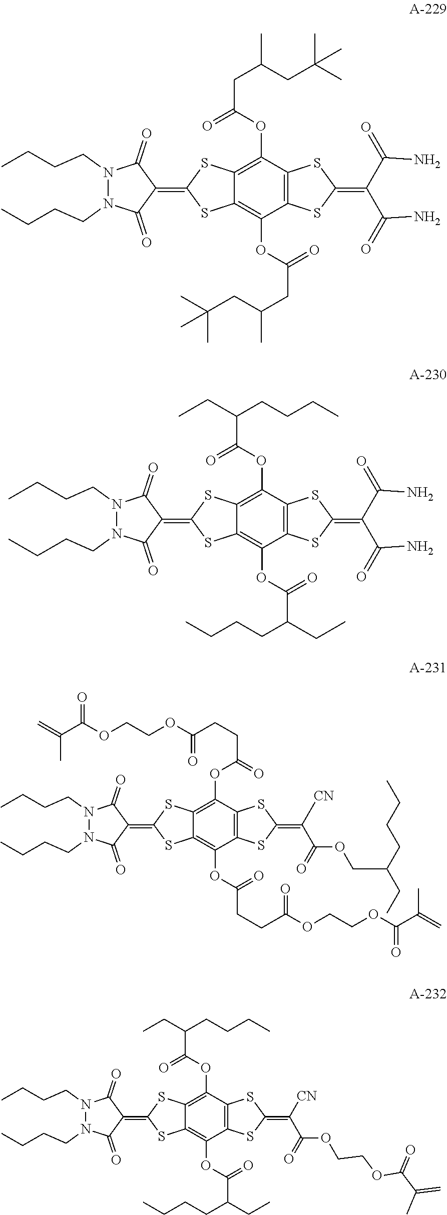

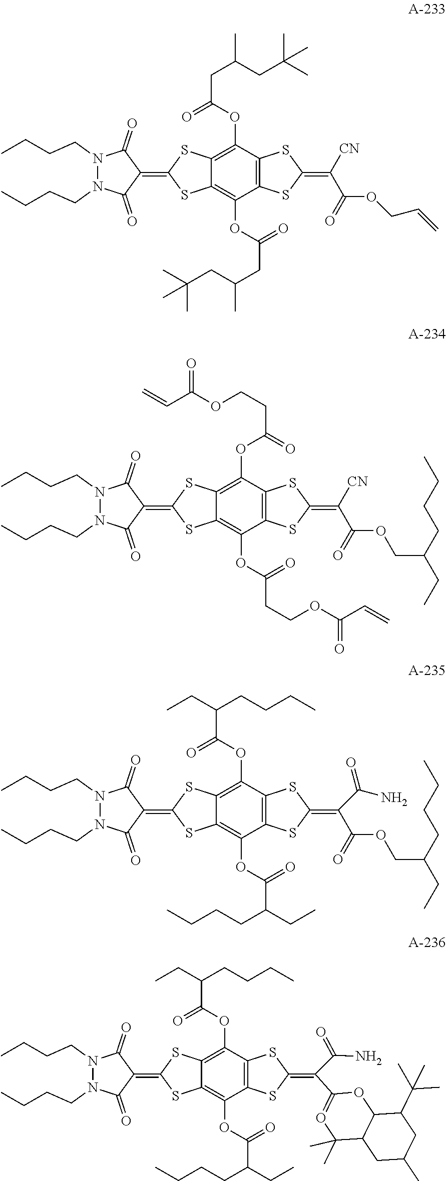

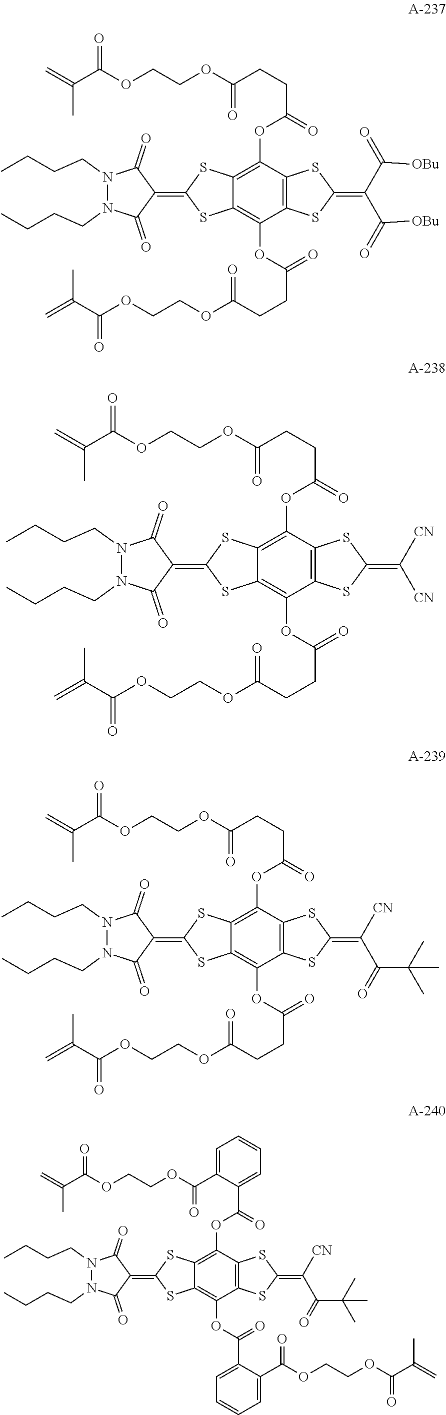

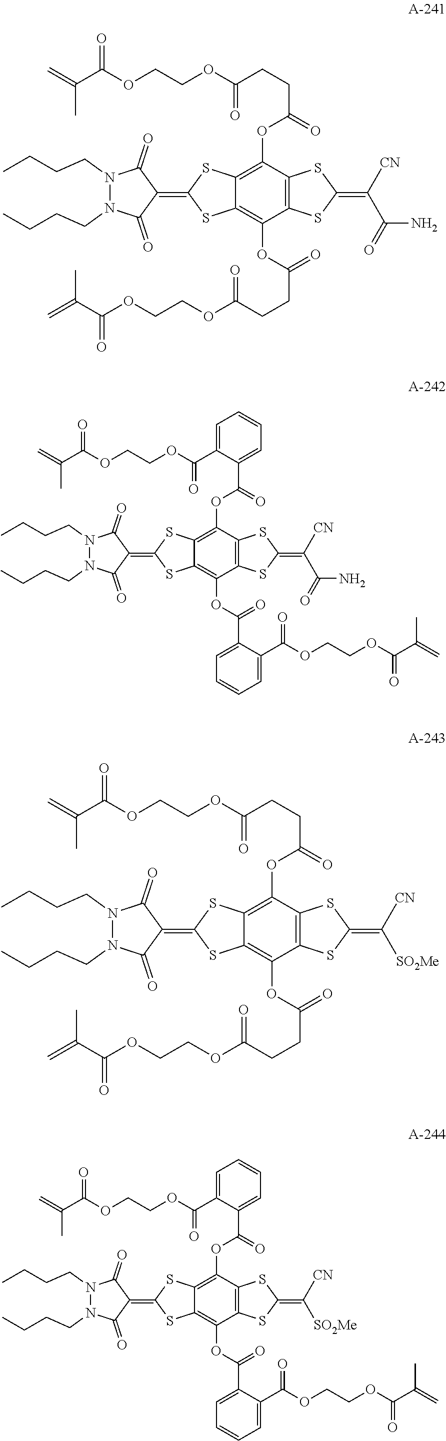

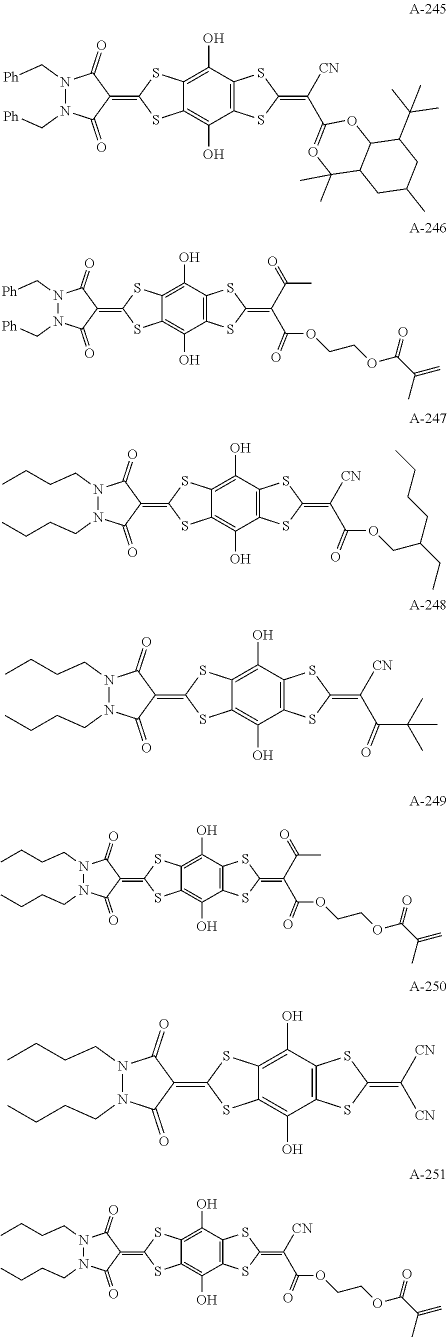

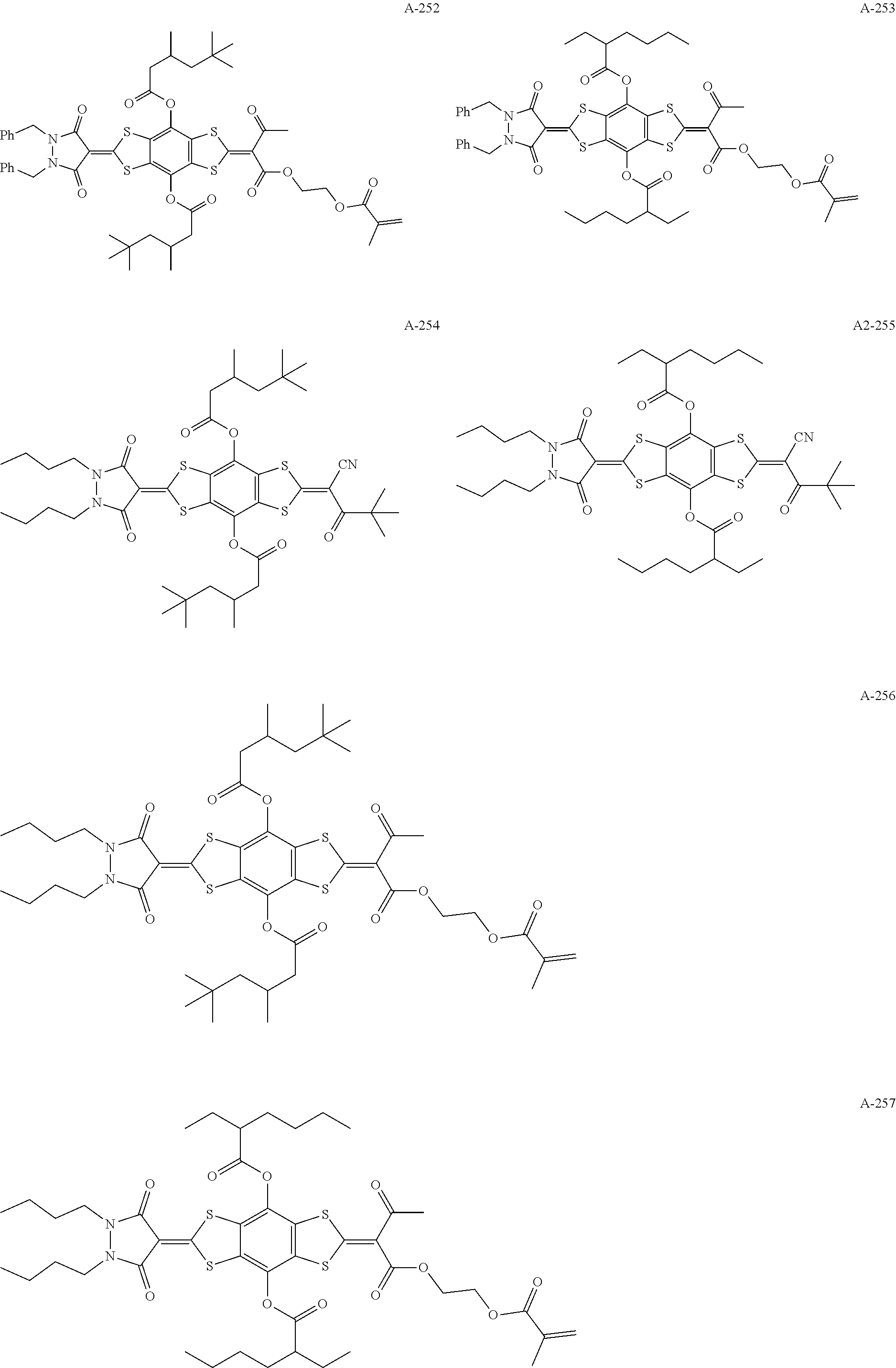

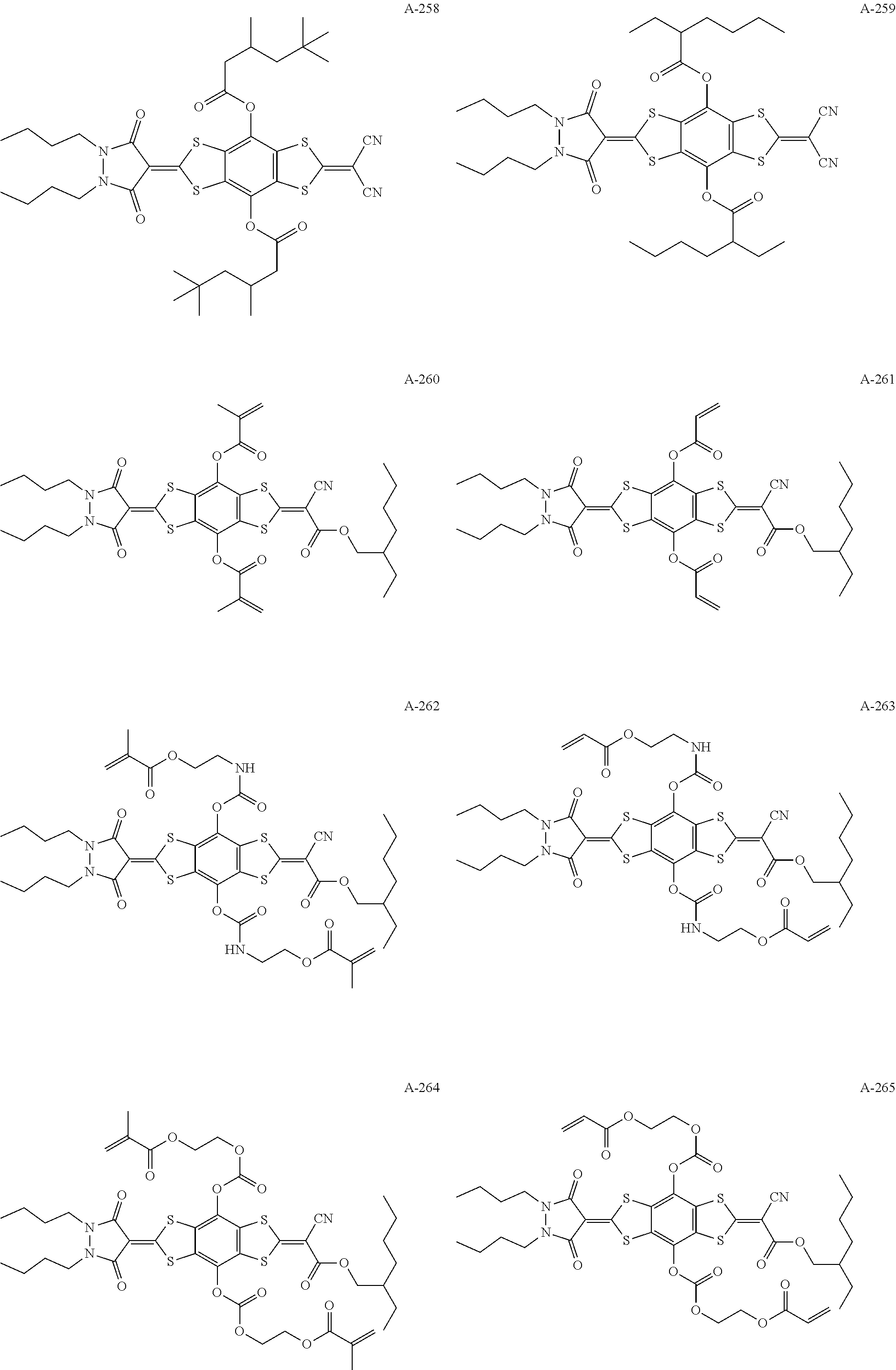

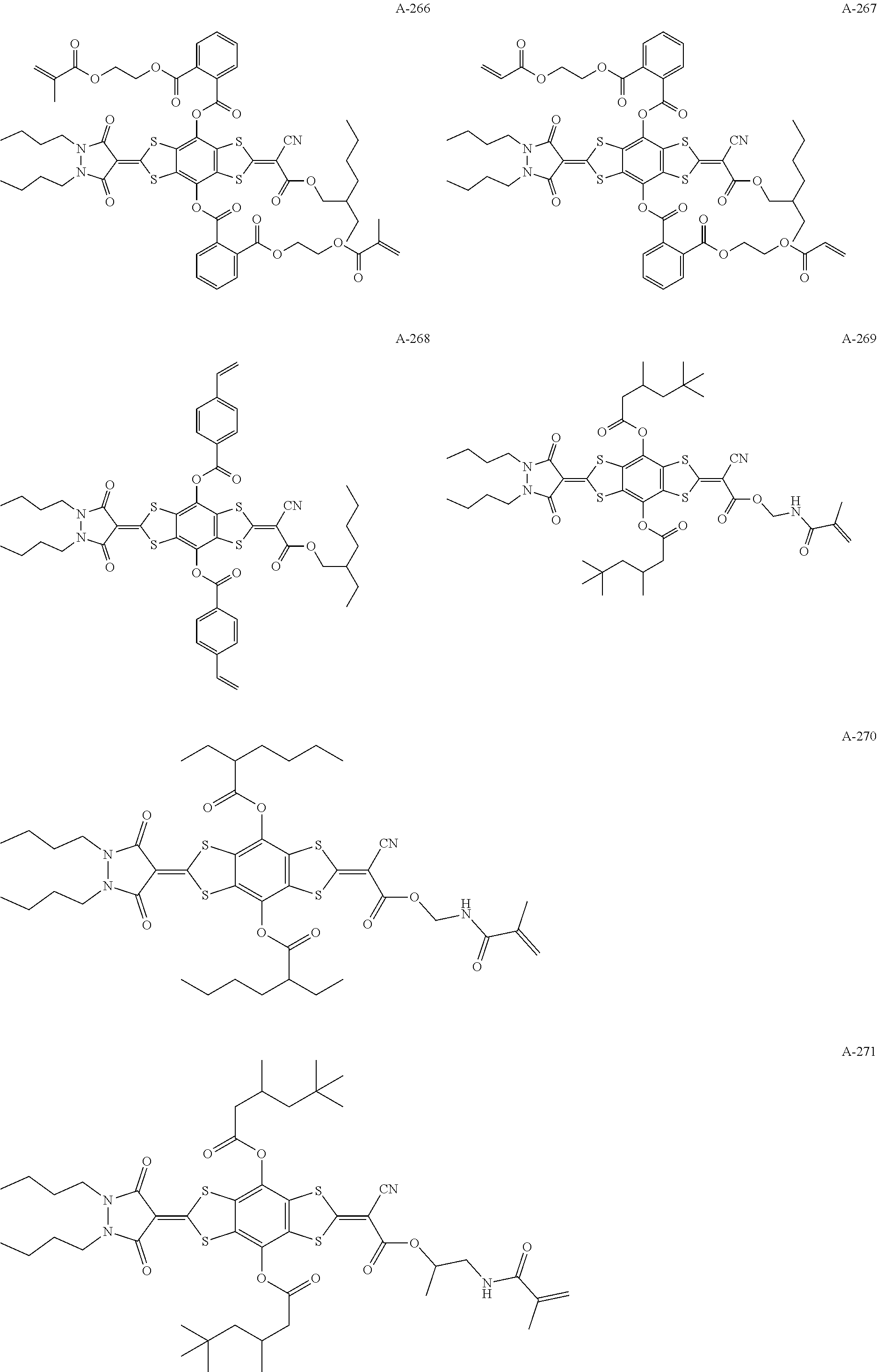

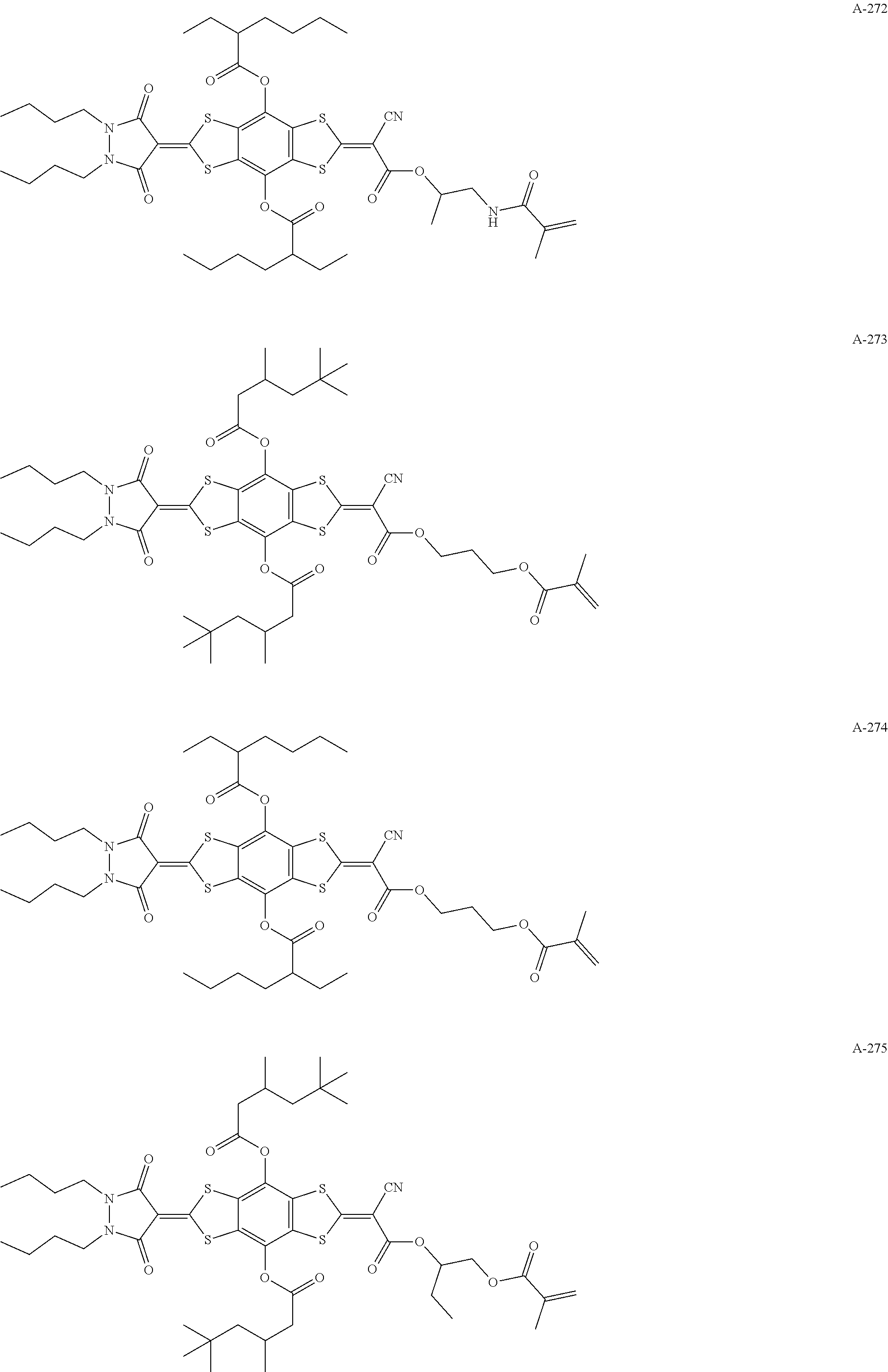

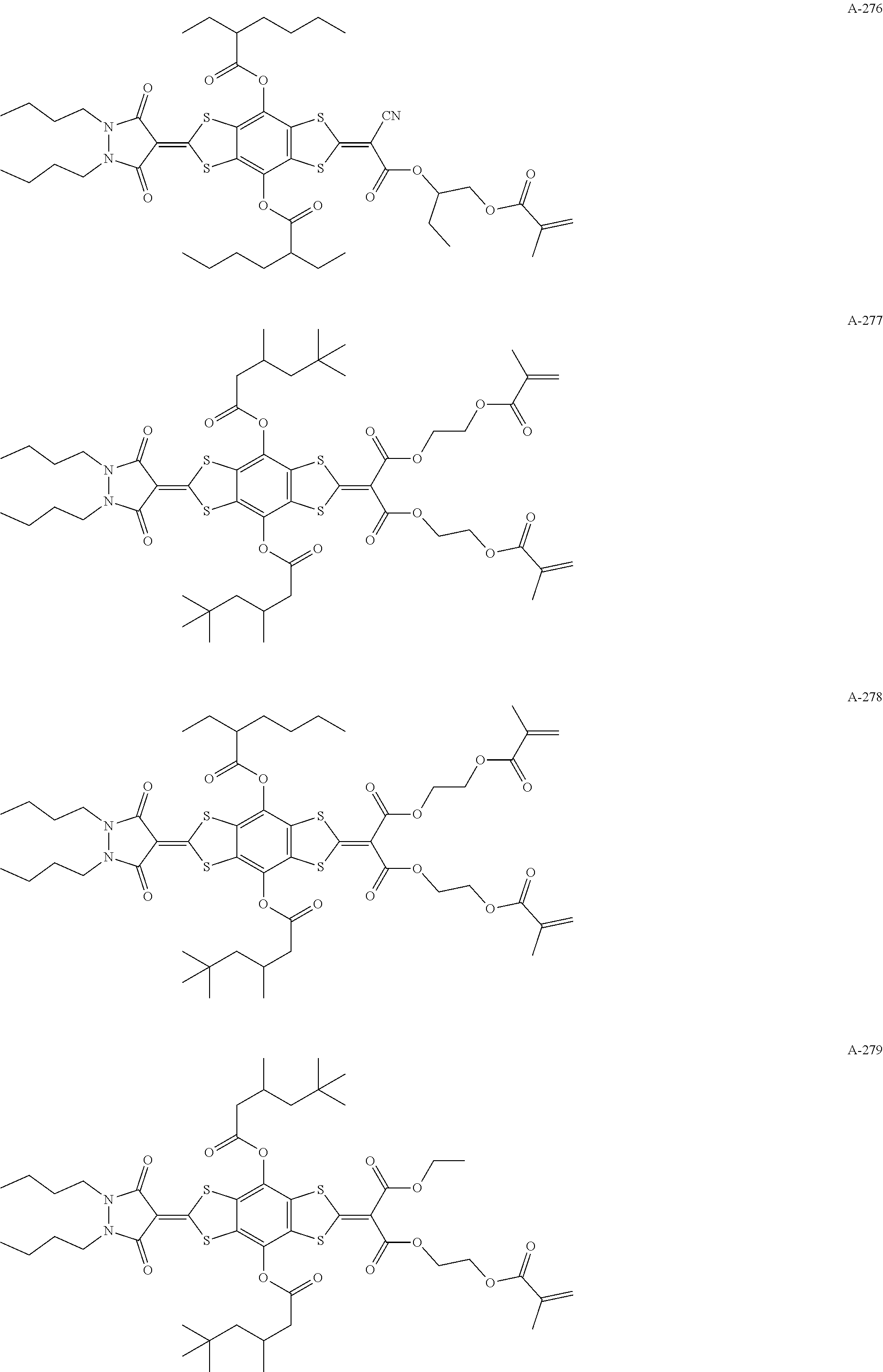

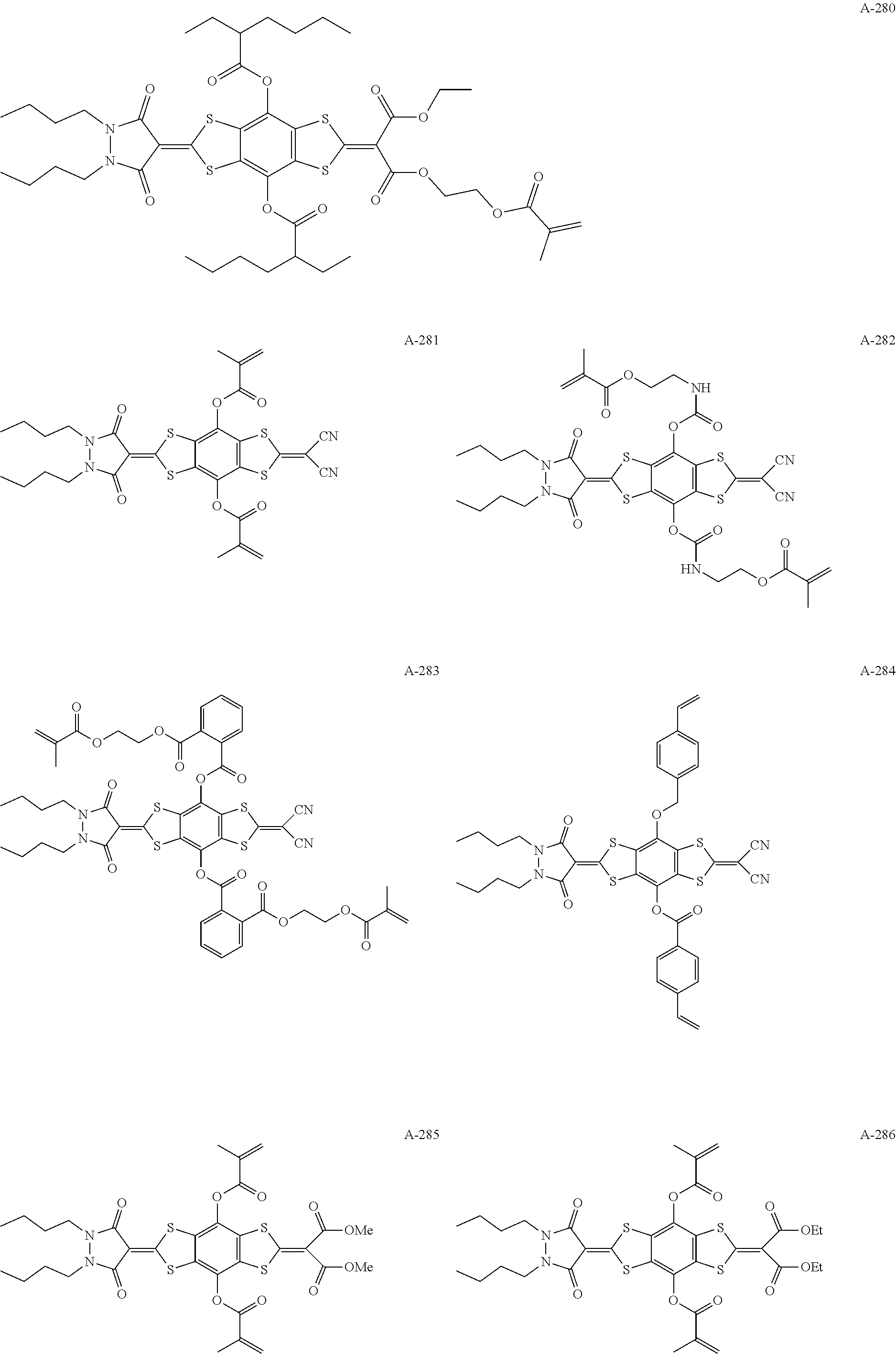

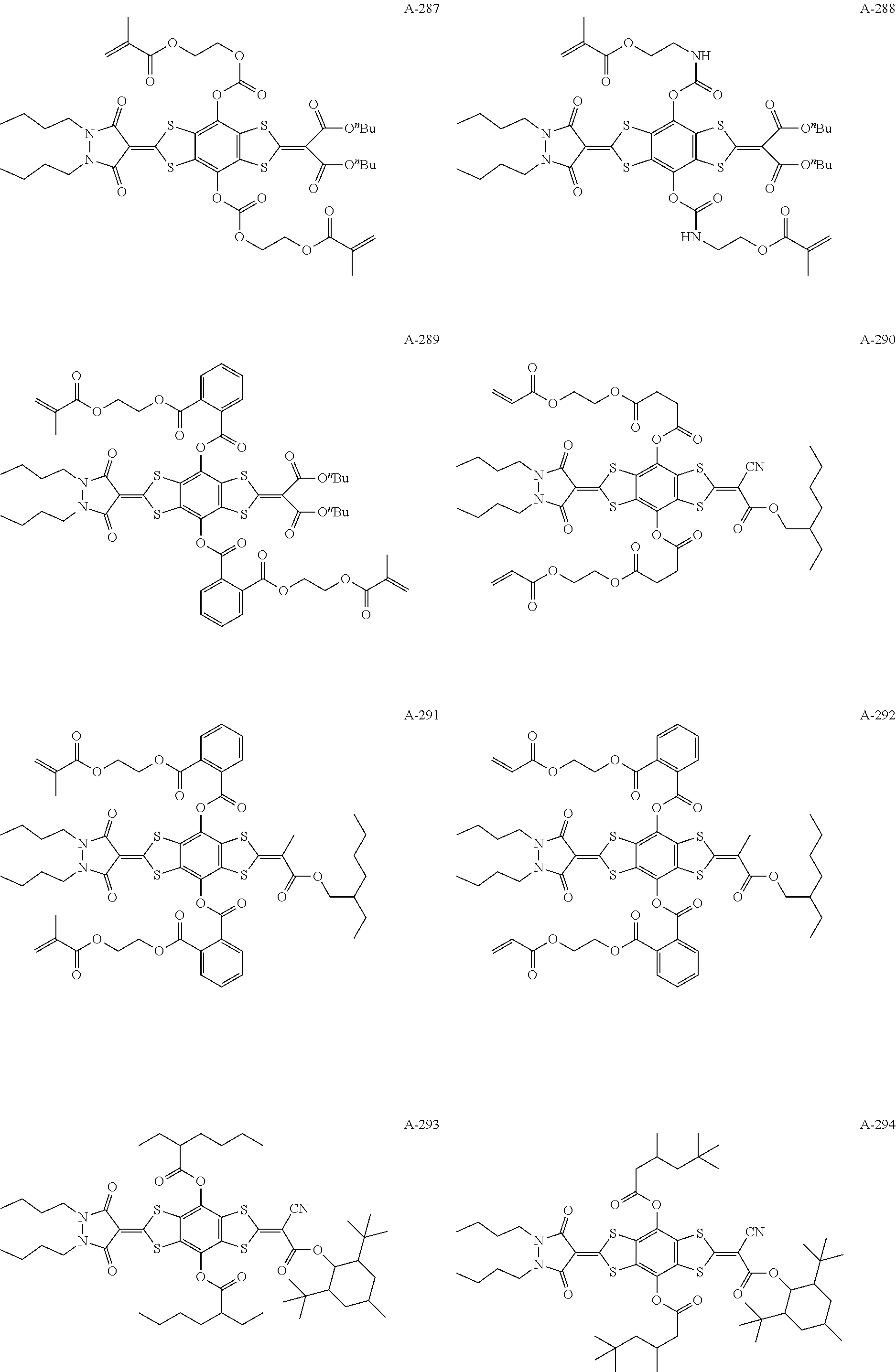

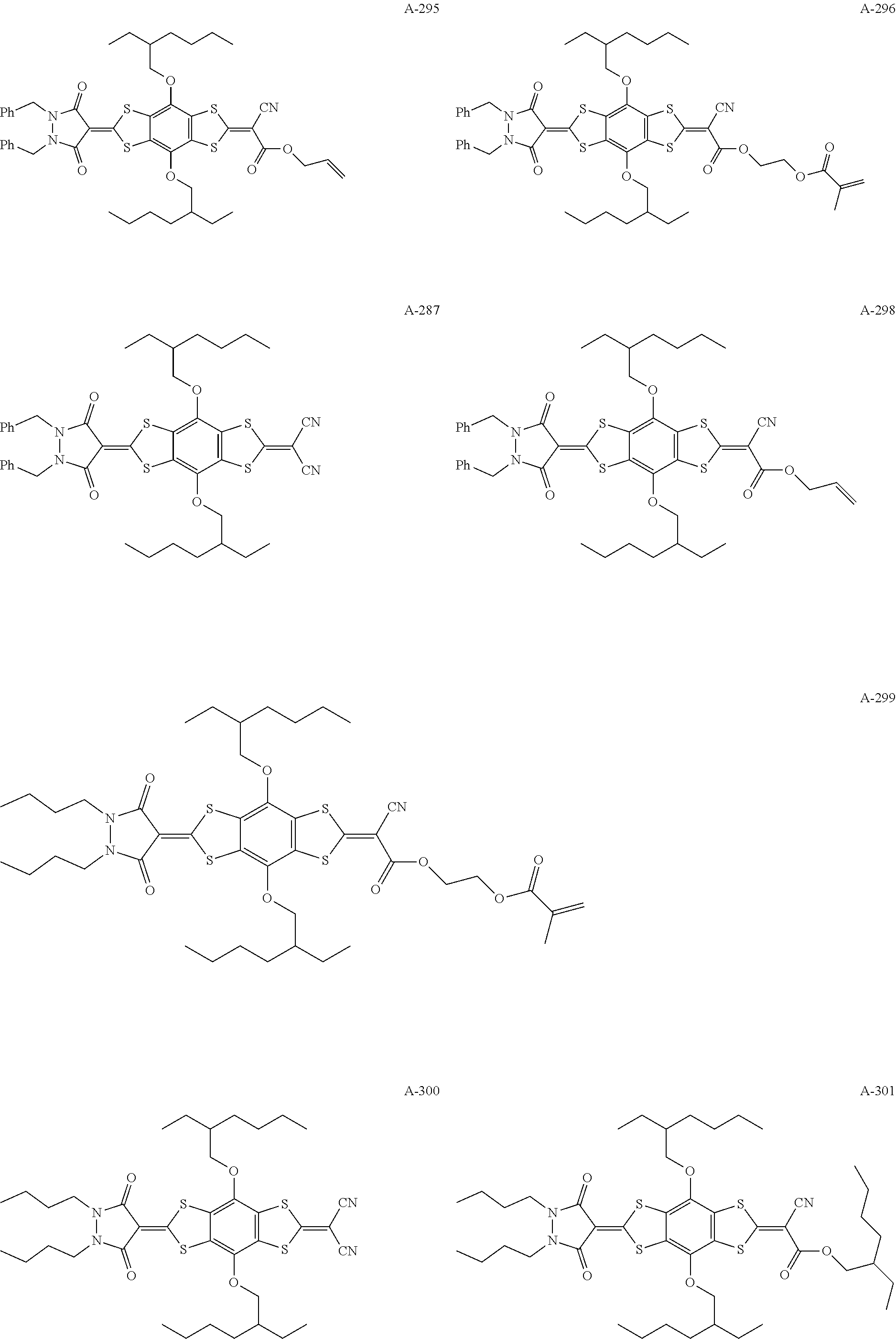

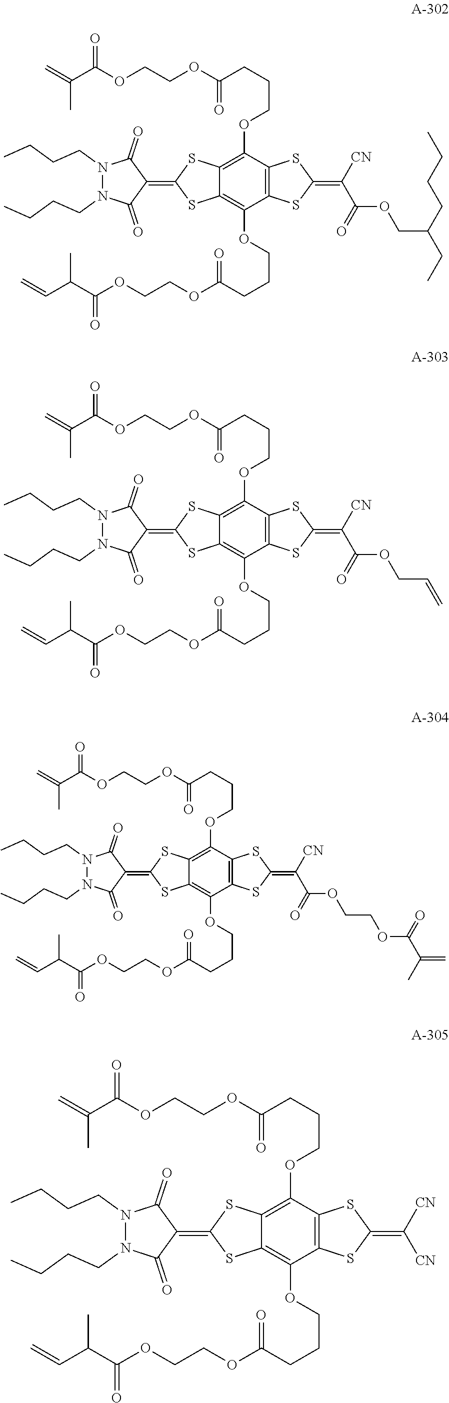

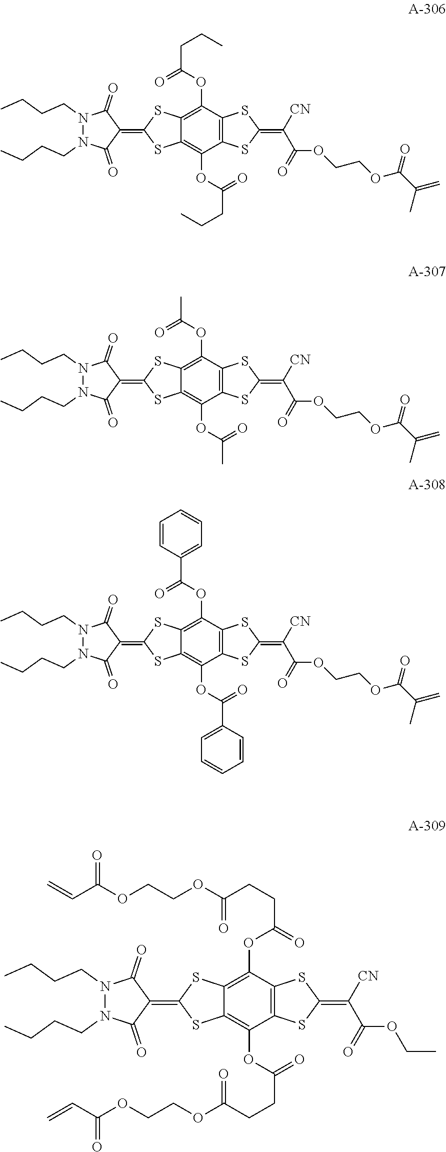

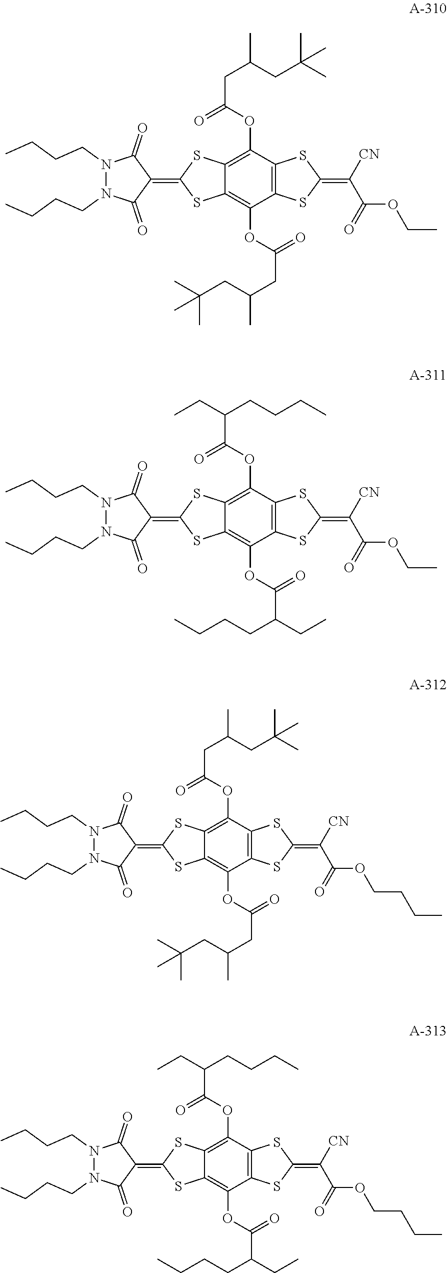

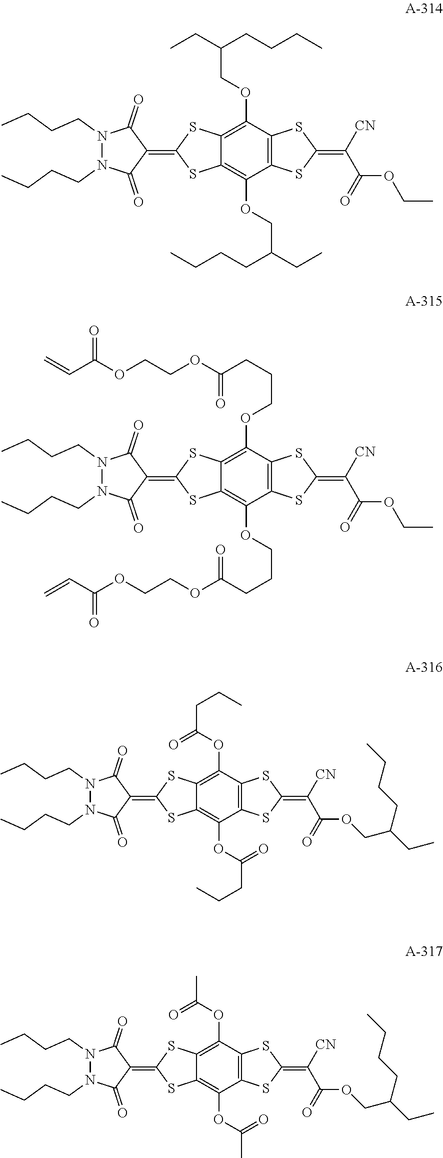

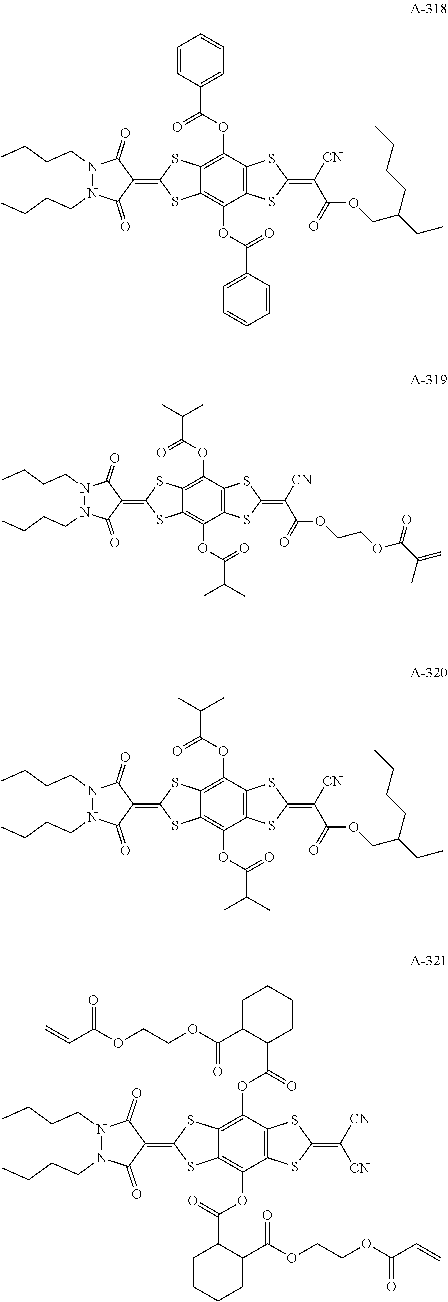

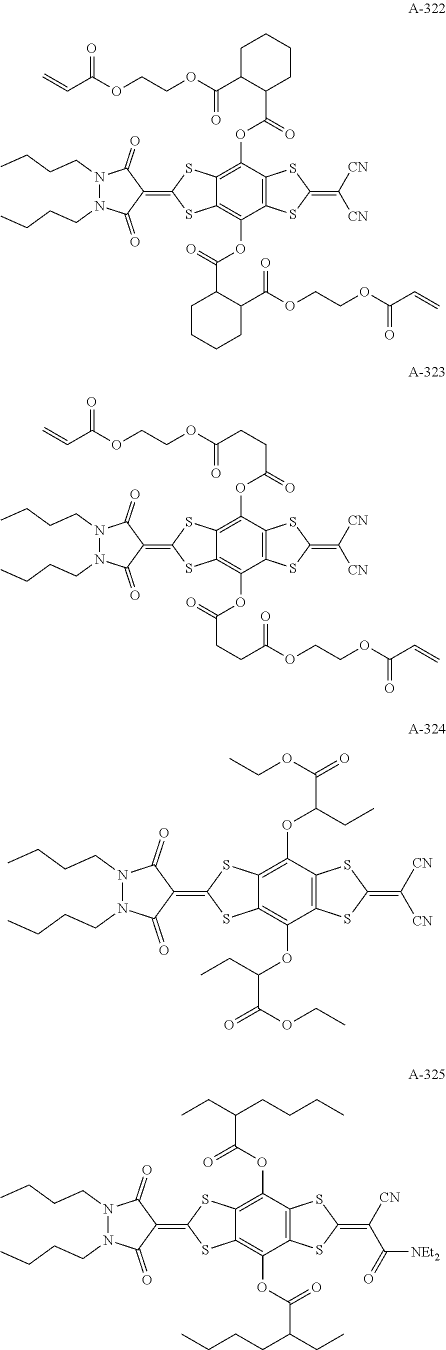

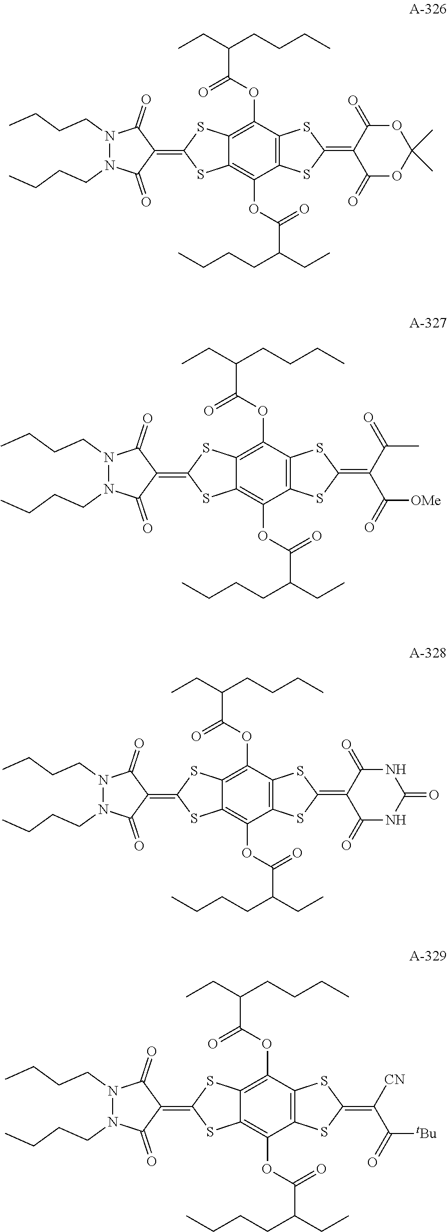

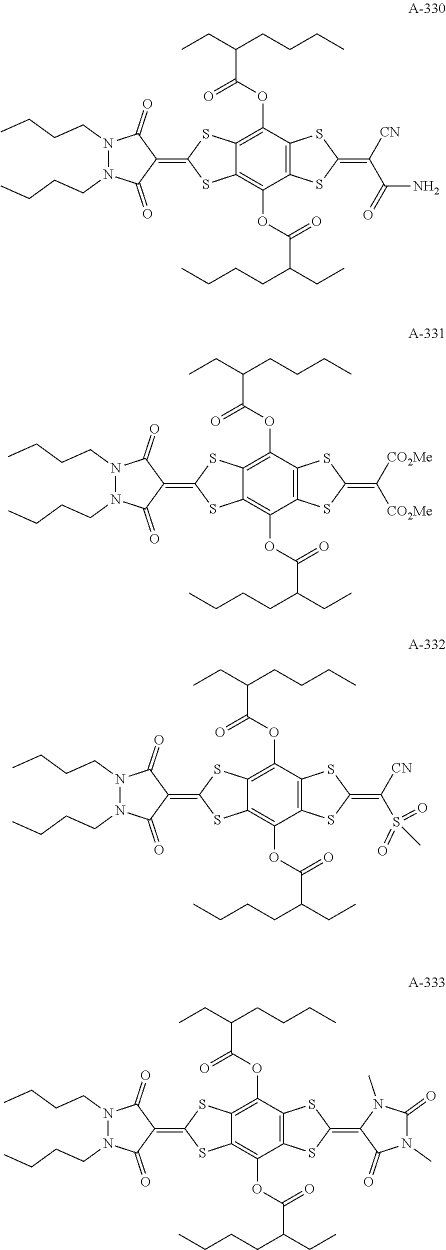 [SMILES: CCCCC(CC)C(=O)Oc1c2c(c(OC(=O)C(CC)CCCC)c3c1SC(=C1C(=O)N(CCCC)N(CCCC)C1=O)S3)SC(=C(C#N)C(N)=O)S2 ; CCCCC(CC)C(=O)Oc1c2c(c(OC(=O)C(CC)CCCC)c3c1SC(=C1C(=O)N(CCCC)N(CCCC)C1=O)S3)SC(=C(C(=O)OC)C(=O)OC)S2 ; CCCCC(CC)C(=O)Oc1c2c(c(OC(=O)C(CC)CCCC)c3c1SC(=C(C#N)S(C)(=O)=O)S3)SC(=C1C(=O)N(CCCC)N(CCCC)C1=O)S2 ; CCCCC(CC)C(=O)Oc1c2c(c(OC(=O)C(CC)CCCC)c3c1SC(=C1C(=O)N(C)C(=O)N1C)S3)SC(=C1C(=O)N(CCCC)N(CCCC)C1=O)S2]

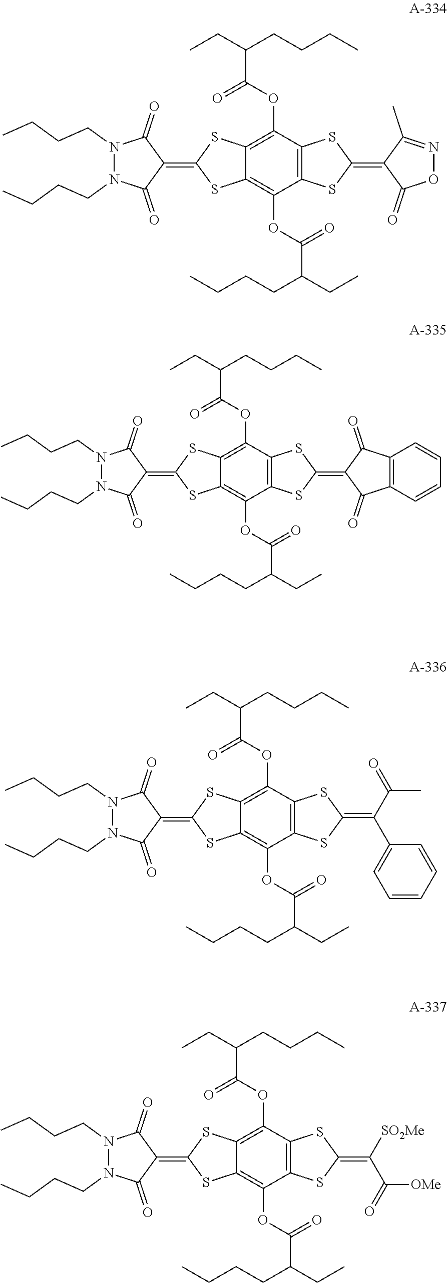 [SMILES: CCCCC(CC)C(=O)Oc1c2c(c(OC(=O)C(CC)CCCC)c3c1SC(=C1C(=O)ON=C1C)S3)SC(=C1C(=O)N(CCCC)N(CCCC)C1=O)S2 ; CCCCC(CC)C(=O)Oc1c2c(c(OC(=O)C(CC)CCCC)c3c1SC(=C1C(=O)N(CCCC)N(CCCC)C1=O)S3)SC(=C1C(=O)c3ccccc3C1=O)S2 ; CCCCC(CC)C(=O)Oc1c2c(c(OC(=O)C(CC)CCCC)c3c1SC(=C(C(C)=O)c1ccccc1)S3)SC(=C1C(=O)N(CCCC)N(CCCC)C1=O)S2 ; CCCCC(CC)C(=O)Oc1c2c(c(OC(=O)C(CC)CCCC)c3c1SC(=C(C(=O)OC)S(C)(=O)=O)S3)SC(=C1C(=O)N(CCCC)N(CCCC)C1=O)S2]

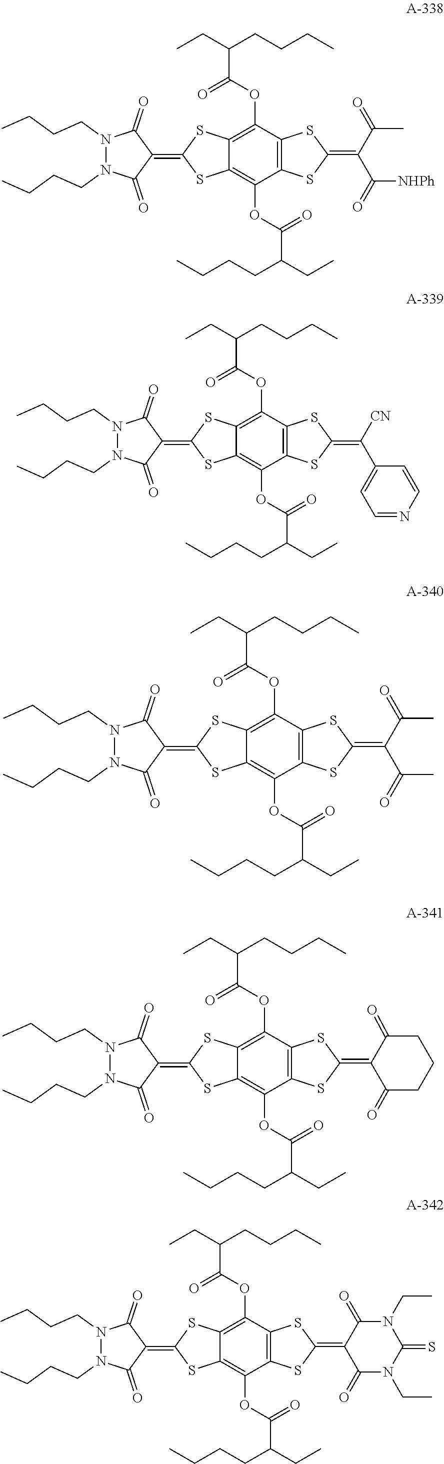

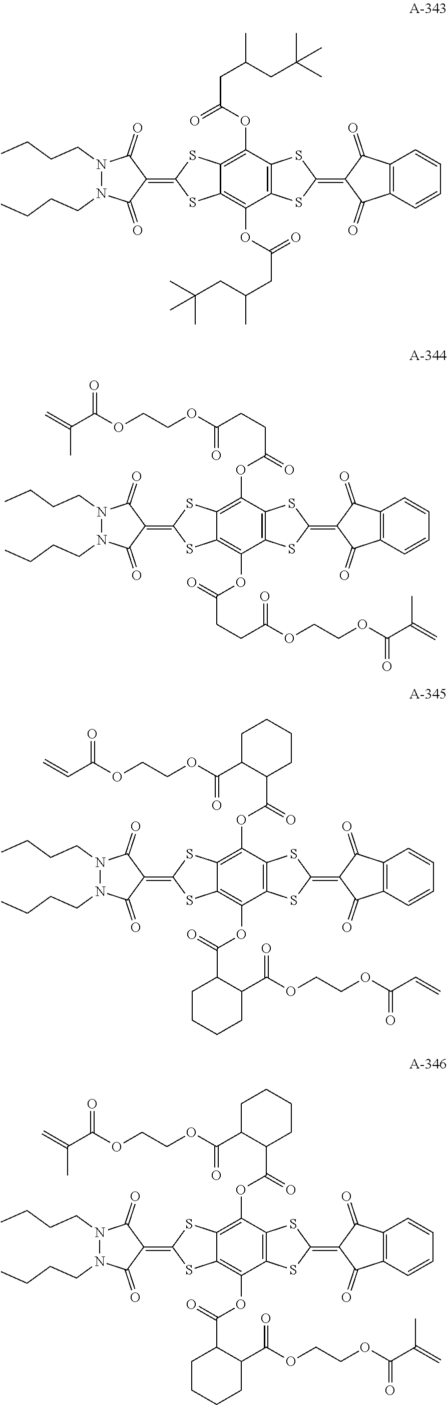

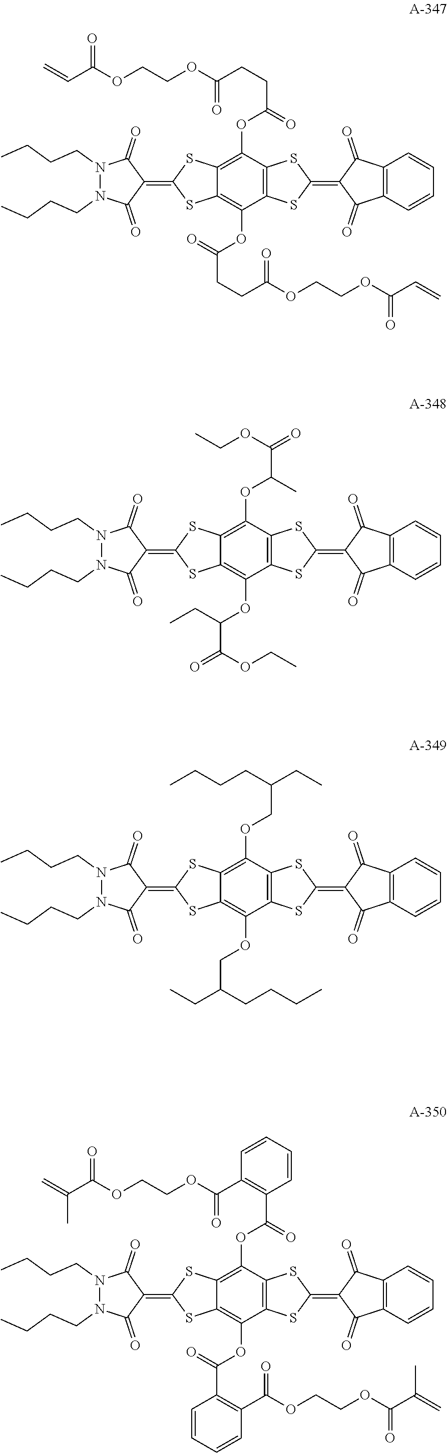

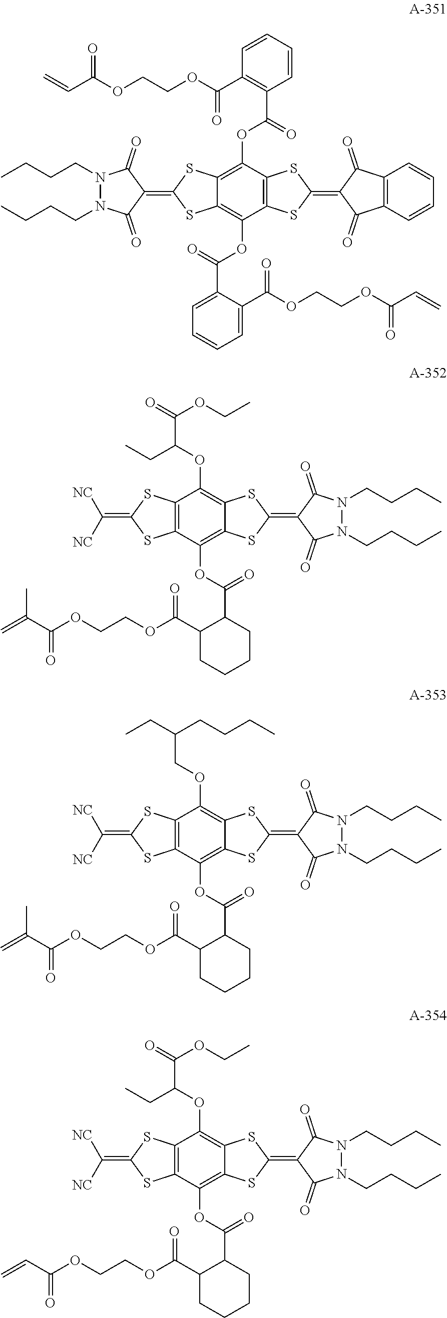

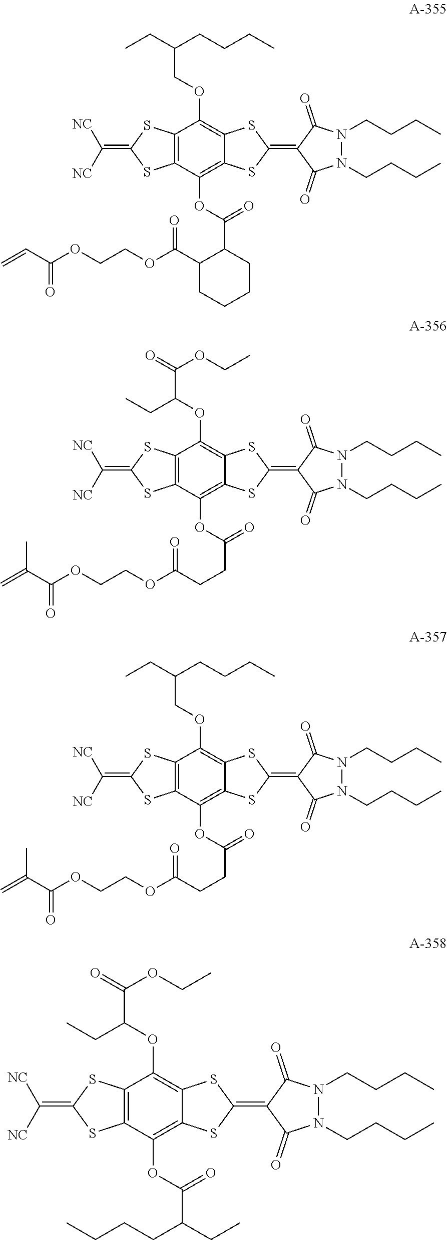

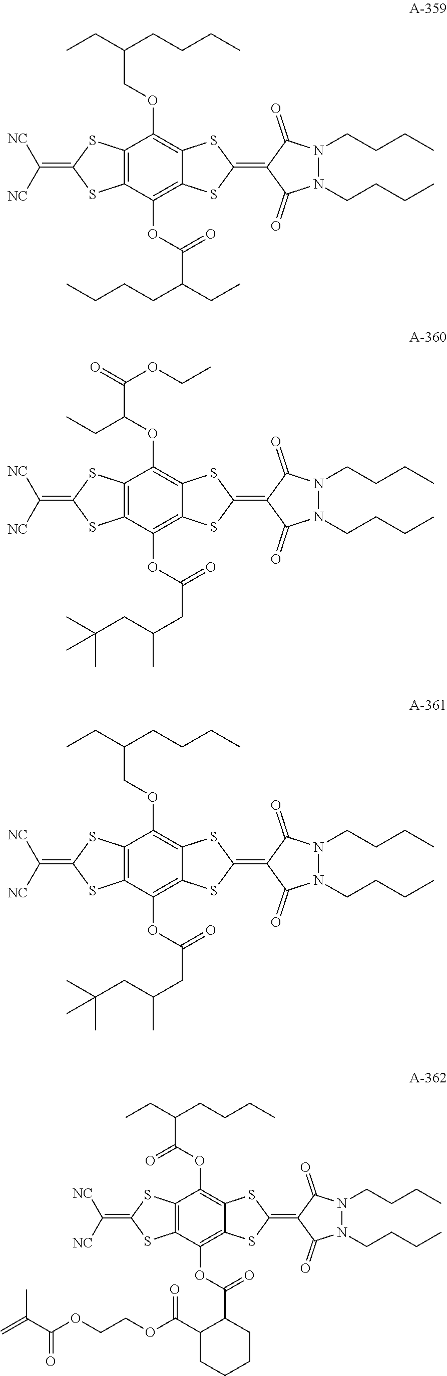

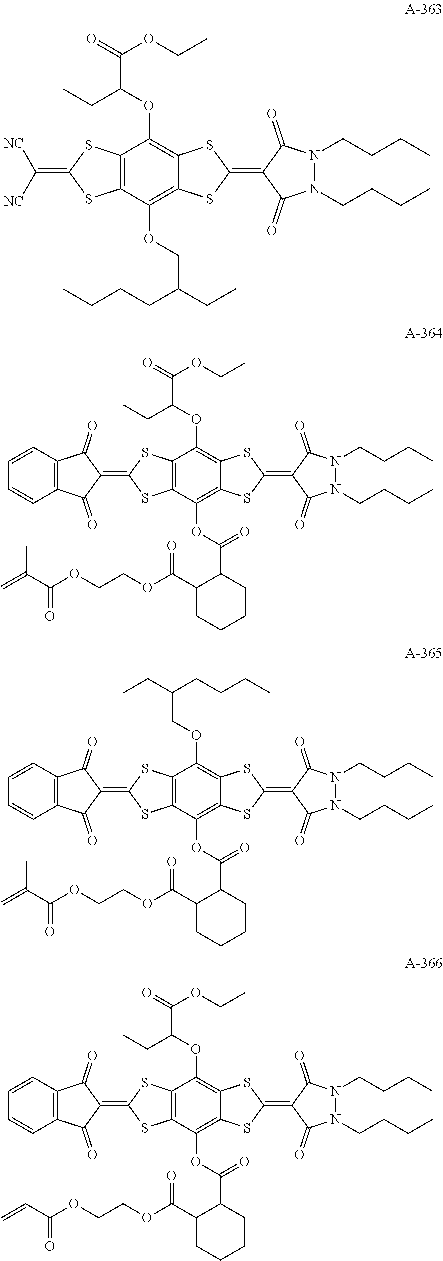

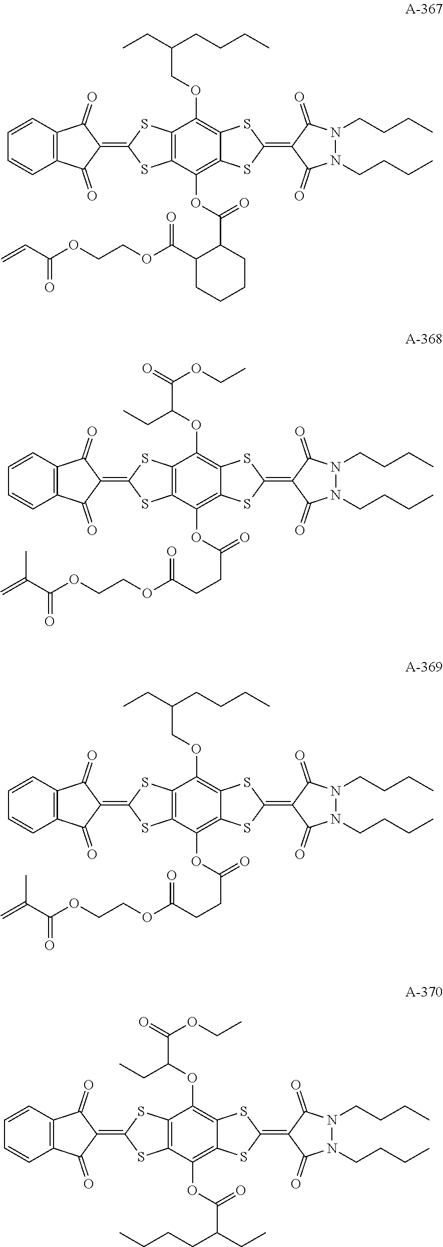

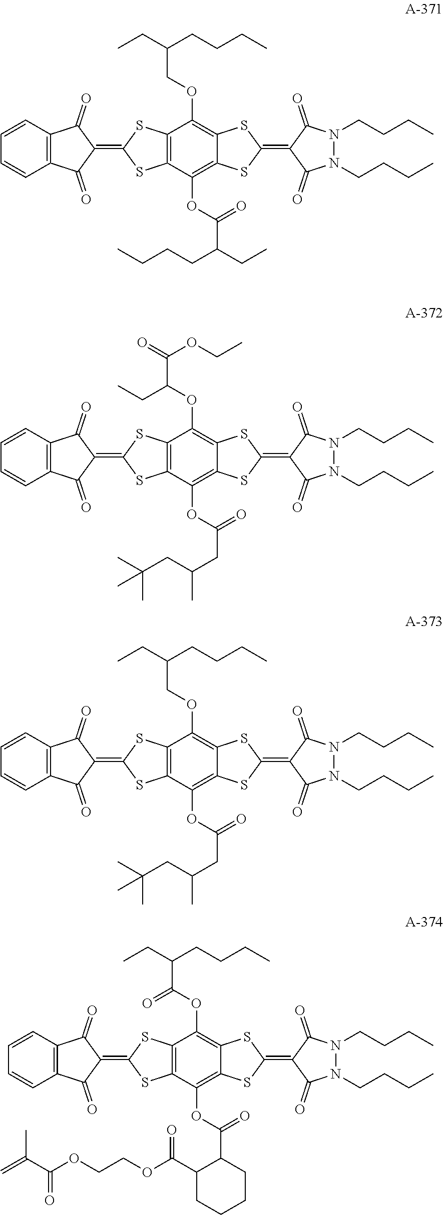

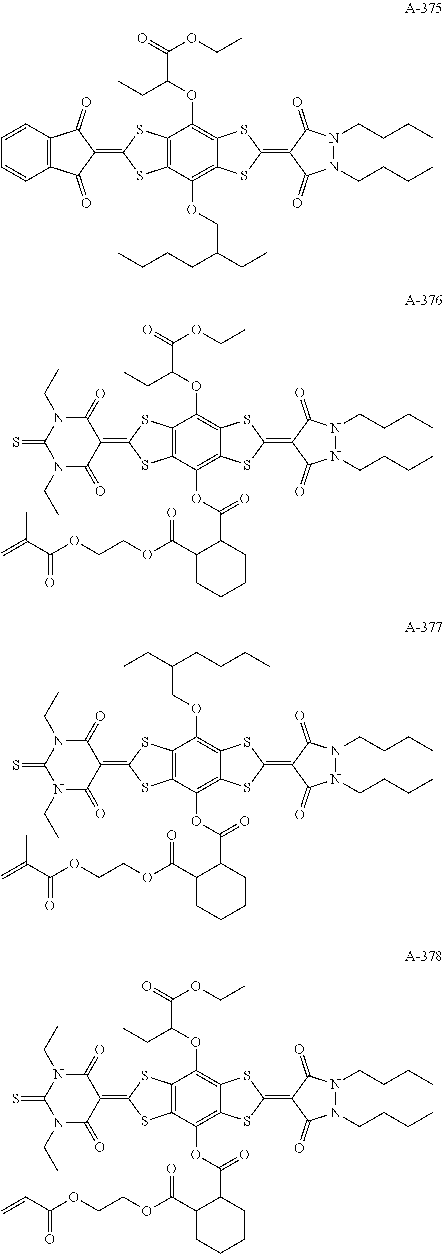

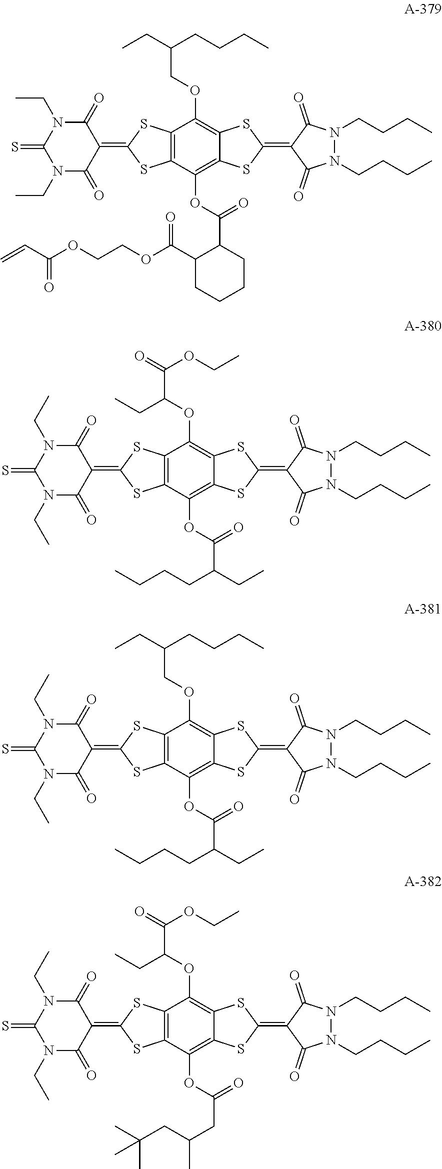 [SMILES: C=CC(=O)OCCOC(=O)C1CCCCC1C(=O)Oc1c2c(c(OCC(CC)CCCC)c3c1SC(=C1C(=O)N(CCCC)N(CCCC)C1=O)S3)SC(=C1C(=O)N(CC)C(=S)N(CC)C1=O)S2 ; CCCCC(CC)C(=O)Oc1c2c(c(OC(CC)C(=O)OCC)c3c1SC(=C1C(=O)N(CCCC)N(CCCC)C1=O)S3)SC(=C1C(=O)N(CC)C(=S)N(CC)C1=O)S2 ; CCCCC(CC)COc1c2c(c(OC(=O)C(CC)CCCC)c3c1SC(=C1C(=O)N(CCCC)N(CCCC)C1=O)S3)SC(=C1C(=O)N(CC)C(=S)N(CC)C1=O)S2 ; CCCCN1C(=O)C(=C2Sc3c(OC(=O)CC(C)CC(C)(C)C)c4c(c(OC(CC)C(=O)OCC)c3S2)SC(=C2C(=O)N(CC)C(=S)N(CC)C2=O)S4)C(=O)N1CCCC]

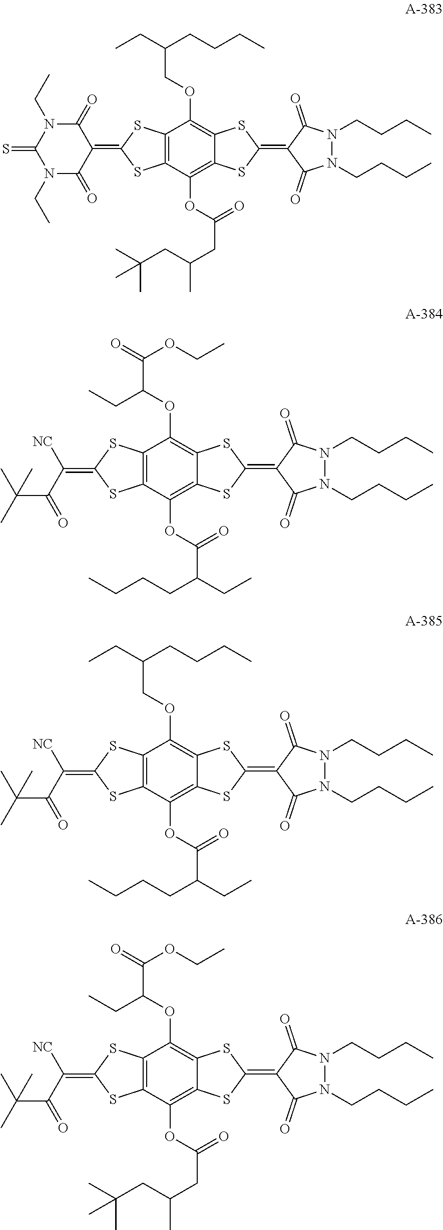

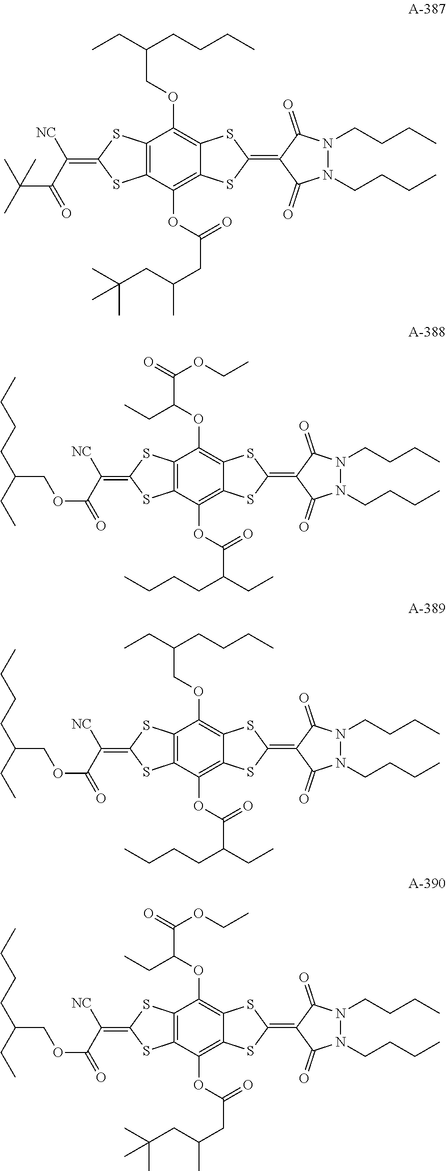 [SMILES: CCCCC(CC)COc1c2c(c(OC(=O)CC(C)CC(C)(C)C)c3c1S/C(=C(\C#N)C(=O)C(C)(C)C)S3)SC(=C1C(=O)N(CCCC)N(CCCC)C1=O)S2 ; CCCCC(CC)COC(=O)/C(C#N)=C1\Sc2c(OC(=O)C(CC)CCCC)c3c(c(OC(CC)C(=O)OCC)c2S1)SC(=C1C(=O)N(CCCC)N(CCCC)C1=O)S3 ; CCCCC(CC)COC(=O)/C(C#N)=C1/Sc2c(OCC(CC)CCCC)c3c(c(OC(=O)C(CC)CCCC)c2S1)SC(=C1C(=O)N(CCCC)N(CCCC)C1=O)S3 ; CCCCC(CC)COC(=O)/C(C#N)=C1\Sc2c(OC(=O)CC(C)CC(C)(C)C)c3c(c(OC(CC)C(=O)OCC)c2S1)SC(=C1C(=O)N(CCCC)N(CCCC)C1=O)S3]

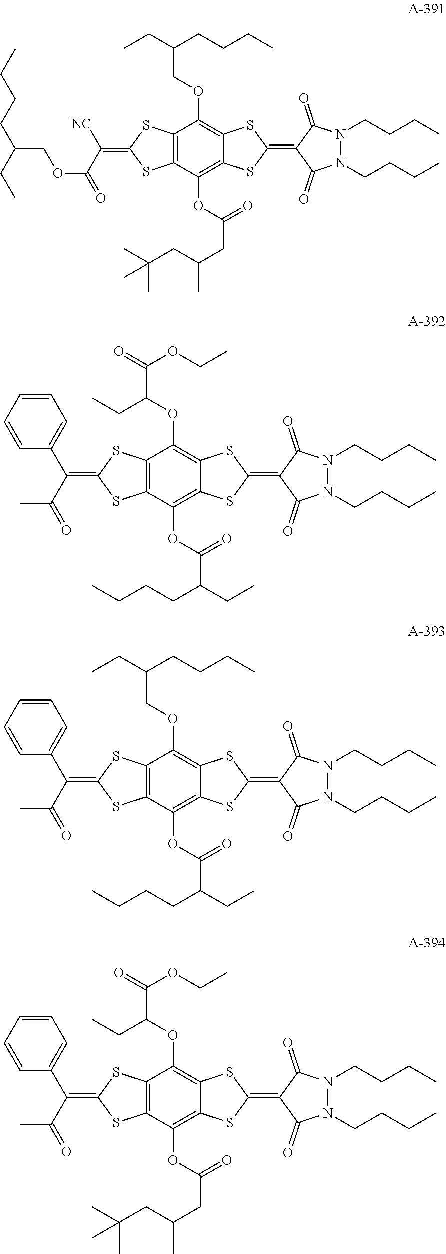

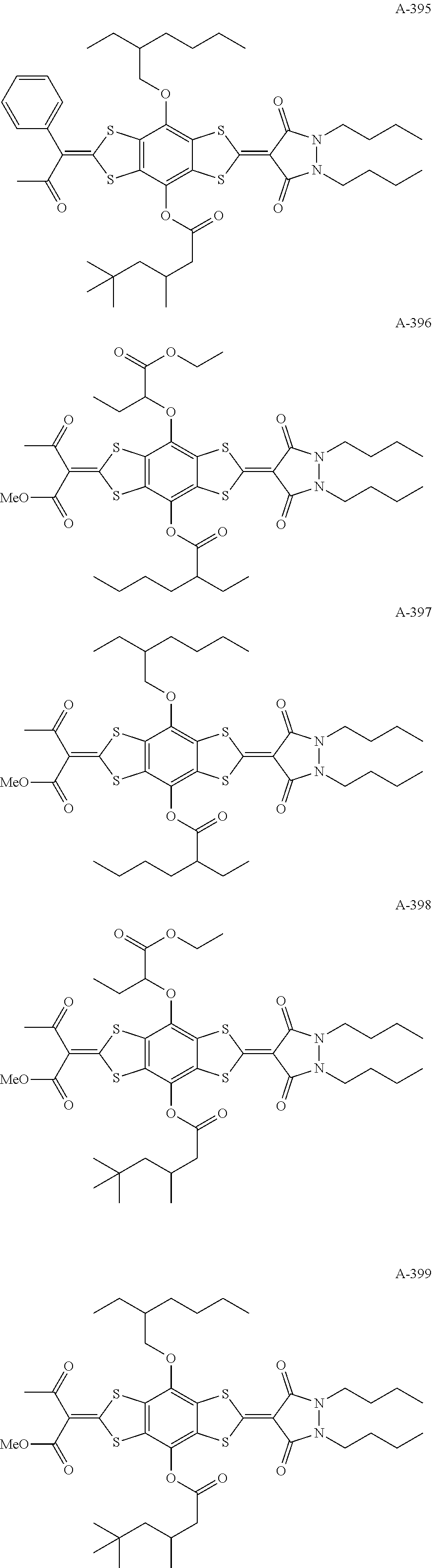

The maximal absorption wavelength of the specific compound is preferably in a wavelength range of 380 to 420 nm, and more preferably in a wavelength range of 390 to 410 nm.

In the specific compound, it is preferable that a value of a ratio of an absorbance at a wavelength of 440 nm to an absorbance at a wavelength of 400 nm in a case where the absorbance at the wavelength of 400 nm is set to 1 is less than 0.02.

A molar absorption coefficient of the specific compound at the maximal absorption wavelength is preferably 80,000 L/mol·cm or more, more preferably 85,000 L/mol·cm or more, and still more preferably 90,000 L/mol·cm or more.

In addition, a molar absorption coefficient at a wavelength of 400 nm is preferably 30,000 L/mol·cm or more, more preferably 40,000 L/mol·cm or more, and still more preferably 50,000 L/mol·cm or more.

In addition, a molar absorption coefficient at a wavelength of 440 nm is preferably 1,000 L/mol·cm or less, more preferably 800 L/mol·cm or less, and still more preferably 600 L/mol·cm or less.

The absorbance, the maximal absorption wavelength, the molar absorption coefficient of the specific compound can be determined by measuring spectral spectrum of a solution which is prepared by dissolving the specific compound in ethyl acetate at room temperature (25° C.), using a 1 cm quartz cell. Examples of a measuring device include a spectrophotometer (UV-1800PC, manufactured by Shimadzu Corporation).

The specific compound can be produced according to a method disclosed in WO2009/022736A.

In addition, among the specific compounds, the compound represented by Formula (6) can also be produced by reacting a compound represented by Formula (4) with a compound represented by Formula (5).

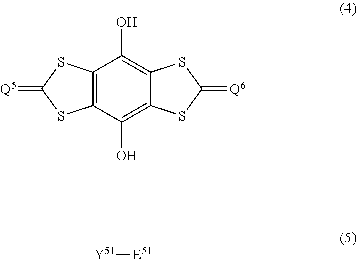

In Formula (4), Q⁵ represents the group represented by Formula (Q-1),
Q⁶ represents ═O, ═S, ═NR^{q21}, or ═CR^{q22}R^{q23},
R^{q21} to R^{q23} each independently represent a hydrogen atom or a substituent, and R^{q22} and R^{q23} may be bonded to each other to form a ring; provided that, in a case where R^{q22} and R^{q23} are bonded to each other to form a ring, ═CR^{q22}R^{q23} is not the same structure as Q⁵, and

In Formula (5), E⁵¹ represents —COCl, —O(C═O)Cl, —NR^{c51}(C═O)Cl, —NCO, —Cl, —Br, —I, or —SO₂R^{e52}
R^{e51} represents a hydrogen atom, an alkyl group, an aralkyl group, or an aryl group,
R^{e52} represents —Cl or an alkoxy group,
Y⁵¹ represents an alkyl group, an aralkyl group, or an aryl group.

Q⁵ and Q⁶ in Formula (4) have the same meaning as Q⁵ and Q⁶ in Formula (6), and preferred ranges thereof are also the same.

The number of carbon atoms in the alkyl group represented by Y⁵¹ in Formula (5) is preferably 1 to 30, more preferably 1 to 20, still more preferably 1 to 15, particularly preferably 1 to 10, and most preferably 1 to 8. The alkyl group may be linear, branched, or cyclic, and preferably linear or branched. The alkyl group may have a substituent. Examples of the substituent include the groups described in the substituent T above.

The number of carbon atoms in the aryl group represented by Y⁵¹ in Formula (5) is preferably 6 to 30, more preferably 6 to 20, still more preferably 6 to 15, particularly preferably 6 to 10, and most preferably 6 to 8. The aryl group may have a substituent. Examples of the substituent include the groups described in the substituent T above.

The number of carbon atoms in an alkyl moiety of the aralkyl group represented by Y⁵¹ in Formula (5) is preferably 1 to 10, more preferably 1 to 5, and still more preferably 1 to 3. The number of carbon atoms in an aryl moiety of the above-described aralkyl group is preferably 6 to 30, more preferably 6 to 20, still more preferably 6 to 15, particularly preferably 6 to 10, and most preferably 6 to 8. The aralkyl group may have a substituent. Examples of the substituent include the groups described in the substituent T above.

Y⁵¹ in Formula (5) is preferably an alkyl group.

The alkyl group, the aralkyl group, and the aryl group, represented by R^{e51} in E⁵¹ in Formula (5), are the same as the groups described in Y⁵¹ in Formula (5).

The number of carbon atoms in the alkoxy group represented by R^{e52} in E⁵¹ in Formula (5) is preferably 1 to 30, more preferably 1 to 20, still more preferably 1 to 15, particularly preferably 1 to 10, and most preferably 1 to 8.

The reaction between the compound represented by Formula (4) and the compound represented by Formula (5) can be carried out in an organic solvent. The organic solvent is not particularly limited, but for example, it is preferably an amide-based solvent such as dimethylformamide, dimethylacetamide, and N-methyl-2-pyrrolidone, tetrahydrofuran, acetonitrile, toluene, methanol, ethanol, isopropyl alcohol, or a mixed solution thereof, and dimethylformamide or dimethylacetamide is particularly preferable. In addition, a reaction ratio of the compound represented by Formula (4) and the compound represented by Formula (5) can be appropriately set according to a structure of the desired compound represented by Formula (6). A reaction temperature is not particularly limited, but is preferably 0° C. to a boiling point of the reaction solvent. A reaction time is not particularly limited, but for example, can be set to 1 hour to 48 hours.

(Polymer (Specific Polymer) Including Structure Derived from Compound Represented by Formula (1))

As the ultraviolet absorber, a polymer (specific polymer) including a structure derived from a compound represented by Formula (1) can also be used.

The specific polymer preferably includes a structure (hereinafter, also referred to as a structure (1)) derived from a compound having a structure in which at least one of R¹, R², Q¹, or Q² in the above-described compound represented by Formula (1) has a group including a polymerizable group having an ethylenically unsaturated bond, and more preferably includes a structure (hereinafter, also referred to as a structure (3)) derived from a compound having a structure in which at least one of R¹¹, R¹², Q³, or Q⁴ in the above-described compound represented by Formula (3) has a group including a polymerizable group having an ethylenically unsaturated bond.

The specific polymer may include a structure derived from a compound (hereinafter, also referred to as another polymerizable compound) other than the compound represented by Formula (1), which has an ethylenically unsaturated bond-containing group, in addition to the structure derived from the compound represented by Formula (1). That is, the specific polymer may be a copolymer of a compound represented by Formula (1) having a structure in which at least one of R¹, R², Q¹, or Q² is a group including a polymerizable group having an ethylenically unsaturated bond, and another polymerizable compound. Examples of the other polymerizable compounds include the polymerizable compounds described as materials used in the composition of the embodiment of the present invention, which will be described later. In addition, as the other polymerizable compounds, an ultraviolet absorber (also referred to as another polymerizable ultraviolet absorber) having a polymerizable group other than the compound represented by Formula (1) can also be used.

Examples of the polymerizable group contained in the other polymerizable ultraviolet absorber include a vinyl group, an allyl group, a (meth)acryloyl group, a (meth)acryloyloxy group, a (meth)acryloylamino group, and a vinylphenyl group. Examples of the other polymerizable ultraviolet absorbers include an aminobutadiene compound, a dibenzoylmethane compound, a benzophenone compound, a benzotriazole compound, and a hydroxyphenyltriazine compound.

The content of the structure derived from the compound represented by Formula (1) in the specific polymer is preferably 0.01% to 100% by mass. The upper limit thereof is more preferably 50% by mass or less and still more preferably 30% by mass or less. The lower limit thereof is more preferably 0.02% by mass or more and still more preferably 0.1% by mass or more.

A weight-average molecular weight of the specific polymer is preferably 5,000 to 150,000, more preferably 10,000 to 120,000, and still more preferably 20,000 to 100,000.

### <<<Other Ultraviolet Absorbers>>>

The composition according to the embodiment of the present invention can contain an ultraviolet absorber other than the above-described specific ultraviolet absorber (hereinafter, also referred to as other ultraviolet absorbers). According to this aspect, a cured substance capable of shielding light having a wavelength in the ultraviolet region over a wide range can be formed.

The maximal absorption wavelengths of the other ultraviolet absorbers are present preferably in a wavelength range of 300 to 380 nm, more preferably in a wavelength range of 300 to 370 nm, still more preferably in a wavelength range of 310 to 360 nm, and particularly preferable in a wavelength range of 310 to 350 nm.

It is also preferable that the other ultraviolet absorbers are compounds having a polymerizable group. Examples of the polymerizable group include a vinyl group, an allyl group, a (meth)acryloyl group, a (meth)acryloyloxy group, a (meth)acryloylamino group, and a vinylphenyl group.

Examples of the other ultraviolet absorbers include an aminobutadiene-based ultraviolet absorbing agent, a dibenzoylmethane-based ultraviolet absorbing agent, a benzotriazole-based ultraviolet absorbing agent, a benzophenone-based ultraviolet absorbing agent, a salicylic acid-based ultraviolet absorbing agent, an acrylate-based ultraviolet absorbing agent, and a triazine-based ultraviolet absorbing agent. Among these, a benzotriazole-based ultraviolet absorbing agent, a benzophenone-based ultraviolet absorbing agent, and a triazine-based ultraviolet absorbing agent are preferable, and a benzotriazole-based ultraviolet absorbing agent and a triazine-based ultraviolet absorbing agents are more preferable. Specific examples of the other ultraviolet absorbers include compounds described in Examples later. In addition, as the other ultraviolet absorbers, compounds and the like described in paragraphs 0065 to 0070 of JP2009-263616A, paragraph 0065 of WO2017/122503A, JP2003-128730A, JP2003-129033A, JP2014-077076A, JP2015-164994A, JP2015-168822A, JP2018-135282A, JP2018-168089A, JP2018-168278A, JP2018-188589A, JP2019-001767A, JP2020-023697A, JP2020-041013A, JP5518613B, JP5868465B, JP6301526B, JP6354665B, JP2017-503905A, WO2015/064674A, WO2015/064675A, WO2017/102675A, WO2018/190281A, WO2018/216750A, WO2019/087983A, EP2379512B, and EP2951163B can be used.

A content of the ultraviolet absorber in the total solid content of the composition is preferably 0.01% to 95% by mass. The lower limit can be set to 0.05% by mass or more, 0.1% by mass or more, 1% by mass or more, 5% by mass or more, 10% by mass or more, 20% by mass or more, 25% by mass or more, or 30% by mass or more. The upper limit thereof can be set to 90% by mass or less, 80% by mass or less, 70% by mass or less, 60% by mass or less, 50% by mass or less, 40% by mass or less, 30% by mass or less, or 20% by mass or less. The content of the specific ultraviolet absorber in the ultraviolet absorbing agent is preferably 5% by mass or more, more preferably 10% by mass or more, and still more preferably 20% by mass or more. The upper limit thereof can be set to 100% by mass or less, 75% by mass or less, or 50% by mass or less.

A content of the specific ultraviolet absorber in the total solid content of the composition is preferably 0.01% to 50% by mass. The lower limit thereof is preferably 0.05% by mass or more and more preferably 0.1% by mass or more. The upper limit thereof is preferably 40% by mass or less, more preferably 30% by mass or less, and still more preferably 20% by mass or less.

In a case where the above-described specific compound (the compound represented by Formula (1)) is used as the specific ultraviolet absorber, the content of the specific compound in the total solid content of the composition is preferably 0.01% to 50% by mass. The lower limit thereof is preferably 0.05% by mass or more and more preferably 0.1% by mass or more. The upper limit thereof is preferably 40% by mass or less, more preferably 30% by mass or less, and still more preferably 20% by mass or less.

In a case where the specific polymer (a polymer including a structure derived from the compound represented by Formula (1)) described above is used as the specific ultraviolet absorber, the content of the specific polymer in the total solid content of the composition is preferably 10% to 95% by mass. The lower limit thereof is preferably 15 mass % or more and more preferably 25 mass % or more. The upper limit thereof is preferably 90% by mass or less, more preferably 85% by mass or less, and still more preferably 75% by mass or less.

The composition according to the embodiment of the present invention may contain only one kind of ultraviolet absorber or two or more kinds thereof. In a case where two or more kinds of ultraviolet absorbers are contained, it is preferable that the total amount thereof is within the above-described range.

### <<Anti-Fading Agent>>

The composition according to the embodiment of the present invention contains an anti-fading agent. As the anti-fading agent, at least one selected from an amine compound, a phenol compound, a hydroquinone compound, a catechol compound, an ascorbic acid compound, a carotenoid compound, a metal complex compound, or a benzolactone compound is used. The amine compound is preferably a hydroxyamine compound, a tertiary amine compound, or an aminoxyl compound, and more preferably a tertiary amine compound or an aminoxyl compound. The metal complex compound is preferably a Ni complex compound or a Co complex compound, and more preferably a Ni complex compound. The anti-fading agent is preferably at least one selected from an amine compound or a benzolactone compound, and more preferably an amine compound.

The anti-fading agent preferably includes at least one selected from a compound represented by Formula (Ao1-1) or a compound represented by Formula (Ao2-1), and more preferably includes a compound represented by Formula (Ao1-1).

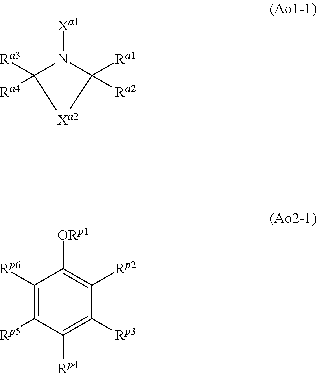

in Formula (Ao1-1), R^{a1} to R^{a4} each independently represent a hydrogen atom, an alkyl group, or an alkenyl group,
X^{a1} represents a hydrogen atom, an alkyl group, an alkenyl group, an alkoxy group, an alkenyloxy group, an alkoxycarbonyl group, an alkenyloxycarbonyl group, an aryloxycarbonyl group, an acyl group, an acyloxy group, an alkoxycarbonyloxy group, an alkenyloxycarbonyloxy group, an aryloxycarbonyloxy group, an alkylsulfonyl group, an alkenylsulfonyl group, an arylsulfonyl group, an alkylsulfinyl group, an alkenylsulfinyl group, an arylsulfinyl group, a sulfamoyl group, a carbamoyl group, a hydroxy group, or an oxyradical group,
X^{a2} represents an atomic group necessary to form a 5- to 7-membered ring,
in Formula (Ao2-1), R^{p1} represents a hydrogen atom, an alkyl group, an alkenyl group, an aryl group, a heterocyclic group, an acyl group, an alkoxycarbonyl group, an alkenyloxycarbonyl group, an aryloxycarbonyl group, an alkylsulfonyl group, an arylsulfonyl group, or —Si(R^{p101})(R^{p102})(R^{p103}), where R^{p101} to R^{p103} each independently represent an alkyl group, an alkenyl group, an aryl group, an alkoxy group, an alkenyloxy group, or an aryloxy group,
R^{p2} to R^{p6} each independently represent a hydrogen atom or a substituent, and
two adjacent groups among R^{p1} to R^{p6} may be bonded to each other to form a ring,
provided that, all of R^{p1} to R^{p6} are not hydrogen atoms.

The above-described group in Formula (Ao1-1) and Formula (Ao1-2) may have a substituent. Examples of the substituent include groups in the description of the substituent T described above.

### —About Compound Represented by Formula (Ao1-1)—

R^{a1} to R^{a4} in Formula (Ao1-1) are each independently preferably an alkyl group or an alkenyl group, and more preferably an alkyl group.

The number of carbon atoms in the alkyl group is preferably 1 to 5 and more preferably 1 to 3. The alkyl group may be linear, branched, or cyclic, but is preferably linear.

The alkyl group may have a substituent. Examples of the substituent include groups in the description of the substituent T described above.

The number of carbon atoms in the alkenyl group is preferably 2 to 5 and more preferably 2 or 3. The alkenyl group is preferably linear or branched, and more preferably linear. The alkenyl group may have a substituent. Examples of the substituent include groups in the description of the substituent T described above.

R^{a1} to R^{a4} are particularly preferably a methyl group.

X^{a1} in Formula (Ao1-1) is preferably a hydrogen atom, an oxyradical group, an alkyl group, an alkoxy group, an alkenyl group, an acyl group, or an aryl group, and more preferably an oxyradical group or an alkoxy group.

The number of carbon atoms in the alkyl group is preferably 1 to 20 and more preferably 1 to 12. The alkyl group may be linear, branched, or cyclic, but is preferably linear. The alkyl group may have a substituent. Examples of the substituent include groups in the description of the substituent T described above.

The number of carbon atoms in the alkenyl group is preferably 2 to 20 and more preferably 2 to 12. The alkenyl group is preferably linear or branched, and more preferably linear. The alkenyl group may have a substituent. Examples of the substituent include groups in the description of the substituent T described above.

The number of carbon atoms in the alkoxy group is preferably 1 to 20 and more preferably 1 to 12. The alkoxy group is preferably linear or branched, and more preferably linear. The alkoxy may have a substituent. Examples of the substituent include groups in the description of the substituent T described above.

The number of carbon atoms in the acyl group is preferably 2 to 20 and more preferably 2 to 14′. The acyl group may have a substituent. Examples of the substituent include groups in the description of the substituent T described above.

The number of carbon atoms in the aryl group is preferably 6 to 20. The aryl group may have a substituent. Examples of the substituent include groups in the description of the substituent T described above.

The compound represented by Formula (Ao1-1) is preferably a compound represented by Formula (Ao1-2).

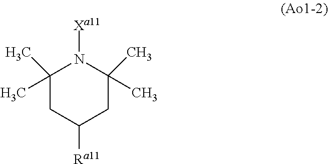

in Formula (Ao1-2), X^{a11} represents a hydrogen atom, an alkyl group, an alkenyl group, an alkoxy group, an alkenyloxy group, an alkoxycarbonyl group, an alkenyloxycarbonyl group, an aryloxycarbonyl group, an acyl group, an acyloxy group, an alkoxycarbonyloxy group, an alkenyloxycarbonyloxy group, an aryloxycarbonyloxy group, an alkylsulfonyl group, an alkenylsulfonyl group, an arylsulfonyl group, an alkylsulfinyl group, an alkenylsulfinyl group, an arylsulfinyl group, a sulfamoyl group, a carbamoyl group, a hydroxy group, or an oxyradical group, and
R^{a11} represents a substituent,
X^{a11} in Formula (Ao1-2) has the same definition as X^{a1} in Formula (Ao1-1), and a preferred range thereof is also the same.

Examples of the substituent represented by R^{a1} in Formula (Ao1-2) include the groups mentioned in the above-described substituent T. Substituent group: a halogen atom, an alkyl group, an alkenyl group, an alkynyl group, an aryl group, a heterocyclic group, a cyano group, a hydroxy group, a nitro group, a carboxy group, an alkoxy group, an aryloxy group, a heterocyclic oxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkylsulfonylamino group, an arylsulfonylamino group, a mercapto group, an alkylthio group, an arylthio group, a heterocyclic thio group, a sulfamoyl group, a sulfo group, an alkylsulfinyl group, an arylsulfinyl group, an alkylsulfonyl group, an arylsulfonyl group, an acyl group, an aryloxycarbonyl group, an alkoxycarbonyl group, a carbamoyl group, an arylazo group, a heterocyclic azo group, an imide group, a phosphino group, a phosphinyl group, a phosphinyloxy group, a phosphinylamino group, and a silyl group.

The compound represented by Formula (Ao1-1) is preferably a compound represented by Formula (Ao1-3).

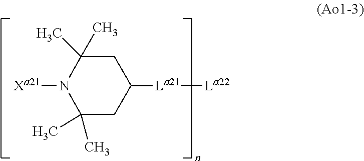

In Formula (Ao1-3), X^{a2}1 represents a hydrogen atom, an alkyl group, an alkenyl group, an alkoxy group, an alkenyloxy group, an alkoxycarbonyl group, an alkenyloxycarbonyl group, an aryloxycarbonyl group, an acyl group, an acyloxy group, an alkoxycarbonyloxy group, an alkenyloxycarbonyloxy group, an aryloxycarbonyloxy group, an alkylsulfonyl group, an alkenylsulfonyl group, an arylsulfonyl group, an alkylsulfinyl group, an alkenylsulfinyl group, an arylsulfinyl group, a sulfamoyl group, a carbamoyl group, a hydroxy group, or an oxyradical group,
L^{a21} represents a single bond or a divalent linking group.
L^{a22} represents an n-valent group,
n represents an integer of 1 or more.
X^{a21} in Formula (Ao1-3) has the same definition as X^{a1} in Formula (Ao1-1), and a preferred range thereof is also the same.

Examples of the divalent linking group represented by L^{a21} in Formula (Ao1-3) include —O—, —CO—, —COO—, —OCO—, —NHCO—, —NHCOO—, —CONH—, —OCONH—, —S—, —SO₂—, and —OSO₂—.

L^{a22} in Formula (Ao1-3) represents an n-valent group. In a case where the n-valent group represented by L^{a22} is a monovalent group (in a case where n is 1), examples of the n-valent group represented by L^{a22} include an alkyl group, an alkenyl group, an alkynyl group, an aryl group, a heterocyclic group, a cyano group, a hydroxy group, a nitro group, a carboxy group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkylsulfonylamino group, an arylsulfonylamino group, a mercapto group, an alkylthio group, an arylthio group, a heterocyclic thio group, a sulfamoyl group, a sulfo group, an alkylsulfinyl group, an arylsulfinyl group, an alkylsulfonyl group, an arylsulfonyl group, an acyl group, an aryloxycarbonyl group, an alkoxycarbonyl group, a carbamoyl group, an arylazo group, a heterocyclic azo group, an imide group, a phosphino group, a phosphinyl group, a phosphinyloxy group, a phosphinylamino group, and a silyl group.

In a case where the n-valent group represented by L^{a22} is a divalent or higher valent group (in a case where n is an integer of 2 or more), examples of the n-valent group represented by L^{a22} include a hydrocarbon group; and a group in which two or more hydrocarbon groups are bonded to each other by —O—, —CO—, —COO—, —OCO—, —NH—, —S—, or a group obtained by combining two or more of these groups.

Examples of the hydrocarbon group include an aliphatic hydrocarbon group and an aromatic hydrocarbon group, and an aliphatic hydrocarbon group is preferable. The number of carbon atoms of the aliphatic hydrocarbon group is preferably 1 to 30, more preferably 1 to 20, and still more preferably 1 to 15. The aliphatic hydrocarbon group may be linear, branched, or cyclic, but is preferably linear or branched. The aliphatic hydrocarbon group may be any of a saturated aliphatic hydrocarbon group or an unsaturated aliphatic hydrocarbon group, but is preferably a saturated aliphatic hydrocarbon group.

The number of carbon atoms of the aromatic hydrocarbon group is preferably 6 to 30, more preferably 6 to 20, and still more preferably 6 to 10.

The hydrocarbon group may have a substituent. Examples of the substituent include a halogen atom, a hydroxy group an alkyl group, and an aryl group.

In a case where the n-valent group is a divalent or higher valent group, the n-valent linking group represented by L^{a22} is preferably a group including a saturated aliphatic hydrocarbon group having 1 to 20 carbon atoms (preferably 2 to 15 carbon atoms and more preferably 2 to 10 carbon atoms).
n in Formula (Ao1-3) represents an integer of 1 or more, preferably represents an integer of 2 or more, more preferably represents an integer of 2 to 6, and still more preferably represents an integer of 2 to 4.
   —Regarding Compound Represented by Formula (Ao2-1)—R^{p6} in Formula (Ao2-1) represents a hydrogen atom, an alkyl group, an alkenyl group, an aryl group, a heterocyclic group, an acyl group, an alkoxycarbonyl group, an alkenyloxycarbonyl group, an aryloxycarbonyl group, an alkylsulfonyl group, an arylsulfonyl group, or —Si(R^{p101})(R^{p102})(R^{p103}), and R^{p101} to R^{p103} each independently represent an alkyl group, an alkenyl group, an aryl group, an alkoxy group, an alkenyloxy group, or an aryloxy group.

R^{p1} is preferably a hydrogen atom, an alkyl group, or an acyl group, and more preferably an alkyl group or an acyl group.

Examples of the substituent represented by R^{p2} to R^{p6} include a halogen atom, an alkyl group, an alkenyl group, an alkynyl group, an aryl group, a heterocyclic group, a cyano group, a hydroxy group, a nitro group, a carboxy group, an alkoxy group, an aryloxy group, a silyloxy group, a heterocyclic oxy group, an acyloxy group, a carbamoyloxy group, an alkoxycarbonyloxy group, aryloxycarbonyloxy, an amino group, an acylamino group, an aminocarbonylamino group, an alkoxycarbonylamino group, an aryloxycarbonylamino group, a sulfamoylamino group, an alkylsulfonylamino group, an arylsulfonylamino group, a mercapto group, an alkylthio group, an arylthio group, a heterocyclic thio group, a sulfamoyl group, a sulfo group, an alkylsulfinyl group, an arylsulfinyl group, an alkylsulfonyl group, an arylsulfonyl group, an acyl group, an aryloxycarbonyl group, an alkoxycarbonyl group, a carbamoyl group, an arylazo group, a heterocyclic azo group, an imide group, a phosphino group, a phosphinyl group, a phosphinyloxy group, a phosphinylamino group, and a silyl group, and an alkyl group, an alkenyl group, an aryl group, an alkoxy group, an alkoxycarbonyl group, an aryloxy group, an aryloxycarbonyl group, an alkylsulfonyl group, or an arylsulfonyl group is preferable, an alkyl group or an alkenyl group is more preferable, and an alkyl group is still more preferable.

The number of carbon atoms in the alkyl group is preferably 1 to 10 and more preferably 1 to 5. The alkyl group may be linear, branched, or cyclic, but is preferably linear or branched and more preferably branched. The alkyl group may have a substituent. Examples of the substituent include groups in the description of the substituent T described above.

The number of carbon atoms in the alkenyl group is preferably 2 to 10 and more preferably 2 to 5. The alkenyl group is preferably linear or branched and more preferably branched. The alkenyl group may have a substituent. Examples of the substituent include groups in the description of the substituent T described above.

It is preferable that at least one of R^{p2} to R^{p6} is a substituent, and it is more preferable that two or more thereof are substituents.

In Formula (Ao2-1), two adjacent groups among R^{p1} to R^{p6} may be bonded to each other to form a ring. The ring formed is preferably a 5-membered ring or a 6-membered ring.

The formed ring may further have a substituent. Examples of the substituent include the groups and the ethylenically unsaturated bond-containing group mentioned in the section of the substituent T. Examples of the ethylenically unsaturated bond-containing group include a vinyl group.

Examples of a preferred aspect of Formula (Ao2-1) include an aspect in which R^{p1} and R^{p2} are bonded to each other to form a ring. The ring formed is preferably a 5-membered ring or a 6-membered ring. It is preferable that the ring to be formed further has a substituent.

Examples of the substituent include an aryl group, a heteroaryl group, and an ethylenically unsaturated bond-containing group, and an aryl group or an ethylenically unsaturated bond-containing group is preferable and an aryl group is more preferable. The aryl group and the heteroaryl group may further have a substituent. Examples of the further substituent include an alkyl group, an alkenyl group, an aryl group, an alkoxy group, an alkenyloxy group, an aryloxy group, an acyl group, and a hydroxy group.

In Formula (Ao2-1), all of R^{p1} to R^{p6} are not hydrogen atoms.

The total number of carbon atoms in R^{p1} to R^{p6} is preferably 10 or more and more preferably 16 or more.

The compound represented by Formula (Ao2-1) is preferably a compound represented by Formula (Ao2-2).

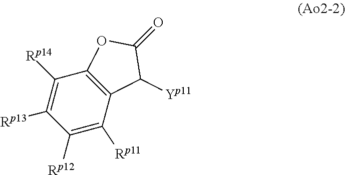

in Formula (Ao2-2), R^{p11} to R^{p14} each independently represent a hydrogen atom, an alkyl group, or an alkenyl group, and
Y^{p11} represents an aryl group, a heteroaryl group, or an ethylenically unsaturated bond-containing group.

The number of carbon atoms in the alkyl group represented by R^{p11} to R^{p14} is preferably 1 to 10 and more preferably 1 to 5. The alkyl group may be linear, branched, or cyclic, but is preferably linear or branched and more preferably branched. The alkyl group may have a substituent. Examples of the substituent include groups in the description of the substituent T described above.

The number of carbon atoms in the alkenyl group represented by R^{p11} to R^{p14} is preferably 2 to 10, and more preferably 2 to 5. The alkenyl group is preferably linear or branched and more preferably branched. The alkenyl group may have a substituent.

Examples of the substituent include groups in the description of the substituent T described above.

It is preferable that at least one of R^{p11} to R^{p14} is an alkyl group or an alkenyl group, and it is more preferable that two or more thereof are an alkyl group or an alkenyl group.

Among these, at least one of R^{p11} to R^{p14} is preferably an alkyl group, and two or more thereof are more preferably alkyl groups.

Y^{p11} represents an aryl group, a heteroaryl group, or an ethylenically unsaturated bond-containing group, and is preferably an aryl group.

The aryl group and the heteroaryl group may further have a substituent. Examples of the further substituent include an alkyl group, an alkenyl group, an aryl group, an alkoxy group, an alkenyloxy group, an aryloxy group, an acyl group, and a hydroxy group.

Examples of the above-described ethylenically unsaturated bond-containing group include a vinyl group.

The compound represented by Formula (Ao2-1) is preferably a compound represented by Formula (Ao2-3).

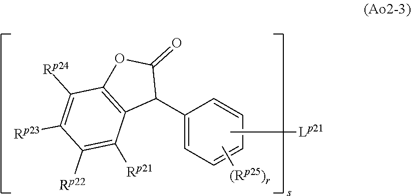

In Formula (Ao2-3), R^{p21} to R^{p24} each independently represent a hydrogen atom, an alkyl group, or an alkenyl group,
R^{p25} represents an alkyl group, an alkenyl group, or a hydroxy group,
L^{p21} represents an s-valent group,
r represents an integer of 0 to 4,
s represents an integer of 1 or more,

In a case where r is an integer of 2 or more, r pieces of R^{p25}'s may be the same as or different from each other.

R^{p21} to R^{p24} in Formula (Ao2-3) have the same definitions as R^{p11} to R^{p14} in Formula (Ao2-2), and preferred ranges thereof are also the same.

It is preferable that at least one of R^{p21} to R^{p24} is an alkyl group or an alkenyl group, and it is more preferable that two or more thereof are an alkyl group or an alkenyl group.

Among these, at least one of R^{p21} to R^{p24} is preferably an alkyl group, and more preferably two or more thereof are alkyl groups.

R^{p25} in Formula (Ao2-3) is preferably an alkyl group or an alkenyl group, and more preferably an alkyl group.

The number of carbon atoms in the alkyl group is preferably 1 to 10 and more preferably 1 to 5. The alkyl group may be linear, branched, or cyclic, but is preferably linear or branched and more preferably branched. The alkyl group may have a substituent.

Examples of the substituent include groups in the description of the substituent T described above.

The number of carbon atoms in the alkenyl group is preferably 2 to 10 and more preferably 2 to 5. The alkenyl group is preferably linear or branched and more preferably branched. The alkenyl group may have a substituent. Examples of the substituent include groups in the description of the substituent T described above.

L^{p21} represents an s-valent group. In a case where the s-valent group represented by L^{p21} is a monovalent group, examples of the s-valent group represented by L^{p21} include an alkyl group, an alkenyl group, an alkoxy group, and an alkynyl group, and an alkyl group or an alkoxy group is preferable.

In a case where the s-valent group represented by L^{p21} is a divalent or higher valent group, examples of the s-valent group represented by L^{p21} include a hydrocarbon group, —O—, —CO—, —COO—, —OCO—, —NHCO—, —NHCOO—, —CONH—, —OCONH—, —S—, —SO₂—, —OSO₂—, and a group obtained by combining two or more of these groups.

Examples of the hydrocarbon group include an aliphatic hydrocarbon group and an aromatic hydrocarbon group. The number of carbon atoms of the aliphatic hydrocarbon group is preferably 1 to 30, more preferably 1 to 20, and still more preferably 1 to 15. The aliphatic hydrocarbon group may be linear, branched, or cyclic, but is preferably linear or branched. The aliphatic hydrocarbon group may be any of a saturated aliphatic hydrocarbon group or an unsaturated aliphatic hydrocarbon group, but is preferably a saturated aliphatic hydrocarbon group.

The number of carbon atoms of the aromatic hydrocarbon group is preferably 6 to 30, more preferably 6 to 20, and still more preferably 6 to 10.

The hydrocarbon group may have a substituent. Examples of the substituent include a halogen atom, a hydroxy group an alkyl group, and an aryl group.
r represents an integer of 0 to 4, and is preferably an integer of 0 to 2.
s represents an integer of 1 or more, preferably an integer of 1 to 4, and more preferably 1 or 2.

Specific examples of the anti-fading agent include compounds described in Examples described later, compounds described in paragraphs 0157 to 0171 of JP2009-067984A, and compounds having the structures shown below.

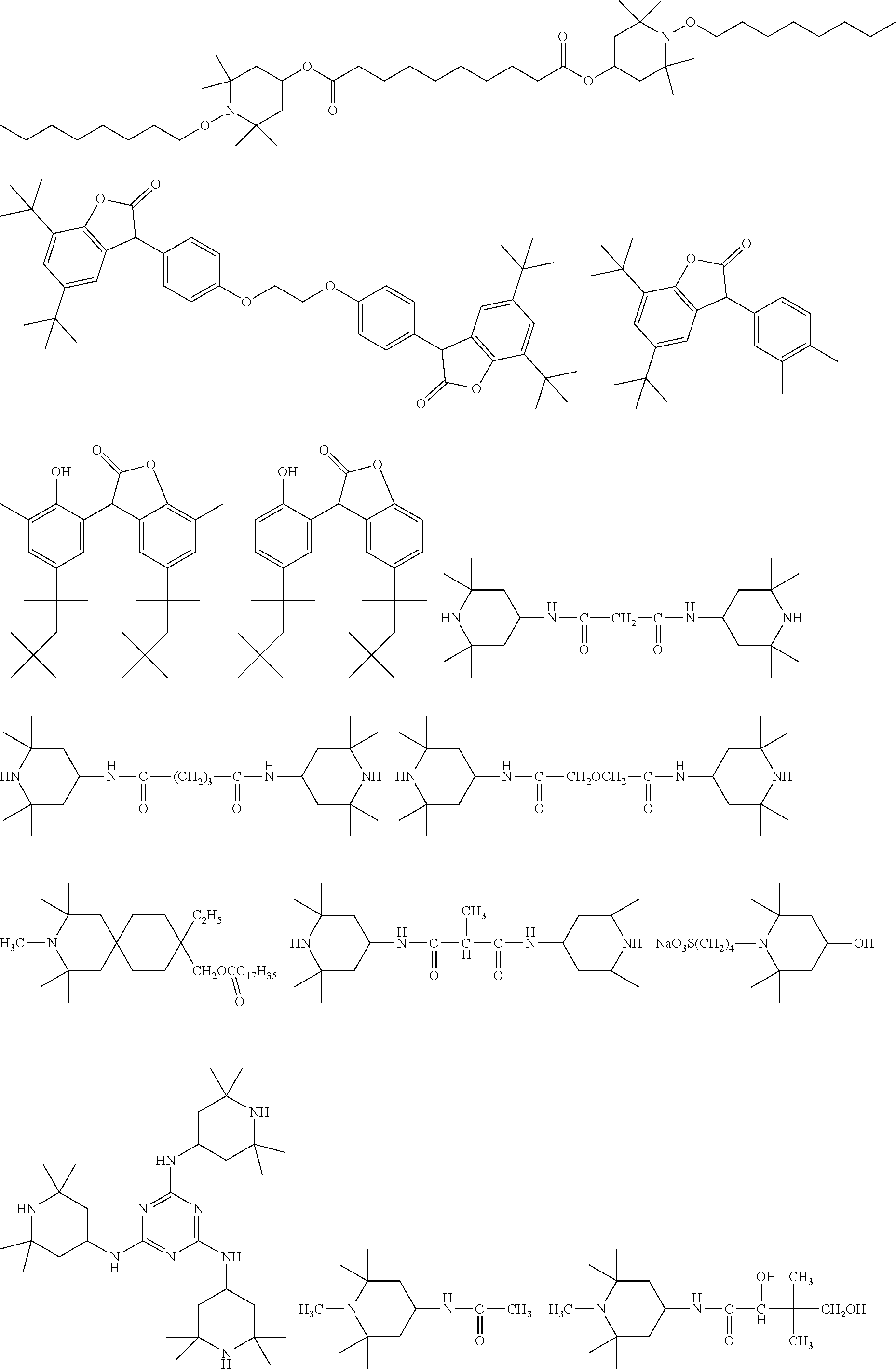

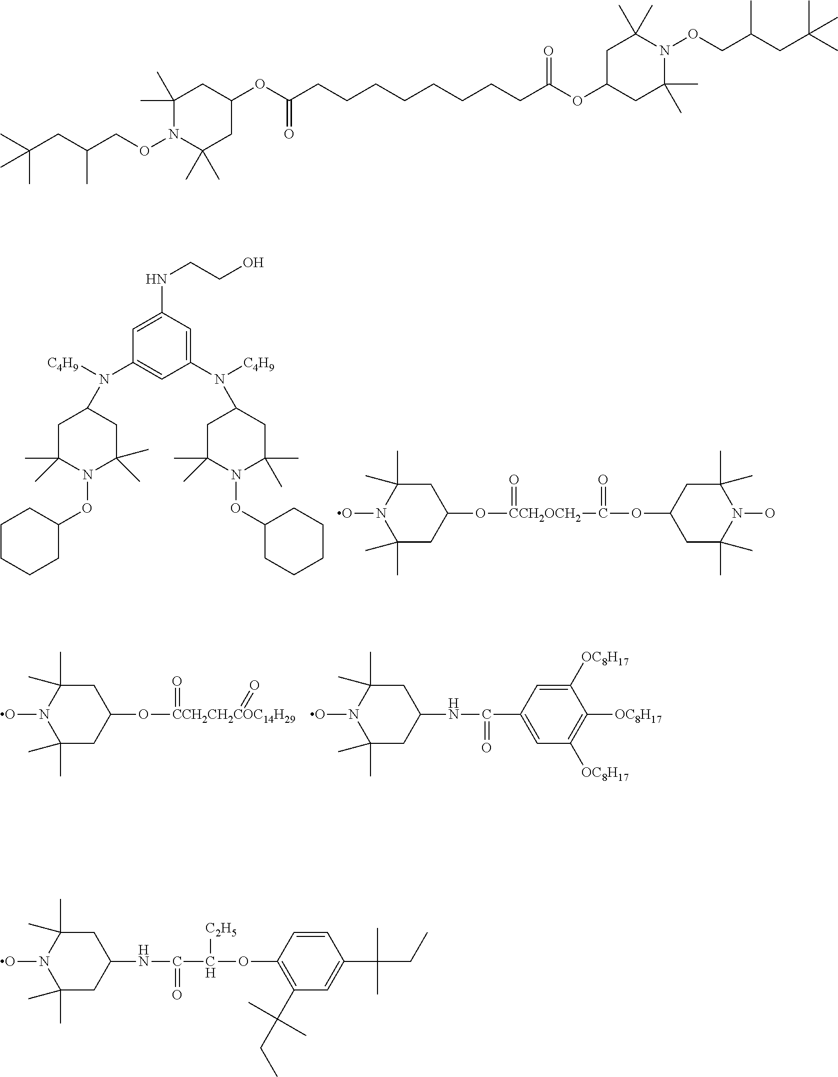

A content of the anti-fading agent in the total solid content of the composition is preferably 0.01% to 50% by mass. The lower limit thereof is preferably 0.05% by mass or more and more preferably 0.1% by mass or more. The upper limit thereof is preferably 40% by mass or less, more preferably 30% by mass or less, and still more preferably 20% by mass or less.

The content of the anti-fading agent is preferably 0.1 to 500 parts by mass with respect to 100 parts by mass of the ultraviolet absorber. The upper limit thereof is preferably 300 parts by mass or less and more preferably 200 parts by mass or less. The lower limit is preferably 1 parts by mass or more and more preferably 10 parts by mass or more.

The content of the anti-fading agent is preferably 0.1 to 500 parts by mass with respect to 100 parts by mass of the above-described specific ultraviolet absorber. The upper limit thereof is preferably 300 parts by mass or less and more preferably 200 parts by mass or less. The lower limit is preferably 1 parts by mass or more and more preferably 10 parts by mass or more.

The composition according to the embodiment of the present invention may contain only one kind of the anti-fading agent or two or more kinds thereof. In a case where two or more kinds of anti-fading agents are contained, the total amount thereof is preferably within the above-described range.

<<Curable Compound>>

The composition according to the embodiment of the present invention contains a curable compound. Examples of the curable compound include a polymerizable compound and a resin. The resin may be a non-polymerizable resin (resin not having a polymerizable group), or may be a polymerizable resin (resin having a polymerizable group). Examples of the polymerizable group include an ethylenically unsaturated bond-containing group and a cyclic ether group. Examples of the ethylenically unsaturated bond-containing group include a vinyl group, a vinylphenyl group, a (meth)allyl group, a (meth)acryloyl group, a (meth)acryloyloxy group, and a (meth)acryloylamide group. Among these, a (meth)allyl group, a (meth)acryloyl group, or a (meth)acryloyloxy group is preferable, and a (meth)acryloyloxy group is more preferable. Examples of the cyclic ether group include an epoxy group and an oxetanyl group, and an epoxy group is preferable.

It is preferable that the curable compound contained in the composition according to the embodiment of the present invention contains at least one selected from a resin or a polymerizable compound. A curable compound may be used in combination with a resin and a polymerizable compound.

### (Polymerizable Compound)

As the polymerizable compound, a compound that can be polymerized and cured by applying energy can be used without limitation. The polymerizable compound may be a radically polymerizable compound or a cationically polymerizable compound.

It is preferable that the polymerizable compound may be any one of a monomer, a prepolymer (that is, a dimer, a trimer, or an oligomer), a mixture thereof, and a (co)polymer of a compound selected from the monomer and the prepolymer.

The molecular weight of the polymerizable compound is preferably 100 to 3,000. The upper limit thereof is preferably 2,000 or less and more preferably 1,500 or less. The lower limit thereof is preferably 150 or more and more preferably 250 or more.

### —Radically Polymerizable Compound—

Examples of the radically polymerizable compound include a compound containing an ethylenically unsaturated bond-containing group. Examples of the radically polymerizable compound include an unsaturated carboxylic acid (such as acrylic acid, methacrylic acid, itaconic acid, crotonic acid, isocrotonic acid, or maleic acid), an ester of an unsaturated carboxylic acid, an amide of an unsaturated carboxylic acid, and a (co)polymer of the unsaturated carboxylic acid, the ester thereof, or the amide thereof. Among these, esters of an unsaturated carboxylic acid and an aliphatic polyhydric alcohol, amides of an unsaturated carboxylic acid and an aliphatic polyvalent amine, and homopolymers or copolymers thereof are preferable.

As the radically polymerizable compound, an addition reactant of an unsaturated carboxylic acid ester or unsaturated carboxylic acid amide having a nucleophilic substituent (for example, a hydroxy group, an amino group, a mercapto group, and the like) and a monofunctional or polyfunctional isocyanate compound or epoxy compound; a dehydration condensation reactant of an unsaturated carboxylic acid ester or unsaturated carboxylic acid amide having a nucleophilic substituent and a monofunctional or polyfunctional carboxylic acid; an addition reactant of an unsaturated carboxylic acid ester or unsaturated carboxylic acid amide having an electrophilic substituent (for example, an isocyanate group, an epoxy group, and the like) and a monofunctional or polyfunctional alcohol, amine, or thiol; and a substitution reactant of an unsaturated carboxylic acid ester or unsaturated carboxylic acid amide having a leaving substituent (for example, a halogen atom, a tosyloxy group, and the like) and a monofunctional or polyfunctional alcohol, amine, or thiol can also be used. Further, a compound obtained by substituting the above-described unsaturated carboxylic acid with an unsaturated phosphonic acid, styrene, vinyl ether, or the like can also be used.

A plurality of compounds with different numbers of functional groups or a plurality of compounds with different kinds of polymerizable groups (for example, acrylic acid ester, methacrylic acid ester, a styrene compound, a vinyl ether compound, and the like) may be used in combination as the radically polymerizable compound.

As the radically polymerizable compound, a (meth)acrylate compound is preferable, a bifunctional or higher functional (meth)acrylate compound is more preferable, a bifunctional to pentadecafunctional (meth)acrylate compound is still more preferable, a bifunctional to decafunctional (meth)acrylate compound is even still more preferable, and a bifunctional to hexafunctional (meth)acrylate compound is particularly preferable.

Specific examples of the radically polymerizable compound include pentaerythritol tri(meth)acrylate, pentaerythritol tetra(meth)acrylate, dipentaerythritol penta(meth)acrylate, dipentaerythritol hexa(meth)acrylate, tri((meth)acryloyloxyethyl) isocyanurate, a pentaerythritol tetra(meth)acrylate ethylene oxide (EO)-modified product, a dipentaerythritol hexa(meth)acrylate ethylene oxide (EO)-modified product, and benzyl (meth)acrylate.

Examples of a commercially available product of the radically polymerizable compound include polyfunctional (meth)acrylate compounds such as KAYARAD Series (for example, D-330, D-320, D-310, PET-30, TPA-330, and DPHA) manufactured by Nippon Kayaku Co., Ltd.; NK Ester Series (for example, A-DPH-12E, A-TMMT, and A-TMM-3) manufactured by Shin-Nakamura Chemical Co., Ltd.; Light Acrylate Series (for example, DCP-A) manufactured by Kyoeisha Chemical Co., Ltd.; ARONIX Series (for example, M-305, M-306, M-309, M-450, M-402, and TO-1382) manufactured by TOAGOSEI CO., LTD.; and VISCOAT Series (for example, V #802) manufactured by Osaka Organic Chemical Industry Ltd.

As the radically polymerizable compound, the (meth)acrylate compounds described in JP1973-064183A (JP-S48-064183A), JP1974-043191B (JP-S49-043191B), and JP1977-030490B (JP-S52-030490B), and the compounds introduced as photocurable monomers and oligomers in The Adhesion Society of Japan, vol. 20, No. 7, pp. 300 to 308 (1984) can be used.

### —Cationically Polymerizable Compound—

Examples of the cationically polymerizable compound include a compound having a cationically polymerizable group. Examples of the cationically polymerizable group include a cyclic ether group such as an epoxy group or an oxetanyl group, and a vinyl ether group. Among these, a cyclic ether group is preferable. Further, it is preferable that the cationically polymerizable compound is a polyfunctional cationically polymerizable compound containing two or more cationically polymerizable groups.

Examples of the cationically polymerizable compound include a polyfunctional alicyclic epoxy compound, a polyfunctional heterocyclic epoxy compound, a polyfunctional oxetane compound, alkylene glycol diglycidyl ether, and an alkylene glycol monovinyl monoglycidyl ether.

Specific examples of the cationically polymerizable compound include 3′,4′-epoxycyclohexylmethyl 3,4-epoxycyclohexane carboxylate, bisphenol A diglycidyl ether, hydrogenated bisphenol A diglycidyl ether, 3,4-epoxycyclohexylmethyl-3′,4′-epoxycyclohexane carboxylate, a 1,2-epoxy-4-(2-oxiranyl)cyclohexane adduct of 2,2-bis(hydroxymethyl)-1-butanol, xylylene bisoxetane, 3-ethyl-3-hydroxymethyl oxetane, 3-ethyl-3-{[(3-ethyloxetane-3-yl)methoxy]methyl}oxetane, cyclohexanedimethanol divinyl ether, 2-ethylhexyl vinyl ether, cyclohexanedimethanol monovinyl ether, and 4-hydroxybutyl vinyl ether, and the compounds described in paragraphs 0029 to 0058 of JP2012-046577A.

As the cationically polymerizable compound, a (meth)acrylate compound containing a cationically polymerizable group can also be used. Specific examples of the (meth)acrylate compound containing a cationically polymerizable group include 3,4-epoxycyclohexylmethyl methacrylate. Examples of a commercially available product thereof include CYCLOMER M100 (manufactured by Daicel Corporation).

As the cationically polymerizable compound, ARON OXETANE series (such as OXT-101, OXT-121, or OXT-221, manufactured by Toagosei Company, Limited), CELLOXIDE series (2021P, manufactured by Daicel Corporation), and alkyl divinyl ether CHDVE, alkyl monovinyl ether EHVE, hydroxyalkyl vinyl ether CHMVE, and hydroxyalkyl vinyl ether HBVE (all manufactured by Nippon Carbide Industries Co., Inc.). Further, those exemplified as specific examples of the epoxy resin described below can also be used.

### (Resin)

Examples of the resin include a (meth)acrylic resin, an ene-thiol resin, a polyester resin, a polycarbonate resin, a vinyl polymer [for example, a polydiene resin, a polyalkene resin, a polystyrene resin, a polyvinyl ether resin, a polyvinyl alcohol resin, a polyvinyl ketone resin, a polyfluorovinyl resin, a polyvinyl bromide resin, and the like], a polythioether resin, a polyphenylene resin, a polyurethane resin, a polysulfonate resin, a nitroso polymer resin, a polysiloxane resin, a polysulfide resin, a polythioester resin, a polysulfone resin, a polysulfonamide resin, a polyamide resin, a polyimine resin, a polyurea resin, a polyphosphazene resin, a polysilane resin, a polysilazane resin, a polyfuran resin, a polybenzoxazole resin, a polyoxadiazole resin, a polybenzothiazinophenothiazine resin, a polybenzothiazole resin, a polypyrazinoquinoxaline resin, a polyquinoxaline resin, a polybenzoimidazoline resin, a polyoxoisoindoline resin, a polydioxoisoindoline resin, a polytriazine resin, a polypyridazine resin, a polypiperazine resin, a polypyridine resin, a polypiperidine resin, a polytriazole resin, a polypyrazole resin, a polypyrrolidine resin, a polycarborane resin, a polyoxabicyclononane resin, a polydibenzofuran resin, a polyphthalide resin, a polyacetal resin, a polyimide resin, a polyamide imide resin, an olefin resin, a cyclic olefin resin, an epoxy resin, and a cellulose acylate resin.

Examples of the (meth)acrylic resin include a polymer having a constitutional unit derived from (meth)acrylic acid and/or an ester thereof. Specific examples thereof include a polymer obtained by carrying out a polymerization reaction on at least one compound selected from the group consisting of (meth)acrylic acid, (meth)acrylic acid ester, (meth)acrylamide, and (meth)acrylonitrile.

Examples of the polyester resin include polymers obtained by reacting a polyol (such as ethylene glycol, propylene glycol, glycerin, and trimethylolpropane) with a polybasic acid (such as aromatic dicarboxylic acid (for example, terephthalic acid, isophthalic acid, naphthalenedicarboxylic acid, and dicarboxylic acid in which a hydrogen atom of these aromatic rings is replaced with a methyl group, an ethyl group, or a phenyl group), aliphatic dicarboxylic acid having 2 to 20 carbon atoms (for example, adipic acid, sebacic acid, and dodecanedicarboxylic acid), and alicyclic dicarboxylic acid (for example, cyclohexanedicarboxylic acid)); and polymers obtained by ring-opening polymerization of a cyclic ester compound such as caprolactone monomers (for example, polycaprolactone).

Specific examples of the polyester resin include polyethylene terephthalate and polyethylene naphthalate.

Examples of the epoxy resin include a bisphenol A type epoxy resin, a bisphenol F type epoxy resin, a phenol novolak type epoxy resin, a cresol novolak type epoxy resin, and an aliphatic epoxy resin. As the epoxy resin, a commercially available product on the market may be used, and examples of the commercially available product include the following products.

Examples of a commercially available product of the bisphenol A type epoxy resins include jER825, jER827, jER828, jER834, jER1001, jER1002, jER1003, jER1055, jER1007, jER1009, and jER1010 (all manufactured by Mitsubishi Chemical Corporation), and EPICLON860, EPICLON1050, EPICLON1051, and EPICLON1055 (all manufactured by DIC Corporation). Examples of a commercially available product of the bisphenol F type epoxy resins include jER806, jER807, jER4004, jER4005, jER4007, and jER4010 (all manufactured by Mitsubishi Chemical Corporation), EPICLON830 and EPICLON835 (both manufactured by DIC Corporation), and LCE-21 and RE-602S (both manufactured by Nippon Kayaku Co., Ltd.). Examples of a commercially available product of the phenol novolak type epoxy resins include jER152, jER154, jER157S70, and jER157S65 (all manufactured by Mitsubishi Chemical Corporation), and EPICLON N-740, EPICLON N-770, and EPICLON N-775 (all manufactured by DIC Corporation). Examples of the commercially available product of the cresol novolak type epoxy resins include EPICLON N-660, EPICLON N-665, EPICLON N-670, EPICLON N-673, EPICLON N-680, EPICLON N-690, and EPICLON N-695 (all manufactured by DIC Corporation), and EOCN-1020 (manufactured by Nippon Kayaku Co., Ltd.). Examples of the commercially available product of the aliphatic epoxy resin include ADEKA RESIN EP Series (such as EP-40805, EP-40855, and EP-40885; manufactured by ADEKA Corporation), CELLOXIDE 2021P, CELLOXIDE 2081, CELLOXIDE 2083, CELLOXIDE 2085, EHPE3150, EPOLEAD PB 3600, and EPOLEAD PB 4700 (all manufactured by Daicel Corporation), DENACOL EX-212L, EX-214L, EX-216L, EX-321L, and EX-850L (all manufactured by Nagase ChemteX Corporation), ADEKA RESIN EP Series (such as EP-40005, EP-40035, EP-40105, and EP-40115; manufactured by ADEKA Corporation), NC-2000, NC-3000, NC-7300, XD-1000, EPPN-501, and EPPN-502 (all manufactured by ADEKA Corporation), and jER1031S (manufactured by Mitsubishi Chemical Corporation). Further, examples of a commercially available product of the epoxy resins include MARPROOF G-0150M, G-0105SA, G-0130SP, G-0250SP, G-10055, G-1005SA, G-1010S, G-2050M, G-01100, and G-01758 (all manufactured by NOF Corporation, epoxy group-containing polymer).

As the cellulose acylate resin, the cellulose acylate described in paragraphs 0016 to 0021 of JP2012-215689A is preferably used. As the polyester resin, a commercially available product such as the VYLON Series (for example, VYLON 500, manufactured by Toyobo Co., Ltd.) can also be used. As a commercially available product of the (meth)acrylic resin, SK Dyne Series (for example, SK Dyne-SF2147, manufactured by Soken Chemical & Engineering Co., Ltd.) can also be used.

As the polystyrene resin, a resin having 50% by mass or greater of a repeating unit derived from a styrene-based monomer is preferable, a resin having 70% by mass or greater of a repeating unit derived from a styrene-based monomer is more preferable, and a resin having 85% by mass or greater of a repeating unit derived from a styrene-based monomer is still more preferable.

Specific examples of the styrene-based monomer include styrene and a derivative thereof. Here, the styrene derivative is a compound in which another group is bonded to styrene, and examples thereof include alkylstyrene such as o-methylstyrene, m-methylstyrene, p-methylstyrene, 2,4-dimethylstyrene, o-ethylstyrene, or p-ethylstyrene, and substituted styrene in which a hydroxyl group, an alkoxy group, a carboxy group, or halogen is introduced to a benzene nucleus of styrene such as hydroxystyrene, tert-butoxystyrene, vinyl benzoic acid, o-chlorostyrene, or p-chlorostyrene.

In addition, the polystyrene resin may have a repeating unit derived from a monomer other than the styrene-based monomer. Examples of other monomers include alkyl (meth)acrylate such as methyl (meth)acrylate, cyclohexyl (meth)acrylate, methylphenyl (meth)acrylate, or isopropyl (meth)acrylate; an unsaturated carboxylic acid monomer such as methacrylic acid, acrylic acid, itaconic acid, maleic acid, fumaric acid, or cinnamic acid; an unsaturated dicarboxylic acid anhydride monomer which is an anhydride of maleic acid, itaconic acid, ethylmaleic acid, methylitaconic acid, or chloromaleic acid; an unsaturated nitrile monomer such as acrylonitrile or methacrylonitrile; and a conjugated diene such as 1,3-butadiene, 2-methyl-1,3-butadiene (isoprene), 2,3-dimethyl-1,3-butadiene, 1,3-pentadiene, or 1,3-hexadiene.

Examples of a commercially available product of the polystyrene resin include AS-70 (acrylonitrile-styrene copolymer resin) manufactured by NIPPON STEEL & SUMITOMO METAL CORPORATION; SMA2000P (styrene-maleic acid copolymer) of KAWAHARA PETROCHEMICAL CO., LTD.; CLEAREN 530L and CLEAREN 730L manufactured by Denka Company Limited; TUFPRENE 126S and ASAPRENE T411 manufactured by Asahi Kasei Corporation; KRATON D1102A and KRATON D1116A manufactured by Kraton Corporation; STYROLUX S and STYROLUX T manufactured by INEOS Styrolution Group GmbH; ASAFLEX 840 and ASAFLEX 860 manufactured by Asahi Kasei Corporation; 679, HF77, SGP-10, 475D, H0103, and HT478 manufactured by PS Japan Corporation; and DICSTYRENE XC-515, DICSTYRENE XC-535, and DICSTYRENE GH-8300-5 manufactured by DIC Corporation. In addition, examples of a commercially available product of the hydrogenated polystyrene resin include TUFTEC H Series manufactured by Asahi Kasei Corporation, KRATON G Series manufactured by Shell Japan Limited, DYNARON (hydrogenated styrene-butadiene random copolymer) manufactured by JSR Corporation, and SEPTON manufactured by Kuraray Co., Ltd. In addition, examples of a commercially available product of the modified polystyrene resin include TUFTEC M Series manufactured by Asahi Kasei Corporation, EPOFRIEND manufactured by Daicel Corporation, polar group-modified DYNARON manufactured by JSR Corporation, and RESEDA manufactured by TOAGOSEI CO., LTD.

Examples of the cyclic olefin resin include (R1) polymers including a structural unit derived from a norbornene compound, (R2) polymers including a structural unit derived from a monocyclic olefin compound which is not a norbornene compound, (R3) polymers including a structural unit derived from a cyclic conjugated diene compound, (R4) polymers including a structural unit derived from a vinyl alicyclic hydrocarbon compound, and hydrides of polymers including a structural unit derived from each of the compounds (R1) to (R4). In the present specification, the polymer having a structural unit derived from a norbornene compound and the polymer having a structural unit derived from a monocyclic olefin compound contains ring-opening polymers of the respective compounds.

The cyclic olefin resin is not particularly limited, but is preferably a polymer having a structural unit derived from a norbornene compound, which is represented by Formula (A-II) or Formula (A-III). The polymer having the structural unit represented by Formula (A-II) is an addition polymer of a norbornene compound, and the polymer having the structural unit represented by Formula (A-III) is a ring-opening polymer of a norbornene compound.

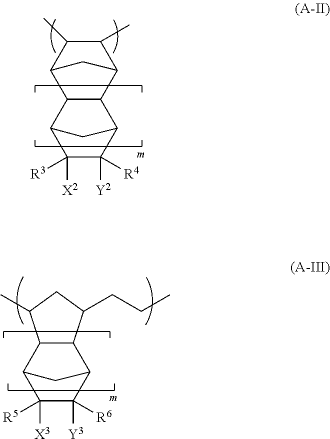

In Formulae (A-II) and (A-III), m represents an integer of 0 to 4 and is preferably 0 or 1.

R³ to R⁶ in Formulae (A-II) and (A-III) each independently represent a hydrogen atom or a hydrocarbon group having 1 to 10 carbon atoms.

Examples of the hydrocarbon group represented by R³ to R⁶ include an alkyl group, an alkenyl group, an alkynyl group, and an aryl group, and an alkyl group or an aryl group is preferable.

X² and X³, Y² and Y³ each independently represent a hydrogen atom, a hydrocarbon group having 1 to 10 carbon atoms, a halogen atom, a hydrocarbon group having 1 to 10 carbon atoms, which is substituted with a halogen atom, —(CH₂)ₙCOOR¹¹, —(CH₂)ₙOCOR¹², —(CH₂)ₙNCO, —(CH₂)ₙNO₂, —(CH₂)ₙCN, —(CH₂)ₙCONR¹³R¹⁴, —(CH₂)ₙNR¹³R¹⁴, —(CH₂)ₙOZ¹, —(CH₂)ₙW¹, or (—CO)₂O or (—CO)₂NR¹⁵ which is formed by bonding X² and Y², or X³ and Y³.

Here, R¹¹ to R¹⁵ in the above-described groups which can be adopted as X², X³, Y², and Y³ each independently represent a hydrogen atom or a hydrocarbon group having 1 to 20 carbon atoms, Z¹ represents a hydrocarbon group or a hydrocarbon group substituted with halogen, and W¹ represents Si(R¹⁶)ₚD₍₃₋ₚ₎ (R¹⁶ represents a hydrocarbon group having 1 to 10 carbon atoms, and D represents a halogen atom, —OCOR¹⁷, or —OR¹⁷ (R¹⁷ represents a hydrocarbon group having 1 to 10 carbon atoms), and p is an integer of 0 to 3). n is an integer of 0 to 10, preferably 0 to 8, and more preferably 0 to 6.

In Formulae (A-II) and (A-III), R³ to R⁶ are each independently preferably a hydrogen atom or —CH₃, and from the viewpoint of moisture permeability, still more preferably a hydrogen atom.

Each of X² and X³ is preferably a hydrogen atom, —CH₃, or —C₂H₅, and from the viewpoint of moisture permeability, still more preferably a hydrogen atom.

Y² and Y³ are each independently preferably a hydrogen atom, a halogen atom (particularly a chlorine atom), or —(CH₂)ₙCOOR¹¹ (particularly —COOCH₃), and from the viewpoint of moisture permeability, still more preferably a hydrogen atom.

Other groups are appropriately selected.

The polymer having the structural unit represented by Formula (A-II) or Formula (A-III) may further include one or more of a structural unit represented by Formula (A-I).

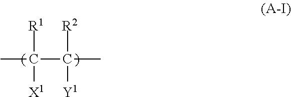

In Formula (A-I), R¹ and R² each independently represent a hydrogen atom or a hydrocarbon group having 1 to 10 carbon atoms, and X¹ and Y¹ each independently represent a hydrogen atom, a hydrocarbon group having 1 to 10 carbon atoms, a halogen atom, a hydrocarbon group having 1 to 10 carbon atoms, which is substituted with a halogen atom, —(CH₂)ₙC₀₀R¹¹, —(CH₂)ₙOC₀R¹², —(CH₂)ₙNCO, —(CH₂)ₙNO₂, —(CH₂)ₙCN, —(CH₂)ₙCONR¹³R¹⁴, —(CH₂)ₙNR¹³R¹⁴, —(CH₂)ₙOZ¹, —(CH₂)ₙW¹, or (—CO)₂O or (—CO)₂NR¹⁵ formed by X² and Y² or X³ and Y³ being bonded to each other. R¹¹ to R¹⁵ in the above-described groups which can be adopted as X¹ and Y¹ each independently represent a hydrogen atom or a hydrocarbon group having 1 to 20 carbon atoms, Z¹ represents a hydrocarbon group or a hydrocarbon group substituted with halogen, and W¹ represents Si(R¹⁶)ₚD₍₃₋ₚ₎ (R¹⁶ represents a hydrocarbon group having 1 to 10 carbon atoms, and D represents a halogen atom, —OCOR¹⁷, or —OR¹⁷ (R¹⁷ represents a hydrocarbon group having 1 to 10 carbon atoms), and p is an integer of 0 to 3). n represents an integer of 0 to 10.

A content of the structural unit represented by Formula (A-II) or Formula (A-III) in the cyclic polyolefin resin is preferably 90% by mass or less, more preferably 30% to 85% by mass, still more preferably 50% to 79% by mass, and even more preferably 60% to 75% by mass.

The cyclic olefin resin is described in JP1998-007732A (JP-H10-007732A), JP2002-504184A, WO2004/070463A, and the like, the contents of which can be referred to as appropriate.

The cyclic olefin resin is obtained by performing an addition polymerization of a norbornene compound (for example, a polycyclic unsaturated compound of norbornene) with each other.

Examples of a commercially available product of the cyclic olefin resin include ARTON series (for example, ARTON G, ARTON F, and ARTON RX4500) manufactured by JSR Corporation, and Zeonor ZF14, Zeonor ZF16, Zeonex 250, and Zeonex 280 (manufactured by Zeon Corporation).

In addition, examples of the cyclic olefin resin include copolymers obtained by an addition copolymerization of, as necessary, a norbornene compound, and olefin such as ethylene, propylene, and butene, conjugated diene such as butadiene and isoprene, unconjugated diene such as ethylidene norbornene, or an ethylenically unsaturated compound such as acrylonitrile, acrylic acid, methacrylic acid, maleic acid anhydride, acrylic acid ester, methacrylic acid ester, maleimide, vinyl acetate, and vinyl chloride. Among these, a copolymer with ethylene is preferable. Examples of the above-described addition (co)polymers of a norbornene compound include APL8008T (Tg: 70° C.), APL6011T (Tg: 105° C.), APL6013T (Tg: 125° C.), and APL6015T (Tg: 145° C.) which are sold by Mitsui Chemicals, Inc. under a trade name of APL and have different glass transition temperatures (Tg). In addition, pellets such as TOPAS8007, TOPAS6013, and TOPAS6015 are commercially available from Polyplastics Co., Ltd. Furthermore, Appear3000 is commercially available from Film Ferrania S. R. L.

In addition, the hydride of the cyclic olefin resin can be synthesized by an addition polymerization or a ring-opening metathesis polymerization of a norbornene compound or the like and then an addition of hydrogen. The synthesis method is described, for example, in JP1989-240517A (JP-H01-240517A), JP1995-196736A (JP-H07-196736A), JP1985-026024A (JP-S60-026024A), JP1987-019801A (JP-S62-019801A), JP2003-159767A, and JP2004-309979A.

A weight-average molecular weight of the cyclic olefin resin is preferably 5,000 to 500,000, more preferably 8,000 to 200,000, and still more preferably 10,000 to 100,000.

Examples of the polycarbonate resin include a reaction product of a polyhydric phenol compound and phosgene or a carbonic ester compound.

Examples of the polyhydric phenol compound include hydroquinone, resorcinol, 4,4′-dihydroxydiphenyl, bis(4-hydroxyphenyl)methane, 1,1-bis(4-hydroxyphenyl)ethane, 1,1-bis(4-hydroxyphenyl)-1-phenylethane, bisphenol A, bisphenol C, bisphenol E, bisphenol F, bisphenol M, bisphenol P, bisphenol S, bisphenol Z, 2,2-bis(3-methyl-4-hydroxyphenyl)propane, 1,1-bis(4-hydroxyphenyl)cyclohexane, 2,2-bis(3-phenyl-4-hydroxyphenyl)propane, 2,2-bis(3-isopropyl-4-hydroxyphenyl)propane, 2,2-bis(4-hydroxyphenyl)butane, 2,2-bis(3,5-dimethyl-4-hydroxyphenyl)propane, 2,2-bis(3,5-dibromo-4-hydroxyphenyl)propane, 4,4′-dihydroxydiphenyl sulfone, 4,4′-dihydroxydiphenyl sulfoxide, 4,4′-dihydroxydiphenyl sulfide, 3,3′-dimethyl-4,4′-dihydroxydiphenyl sulfide, and 4,4′-dihydroxydiphenyl oxide. Among these, hydroquinone, resorcinol, 4,4′-dihydroxydiphenyl, or bisphenol Ais preferable.

Examples of the carbonic ester compound include phosgene, diphenyl carbonate, bis(chlorophenyl) carbonate, dinaphthyl carbonate, bis(diphenyl) carbonate, dimethyl carbonate, diethyl carbonate, and dibutyl carbonate; and bis(diphenyl) carbonate, dimethyl carbonate, or diethyl carbonate is preferable.

Examples of a commercially available product of the polycarbonate resin include PANLITE L-1250WP and PANLITE SP-1516 manufactured by Teijin Limited; Iupizeta EP-5000 and Iupizeta EP-4000 manufactured by Mitsubishi Gas Chemical Company Inc.; and Calibre 301-30 manufactured by Sumika Polycarbonate Ltd.

Examples of the thiourethane resin include a reactant of an isocyanate compound and a polythiol compound and a reactant of a thiourethane resin precursor. Examples of a commercially available product of the thiourethane resin precursor include MR-7, MR-8, MR-10, and MR-174 (manufactured by Mitsui Chemicals, Inc.).

Examples of the polyamide resin include an aliphatic polyamide resin and an aromatic polyamide resin. Examples of the aliphatic polyamide resin include Nylon 6, Nylon 11, Nylon 12, Nylon 46, Nylon 66, Nylon 666, Nylon 610, and Nylon 612. Examples of the aromatic polyamide resin include a resin which is polymerized by dehydration condensation of a diamine and a dicarboxylic acid and for which at least one of a diamine or a dicarboxylic acid having an aromatic ring is used. Specific examples of the aromatic polyamide resin include a condensation polymer of m-xylylenediamine and adipic acid or an adipic acid halide.

The resin may have an acid group. Examples of the acid group include a carboxy group, a phosphoric acid group, a sulfonic acid group, and a phenolic hydroxy group. The acid group may be used alone or in combination of two or more kinds thereof. The resin having an acid group can be used as an alkali-soluble resin or as a dispersant.

As the resin having an acid group, reference can be made to the description in paragraphs 0558 to 0571 of JP2012-208494A (paragraphs 0685 to 0700 of the corresponding US2012/0235099A) and the description in paragraphs 0076 to 0099 of JP2012-198408A, the contents of which are incorporated herein by reference. In addition, as the resin having an acid group, ACRYBASE FF-426 (manufactured by NIPPON SHOKUBAI CO., LTD.) can also be used.

The acid value of the resin having an acid group is preferably 30 to 200 mgKOH/g. The lower limit or the acid value is preferably 50 mgKOH/g or more and more preferably 70 mgKOH/g or more. Further, the upper limit of the acid value is preferably 150 mgKOH/g or less and more preferably 120 mgKOH/g or less. The acid value of the resin is a value calculated by performing measurement in conformity with JIS K 0070 (1992) and converting the measured value in terms of 1 mmol/g=56.1 mgKOH/g.

The resin may have a polymerizable group. Examples of the polymerizable group include an ethylenically unsaturated bond-containing group and a cyclic ether group. Examples of the ethylenically unsaturated bond-containing group include a vinyl group, a styrene group, an allyl group, a methallyl group, and a (meth)acryloyl group. Examples of the cyclic ether group include an epoxy group and an oxetanyl group.

Examples of a commercially available product of the resin containing a polymerizable group include Dianal BR Series (poly(methyl methacrylate) (PMMA), for example Dianal BR-80, BR-83, and BR-87, manufactured by Mitsubishi Chemical Corporation); Photomer 6173 (COOH-containing polyurethane acrylic oligomer, Diamond Shamrock Co., Ltd.); Viscoat R-264 and KS Resist 106 (both manufactured by Osaka Organic Chemical Industry Ltd.); Cyclomer P series (for example, ACA230AA) and Placcel CF 200 series (all manufactured by Daicel Corporation); Ebecryl 3800 (manufactured by Daicel UCB Company, Ltd.); and Acrycure RD-F8 (manufactured by Nippon Shokubai Co., Ltd.). In addition, examples thereof also include commercially available products such as the products described in the section of the epoxy resin described above.

In a case where the composition according to the embodiment of the present invention is used for a lens (for example, an eyeglass lens), as the resin, a thermoplastic resin such as a carbonate resin and a (meth)acrylic resin, or a thermosetting resin such as a urethane resin is suitable.

As the resin, a pressure sensitive adhesive or an adhesive can also be used. Examples of the pressure sensitive adhesive include an acrylic pressure sensitive adhesive, a rubber-based pressure sensitive adhesive, and a silicone-based pressure sensitive adhesive.

The acrylic pressure sensitive adhesive denotes a pressure sensitive adhesive containing a polymer of a (meth)acrylic monomer ((meth)acrylic polymer). Examples of the adhesive include a urethane resin adhesive, a polyester adhesive, an acrylic resin adhesive, an ethylene vinyl acetate resin adhesive, a polyvinyl alcohol adhesive, a polyamide adhesive, and a silicone adhesive. Among these, from the viewpoint of excellent adhesive strength, a urethane resin adhesive or a silicone adhesive is preferable as the adhesive. As the adhesive, a commercially available product on the market may be used, and examples of the commercially available product thereof include a urethane resin adhesive (LIS-073-50U: trade name, manufactured by of Toyo Ink Co., Ltd.) and an acrylic pressure sensitive adhesive (SK Dyne-SF2147: trade name, manufactured by Soken Chemical & Engineering Co., Ltd.).

The resin is preferably at least one selected from a (meth)acrylic resin, a polystyrene resin, a polyester resin, a polyurethane resin, a thiourethane resin, a polyimide resin, a polyamide resin, an epoxy resin, a polycarbonate resin, a phthalate resin, a cellulose acylate resin, or a cyclic olefin resin; and from the reason that compatibility with the specific compound is favorable and a cured substance in which surface unevenness is suppressed is easily obtained, it is more preferable to be at least one selected from a (meth)acrylic resin, a polystyrene resin, a polyester resin, a polyurethane resin, or a cyclic olefin resin.

A weight-average molecular weight (Mw) of the resin is preferably 2,000 to 2,000,000. The lower limit of Mw of the resin is preferably 5,000 or more, more preferably 10,000 or more, and still more preferably 50,000 or more. The upper limit of Mw of the resin is preferably 1,000,000 or less, more preferably 500,000 or less, and still more preferably 200,000 or less. In addition, in a case of using an epoxy resin, the weight-average molecular weight (Mw) of the epoxy resin is preferably 100 or more, and more preferably 200 to 2,000,000. The upper limit of Mw of the epoxy resin is preferably 1,000,000 or less and more preferably 500,000 or less. The lower limit of Mw of the epoxy resin is preferably 2000 or more.

The weight-average molecular weight (Mw) is a value measured by gel permeation chromatography (GPC). In the measurement by GPC, HLC (registered trademark)-8020GPC (manufactured by Tosoh Corporation) is used as a measuring device, three columns of TSKgel (registered trademark) Super Multipore HZ-H (4.6 mmID×15 cm, manufactured by Tosoh Corporation) are used as a column, and tetrahydrofuran (TIF) is used as an eluent. Further, the measurement is performed under measurement conditions of a sample concentration of 0.45% by mass, a flow rate of 0.35 ml/min, a sample injection volume of 10 μl, and a measurement temperature of 40° C. using an RI detector. Further, the calibration curve is prepared using eight samples of “F-40”, “F-20”, “F-4”, “F-1”, “A-5000”, “A-2500”, “A-1000”, and “n-propylbenzene” which are “Standard Samples TSK standard, polystyrene” (manufactured by Tosoh Corporation).

The total light transmittance of the resin is preferably 80% or greater, more preferably 85% or greater, and still more preferably 90% or greater. In the present specification, the total light transmittance of the resin is a value measured based on the contents described in “The Fourth Series of Experimental Chemistry 29 Polymer Material” (Maruzen, 1992), pp. 225 to 232, edited by the Chemical Society of Japan.

A content of the curable compound in the total solid content of the composition is preferably 1% to 99.9% by mass. The lower limit thereof is preferably 30% by mass or more, more preferably 50% by mass or more, and still more preferably 70% by mass or more. The upper limit thereof is preferably 95% by mass or less, more preferably 90% by mass or less, and still more preferably 80% by mass or less. The composition may contain only one kind of the curable compound or two or more kinds thereof. In a case where the curable compound contains two or more kinds of polymerizable compounds, it is preferable that the total amount thereof is in the above-described ranges.

In a case where the curable compound contained in the composition according to the embodiment of the present invention includes a resin, the content of the resin in the total solid content of the composition is preferably 1% to 99.9% by mass. The lower limit thereof is preferably 30% by mass or more, more preferably 50% by mass or more, and still more preferably 70% by mass or more. The upper limit thereof is preferably 95% by mass or less, more preferably 90% by mass or less, and still more preferably 80% by mass or less. The composition may contain only one kind of the resin or two or more kinds thereof. In a case where the polymerization composition contains two or more kinds of resins, it is preferable that the total amount thereof is in the above-described range.

In a case where the curable compound contained in the composition according to the embodiment of the present invention includes a polymerizable compound, the content of the polymerizable compound in the total solid content of the composition is preferably 0.1% to 90% by mass. The lower limit thereof is preferably 1% by mass or more and more preferably 5% by mass or more. The upper limit thereof is preferably 80% by mass or less and more preferably 70% by mass or less. The composition according to the embodiment of the present invention may contain only one kind of polymerizable compound or two or more kinds thereof. In a case where the polymerizable composition contains two or more kinds of polymerizable compounds, it is preferable that the total amount thereof is in the above-described ranges.

<<Polymerization Initiator>>

The composition according to the embodiment of the present invention may contain a polymerization initiator. As the polymerization initiator, a compound capable of generating an initiating species required for the polymerization reaction by applying energy can be used.

Examples of the polymerization initiator include a radical polymerization initiator and a cationic polymerization initiator. In a case where the radically polymerizable compound is used as the polymerizable compound, the polymerization initiator is preferably a radical polymerization initiator. In a case where the cationically polymerizable compound is used as the polymerizable compound, the polymerization initiator is preferably a cationic polymerization initiator.

The polymerization initiator can be appropriately selected from, for example, a photopolymerization initiator and a thermal polymerization initiator, and a photopolymerization initiator is preferable. The photopolymerization initiator is a compound which is photosensitized by exposure light and initiates or promotes the polymerization of the polymerizable compound. Examples of the photopolymerization initiator include a photoradical polymerization initiator and a photocationic polymerization initiator, and a photoradical polymerization initiator is preferable. It is preferable that the photoradical polymerization initiator is a compound that is sensitive to actinic rays having a wavelength of 300 nm or greater to generate a radical.

### (Photoradical Polymerization Initiator)

Examples of the photoradical polymerization initiator include an oxime compound, a halogenated hydrocarbon derivative (for example, a compound having a triazine skeleton, a compound having an oxadiazole skeleton, and the like), an oxadiazole compound, a carbonyl compound, a ketal compound, a benzoin compound, an acridine compound, an organic peroxide, an azo compound, a coumarin compound, an azide compound, a metallocene compound, a hexaarylbiimidazole compound, an organic borate compound, a disulfonate compound, an onium salt compound, an acetophenone compound, an acylphosphine compound, and a benzophenone compound.

Examples of the acetophenone compound include an aminoacetophenone compound and a hydroxyacetophenone compound. Examples of the acetophenone compound include acetophenone compounds described in JP2009-191179A and JP1998-291969A (JP-H10-291969A). Examples of commercially available products of the aminoacetophenone compound include Omnirad 907, Omnirad 369, Omnirad 369E, and Omnirad 379EG (all manufactured by IGM Resins B. V). Examples of commercially available products of the hydroxyacetophenone compound include Omnirad 184, Omnirad 1173, Omnirad 2959, and Omnirad 127 (all manufactured by IGM Resins B. V.).

Examples of the acylphosphine compound include the acylphosphine compound described in JP4225898B. Examples of commercially available products of the acylphosphine compound include Omnirad 819 and Omnirad TPO (both manufactured by IGM Resins B. V).

Examples of the benzophenone compound include benzophenone, 2-methylbenzophenone, 3-methylbenzophenone, 4-methylbenzophenone, 4-methoxybenzophenone, 2-chlorobenzophenone, 4-chlorobenzophenone, 4-bromobenzophenone, 2-carboxybenzophenone, 2-ethoxycarbonylbenzophenone, benzophenone tetracarboxylic acid or tetramethyl ester thereof, 4,4′-bis(dialkylamino)benzophenones (for example, 4,4′-bis(dimethylamino)benzophenone, 4,4′-bis(dicyclohexylamino)benzophenone, 4,4′-bis(diethylamino)benzophenone, and 4,4′-bis(dihydroxyethylamino)benzophenone), 4-methoxy-4′-dimethylaminobenzophenone, 4,4′-dimethoxybenzophenone, 4-dimethylaminobenzophenone, and 4-dimethylaminoacetophenone. Among these, from the viewpoints of the sensitivity and the light resistance of a cured substance to be obtained, 4,4′-bis(diethylamino)benzophenone is preferable.

Examples of the oxime compound include the compounds described in JP2001-233842A, the compounds described in JP2000-080068A, the compounds described in JP2006-342166A, and the compounds described in paragraphs 0073 to 0075 of JP2016-006475A. Among the examples of the oxime compound, an oxime ester compound is preferable. Examples of commercially available products of the oxime compound include Irgacure OXE01, Irgacure OXE02 (manufactured by BASF SE), and Irgacure OXE03 (manufactured by BASF SE).

Examples of the halogenated hydrocarbon derivative include the compounds disclosed in Wakabayashi et al., “Bull Chem. Soc. Japan” 42, 2924 (1969), U.S. Pat. No. 3,905,815A, JP1971-004605B (JP-S46-004605B), JP1973-036281A (JP-S48-036281A), JP1980-32070A (JP-S55-032070A), JP1985-239736A (JP-S60-239736A), JP1986-169835A (JP-S61-169835A), JP-1986-169837A (JP-S61-169837A), JP1987-058241A (JP-S62-058241A), JP1987-212401A (JP-S62-212401A), JP1988-070243A (JP-S63-070243A), JP1988-298339A (JP-S63-298339A), and M. P. Hutt “Journal of Heterocyclic Chemistry” 1 (No. 3), (1970), and an oxazole compound or a triazine compound substituted with a trihalomethyl group is preferable.

Examples of the hexaarylbiimidazole compound include the compounds described in JP1994-029285B (JP-H06-029285B), U.S. Pat. Nos. 3,479,185A, 4,311,783A, and 4,622,286A. Specific examples thereof include 2,2′-bis(o-chlorophenyl)-4,4′,5,5′-tetraphenylbiimidazole, 2,2′-bis(o-bromophenyl)-4,4′,5,5′-tetraphenylbiimidazole, 2,2′-bis(o,p-dichlorophenyl)-4,4′,5,5′-tetraphenylbiimidazole, 2,2′-bis(o-chlorophenyl)-4,4′,5,5′-tetra(m-methoxyphenyl)biimidazole, 2,2′-bis(o,o′-dichlorophenyl)-4,4′,5,5′-tetraphenylbiimidazole, 2,2′-bis(o-nitrophenyl)-4,4′,5,5′-tetraphenylbiimidazole, 2,2′-bis(o-methylphenyl)-4,4′,5,5′-tetraphenylbiimidazole, and 2,2′-bis(o-trifluorophenyl)-4,4′,5,5′-tetraphenylbiimidazole.

### (Photocationic Polymerization Initiator)

The photocationic polymerization initiator is not particularly limited as long as the photocationic polymerization initiator is a compound that generates a protonic acid or a Lewis acid by being irradiated with light. As the photoacid generator, a compound that is sensitive to actinic rays having a wavelength of 300 nm or greater and more preferably 300 to 450 nm and generates an acid is preferable. The photoacid generator is preferably a compound that generates an acid having a pKa of 4 or less, more preferably a compound that generates an acid having a pKa of 3 or less, and still more preferably a compound that generates an acid having a pKa of 2 or less in a case of being irradiated with light.

Examples of the photocationic polymerization initiator include an oxime sulfonate compound, a triazine compound, a sulfonium salt, an iodonium salt, a quaternary ammonium salt, a diazomethane compound, a sulfone compound, a sulfonic acid ester compound, an iminosulfonic acid ester compound, a carboxylic acid ester compound, and a sulfonimide compound.

Specific examples of the photocationic polymerization initiator include the compounds described in paragraphs 0061 to 0108 of JP2012-046577A and paragraphs 0029 and 0030 of JP2002-122994A, the compounds described in paragraphs 0037 to 0063 of JP2002-122994A, and the oxime sulfonate compounds described in paragraphs 0081 to 0108 of JP2013-210616A. Examples of a commercially available product of the photocationic polymerization initiator include WPAG-469 (manufactured by FUJIFILM Wako Pure Chemical Corporation), CPI-100P (manufactured by San-Apro Ltd.), CPI-210S (manufactured by San-Apro Ltd.), and Irgacure 290 (manufactured by BASF SE).

### (Thermal Polymerization Initiator)

The thermal polymerization initiator is not particularly limited, and a known thermal polymerization initiator can be used. Examples thereof include azo-based compounds such as dimethyl 2,2′-azobis(isobutyrate), 2,2′-azobisisobutyronitrile, 2,2′-azobis(2,4-dimethyl-4-methoxyvaleronitrile), 2,2′-azobis(2,4-dimethylvaleronitrile), dimethyl-2,2′-azobis(2-methylpropionate), 2,2′-azobis(2-methylbutyronitrile), 1,1′-azobis(cyclohexane-1-carbonitrile), 2,2′-azobis(N-butyl-2-methylpropionamide), dimethyl-1,1′-azobis(1-cyclohexanecarboxylate), and 2,2′-azobis[2-(2-imidazolin-2-yl)propane]dihydrochloride;
organic peroxides such as 1,1-di(t-hexylperoxy)cyclohexane, 1,1-di(t-butylperoxy)cyclohexane, 2,2-di(4,4-di-(t-butylperoxy)cyclohexyl)propane, t-hexyl peroxy isopropyl monocarbonate, t-butyl peroxy-3,5,5-trimethyl hexanoate, t-butyl peroxy laurate, dicumyl peroxide, di-t-butyl peroxide, t-butyl peroxy-2-ethyl hexanoate, t-hexyl peroxy-2-ethyl hexanoate, cumene hydroperoxide, and t-butyl hydroperoxide; and inorganic peroxides such as potassium persulfate, ammonium persulfate, and hydrogen peroxide.

In a case where the composition according to the embodiment of the present invention contains a polymerization initiator, a content of the polymerization initiator in the total solid content of the composition is preferably 0.1% to 20% by mass. The lower limit thereof is preferably 0.3% by mass or more and more preferably 0.4% by mass or more. The upper limit thereof is preferably 15% by mass or less and more preferably 10% by mass or less.

The composition according to the embodiment of the present invention may contain only one kind of polymerization initiator or two or more kinds thereof. In a case where the polymerization composition contains two or more kinds of polymerization initiators, it is preferable that the total amount thereof is in the above-described range.

### <<Catalyst>>

The composition according to the embodiment of the present invention can contain a catalyst. Examples of the catalyst include an acid catalyst such as hydrochloric acid, sulfuric acid, acetic acid, or propionic acid and a base catalyst such as sodium hydroxide, potassium hydroxide, or triethylamine. In a case where the composition according to the embodiment of the present invention contains a catalyst, a content of the catalyst is preferably 0.1 to 100 parts by mass, more preferably 0.1 to 50 parts by mass, and still more preferably 0.1 to 20 parts by mass with respect to 100 parts by mass of the resin. The composition according to the embodiment of the present invention may contain only one kind of catalyst or two or more kinds thereof. In a case where the resin composition contains two or more kinds of catalysts, it is preferable that the total amount thereof is in the above-described range.

### <<Silane Coupling Agent>>

The composition according to the embodiment of the present invention may contain a silane coupling agent. According to this aspect, adhesiveness of a film to be obtained with a support can be further improved. In the present invention, the silane coupling agent means a silane compound having a hydrolyzable group and other functional groups. Further, the hydrolyzable group denotes a substituent that is directly bonded to a silicon atom and can form a siloxane bond by at least one of a hydrolysis reaction or a condensation reaction. Examples of the hydrolyzable group include a halogen atom, an alkoxy group, and an acyloxy group. Among these, an alkoxy group is preferable. That is, it is preferable that the silane coupling agent is a compound containing an alkoxysilyl group. Examples of the functional group other than the hydrolyzable group include a vinyl group, a (meth)allyl group, a (meth)acryloyl group, a mercapto group, an epoxy group, an oxetanyl group, an amino group, a ureido group, a sulfide group, and an isocyanate group, and a phenyl group. Among these, an amino group, a (meth)acryloyl group, and an epoxy group are preferable. Specific examples of the silane coupling agent include the compounds described in paragraphs 0018 to 0036 of JP2009-288703A and the compounds described in paragraphs 0056 to 0066 of JP2009-242604A, and the contents thereof are incorporated in the present specification. Examples of commercially available products of the silane coupling agent include A-50 (organosilane) (manufactured by Soken Chemical & Engineering Co., Ltd.). A content of the silane coupling agent in the total solid content of the composition according to the embodiment of the present invention is preferably 0.1% to 5% by mass. The upper limit thereof is preferably 3% by mass or less and more preferably 2% by mass or less. The lower limit thereof is preferably 0.5% by mass or more and more preferably 1% by mass or more. The silane coupling agent may be used alone or in combination of two or more kinds thereof. In a case where two or more kinds of silane coupling agents are used, it is preferable that the total amount is in the above-described range.

### <<Surfactant>>

The composition according to the embodiment of the present invention can contain a surfactant. Examples of the surfactant include the surfactants described in paragraph 0017 of JP4502784B and paragraphs 0060 to 0071 of JP2009-237362A.

As the surfactant, a nonionic surfactant, a fluorine-based surfactant, or a silicone-based surfactant is preferable.

Examples of commercially available products of the fluorine-based surfactant include MEGAFACE F-171, F-172, F-173, F-176, F-177, F-141, F-142, F-143, F-144, F-437, F-475, F-477, F-479, F-482, F-551-A, F-552, F-554, F-555-A, F-556, F-557, F-558, F-559, F-560, F-561, F-565, F-563, F-568, F-575, F-780, EXP, MFS-330, R-41, R-41-LM, R-01, R-40, R-40-LM, RS-43, TF-1956, RS-90, R-94, RS-72-K, and DS-21 (all manufactured by DIC Corporation), FLUORARD FC430, FC431, and FC171 (all manufactured by Sumitomo 3M Ltd.), SURFLON S-382, SC-101, SC-103, SC-104, SC-105, SC-1068, SC-381, SC-383, S-393, and KH-40 (all manufactured by AGC Inc.), PolyFox PF636, PF656, PF6320, PF6520, and PF7002 (all manufactured by OMNOVA Solutions Inc.), and FTERGENT 710FM, 610FM, 601AD, 601ADH2, 602A, 215M, 245F, 251, 212M, 250, 209F, 222F, 208G, 710LA, 710FS, 730LM, 650AC, and 681 (all manufactured by NEOS Company Limited).

The fluorine-based surfactant has a molecular structure containing a functional group having a fluorine atom, and an acrylic compound in which a portion of the functional group having a fluorine atom is cleaved in a case where heat is applied thereto so that the fluorine atom volatilizes can also be suitably used. Examples of the fluorine-based surfactant include MEGAFACE DS Series (manufactured by DIC Corporation (The Chemical Daily (Feb. 22, 2016) and Nikkei Sangyo Daily (Feb. 23, 2016)) such as MEGAFACE DS-21.

A polymer of a fluorine atom-containing vinyl ether compound containing a fluorinated alkyl group or a fluorinated alkylene ether group and a hydrophilic vinyl ether compound is also preferably used as the fluorine-based surfactant.

A block polymer can also be used as the fluorine-based surfactant.

A fluorine-containing polymer compound having a repeating unit derived from a (meth)acrylate compound having a fluorine atom and a repeating unit derived from a (meth)acrylate compound containing 2 or more (preferably 5 or more) alkyleneoxy groups (preferably ethyleneoxy groups or propyleneoxy groups) can also be used as the fluorine-based surfactant.

A fluorine-containing polymer containing an ethylenically unsaturated bond-containing group in a side chain can also be used as the fluorine-based surfactant. Examples of commercially available products thereof include MEGAFACE RS-101, RS-102, RS-718K, and RS-72-K (all manufactured by DIC Corporation).

Due to concerns of the environmental suitability in a case of a compound containing a linear perfluoroalkyl group having 7 or more carbon atoms, an alternative material for perfluorooctanoic acid (PFOA) or perfluorooctanesulfonic acid (PFOS) is preferably used as the fluorine-based surfactant.

Examples of the silicone-based surfactant include a linear polymer consisting of a siloxane bond and a modified siloxane polymer in which an organic group is introduced into a side chain or a terminal. Examples of commercially available products of the silicone-based surfactant include DOWSIL 8032 ADDITIVE, Toray Silicone DC3PA, Toray Silicone SH7PA, Toray Silicone DC11PA, Toray Silicone SH21PA, Toray Silicone SH28PA, Toray Silicone SH29PA, Toray Silicone SH30PA, and Toray Silicone SH8400 (all manufactured by Dow Toray Co., Ltd.), X-22-4952, X-22-4272, X-22-6266, KF-351A, K354L, KF-355A, KF-945, KF-640, KF-642, KF-643, X-22-6191, X-22-4515, KF-6004, KP-341, KF-6001, and KF-6002 (all manufactured by Shin-Etsu Chemical Co., Ltd.), F-4440, TSF-4300, TSF-4445, TSF-4460, and TSF-4452 (all manufactured by Momentive Performance Materials Inc.), and BYK-307, BYK-323, and BYK-330 (all manufactured by BYK-Chemie GmbH).

Examples of the nonionic surfactant include glycerol, trimethylolpropane, trimethylolethane, ethoxylate and propoxylate thereof (such as glycerol propoxylate or glycerol ethoxylate), polyoxyethylene lauryl ether, polyoxyethylene stearyl ether, polyoxyethylene oleyl ether, polyoxyethylene octyl phenyl ether, polyoxyethylene nonyl phenyl ether, polyethylene glycol dilaurate, polyethylene glycol distearate, and sorbitan fatty acid ester. Examples of commercially available products of the nonionic surfactant include PLURONIC L10, L31, L61, L62, 10R5, 17R2, and 25R2 (all manufactured by BASF SE), TETRONIC 304, 701, 704, 901, 904, and 150R1 (all manufactured by BASF SE), SOLSPERSE 20000 (manufactured by The Lubrizol Corporation), NCW-101, NCW-1001, and NCW-1002 (all manufactured by FUJIFILM Wako Pure Chemical Corporation), PIONIN D-6112, D-6112-W, and D-6315 (all manufactured by Takemoto Oil & Fat Co., Ltd.), and OLFINE E1010, SURFINOL 104, 400, and 440 (all manufactured by Nisshin Chemical Co., Ltd.).

A content of the surfactant in the total solid content of the composition according to the embodiment of the present invention is preferably 0.01% to 3.0% by mass, more preferably 0.05% to 1.0% by mass, and still more preferably 0.10% to 0.80% by mass. The surfactant may be used alone or two or more kinds thereof. In a case where two or more kinds of silane coupling agents are used, it is preferable that the total amount is in the above-described range.

### <<Solvent>>

It is preferable that the composition according to the embodiment of the present invention further contains a solvent. The solvent is not particularly limited, and examples thereof include water and an organic solvent. It is preferable that the solvent is an organic solvent.

Examples of the organic solvent include an alcohol-based solvent, an ester-based solvent, an ether-based solvent, a ketone-based solvent, an amide-based solvent, a hydrocarbon-based solvent, and a halogen-based solvent.

Specific examples of the alcohol-based solvent include methanol, ethanol, 1-propanol, 2-propanol, 1-butanol, 2-butanol, 2-methyl-1-propanol, 1-methoxy-2-propanol, 2-ethoxyethanol, 2-butoxyethanol, ethylene glycol, propylene glycol, and glycerin.

Specific examples of the ester-based solvent include methyl acetate, ethyl acetate, n-butyl acetate, isobutyl acetate, amyl formate, isoamyl acetate, isobutyl acetate, butyl propionate, isopropyl butyrate, ethyl butyrate, butyl butyrate, methyl lactate, ethyl lactate, alkoxyacetic acid alkyl esters (such as methyl alkoxy acetate, ethyl alkoxy acetate, butyl alkoxy acetate (specific examples thereof include methyl methoxyacetate, ethyl methoxyacetate, butyl methoxyacetate, methyl ethoxyacetate, and ethyl ethoxyacetate)), 3-oxypropionic acid alkyl esters, 2-oxypropionic acid alkyl esters, methyl 2-oxy-2-methyl propionate, ethyl 2-oxy-2-methyl propionate, methyl pyruvate, ethyl pyruvate, propyl pyruvate, methyl acetoacetate, ethyl acetoacetate, methyl 2-oxobutanoate, ethyl 2-oxobutanoate, methyl cellosolve acetate, ethyl cellosolve acetate, propylene glycol monomethyl ether acetate, propylene glycol monoethyl ether acetate, propylene glycol monopropyl ether acetate, methyl 3-ethoxy propionate, ethyl 3-ethoxy propionate, and ethylene carbonate.

Specific examples of the ether-based solvent include diethylene glycol dimethyl ether, tetrahydrofuran, ethylene glycol monomethyl ether, ethylene glycol monoethyl ether, diethylene glycol monomethyl ether, diethylene glycol monoethyl ether, diethylene glycol monobutyl ether, propylene glycol monomethyl ether, polyethylene glycol monoalkyl ether, polypropylene glycol monoalkyl ether, polyethylene glycol, polypropylene glycol, ethylene glycol dialkyl ether, propylene glycol dialkyl ether, polyethylene glycol dialkyl ether, polypropylene glycol dialkyl ether, and dioxane.

Specific examples of the amide-based solvent include N-methylpyrrolidone, dimethylformamide, and dimethylacetamide.

Examples of the ketone-based solvent include methyl ethyl ketone, cyclohexanone, cyclopentanone, 2-heptanone, and 3-heptanone.

Specific examples of the hydrocarbon-based solvent include toluene and xylene.

Specific examples of the halogen-based solvent include chloroform and methylene chloride.

The above-described organic solvents may be used in combination of two or more kinds thereof.

It is preferable that the organic solvents include at least one selected from methyl 3-ethoxy propionate, ethyl 3-ethoxy propionate, ethyl cellosolve acetate, ethyl lactate, diethylene glycol dimethyl ether, butyl acetate, methyl 3-methoxy propionate, 2-heptanone, cyclohexanone, cyclopentanone, ethyl carbitol acetate, butyl carbitol acetate, propylene glycol monomethyl ether, and propylene glycol monomethyl ether acetate.

A content of the solvent in the composition according to the embodiment of the present invention is preferably 10% to 90% by mass, more preferably 30% to 90% by mass, and still more preferably 50% to 90% by mass. The composition according to the embodiment of the present invention may contain only one kind of solvent or two or more kinds thereof. In a case where the photopolymerizable composition contains two or more kinds of solvents, it is preferable that the total amount thereof is in the above-described ranges.

In addition, in a case where the composition according to the embodiment of the present invention is used as a kneaded material, a content of the organic solvent in the composition is preferably 0.1% by mass or less and more preferably 0.01% by mass or less.

### <<Plasticizer>>

In a case where the composition according to the embodiment of the present invention is used as a kneaded material, the composition according to the embodiment of the present invention can contain a plasticizer. Examples of the plasticizer include a phthalic acid ester-based plasticizer, a phosphoric acid ester-based plasticizer, a trimellitic acid ester-based plasticizer, a fatty acid ester-based plasticizer, a polyester-based plasticizer, a glycerin-based plasticizer, and a polyalkylene glycol-based plasticizer; and a phthalic acid ester-based plasticizer or a phosphoric acid ester-based plasticizer is preferable.

Examples of the phthalic acid ester-based plasticizer include dimethyl phthalate, diethyl phthalate, diisopropyl phthalate, dibutyl phthalate, diisobutyl phthalate, dihexyl phthalate, dicyclohexyl phthalate, diphenyl phthalate, bis(2-ethylhexyl) phthalate, diisononyl phthalate, diisodecyl phthalate, and diundecyl phthalate.

Examples of the phosphoric acid ester-based plasticizer include trimethyl phosphate, triethyl phosphate, tributyl phosphate, triphenyl phosphate, and tricresyl phosphate.

Examples of the trimellitic acid ester-based plasticizer include tributyl trimellitate and tris(2-ethylhexyl) trimellitate.

Examples of the fatty acid ester-based plasticizer include dimethyl adipate, diethyl adipate, dipropyl adipate, diisopropyl adipate, dibutyl adipate, diisobutyl adipate, dimethyl dodecanoate, dibutyl maleate, and ethyl oleate.

Examples of the polyester-based plasticizer include polyester consisting of an acid component such as adipic acid, sebacic acid, terephthalic acid, isophthalic acid, naphthalenedicarboxylic acid, diphenyldicarboxylic acid, or rosin and a diol component such as propylene glycol, 1,3-butanediol, 1,4-butanediol, 1,6-hexanediol, ethylene glycol, or diethylene glycol, and polyester consisting of hydroxycarboxylic acid such as polycaprolactone. The terminals of these polyesters may be blocked with a monofunctional carboxylic acid or a monofunctional alcohol, or the terminals thereof may be blocked with an epoxy compound or the like.

Examples of the glycerin-based plasticizer include glycerin monoacetomonolaurate, glycerin diacetomonolaurate, glycerin monoacetomonostearate, glycerin diacetomonooleate, and glycerin monoacetomonomontanate.

Examples of the polyalkylene glycol-based plasticizer include polyalkylene glycol such as polyethylene glycol, polypropylene glycol, polytetramethylene glycol, an ethylene oxide addition polymer of bisphenols, a propylene oxide addition polymer of bisphenols, or a tetrahydrofuran addition polymer of bisphenols, and a terminal epoxy-modified compound, a terminal ester-modified compound, and a terminal ether-modified compound thereof.

The molecular weight of the plasticizer is preferably less than 3000, more preferably 2000 or less, and still more preferably 1500 or less.

A content of the plasticizer in the composition according to the embodiment of the present invention is preferably 0.001% to 30% by mass. The lower limit thereof is preferably 0.005% by mass or more and more preferably 0.01% by mass or more. The upper limit thereof is preferably 20% by mass or less and more preferably 10% by mass or less.

The kneaded material may contain only one kind of the plasticizer or two or more kinds thereof. In a case where the kneaded material contains two or more kinds of the plasticizers, it is preferable that the total amount thereof is in the above-described ranges.

### <<Other Additives>>

The composition according to the embodiment of the present invention may appropriately contain optional additives such as a processing stabilizer, an anti-aging agent, and a compatibilizer as necessary. By allowing the resin composition to appropriately contain these components, various characteristics of the cured substance to be obtained can be appropriately adjusted.

### <Use Application of Composition>

The composition according to the embodiment of the present invention can also be suitably used for applications, in a case where the resin composition may be exposed to sunlight or light including ultraviolet rays. Specific examples include coating materials or films for window glass of houses, facilities, and transportation equipment; interior/exterior materials and interior/exterior paints of houses, facilities, and transportation equipment; members for light sources that emit ultraviolet rays, such as a fluorescent lamp and a mercury lamp; solar cells, precision machineries, electronic and electrical equipment, and members for a display device; containers or packaging materials for food, chemicals, and drugs; agricultural and industrial sheets; clothing textile products and fibers such as sportswear, stockings, and hats; lenses such as plastics lenses, contact lenses, glasses, and artificial eyes, or coating materials thereof, optical supplies such as optical filters, prisms, mirrors, and photographic materials; stationery such as tapes and inks; and marking boards, marking devices, and the surface coating materials thereof. For details thereof, reference can be made to paragraphs 0158 to 0218 of JP2009-263617A and paragraphs 0161 to 0194 of JP2009-096971A, the contents of which are incorporated herein by reference.

The composition according to the embodiment of the present invention can be preferably used for an optical member or the like. For example, the composition according to the embodiment of the present invention is preferably used for an ultraviolet cut filter, a lens, a protective material, or the like. The form of the protective material is not particularly limited, and examples thereof include a coating film, a film, and a sheet. In addition, the composition according to the embodiment of the present invention can also be used as a pressure sensitive adhesive or an adhesive.

In addition, the composition according to the embodiment of the present invention can also be used for various members of a display device. For example, in a case of a liquid crystal display device, the resin composition can be used for each member constituting the liquid crystal display device such as an antireflection film, a polarizing plate protective film, an optical film, a retardation film, a pressure sensitive adhesive, and an adhesive. Further, in a case of an organic electroluminescence display device, the polymerizable composition can be used for each member constituting the organic electroluminescence display device such as an optical film, a polarizing plate protective film in a circularly polarizing plate, a retardation film such as a quarter wave plate, and an adhesive or a pressure sensitive adhesive.

### <Cured Substance and Applications>

The cured substance according to the embodiment of the present invention is formed of the above-described composition according to the embodiment of the present invention.

The “cured substance” in the present specification includes a dried substance obtained by drying and solidifying the composition, and a cured substance obtained by curing the composition in a case where the composition undergoes a curing reaction.

The cured substance according to the embodiment of the present invention may be obtained as a molded body formed by molding the composition into a desired form. The shape of the molded body can be appropriately selected according to the intended use and the purpose. Examples of the shape thereof include a coating film, a film, a sheet, a plate, a lens, a tube, and a fiber.

The cured substance according to the embodiment of the present invention is preferably used as an optical member. Examples of the optical member include an ultraviolet cut filter, a lens, and a protective material. Further, the optical member can also be used as a polarizing plate or the like.

The ultraviolet cut filter can be used for an article such as an optical filter, a display device, a solar cell, or window glass. The kind of display device is not particularly limited, and examples thereof include a liquid crystal display device and an organic electroluminescence display device.

In a case where the cured substance according to the embodiment of the present invention is used for a lens, the cured substance according to the embodiment of the present invention may be formed into a lens shape and used. Further, the cured substance according to the embodiment of the present invention may be used for a coating film on a surface of a lens, an interlayer (adhesive layer) of a cemented lens, or the like. Examples of the cemented lens include those described in paragraphs 0094 to 0102 of WO2019/131572A, and the contents of which are incorporated in the present specification.

The kind of the protective material is not particularly limited, and examples thereof include a protective material for a display device, a protective material for a solar cell, a protective material for window glass, and an organic electroluminescence display device. The shape of the protective material is not particularly limited, and examples thereof include a coating film, a film, and a sheet.

### <Optical Member>

The optical member according to the embodiment of the present invention includes a cured substance formed of the above-described composition according to the embodiment of the present invention. The cured substance according to the embodiment of the present invention may be obtained as a molded product formed by molding the above-described composition according to the embodiment of the present invention into a desired form. The shape of the molded body can be appropriately selected according to the intended use and the purpose. Examples of the shape thereof include a coating film, a film, a sheet, a plate, a lens, a tube, and a fiber.

Examples of the type of optical member include an ultraviolet cut filter, a lens, and a protective material.

The ultraviolet cut filter can be used for an article such as an optical filter, a display device, a solar cell, or window glass. The kind of display device is not particularly limited, and examples thereof include a liquid crystal display device and an organic electroluminescence display device.

Examples of the lens include those obtained by forming the cured substance according to the embodiment of the present invention into a lens shape, and those using the cured substance according to the embodiment of the present invention as a coating film on a surface of the lens or as an interlayer (an adhesive layer or a pressure-sensitive adhesive layer) of a cemented lens.

The kind of the protective material is not particularly limited, and examples thereof include a protective material for a display device, a protective material for a solar cell, and a protective material for window glass. The shape of the protective material is not particularly limited, and examples thereof include a coating film, a film, and a sheet.

Further, a resin film is exemplified as one form of the optical member. The resin film can be formed using the composition according to the embodiment of the present invention, which contains a resin as a curable compound. Examples of the resin used in a composition for forming the resin film include the above-described resins; and a (meth)acrylic resin, a polyester fiber, a cyclic olefin resin, or a cellulose acylate resin is preferable, and a cellulose acylate resin is more preferable. The composition for forming a resin film can contain additives described in paragraphs 0022 to 0067 of JP2012-215689A. Examples of such additives include sugar esters. By adding a sugar ester compound to the composition for forming a resin film containing the cellulose acylate resin, the total haze and inside haze can be decreased without impairing expression of optical properties even in a case where a heat treatment is not performed before a stretching step. Further, the resin film (cellulose acylate film) formed of the polymerizable composition containing the cellulose acylate resin can be produced by the method described in paragraphs 0068 to 0096 of JP2012-215689A. Further, the hard coat layer described in paragraphs 0097 to 0113 of JP2012-215689A may be further laminated on the resin film.

Further, examples of other forms of the optical member include an optical member having a laminate of a support and a resin layer. In the optical member, at least one of the support or the resin layer includes the above-described cured substance according to the embodiment of the present invention.

The thickness of the resin layer in the laminate is preferably 1 μm to 2500 μm and more preferably 10 μm to 500 μm.

A material having transparency within a range where the optical performance is not impaired is preferable as the support in the laminate. The support having transparency denotes that the support is optically transparent and specifically denotes that the total light transmittance of the support is 85% or greater. The total light transmittance of the support is preferably 90% or greater and more preferably 95% or greater.

Suitable examples of the support include a resin film. Examples of the resin constituting a resin film include an ester resin (such as polyethylene terephthalate (PET), polyethylene naphthalate (PEN), polybutylene terephthalate (PBT), or polycyclohexane dimethylene terephthalate (PCT)), an olefin resin (such as polypropylene (PP) or polyethylene (PE)), polyvinyl chloride (PVA), and tricellulose acetate (TAC). Among these, PET is preferable in terms of general purpose properties.

The thickness of the support can be appropriately selected according to the applications, the purpose, and the like. In general, the thickness thereof is preferably 5 μm to 2500 μm and more preferably 20 μm to 500 μm.

In addition, a peelable support can also be used as the support. Such a laminate is preferably used as a polarizing plate or the like. Here, the peelable support is a support from which the support can be peeled off from the resin film. A stress in a case of peeling the support from the resin film is preferably 0.05 N/25 mm or more and 2.00 N/25 mm or less, more preferably 0.08 N/25 mm or more and 0.50 N/25 mm or less, and still more preferably 0.11 N/25 mm or more and 0.20 N/25 mm or less. The stress in a case of peeling the support from the resin film is evaluated by bonding and fixing the surface of the laminate cut to have a size of a width of 25 mm and a length of 80 mm to a glass substrate through an acrylic pressure sensitive adhesive sheet, grasping one end (one side with a width of 25 mm) of a test piece in the length direction using a tension tester (RTF-1210, manufactured by A & D Co., Ltd.), and performing a 90° peeling test (in conformity with Japanese Industrial Standards (JIS) K 6854-1: 1999 “Adhesive-Determination of peel strength of bonded assemblies-Part 1: 90° peeling”) in an atmosphere of a temperature of 23° C. and a relative humidity of 60% at a crosshead speed (grasping movement speed) of 200 mm/min.

A support containing polyethylene terephthalate (PET) as a main component (the component having the highest content in terms of mass among the components constituting the support) is preferable as the peelable support. From the viewpoint of mechanical strength, the weight-average molecular weight of PET is preferably 20000 or greater, more preferably 30000 or greater, and still more preferably 40000 or greater. The weight-average molecular weight of PET can be determined by dissolving the support in hexafluoroisopropanol (HFIP) using the above-described GPC method. The thickness of the support is not particularly limited, but is preferably 0.1 to 100 μm, more preferably 0.1 to 75 μm, still more preferably 0.1 to 55 μm, and particularly preferably 0.1 to 10 μm. Further, the support may be subjected to a corona treatment, a glow discharge treatment, undercoating, or the like as a known surface treatment.

Further, examples of other forms of the optical member include a laminate obtained by laminating a hard coat layer, a transparent support, and a pressure sensitive adhesive layer or an adhesive layer in this order. Such a laminate is preferably used as an ultraviolet cut filter or a protective material (a protective film or a protective sheet). The optical member in this form is not limited as long as any of the support, the hard coat layer, or the pressure sensitive adhesive layer or the adhesive layer contains the above-described cured substance according to the embodiment of the present invention.

As the hard coat layer, any of the hard coat layers described in JP2013-045045A, JP2013-043352A, JP2012-232459A, JP2012-128157A, JP2011-131409A, JP2011-131404A, JP2011-126162A, JP2011-075705A, JP2009-286981A, JP2009-263567A, JP2009-075248A, JP2007-164206A, JP2006-096811A, JP2004-075970A, JP2002-156505A, JP2001-272503A, WO2012/018087A, WO2012/098967A, WO2012/086659A, and WO2011/105594A can be applied. From the viewpoint of further improving the scratch resistance, a thickness of the hardcoat layer is preferably 5 to 100 μm.

The optical member in this form has a pressure sensitive adhesive layer or an adhesive layer on a side of the support opposite to a side where the hard coat layer is provided. The kind of the pressure sensitive adhesive or the adhesive used for the pressure sensitive adhesive layer or the adhesive layer is not particularly limited, and a known pressure sensitive adhesive or adhesive can be used. As the pressure sensitive adhesive or the adhesive, those containing the acrylic resin described in paragraphs 0056 to 0076 of JP2017-142412A and the crosslinking agent described in paragraphs 0077 to 0082 of JP2017-142412A are also preferably used. Further, the pressure sensitive adhesive or the adhesive may contain the adhesiveness improver (silane compound) described in paragraphs 0088 to 0097 of JP2017-142412A and the additives described in paragraph 0098 of JP2017-142412A.

Further, the pressure sensitive adhesive layer or the adhesive layer can be formed by the method described in paragraphs 0099 and 0100 of JP2017-142412A. The thickness of the pressure sensitive adhesive layer or the adhesive layer is preferably 5 μm to 100 μm from the viewpoint of achieving both adhesive strength and handleability.

The optical member according to the embodiment of the present invention can be preferably used as a constituent member of a display such as a liquid crystal display device (LCD) or an organic electroluminescence display device (OLED).

Examples of the liquid crystal display device include a liquid crystal display device in which a member such as an antireflection film, a polarizing plate protective film, an optical film, a retardation film, a pressure sensitive adhesive, or an adhesive contains the cured substance according to the embodiment of the present invention. The optical member including the cured substance according to the embodiment of the present invention may be disposed on a visual observer side (front side) or a backlight side with respect to the liquid crystal cell, and may be disposed on an outer side or an inner side with respect to the polarizer.

Examples of the organic electroluminescence display device include an organic electroluminescence display device in which a member such as an optical film, a polarizing plate protective film in a circularly polarizing plate, a retardation film such as a quarter wave plate, an adhesive, or a pressure sensitive adhesive contains the cured substance according to the embodiment of the present invention. In a case of using the cured substance according to the embodiment of the present invention with the above-described configuration, deterioration of the organic electroluminescent display device due to external light can be suppressed.

### EXAMPLES

Hereinafter, the present invention will be described in more detail with reference to Examples. The materials, the used amounts, the ratios, the treatment contents, the treatment procedures, and the like described in the following examples can be appropriately changed without departing from the gist of the present invention. Therefore, the scope of the present invention is not limited to the following specific examples. In addition, in the structural formulae shown below, Me is n methyl group, Et is an ethyl group, ⁿBu is an n-butyl group, ^{t}Bu is a tert-butyl group, and Ph is a phenyl group.

### Synthesis Examples

### (Synthesis Example 1) Synthesis of Intermediate 1-3

An intermediate 1-1 was synthesized according to the following scheme. In the following scheme, the synthesis of the intermediate 1-1 was carried out with reference to a method described in paragraphs 0222 and 0223 of JP2009-263617A, using 1,2-dibenzylpyrazolidine-3,5-dione instead of 1,2-dibutylpyrazolidine-3,5-dione, thereby obtaining 77 g (yield: 78%) of the intermediate 1-1.

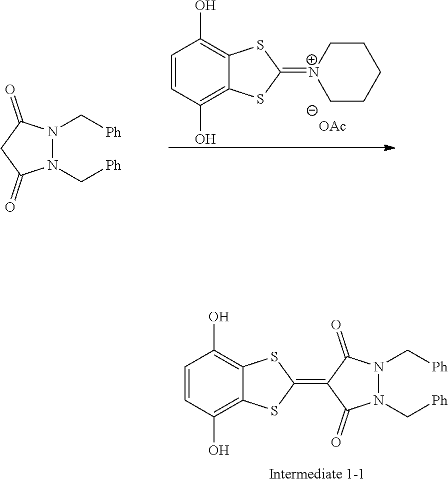 [SMILES: O=C1CC(=O)N(Cc2ccccc2)N1Cc1ccccc1 ; Oc1ccc(O)c2sc(=[N+]3CCCCC3)sc12 ; O=C1C(=C2Sc3c(O)ccc(O)c3S2)C(=O)N(Cc2ccccc2)N1Cc1ccccc1 ; "OAc"]

Next, an intermediate 1-2 was synthesized according to the following synthesis scheme. 50 g of the intermediate 1-1, 24.5 g of 2,3-dichloro-5,6-dicyano-p-benzoquinone, and 500 ml of tetrahydrofuran were added and mixed with each other, and the mixture was stirred at 20° C. for 1 hour. After completion of the reaction, 500 mL of hexane was added thereto, and the precipitated solid was collected by filtration and then washed with 150 mL of hexane to obtain 42 g (yield: 84%) of the intermediate 1-2.

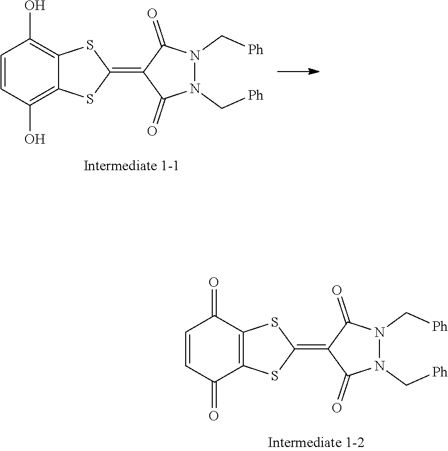 [SMILES: O=C1C(=C2Sc3c(O)ccc(O)c3S2)C(=O)N(Cc2ccccc2)N1Cc1ccccc1 ; O=C1C=CC(=O)C2=C1SC(=C1C(=O)N(Cc3ccccc3)N(Cc3ccccc3)C1=O)S2]

Next, an intermediate 1-3 was synthesized according to the following synthesis scheme. 30 g of the intermediate 1-2, 8 g of piperidinium pentamethylenedithiocarbamate, 360 mL of N-methyl-2-pyrrolidone, 160 mL of acetic acid, and 54 mL of acetone were added and mixed with each other, and the mixture was stirred at 60° C. for 1 hour. The precipitated solid was collected by filtration and then washed with 300 ml of acetone to obtain 8.0 g (yield: 36%) of the intermediate 1-3.

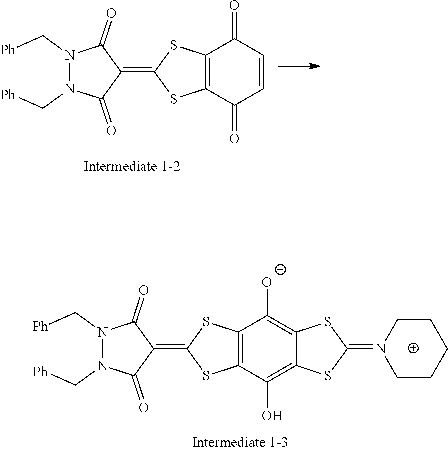

### (Synthesis Example 2) Synthesis of Compound A-104

A compound A-104 was synthesized according to the following synthesis scheme. 3.0 g of the intermediate 1-3, 0.58 g of malononitrile, and 150 ml of N-methyl-2-pyrrolidone were added and mixed with each other, and the mixture was stirred at 80° C. for 1 hour. After cooling to room temperature, 1 mL of hydrochloric acid and 150 ml of water were added thereto, and the mixture was stirred for 30 minutes. The precipitated solid was collected by filtration, 200 ml of acetonitrile was added thereto, and the mixture was heated and refluxed under a nitrogen atmosphere for 1 hour. After cooling to room temperature and stirring at room temperature for 1 hour, the solid was collected by filtration and then washed with 100 ml of acetonitrile to obtain 2.1 g (yield: 79%) of the compound A-104. In addition, in a proton nuclear magnetic resonance (¹H-NMR, solvent: deuterated dimethyl sulfoxide (dDMSO)) of the obtained compound A-104, chemical shifts 6 were 11.6 (s, 2H), 7.29 (m, 10H), and 4.80 (s, 4H).

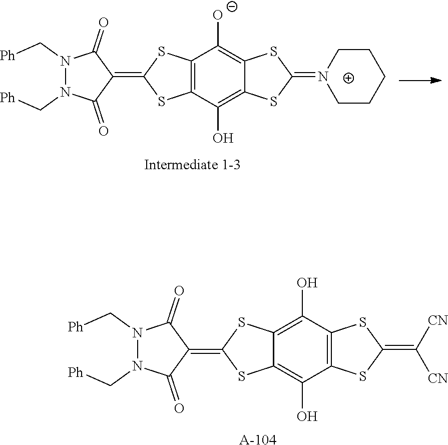

### (Synthesis Example 3) Synthesis of Compound A-1

A compound A-1 was synthesized according to the following synthesis scheme. 1.5 g of the compound A-104, 0.76 g of triethylamine, 1.0 g of 2-ethylhexanoyl chloride, and 30 ml of dimethylacetamide were added and mixed with each other, and the mixture was stirred at 20° C. for 1 hour. After completion of the reaction, 30 ml of water was added thereto, followed by stirring for 30 minutes. The precipitated solid was collected by filtration, washed with 30 ml of methanol, and then purified by silica gel column chromatography to obtain 1.6 g (yield: 75%) of the compound A-1. In addition, in a proton nuclear magnetic resonance (¹H-NMR, solvent: deuterated chloroform (CDCl₃)) of the obtained compound A-1, chemical shifts 6 were 7.27 (m, 6H), 7.10 (m, 4H), 4.75 (s, 4H), 2.69 (m, 2H), 1.8 to 1.6 (m, 8H), 1.5 to 1.3 (m, 8H), 1.10 (m, 6H), and 0.94 (m, 6H).

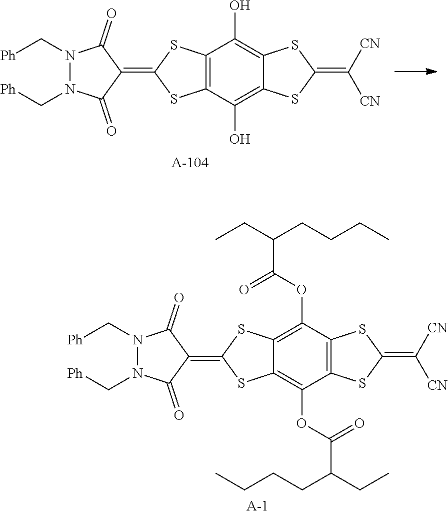

### <Regarding Ultraviolet Absorber>

In each of the test examples shown below, the exemplary compounds (1) to (26) and the comparative compounds (1) and (2) used as the ultraviolet absorbers are compounds having the structures shown below, respectively.

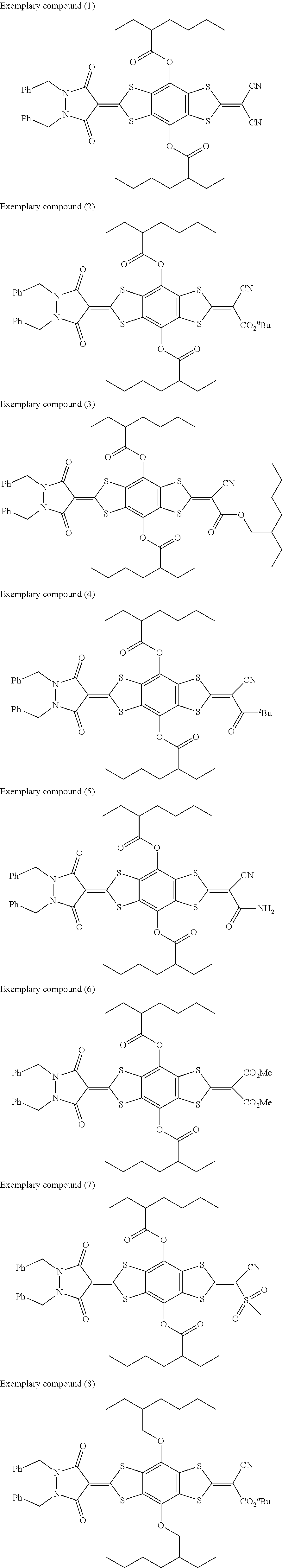

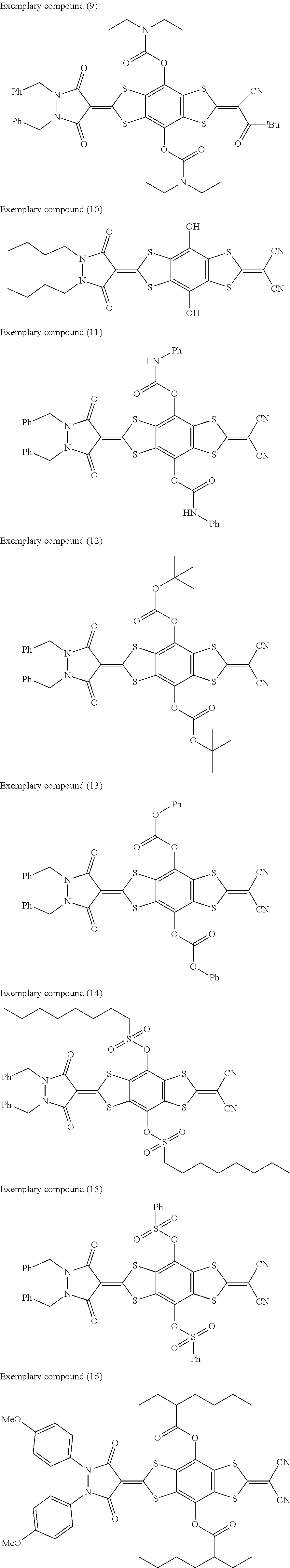

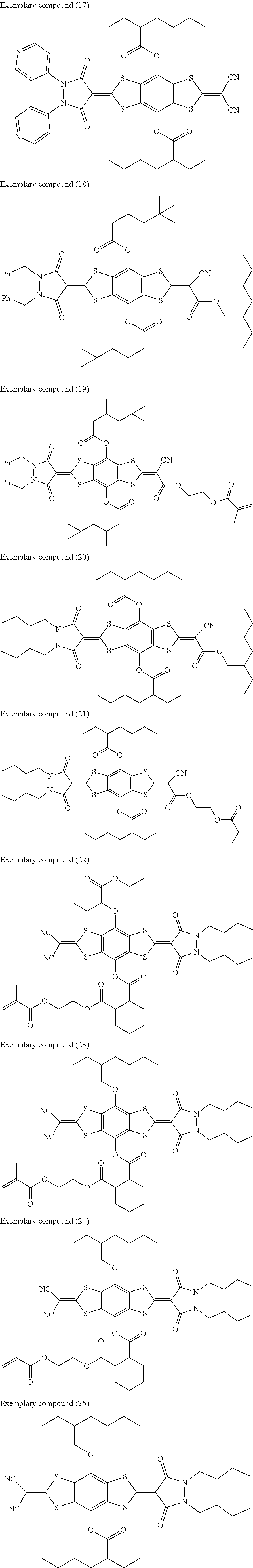

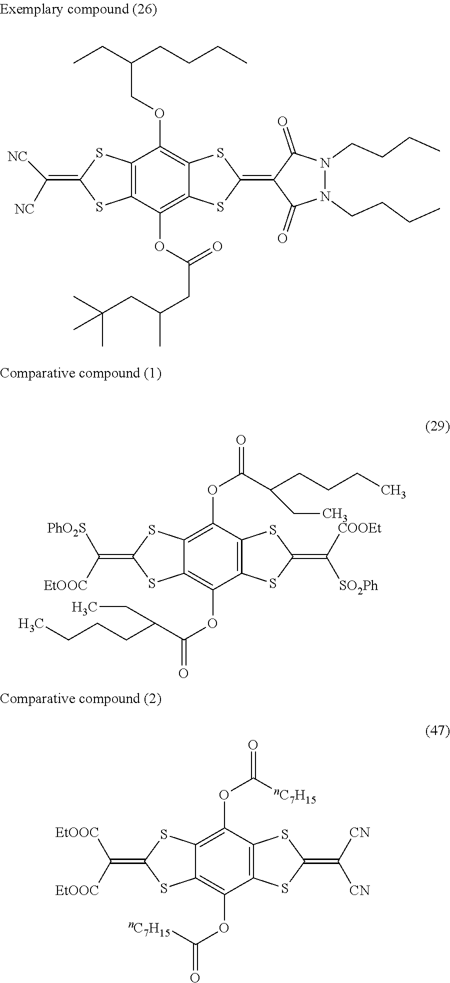

### <About Anti-Fading Agent>

In each of the test examples shown below, the compounds T-1 to T-12 used as the anti-fading agent are compounds having the structures shown below, respectively.

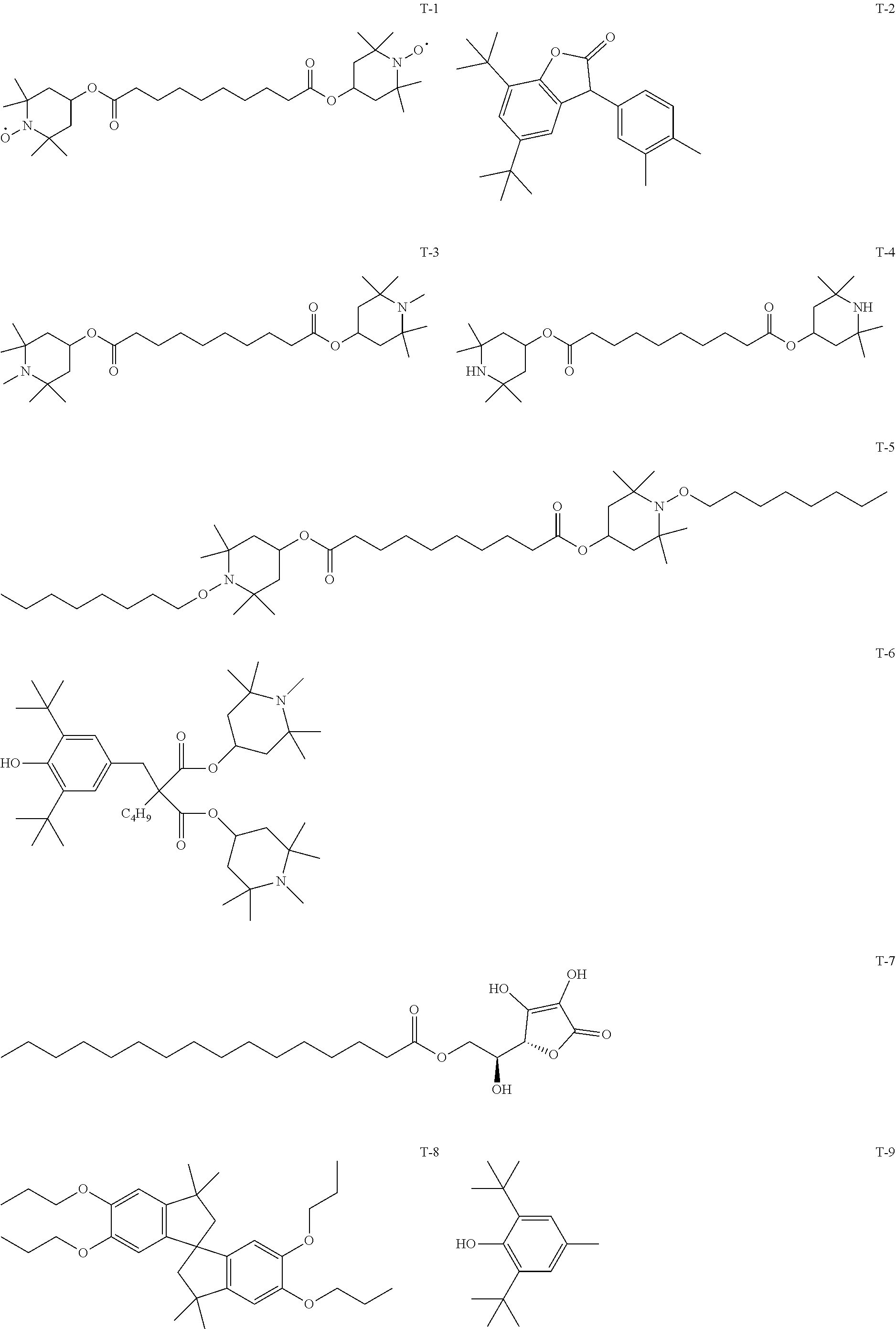

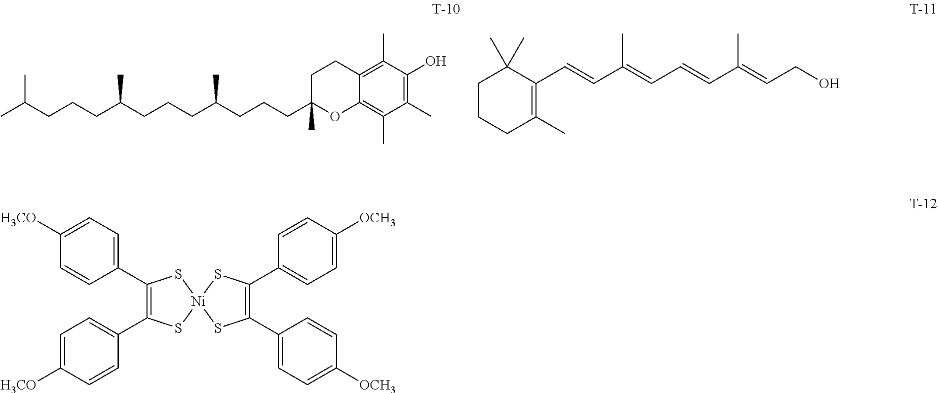

### Test Example 1

The following components were mixed to prepare each composition.

Ultraviolet absorber (compounds (exemplary compounds (1) to (21), comparative compounds (1) and (2)) described in the table below) . . . 1.1 parts by mass

Resin (Dianal BR-80, manufactured by Mitsubishi Chemical Corporation., containing 60% by mass or more of methyl methacrylate as a monomer unit, weight-average molecular weight: 95000) . . . 12.6 parts by mass

Anti-fading agent (compound shown in the table below) . . . 1.1 parts by mass

Solvent (chloroform) . . . 85.2 parts by mass

TABLE 1

| | Ultraviolet absorber | Anti-fading agent |
|---|---|---|
| Example 101 | Exemplary compound (1) | T-1 |
| Example 102 | Exemplary compound (1) | T-2 |
| Example 103 | Exemplary compound (1) | T-7 |
| Example 104 | Exemplary compound (1) | T-8 |
| Example 105 | Exemplary compound (1) | T-9 |
| Example 106 | Exemplary compound (1) | T-10 |
| Example 107 | Exemplary compound (2) | T-1 |
| Example 108 | Exemplary compound (3) | T-3 |
| Example 109 | Exemplary compound (4) | T-4 |
| Example 110 | Exemplary compound (5) | T-5 |
| Example 111 | Exemplary compound (6) | T-6 |
| Example 112 | Exemplary compound (7) | T-2 |
| Example 113 | Exemplary compound (8) | T-1 |
| Example 114 | Exemplary compound (9) | T-3 |
| Example 115 | Exemplary compound (10) | T-4 |
| Example 116 | Exemplary compound (11) | T-5 |
| Example 117 | Exemplary compound (12) | T-6 |
| Example 118 | Exemplary compound (13) | T-2 |
| Example 119 | Exemplary compound (14) | T-1 |
| Example 120 | Exemplary compound (15) | T-3 |
| Example 121 | Exemplary compound (16) | T-4 |
| Example 122 | Exemplary compound (17) | T-5 |
| Example 123 | Exemplary compound (18) | T-1 |
| Example 124 | Exemplary compound (18) | T-2 |
| Example 125 | Exemplary compound (18) | T-3 |
| Example 126 | Exemplary compound (18) | T-7 |
| Example 127 | Exemplary compound (18) | T-8 |
| Example 128 | Exemplary compound (18) | T-9 |
| Example 129 | Exemplary compound (18) | T-10 |
| Example 130 | Exemplary compound (19) | T-1 |
| Example 131 | Exemplary compound (19) | T-2 |
| Example 132 | Exemplary compound (19) | T-4 |
| Example 133 | Exemplary compound (19) | T-7 |
| Example 134 | Exemplary compound (19) | T-8 |
| Example 135 | Exemplary compound (19) | T-9 |
| Example 136 | Exemplary compound (19) | T-10 |
| Example 137 | Exemplary compound (20) | T-1 |
| Example 138 | Exemplary compound (20) | T-2 |
| Example 139 | Exemplary compound (20) | T-5 |
| Example 140 | Exemplary compound (20) | T-7 |

TABLE 2

| | Ultraviolet absorber | Anti-fading agent |
|---|---|---|
| Example 141 | Exemplary compound (20) | T-8 |
| Example 142 | Exemplary compound (20) | T-9 |
| Example 143 | Exemplary compound (20) | T-10 |
| Example 144 | Exemplary compound (21) | T-1 |
| Example 145 | Exemplary compound (21) | T-2 |
| Example 146 | Exemplary compound (21) | T-6 |
| Example 147 | Exemplary compound (21) | T-7 |
| Example 148 | Exemplary compound (21) | T-8 |
| Example 149 | Exemplary compound (21) | T-9 |
| Example 150 | Exemplary compound (21) | T-10 |
| Example 151 | Exemplary compound (18) | T-11 |
| Example 152 | Exemplary compound (19) | T-11 |
| Example 153 | Exemplary compound (21) | T-11 |
| Example 154 | Exemplary compound (18) | T-12 |
| Example 155 | Exemplary compound (19) | T-12 |
| Example 156 | Exemplary compound (21) | T-12 |
| Example 157 | Exemplary compound (22) | T-1 |
| Example 158 | Exemplary compound (22) | T-2 |
| Example 159 | Exemplary compound (23) | T-1 |
| Example 160 | Exemplary compound (23) | T-2 |
| Example 161 | Exemplary compound (24) | T-1 |
| Example 162 | Exemplary compound (24) | T-2 |
| Example 163 | Exemplary compound (25) | T-1 |
| Example 164 | Exemplary compound (25) | T-2 |
| Example 165 | Exemplary compound (26) | T-1 |
| Example 166 | Exemplary compound (26) | T-2 |
| Comparative | Comparative compound (1) | T-1 |
| Example 101 | | |
| Comparative | Comparative compound (1) | T-2 |
| Example 102 | | |
| Comparative | Comparative compound (2) | T-1 |
| Example 103 | | |
| Comparative | Comparative compound (2) | T-2 |
| Example 104 | | |

Each of the obtained compositions was applied onto a glass substrate by spin coating and then dried at 110° C. for 2 minutes to produce a film.

### (Evaluation of Absorption Ability of UV-A)

The absorbance of the obtained film was measured using a spectrophotometer (UV-1800PC, manufactured by Shimadzu Corporation). A maximal absorption wavelength (λₘₐₓ) was measured from absorption spectrum of each sample solution, and absorption ability of UV-A was evaluated according to the following standard. The numerical value in the column of absorption ability of UV-A is the value of λₘₐₓ and the numerical value in the column of colorability is the value of the absorbance ratio A₄₄₀.

### —Evaluation Standard for Absorption Ability of UV-A—

A: λₘₐₓ was 390 nm or more.
B: λₘₐₓ was 380 nm or more and less than 390 nm.
C: λₘₐₓ was less than 380 nm.

### (Evaluation of Colorability)

The absorbance of the obtained film was measured using a spectrophotometer (UV-1800PC, manufactured by Shimadzu Corporation), and a value of a ratio of the absorbance at a wavelength of 440 nm in a case where the absorbance at a wavelength of 400 nm was set to 1 (absorbance ratio A₄₄₀) was calculated, and the colorability (colorability 1) was evaluated according to the following standard. As the value of the absorbance ratio A₄₄₀ is smaller, the coloration less occurs.

Next, the obtained film was subjected to a light resistance test under the following condition 1, the absorbance of the film after the light resistance test was measured using a spectrophotometer (UV-1800PC, manufactured by Shimadzu Corporation), and a value of a ratio of the absorbance at a wavelength of 440 nm in a case where the absorbance at a wavelength of 400 nm was set to 1 (absorbance ratio A₄₄₀) was calculated, and the colorability was evaluated according to the following standard. The numerical value in parentheses in the column of “Coloring properties” is a value of the absorbance ratio A₄₄₀. As the value of the absorbance ratio A₄₄₀ is smaller, the coloration less occurs.

### —Evaluation Standard of Colorability—

A: absorbance ratio A₄₄₀ was 0.01 or less.
B: absorbance ratio A₄₄₀ was more than 0.01.
C: absorbance ratio A₄₄₀ was more than 0.02.

### —Condition 1—

Equipment: low temperature cycle xenon weather meter (XL75, manufactured by Suga Test Instruments Co., Ltd.)

Illuminance: 90 klx (40 w/m²)

Time: 4 weeks

Environment: 23° C. at relative humidity of 50%

### (Evaluation of Light Resistance)

For each film, a light resistance test was performed under the above-described condition 1, and the retention rate of the absorbance at the maximal absorption wavelength (λₘₐₓ) was determined to evaluate the light resistance. Specifically, an absorbance of the film at λₘₐₓ was measured using a spectrophotometer (UV-1800PC, manufactured by Shimadzu Corporation), the film was subjected to a light resistance test under the condition 1, and the absorbance of the film after the light resistance test at λₘₐₓ was measured. Next, an absorbance retention rate (%) was calculated from the values of the absorbance of the film before and after the light resistance test at λₘₐₓ using the following expression, and light resistance was evaluated according to the following standard. As the absorbance retention rate is higher, the light resistance is more excellent. The evaluation results are shown in the tables below. The numerical value in the column of light resistance is the value of the absorbance retention rate.

$$Absorbance retention rate ( \% ) = ( Absorbance of film after light resistance test at {λ}_{max} / Absorbance of film before light resistance test at {λ}_{max} ) \times 100$$

### —Evaluation Standards—

AA: absorbance retention rate was 90% or more.
A: absorbance retention rate was 85% or more.
B: absorbance retention rate was 80% or more and less than 85%.
C: absorbance retention rate was less than 80%.

TABLE 3

| | | Colorability | | |
|---|---|---|---|---|
| | | before | | |
| | | light | after light | Light |
| | Absorption | resistance | resistance | resistance |
| | ability of | test | test | (absorbance |
| | UV-A | (absorbance | (absorbance | retention |
| | (λₘₐₓ (nm)) | ratio A₄₄₀) | ratio A₄₄₀) | rate (%)) |
| Example | A(397) | A(0.005) | A(0.005) | AA(93) |
| 101 | | | | |
| Example | A(397) | A(0.005) | A(0.005) | AA(91) |
| 102 | | | | |
| Example | A(397) | A(0.005) | A(0.006) | A(88) |
| 103 | | | | |
| Example | A(397) | A(0.005) | A(0.006) | A(89) |
| 104 | | | | |
| Example | A(397) | A(0.005) | A(0.006) | A(87) |
| 105 | | | | |
| Example | A(397) | A(0.005) | A(0.006) | A(89) |
| 106 | | | | |
| Example | A(398) | A(0.006) | A(0.006) | AA(91) |
| 107 | | | | |
| Example | A(398) | A(0.006) | A(0.006) | AA(91) |
| 108 | | | | |
| Example | A(405) | A(0.007) | A(0.007) | AA(91) |
| 109 | | | | |
| Example | A(398) | A(0.007) | A(0.007) | AA(93) |
| 110 | | | | |
| Example | A(400) | A(0.008) | A(0.008) | AA(94) |
| 111 | | | | |
| Example | A(395) | A(0.007) | A(0.007) | AA(93) |
| 112 | | | | |
| Example | A(400) | A(0.006) | A(0.006) | AA(92) |
| 113 | | | | |
| Example | A(407) | A(0.007) | A(0.007) | AA(93) |
| 114 | | | | |
| Example | A(395) | A(0.010) | A(0.010) | AA(95) |
| 115 | | | | |
| Example | A(400) | A(0.008) | A(0.008) | AA(91) |
| 116 | | | | |
| Example | A(396) | A(0.005) | A(0.005) | AA(92) |
| 117 | | | | |
| Example | A(396) | A(0.005) | A(0.005) | AA(93) |
| 118 | | | | |
| Example | A(395) | A(0.004) | A(0.004) | AA(95) |
| 119 | | | | |
| Example | A(395) | A(0.007) | A(0.007) | AA(95) |
| 120 | | | | |
| Example | A(397) | A(0.009) | A(0.009) | AA(90) |
| 121 | | | | |
| Example | A(399) | A(0.010) | A(0.010) | AA(90) |
| 122 | | | | |
| Example | A(398) | A(0.006) | A(0.006) | AA(92) |
| 123 | | | | |
| Example | A(398) | A(0.006) | A(0.006) | AA(92) |
| 124 | | | | |
| Example | A(398) | A(0.006) | A(0.006) | AA(91) |
| 125 | | | | |
| Example | A(398) | A(0.006) | A(0.007) | A(88) |
| 126 | | | | |
| Example | A(398) | A(0.006) | A(0.007) | A(88) |
| 127 | | | | |
| Example | A(398) | A(0.006) | A(0.007) | A(88) |
| 128 | | | | |
| Example | A(398) | A(0.006) | A(0.007) | A(89) |
| 129 | | | | |
| Example | A(398) | A(0.006) | A(0.006) | AA(92) |
| 130 | | | | |
| Example | A(398) | A(0.006) | A(0.006) | AA(91) |
| 131 | | | | |
| Example | A(398) | A(0.006) | A(0.006) | AA(90) |
| 132 | | | | |
| Example | A(398) | A(0.006) | A(0.007) | A(88) |
| 133 | | | | |
| Example | A(398) | A(0.006) | A(0.007) | A(89) |
| 134 | | | | |
| Example | A(398) | A(0.006) | A(0.007) | A(87) |
| 135 | | | | |
| Example | A(398) | A(0.006) | A(0.007) | A(89) |
| 136 | | | | |
| Example | A(395) | A(0.006) | A(0.006) | AA(91) |
| 137 | | | | |
| Example | A(395) | A(0.006) | A(0.006) | AA(92) |
| 138 | | | | |
| Example | A(395) | A(0.006) | A(0.006) | AA(91) |
| 139 | | | | |
| Example | A(395) | A(0.006) | A(0.007) | A(88) |
| 140 | | | | |

TABLE 4

| | | Colorability | | |
|---|---|---|---|---|
| | | before | after | |
| | | light | light | Light |
| | Absorption | resistance | resistance | resistance |
| | ability of | test | test | (absorbance |
| | UV-A | (absorbance | (absorbance | retention |
| | (λₘₐₓ (nm)) | ratio A₄₄₀) | ratio A₄₄₀) | rate (%)) |
| Example 141 | A(395) | A(0.006) | A(0.007) | A(87) |
| Example 142 | A(395) | A(0.006) | A(0.007) | A(87) |
| Example 143 | A(395) | A(0.006) | A(0.007) | A(89) |
| Example 144 | A(395) | A(0.006) | A(0.006) | AA(93) |
| Example 145 | A(395) | A(0.006) | A(0.006) | AA(92) |
| Example 146 | A(395) | A(0.006) | A(0.006) | AA(91) |
| Example 147 | A(395) | A(0.006) | A(0.007) | A(89) |
| Example 148 | A(395) | A(0.006) | A(0.007) | A(89) |
| Example 149 | A(395) | A(0.006) | A(0.007) | A(88) |
| Example 150 | A(395) | A(0.006) | A(0.007) | A(89) |
| Example 151 | A(398) | A(0.006) | A(0.007) | A(88) |
| Example 152 | A(398) | A(0.006) | A(0.007) | A(87) |
| Example 153 | A(395) | A(0.006) | A(0.007) | A(88) |
| Example 154 | A(398) | A(0.006) | A(0.007) | A(88) |
| Example 155 | A(398) | A(0.006) | A(0.007) | A(88) |
| Example 156 | A(397) | A(0.006) | A(0.007) | A(87) |
| Example 157 | A(397) | A(0.005) | A(0.005) | AA(93) |
| Example 158 | A(397) | A(0.005) | A(0.005) | AA(91) |
| Example 159 | A(397) | A(0.005) | A(0.005) | AA(92) |
| Example 160 | A(397) | A(0.005) | A(0.005) | AA(90) |
| Example 161 | A(397) | A(0.005) | A(0.005) | AA(93) |
| Example 162 | A(397) | A(0.005) | A(0.005) | AA(91) |
| Example 163 | A(397) | A(0.005) | A(0.005) | AA(93) |
| Example 164 | A(397) | A(0.005) | A(0.005) | AA(91) |
| Example 165 | A(397) | A(0.005) | A(0.005) | AA(93) |
| Example 166 | A(397) | A(0.005) | A(0.005) | AA(91) |
| Comparative | C(379) | B(0.012) | B(0.017) | C(65) |
| Example 101 | | | | |
| Comparative | C(379) | B(0.012) | B(0.015) | C(62) |
| Example 102 | | | | |
| Comparative | B(385) | B(0.015) | B(0.017) | C(51) |
| Example 103 | | | | |
| Comparative | B(385) | B(0.015) | B(0.018) | C(55) |
| Example 104 | | | | |

As shown in the above table, Examples 101 to 166 had excellent absorption ability of UV-A. In addition, the value of the absorbance ratio A₄₄₀ was small in both before and after the light resistance test, and the colorability was excellent. Further, the absorbance retention rate after the light resistance test was also high, and the evaluation of the light resistance was also excellent.

### Test Example 2

The following components were mixed to prepare a composition.

Ultraviolet absorber (compound shown in the following table) . . . 0.6 parts by mass

Polymerizable compound (KAYARAD DPHA (manufactured by Nippon Kayaku Co., Ltd.), compound having two or more ethylenically unsaturated bond-containing groups) . . . 10.9 parts by mass

Resin (Dianal BR-80 (manufactured by Mitsubishi Chemical Corporation)) . . . 13.4 parts by mass

Photopolymerization initiator (compound shown in the following table) . . . 0.6 parts by mass

Anti-fading agent (compound shown in the following table) . . . 0.8 parts by mass

Solvent (propylene glycol monomethyl ether acetate): . . . 73.7 parts by mass

TABLE 5

| | | Anti-fading | Photopolymerization |
|---|---|---|---|
| | Ultraviolet absorber | agent | initiator |
| Example 201 | Exemplary | T-1 | V-1 |
| | compound (1) | | |
| Example 202 | Exemplary | T-2 | V-1 |
| | compound (1) | | |
| Example 203 | Exemplary | T-7 | V-1 |
| | compound (1) | | |
| Example 204 | Exemplary | T-8 | V-1 |
| | compound (1) | | |
| Example 205 | Exemplary | T-9 | V-1 |
| | compound (1) | | |
| Example 206 | Exemplary | T-10 | V-1 |
| | compound (1) | | |
| Example 207 | Exemplary | T-1 | V-2 |
| | compound (1) | | |
| Example 208 | Exemplary | T-1 | V-1 |
| | compound (18) | | |
| Example 209 | Exemplary | T-2 | V-1 |
| | compound (18) | | |
| Example 210 | Exemplary | T-1 | V-2 |
| | compound (18) | | |
| Example 211 | Exemplary | T-1 | V-3 |
| | compound (18) | | |
| Example 212 | Exemplary | T-7 | V-1 |
| | compound (18) | | |
| Example 213 | Exemplary | T-8 | V-3 |
| | compound (18) | | |
| Example 214 | Exemplary | T-9 | V-1 |
| | compound (18) | | |
| Example 215 | Exemplary | T-10 | V-3 |
| | compound (18) | | |
| Example 216 | Exemplary | T-1 | V-1 |
| | compound (19) | | |
| Example 217 | Exemplary | T-2 | V-1 |
| | compound (19) | | |
| Example 218 | Exemplary | T-1 | V-2 |
| | compound (19) | | |
| Example 219 | Exemplary | T-1 | V-3 |
| | compound (19) | | |
| Example 220 | Exemplary | T-7 | V-1 |
| | compound (19) | | |
| Example 221 | Exemplary | T-8 | V-3 |
| | compound (19) | | |
| Example 222 | Exemplary | T-9 | V-1 |
| | compound (19) | | |
| Example 223 | Exemplary | T-10 | V-3 |
| | compound (19) | | |
| Example 224 | Exemplary | T-1 | V-1 |
| | compound (20) | | |
| Example 225 | Exemplary | T-2 | V-1 |
| | compound (20) | | |
| Example 226 | Exemplary | T-1 | V-2 |
| | compound (20) | | |
| Example 227 | Exemplary | T-1 | V-3 |
| | compound (20) | | |
| Example 228 | Exemplary | T-7 | V-1 |
| | compound (20) | | |
| Example 229 | Exemplary | T-8 | V-3 |
| | compound (20) | | |
| Example 230 | Exemplary | T-9 | V-1 |
| | compound (20) | | |
| Example 231 | Exemplary | T-10 | V-3 |
| | compound (20) | | |
| Example 232 | Exemplary | T-1 | V-1 |
| | compound (21) | | |
| Example 233 | Exemplary | T-2 | V-1 |
| | compound (21) | | |
| Example 234 | Exemplary | T-1 | V-2 |
| | compound (21) | | |
| Example 235 | Exemplary | T-1 | V-3 |
| | compound (21) | | |
| Example 236 | Exemplary | T-7 | V-1 |
| | compound (21) | | |
| Example 237 | Exemplary | T-8 | V-3 |
| | compound (21) | | |
| Example 238 | Exemplary | T-9 | V-1 |
| | compound (21) | | |
| Example 239 | Exemplary | T-10 | V-3 |
| | compound (21) | | |

TABLE 6

| | Ultraviolet | Anti-fading | Photopolymerization |
|---|---|---|---|
| | absorber | agent | initiator |
| Example 240 | Exemplary | T-11 | V-1 |
| | compound (19) | | |
| Example 241 | Exemplary | T-12 | V-1 |
| | compound (19) | | |
| Example 242 | Exemplary | T-11 | V-1 |
| | compound (21) | | |
| Example 243 | Exemplary | T-12 | V-1 |
| | compound (21) | | |
| Example 244 | Exemplary | T-1 | V-1 |
| | compound (22) | | |
| Example 245 | Exemplary | T-2 | V-1 |
| | compound (22) | | |
| Example 246 | Exemplary | T-1 | V-1 |
| | compound (23) | | |
| Example 247 | Exemplary | T-2 | V-1 |
| | compound (23) | | |
| Example 248 | Exemplary | T-1 | V-1 |
| | compound (24) | | |
| Example 249 | Exemplary | T-2 | V-1 |
| | compound (24) | | |
| Example 250 | Exemplary | T-1 | V-1 |
| | compound (25) | | |
| Example 251 | Exemplary | T-2 | V-1 |
| | compound (25) | | |
| Example 252 | Exemplary | T-1 | V-1 |
| | compound (26) | | |
| Example 253 | Exemplary | T-2 | V-1 |
| | compound (26) | | |
| Comparative | Comparative | T-1 | V-1 |
| Example 201 | compound (1) | | |
| Comparative | Comparative | T-2 | V-1 |
| Example 202 | compound (1) | | |
| Comparative | Comparative | T-1 | V-2 |
| Example 203 | compound (1) | | |
| Comparative | Comparative | T-1 | V-1 |
| Example 204 | compound (2) | | |
| Comparative | Comparative | T-2 | V-1 |
| Example 205 | compound (2) | | |
| Comparative | Comparative | T-1 | V-2 |
| Example 206 | compound (2) | | |
| Comparative | Comparative | T-2 | V-3 |
| Example 207 | compound (3) | | |

### (Photopolymerization Initiator)

V-1: Irgacure OXE01 (manufactured by BASF SE, oxime compound, photoradical polymerization initiator)

V-2: Omnirad 2959 (manufactured by IGM Resins B. V, hydroxyacetophenone compound, photoradical polymerization initiator)

V-3: Omnirad TPO (manufactured by IGM Resins B. V, acylphosphine compound, photoradical polymerization initiator)

Each composition was spin-coated on a glass substrate (1737, manufactured by Corning) having a size of 50 mm×50 mm such that a film thickness after film formation was 1.5 μm, and dried at 120° C. for 5 minutes to form a composition layer. Thereafter, the composition layer was entirely exposed to an i-ray stepper exposure device (UX-1000SM-EH04, manufactured by USHIO INC.) with an exposure amount of 1,000 mJ/cm², thereby producing film. In Examples 201 to 243, the degree of change in transmittance at the maximum absorption wavelength (λₘₐₓ) of the composition layer before and after exposure was 5% or less.

### (Evaluation of Colorability)

The colorability of the obtained film was evaluated by the same method and evaluation standards as those for the evaluation of the colorability in Test Example 1.

### (Evaluation of Light Resistance)

The obtained film was subjected to a light resistance test under the following condition 2, and the retention rate of the absorbance at the maximum absorption wavelength (λₘₐₓ) before and after the light resistance test was determined by the same method as the evaluation of the light resistance in Test Example 1, and the light resistance was evaluated according to the following evaluation standard.

### —Condition 2—

Equipment: low temperature cycle xenon weather meter (XL75, manufactured by Suga Test Instruments Co., Ltd.)
Illuminance: 90 klx (40 w/m²)
Time: 7 days
Environment: 23° C. at relative humidity of 50%

### —Evaluation Standard for Light Fastness—

AA: absorbance retention rate was 80% or more.
A: absorbance retention rate was 70% or more.
B: absorbance retention rate was 60% or more and less than 70%.
C: absorbance retention rate was less than 60%.

TABLE 7

Colorability

| | before | after | |
|---|---|---|---|
| | light | light | Light |
| | resistance | resistance | resistance |
| | test | test | (absorbance |
| | (absorbance | (absorbance | retention |
| | ratio A₄₄₀) | ratio A₄₄₀) | rate (%)) |
| Example 201 | A(0.005) | A(0.005) | AA(83) |
| Example 202 | A(0.005) | A(0.006) | AA(82) |
| Example 203 | A(0.005) | A(0.009) | A(78) |
| Example 204 | A(0.005) | A(0.008) | A(77) |
| Example 205 | A(0.005) | A(0.008) | A(79) |
| Example 206 | A(0.005) | A(0.009) | A(76) |
| Example 207 | A(0.005) | A(0.006) | AA(83) |
| Example 208 | A(0.007) | A(0.007) | AA(83) |
| Example 209 | A(0.007) | A(0.008) | AA(81) |
| Example 210 | A(0.007) | A(0.007) | AA(83) |
| Example 211 | A(0.007) | A(0.007) | AA(84) |
| Example 212 | A(0.007) | A(0.009) | A(77) |
| Example 213 | A(0.007) | A(0.008) | A(78) |
| Example 214 | A(0.007) | A(0.008) | A(76) |
| Example 215 | A(0.007) | A(0.009) | A(76) |
| Example 216 | A(0.007) | A(0.007) | AA(82) |
| Example 217 | A(0.007) | A(0.008) | AA(81) |
| Example 218 | A(0.007) | A(0.007) | AA(83) |
| Example 219 | A(0.007) | A(0.007) | AA(83) |
| Example 220 | A(0.007) | A(0.009) | A(76) |
| Example 221 | A(0.007) | A(0.009) | A(77) |
| Example 222 | A(0.007) | A(0.009) | A(76) |
| Example 223 | A(0.007) | A(0.009) | A(78) |
| Example 224 | A(0.006) | A(0.006) | AA(81) |
| Example 225 | A(0.006) | A(0.007) | AA(82) |
| Example 226 | A(0.006) | A(0.006) | AA(83) |
| Example 227 | A(0.006) | A(0.006) | AA(83) |
| Example 228 | A(0.006) | A(0.008) | A(76) |
| Example 229 | A(0.006) | A(0.009) | A(77) |
| Example 230 | A(0.006) | A(0.008) | A(76) |
| Example 231 | A(0.006) | A(0.009) | A(78) |
| Example 232 | A(0.007) | A(0.007) | AA(81) |
| Example 233 | A(0.007) | A(0.007) | AA(81) |
| Example 234 | A(0.007) | A(0.007) | AA(82) |
| Example 235 | A(0.007) | A(0.007) | AA(82) |
| Example 236 | A(0.007) | A(0.009) | A(76) |
| Example 237 | A(0.007) | A(0.009) | A(77) |
| Example 238 | A(0.007) | A(0.008) | A(76) |
| Example 239 | A(0.007) | A(0.009) | A(78) |

TABLE 8

Colorability

| | before | after | |
|---|---|---|---|
| | light | light | Light |
| | resistance | resistance | resistance |
| | test | test | (absorbance |
| | (absorbance | (absorbance | retention |
| | ratio A₄₄₀) | ratio A₄₄₀) | rate (%)) |
| Example 240 | A(0.007) | A(0.009) | A(76) |
| Example 241 | A(0.007) | A(0.009) | A(77) |
| Example 242 | A(0.007) | A(0.008) | A(76) |
| Example 243 | A(0.007) | A(0.009) | A(78) |
| Example 244 | A(0.005) | A(0.005) | AA(83) |
| Example 245 | A(0.005) | A(0.006) | AA(82) |
| Example 246 | A(0.005) | A(0.005) | AA(82) |
| Example 247 | A(0.005) | A(0.006) | AA(81) |
| Example 248 | A(0.005) | A(0.005) | AA(83) |
| Example 249 | A(0.005) | A(0.006) | AA(82) |
| Example 250 | A(0.005) | A(0.005) | AA(83) |
| Example 251 | A(0.005) | A(0.006) | AA(82) |
| Example 252 | A(0.005) | A(0.005) | AA(83) |
| Example 253 | A(0.005) | A(0.006) | AA(82) |
| Comparative | B(0.012) | C(0.032) | C(48) |
| Example 201 | | | |
| Comparative | B(0.012) | C(0.028) | C(47) |
| Example 202 | | | |
| Comparative | B(0.015) | B(0.019) | C(51) |
| Example 203 | | | |
| Comparative | B(0.012) | C(0.031) | C(38) |
| Example 204 | | | |
| Comparative | B(0.015) | C(0.023) | C(44) |
| Example 205 | | | |
| Comparative | B(0.015) | B(0.018) | C(51) |
| Example 206 | | | |
| Comparative | B(0.012) | B(0.018) | C(59) |
| Example 207 | | | |

As shown in the above table, in Examples 201 to 253, the value of the absorbance ratio A₄₄₀ was small before and after the light resistance test, and the colorability was excellent. Further, the absorbance retention rate after the light resistance test was also high, and the evaluation of the light resistance was also excellent.

### <Synthesis of Polymer>

### (Synthesis Example 101) Synthesis of Polymer P-1

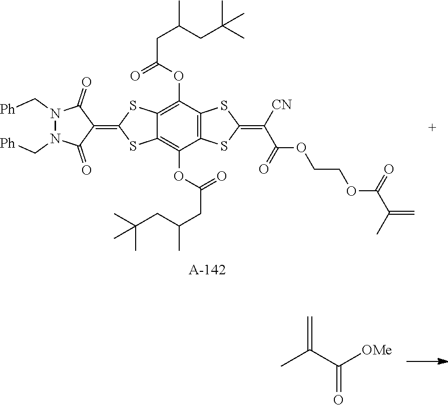

100 mg of the compound A-142 (maximal absorption wavelength (in ethyl acetate solution): 394 nm), 9.9 g of methyl methacrylate, and 40.0 g of propylene glycol monomethyl ether acetate were added to a 200 mL three-neck flask, and the mixture was stirred at 80° C. for 30 minutes under a nitrogen stream. 200 mg of dimethyl 2,2′-azobis(isobutyrate) (V-601, manufactured by FUJIFILM Wako Pure Chemical Corporation (hereinafter, referred to as V-601)) was added to the solution, and the mixture was stirred at 80° C. for 6 hours and then cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 7.0 g of a target polymer P-1. A number-average molecular weight of the obtained polymer P-1 was 27,500 (in terms of polystyrene).

100 mg of the obtained polymer P-1 was dissolved in 100 mL of chloroform, and an absorption spectrum was measured. A maximal absorption wavelength of the polymer P-1 was 399 nm (absorbance: 1.61).

The polymer P-1 was able to sufficiently shield light having a wavelength in the vicinity of 400 nm. In addition, the polymer P-1 had a less coloration.

### (Synthesis Example 102) Synthesis of Polymer P-2

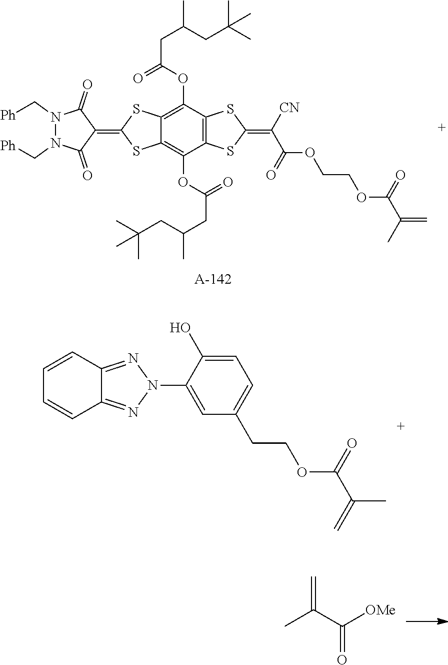

100 mg of the compound A-142 (maximal absorption wavelength (in ethyl acetate solution): 394 nm), 100 mg of 2-[2-hydroxy-5-(2-methacryloyloxyethyl)phenyl]2H-benzo[d][1,2,3]triazole (maximal absorption wavelength (in ethyl acetate solution): 338 nm) as an ultraviolet absorber, 9.8 g of methyl methacrylate, and 40.0 g of propylene glycol monomethyl ether acetate were added to a 200 mL three-neck flask, and the mixture was stirred at 80° C. for 6 hours under a nitrogen stream and cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 5.0 g of a target polymer P-2. A number-average molecular weight of the obtained polymer P-2 was 33,400 (in terms of polystyrene).

150 mg of the obtained polymer P-2 was dissolved in 100 mL of chloroform, and an absorption spectrum was measured. Maximal absorption wavelengths of the polymer P-2 were 399 nm (absorbance: 1.45) and 343 nm (absorbance: 0.83). The polymer P-2 was able to sufficiently shield light having a wavelength in the vicinity of 400 nm. Furthermore, shielding properties against light having a wavelength shorter than 350 nm were also excellent.

In addition, the polymer P-2 had a small coloration.

### (Synthesis Example 103) Synthesis of Polymer P-3

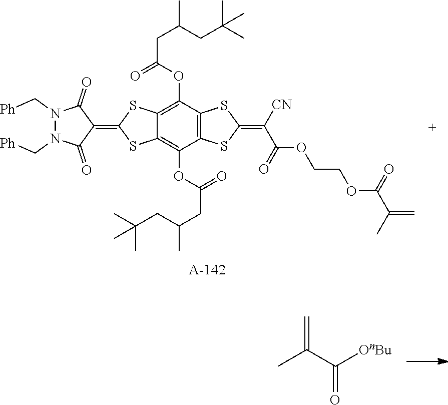

100 mg of the compound A-142 (maximal absorption wavelength (in ethyl acetate solution): 394 nm), 9.9 g of butyl methacrylate, and 40.0 g of propylene glycol monomethyl ether acetate were added to a 200 mL three-neck flask, and the mixture was stirred at 80° C. for 30 minutes under a nitrogen stream. 20 mg of dimethyl 2,2′-azobis(isobutyrate) (V-601, manufactured by FUJIFILM Wako Pure Chemical Corporation (hereinafter, referred to as V-601)) was added to the solution, and the mixture was stirred at 80° C. for 6 hours and then cooled to room temperature. The obtained reaction mixture was slowly added to a mixture of 200 mL of methanol and allowed to stand overnight. The precipitated precipitate was collected by filtration and washed with a mixture of chloroform and methanol. 20 mL of chloroform and 200 mL of methanol were added to the obtained powder, the mixture was stirred at room temperature for 1 hour, and then left to stand at room temperature overnight. The precipitate was collected by filtration, washed with a mixture of chloroform and methanol, and dried at 50° C., thereby obtaining 5.2 g of a target polymer P-3. A number-average molecular weight of the obtained polymer P-3 was 96,000 (in terms of polystyrene).

100 mg of the obtained polymer P-3 was dissolved in 100 mL of chloroform, and an absorption spectrum was measured. A maximal absorption wavelength of the polymer P-3 was 399 nm (absorbance: 1.56). The polymer P-3 was able to sufficiently shield light having a wavelength in the vicinity of 400 nm. In addition, the polymer P-3 had a small coloration.

### (Synthesis Example 104) Synthesis of Polymer P-4

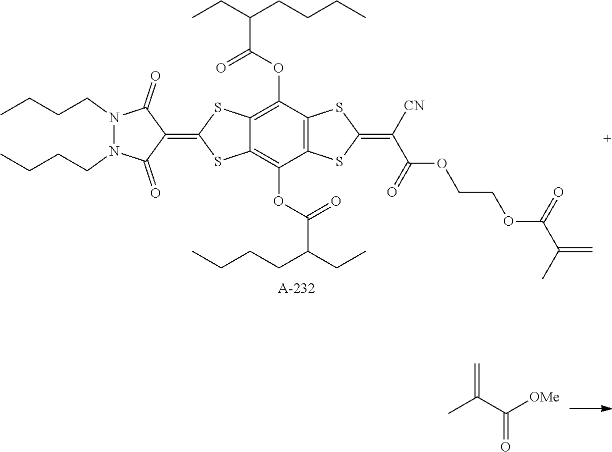

100 mg of the compound A-232 (maximal absorption wavelength (in ethyl acetate solution): 391 nm), 9.9 g of methyl methacrylate, and 40.0 g of propylene glycol monomethyl ether acetate were added to a 200 mL three-neck flask, and the mixture was stirred at 80° C. for 30 minutes under a nitrogen stream. 200 mg of dimethyl 2,2′-azobis(isobutyrate) (V-601, manufactured by FUJIFILM Wako Pure Chemical Corporation (hereinafter, referred to as V-601)) was added to the solution, and the mixture was stirred at 80° C. for 6 hours and then cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 7.4 g of a target polymer P-4. A number-average molecular weight of the obtained polymer P-4 was 29,500 (in terms of polystyrene).

100 mg of the obtained polymer P-4 was dissolved in 100 mL of chloroform, and an absorption spectrum was measured. A maximal absorption wavelength of the polymer P-4 was 397 nm (absorbance: 1.57). The polymer P-4 was able to sufficiently shield light having a wavelength in the vicinity of 400 nm. In addition, the polymer P-4 had a small coloration.

### (Synthesis Example 105) Synthesis of Polymer P-5

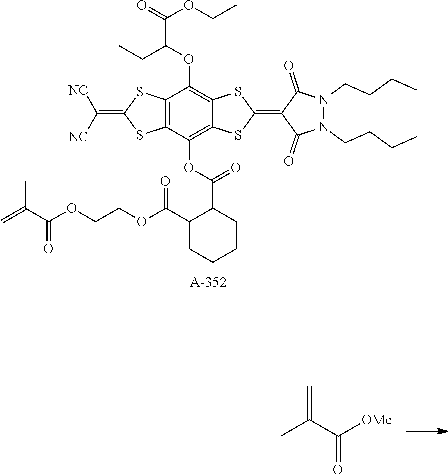

100 mg of the compound A-352 (maximal absorption wavelength (in ethyl acetate solution): 392 nm), 9.9 g of methyl methacrylate, and 40.0 g of propylene glycol monomethyl ether acetate were added to a 200 mL three-neck flask, and the mixture was stirred at 80° C. for 30 minutes under a nitrogen stream. 200 mg of dimethyl 2,2′-azobis(isobutyrate) (V-601, manufactured by FUJIFILM Wako Pure Chemical Corporation (hereinafter, referred to as V-601)) was added to the solution, and the mixture was stirred at 80° C. for 6 hours and then cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 7.8 g of a target polymer P-5. A number-average molecular weight of the obtained polymer P-5 was 45,000 (in terms of polystyrene).

100 mg of the obtained polymer P-5 was dissolved in 100 mL of chloroform, and an absorption spectrum was measured. A maximal absorption wavelength of the polymer P-5 was 397 nm (absorbance: 1.47).

The polymer P-5 was able to sufficiently shield light having a wavelength in the vicinity of 400 nm. In addition, the polymer P-5 had a small coloration.

### (Comparative Synthesis Example 101) Synthesis of Polymer P-6

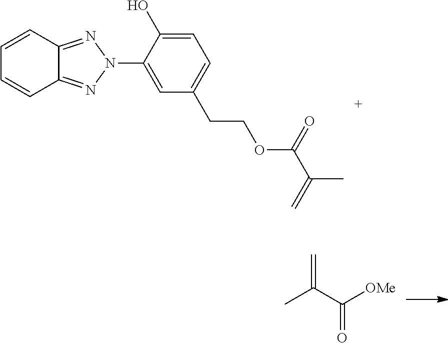

104 mg of 2-[2-hydroxy-5-(2-methacryloyloxyethyl)phenyl]2H-benzo[d][1,2,3]triazole, 9.9 g of methyl methacrylate, and 40.0 g of propylene glycol monomethyl ether acetate were added to a 200 mL three-neck flask, and the mixture was stirred at 80° C. for 30 minutes in a nitrogen stream. 135 mg of V-601 was added to the solution, and the mixture was stirred at 80° C. for 4 hours. Furthermore, 37 mg of V-601 was added thereto, and the mixture was stirred at 90° C. for 2 hours and cooled to room temperature. The obtained reaction mixture was slowly added to a mixture of 140 mL of hexane and 60 mL of isopropyl alcohol. The precipitated precipitate was collected by filtration and washed with a mixture of hexane and isopropyl alcohol. 140 mL of hexane and 60 mL of isopropyl alcohol were added to the obtained powder, the mixture was stirred at room temperature for 3 hours, and the precipitate was collected by filtration, washed with a mixture of hexane and isopropyl alcohol, and then dried at 50° C. to obtain 8.1 g of a target polymer P-6. A number-average molecular weight of the obtained polymer P-6 was 14,100 (in terms of polystyrene). 150 mg of the polymer P-6 was dissolved in 100 mL of chloroform, and an absorption spectrum was measured. A maximal absorption wavelength of the polymer P-6 was 339 nm (absorbance: 0.91). The polymer P-6 had low shielding properties against light having a wavelength of 380 to 400 nm.

### Test Example 3

### Examples 301 to 310 and Comparative Example 301 and 302

500 mg of the polymer described in the table below, 5.1 mg of the anti-fading agent described in the table below, 7.6 g of chloroform, and 1.1 g of a polymethyl methacrylate resin (Dianal BR-80, manufactured by Mitsubishi Chemical Corporation, containing 60% by mass or more of methyl methacrylate as a monomer unit, weight-average molecular weight: 95000, acid value: 0 mgKOH/g) were dissolved to prepare a composition (resin composition (resin solution)). The obtained composition was applied onto a glass substrate by spin coating, and the coating film was dried at 60° C. for 2 minutes to form a film having a thickness of about 10 μm.

The films obtained using the compositions of Examples 301 to 308 had almost no coloration and had excellent light shielding properties for light having a wavelength in the vicinity of 400 nm. On the other hand, the films obtained using the compositions of Comparative Examples 301 and 302 had low light shielding properties for light having a wavelength of 380 to 400 nm.

### (Evaluation of Colorability)

The colorability of the obtained film was evaluated by the same method and evaluation standards as those for the evaluation of the colorability in Test Example 1.

### (Evaluation of Light Resistance)

The obtained film was evaluated for light resistance by the same method and the same evaluation standard as those for the evaluation of the light resistance in Test Example 1, except that the light resistance test was performed under the following condition 3.

### —Condition 3—

Equipment: low temperature cycle xenon weather meter (manufactured by Suga Test Instruments Co., Ltd.; XL75)

Illuminance: 90 klx (40 w/m²)

Time: 4 weeks

Environment: 23° C., relative humidity: 5%

TABLE 9

| | | | Colorability | | |
|---|---|---|---|---|---|
| | | | before | after | |
| | | | light | light | Light |
| | | | resistance | resistance | resistance |
| | Type | Anti- | test | test | (absorbance |
| | of | fading | (absorbance | (absorbance | retention |
| | polymer | agent | ratio A₄₄₀) | ratio A₄₄₀) | rate (%)) |
| Example | P-1 | T-1 | A(0.007) | A(0.007) | AA(94) |
| 301 | | | | | |
| Example | P-1 | T-2 | A(0.007) | A(0.007) | AA(92) |
| 302 | | | | | |
| Example | P-2 | T-1 | A(0.007) | A(0.007) | AA(93) |
| 303 | | | | | |
| Example | P-2 | T-2 | A(0.007) | A(0.007) | AA(93) |
| 304 | | | | | |
| Example | P-3 | T-1 | A(0.007) | A(0.007) | AA(93) |
| 305 | | | | | |
| Example | P-3 | T-2 | A(0.007) | A(0.007) | AA(92) |
| 306 | | | | | |
| Example | P-4 | T-1 | A(0.007) | A(0.007) | AA(95) |
| 307 | | | | | |
| Example | P-4 | T-2 | A(0.007) | A(0.007) | AA(94) |
| 308 | | | | | |
| Example | P-5 | T-1 | A(0.006) | A(0.006) | AA(93) |
| 309 | | | | | |
| Example | P-5 | T-2 | A(0.006) | A(0.006) | AA(91) |
| 310 | | | | | |
| Comparative | P-6 | T-1 | A(0.009) | A(0.009) | A(87) |
| Example | | | | | |
| 301 | | | | | |
| Comparative | P-6 | T-2 | A(0.009) | A(0.009) | A(86) |
| Example | | | | | |
| 302 | | | | | |

As shown in the above table, in Examples 301 to 310, the value of the absorbance ratio A₄₄₀ was small before and after the light resistance test, and the colorability was excellent. Further, the absorbance retention rate after the light resistance test was also high, and the evaluation of the light resistance was also excellent.

### Test Example 4

120 mg of the ultraviolet absorber described in the table below, the anti-fading agent described in the table below, 120 mg of Irgacure OXE01 (manufactured by BASF SE, an oxime compound, a photoradical polymerization initiator), 15.9 g of propylene glycol methyl ether acetate, 2.84 g of a (meth)acrylic resin (Dianal BR-80, manufactured by Mitsubishi Chemical Corporation, containing 60% by mass or more of methyl methacrylate as a monomer unit, Mw: 95,000), and 2.32 g of KAYARAD DPHA (manufactured by Nippon Kayaku Co., Ltd., a compound having two or more ethylenically unsaturated bond-containing groups) were mixed and dissolved to prepare a composition.

TABLE 10

| | | Anti-fading agent | |
|---|---|---|---|
| | | | Addition amount |
| | Ultraviolet absorber | Kind | (mg) |
| Example 401 | Exemplary compound (1) | T-1 | 120 |
| Example 402 | Exemplary compound (1) | T-1 | 60 |
| Example 403 | Exemplary compound (1) | T-1 | 180 |
| Example 404 | Exemplary compound (18) | T-1 | 120 |
| Example 405 | Exemplary compound (18) | T-1 | 60 |
| Example 406 | Exemplary compound (18) | T-1 | 180 |
| Example 407 | Exemplary compound (19) | T-1 | 120 |
| Example 408 | Exemplary compound (19) | T-1 | 60 |
| Example 409 | Exemplary compound (19) | T-1 | 180 |
| Example 410 | Exemplary compound (19) | T-2 | 120 |
| Example 411 | Exemplary compound (19) | T-2 | 60 |
| Example 412 | Exemplary compound (19) | T-2 | 180 |
| Example 413 | Exemplary compound (20) | T-1 | 120 |
| Example 414 | Exemplary compound (20) | T-1 | 60 |
| Example 415 | Exemplary compound (20) | T-1 | 180 |
| Example 416 | Exemplary compound (21) | T-1 | 120 |
| Example 417 | Exemplary compound (21) | T-1 | 60 |
| Example 418 | Exemplary compound (21) | T-1 | 180 |
| Example 419 | Exemplary compound (21) | T-2 | 120 |
| Example 420 | Exemplary compound (21) | T-2 | 60 |
| Example 421 | Exemplary compound (21) | T-2 | 180 |
| Example 422 | Exemplary compound (22) | T-1 | 120 |
| Example 423 | Exemplary compound (22) | T-1 | 60 |
| Example 424 | Exemplary compound (22) | T-1 | 180 |
| Example 425 | Exemplary compound (23) | T-1 | 120 |
| Example 426 | Exemplary compound (23) | T-1 | 60 |
| Example 427 | Exemplary compound (23) | T-1 | 180 |
| Example 428 | Exemplary compound (24) | T-1 | 120 |
| Example 429 | Exemplary compound (24) | T-1 | 60 |
| Example 430 | Exemplary compound (24) | T-1 | 180 |
| Example 431 | Exemplary compound (25) | T-1 | 120 |
| Example 432 | Exemplary compound (25) | T-1 | 60 |
| Example 433 | Exemplary compound (25) | T-1 | 180 |
| Example 434 | Exemplary compound (26) | T-1 | 120 |
| Example 435 | Exemplary compound (26) | T-1 | 60 |
| Example 436 | Exemplary compound (26) | T-1 | 180 |

Each composition was spin-coated on a glass substrate (1737, manufactured by Corning) having a size of 50 mm×50 mm such that a film thickness after film formation was 1.5 μm, and dried at 120° C. for 5 minutes to form a composition layer. Thereafter, the composition layer was entirely exposed to an i-ray stepper exposure device (UX-1000SM-EH04, manufactured by USHIO INC.) with an exposure amount of 1,000 mJ/cm², thereby producing film. In Examples 401 to 421, the degree of change in transmittance at the maximum absorption wavelength (λₘₐₓ) of the composition layer before and after exposure was 5% or less.

### (Evaluation of Colorability)

The colorability of the obtained film was evaluated by the same method and evaluation standards as those for the evaluation of the colorability in Test Example 1.

### (Evaluation of Light Resistance)

The light resistance of the obtained film was evaluated by the same method and evaluation standard as in the evaluation of the light resistance in Test Example 2.

TABLE 11

Colorability

| | before | after | |
|---|---|---|---|
| | light | light | Light |
| | resistance | resistance | resistance |
| | test | test | (absorbance |
| | (absorbance | (absorbance | retention |
| | ratio A₄₄₀) | ratio A₄₄₀) | rate (%)) |
| Example 401 | A(0.005) | A(0.005) | AA(83) |
| Example 402 | A(0.005) | A(0.005) | AA(83) |
| Example 403 | A(0.005) | A(0.005) | AA(85) |
| Example 404 | A(0.007) | A(0.007) | AA(83) |
| Example 405 | A(0.007) | A(0.007) | AA(83) |
| Example 406 | A(0.007) | A(0.007) | AA(84) |
| Example 407 | A(0.007) | A(0.007) | AA(84) |
| Example 408 | A(0.007) | A(0.007) | AA(83) |
| Example 409 | A(0.007) | A(0.007) | AA(85) |
| Example 410 | A(0.007) | A(0.008) | AA(82) |
| Example 411 | A(0.007) | A(0.008) | AA(82) |
| Example 412 | A(0.007) | A(0.008) | AA(83) |
| Example 413 | A(0.006) | A(0.006) | AA(83) |
| Example 414 | A(0.006) | A(0.006) | AA(83) |
| Example 415 | A(0.006) | A(0.006) | AA(84) |
| Example 416 | A(0.007) | A(0.007) | AA(84) |
| Example 417 | A(0.007) | A(0.007) | AA(83) |
| Example 418 | A(0.007) | A(0.007) | AA(85) |
| Example 419 | A(0.007) | A(0.008) | AA(83) |
| Example 420 | A(0.007) | A(0.008) | AA(83) |
| Example 421 | A(0.007) | A(0.008) | AA(84) |
| Example 422 | A(0.005) | A(0.005) | AA(83) |
| Example 423 | A(0.005) | A(0.005) | AA(83) |
| Example 424 | A(0.005) | A(0.005) | AA(85) |
| Example 425 | A(0.005) | A(0.005) | AA(82) |
| Example 426 | A(0.005) | A(0.005) | AA(82) |
| Example 427 | A(0.005) | A(0.005) | AA(84) |
| Example 428 | A(0.005) | A(0.005) | AA(83) |
| Example 429 | A(0.005) | A(0.005) | AA(83) |
| Example 430 | A(0.005) | A(0.005) | AA(85) |
| Example 431 | A(0.005) | A(0.005) | AA(83) |
| Example 432 | A(0.005) | A(0.005) | AA(83) |
| Example 433 | A(0.005) | A(0.005) | AA(85) |
| Example 434 | A(0.005) | A(0.005) | AA(83) |
| Example 435 | A(0.005) | A(0.005) | AA(83) |
| Example 436 | A(0.005) | A(0.005) | AA(85) |

As shown in the above table, in Examples 401 to 436, the value of the absorbance ratio A₄₄₀ was small before and after the light resistance test, and the evaluation of the colorability was excellent. Further, the absorbance retention rate after the light resistance test was also high, and the evaluation of the light resistance was also excellent.

What is claimed is:

1. A composition comprising:
an ultraviolet absorber;
a curable compound; and
an anti-fading agent,
wherein the ultraviolet absorber includes at least one selected from a compound represented by Formula (1) or a polymer including a structure derived from the compound represented by Formula (1), and
the anti-fading agent includes at least one selected from an amine compound, a phenol compound, a hydroquinone compound, a catechol compound, an ascorbic acid compound, a carotenoid compound, a metal complex compound, or a benzolactone compound, 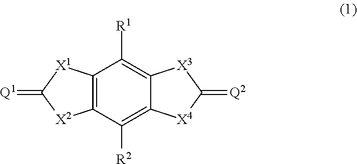
in Formula (1), Q¹ represents a group represented by Formula (Q-1),
Q² represents ═O, ═S, ═NR^{g1}, or ═CR^{q2}R^{q3}, R^{q1} to R^{q3} each independently represent a hydrogen atom or a substituent, and R^{q2} and R^{q3} may be bonded to each other to form a ring,
provided that, in a case where R^{q2} and R^{q3} are bonded to each other to form a ring, ═CR^{q2}R^{q3} does not have the same structure as Q,
R¹ and R² each independently represent a hydrogen atom or a substituent, and
X¹ to X⁴ each independently represent —S—, —NR^{X1}—, or —SO₂—, and R^{X1} represents a hydrogen atom or an alkyl group, 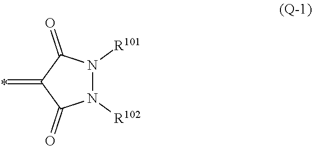
in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.

2. The composition according to claim 1,
wherein the compound represented by Formula (1) is a compound represented by Formula (3), 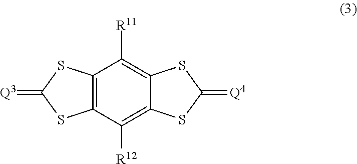
in Formula (3), Q³ represents the group represented by Formula (Q-1),
Q⁴ represents ═O, ═S, ═NR^{q11}, or ═CR^{q12}R¹³, R¹¹ to R^{q13} each independently represent a hydrogen atom or a substituent, and R^{q12} and R^{q13} may be bonded to each other to form a ring, provided that, in a case where R^{q12} and R^{q13} are bonded to each other to form a ring, ═CR^{q12}R^{q13} does not have the same structure as Q³, and
R¹¹ and R¹² each independently represent —OH, —O—Y¹¹, —OC(═O)—Y¹¹, —OC(═O)O—Y¹¹, —OC(═O)NR^{y11}—Y¹¹, —OSO₂—Y¹¹, or a group including a polymerizable group having an ethylenically unsaturated bond, where R^{y11} represents a hydrogen atom, an alkyl group, an aralkyl group, or an aryl group, and Y¹¹ represents an alkyl group, an aralkyl group, or an aryl group.

3. The composition according to claim 1,
wherein the anti-fading agent includes at least one selected from a compound represented by Formula (Ao1-1) or a compound represented by Formula (Ao2-1), 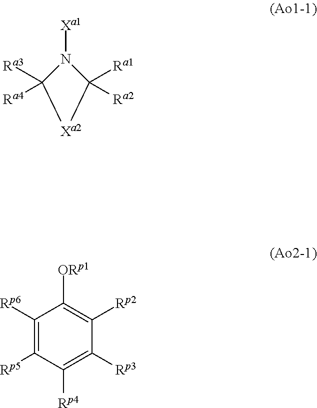
in Formula (Ao1-1), R^{a1} to R^{a4} each independently represent a hydrogen atom, an alkyl group, or an alkenyl group,
X^{a1} represents a hydrogen atom, an alkyl group, an alkenyl group, an alkoxy group, an alkenyloxy group, an alkoxycarbonyl group, an alkenyloxycarbonyl group, an aryloxycarbonyl group, an acyl group, an acyloxy group, an alkoxycarbonyloxy group, an alkenyloxycarbonyloxy group, an aryloxycarbonyloxy group, an alkylsulfonyl group, an alkenylsulfonyl group, an arylsulfonyl group, an alkylsulfinyl group, an alkenylsulfinyl group, an arylsulfinyl group, a sulfamoyl group, a carbamoyl group, a hydroxy group, or an oxyradical group, and
X^{a2} represents an atomic group necessary to form a 5- to 7-membered ring,
in Formula (Ao2-1), R^{p1} represents a hydrogen atom, an alkyl group, an alkenyl group, an aryl group, a heterocyclic group, an acyl group, an alkoxycarbonyl group, an alkenyloxycarbonyl group, an aryloxycarbonyl group, an alkylsulfonyl group, an arylsulfonyl group, or —Si(R^{p101})(R^{p102})(R^{p103}), R^{p101} to R^{p103} each independently represent an alkyl group, an alkenyl group, an aryl group, an alkoxy group, an alkenyloxy group, or an aryloxy group,
R^{p2} to R^{p6} each independently represent a hydrogen atom or a substituent, and
two adjacent groups among R^{p1} to R^{p6} may be bonded to each other to form a ring,
provided that, all of R^{p1} to R^{p6} are not hydrogen atoms.

4. The composition according to claim 1,
wherein the anti-fading agent includes at least one selected from a compound represented by Formula (Ao1-2) or a compound represented by Formula (Ao2-2), 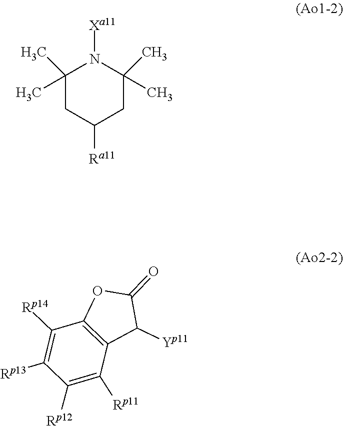
in Formula (Ao1-2), X^{a11} represents a hydrogen atom, an alkyl group, an alkenyl group, an alkoxy group, an alkenyloxy group, an alkoxycarbonyl group, an alkenyloxycarbonyl group, an aryloxycarbonyl group, an acyl group, an acyloxy group, an alkoxycarbonyloxy group, an alkenyloxycarbonyloxy group, an aryloxycarbonyloxy group, an alkylsulfonyl group, an alkenylsulfonyl group, an arylsulfonyl group, an alkylsulfinyl group, an alkenylsulfinyl group, an arylsulfinyl group, a sulfamoyl group, a carbamoyl group, a hydroxy group, or an oxyradical group, and
R^{a11} represents a substituent,
in Formula (Ao2-2), R^{p11} to R^{p14} each independently represent a hydrogen atom, an alkyl group, or an alkenyl group, and
Y^{p11} represents an aryl group, a heteroaryl group, or an ethylenically unsaturated bond-containing group.

5. The composition according to claim 1,
wherein the curable compound includes at least one selected from a resin or a polymerizable compound.

6. The composition according to claim 1,
wherein the curable compound includes a resin, and the resin is at least one selected from a (meth)acrylic resin, a polystyrene resin, a polyester resin, a polyurethane resin, a thiourethane resin, a polyimide resin, an epoxy resin, a polycarbonate resin, a phthalate resin, a cellulose acylate resin, or a cyclic olefin resin.

7. A cured substance which is formed of the composition according to claim 1.

8. An optical member comprising:
the cured substance according to claim 7.
